SEQUENCE LISTING (87 sequences)

SEQ ID NO: 1 - protein, 1738 aa, Homo sapiens
  REGION 1..1738: note = misc_feature - Human NALCN UniProt Q8IZF0
MLKRKQSSRVEAQPVTDFGPDESLSDNADILWINKPWVHSLLRICAIISVISVCMNTPMTFEHYPPLQYVTFTLDTLLMFLYTAEMIAKMHIRGIVKGDSSYVKDRWCVFDGFMVFCLWVSLVLQVFEIADIVDQMSPWGMLRIPRPLIMIRAFRIYFRFELPRTRITNILKRSGEQIWSVSIFLLFFLLLYGILGVQMFGTFTYHCVVNDTKPGNVTWNSLAIPDTHCSPELEEGYQCPPGFKCMDLEDLGLSRQELGYSGFNEIGTSIFTVYEAASQEGWVFLMYRAIDSFPRWRSYFYFITLIFFLAWLVKNVFIAVIIETFAEIRVQFQQMWGSRSSTTSTATTQMFHEDAAGGWQLVAVDVNKPQGRAPACLQKMMRSSVFHMFILSMVTVDVIVAASNYYKGENFRRQYDEFYLAEVAFTVLFDLEALLKIWCLGFTGYISSSLHKFELLLVIGTTLHVYPDLYHSQFTYFQVLRVVRLIKISPALEDFVYKIFGPGKKLGSLVVFTASLLIVMSAISLQMFCFVEELDRFTTFPRAFMSMFQILTQEGWVDVMDQTLNAVGHMWAPVVAIYFILYHLFATLILLSLFVAVILDNLELDEDLKKLKQLKQSEANADTKEKLPLRLRIFEKFPNRPQMVKISKLPSDFTVPKIRESFMKQFIDRQQQDTCCLLRSLPTTSSSSCDHSKRSAIEDNKYIDQKLRKSVFSIRARNLLEKETAVTKILRACTRQRMLSGSFEGQPAKERSILSVQHHIRQERRSLRHGSNSQRISRGKSLETLTQDHSNTVRYRNAQREDSEIKMIQEKKEQAEMKRKVQEEELRENHPYFDKPLFIVGREHRFRNFCRVVVRARFNASKTDPVTGAVKNTKYHQLYDLLGLVTYLDWVMIIVTICSCISMMFESPFRRVMHAPTLQIAEYVFVIFMSIELNLKIMADGLFFTPTAVIRDFGGVMDIFIYLVSLIFLCWMPQNVPAESGAQLLMVLRCLRPLRIFKLVPQMRKVVRELFSGFKEIFLVSILLLTLMLVFASFGVQLFAGKLAKCNDPNIIRREDCNGIFRINVSVSKNLNLKLRPGEKKPGFWVPRVWANPRNFNFDNVGNAMLALFEVLSLKGWVEVRDVIIHRVGPIHGIYIHVFVFLGCMIGLTLFVGVVIANFNENKGTALLTVDQRRWEDLKSRLKIAQPLHLPPRPDNDGFRAKMYDITQHPFFKRTIALLVLAQSVLLSVKWDVEDPVTVPLATMSVVFTFIFVLEVTMKIIAMSPAGFWQSRRNRYDLLVTSLGVVWVVLHFALLNAYTYMMGACVIVFRFFSICGKHVTLKMLLLTVVVSMYKSFFIIVGMFLLLLCYAFAGVVLFGTVKYGENINRHANFSSAGKAITVLFRIVTGEDWNKIMHDCMVQPPFCTPDEFTYWATDCGNYAGALMYFCSFYVIIAYIMLNLLVAIIVENFSLFYSTEEDQLLSYNDLRHFQIIWNMVDDKREGVIPTFRVKFLLRLLRGRLEVDLDKDKLLFKHMCYEMERLHNGGDVTFHDVLSMLSYRSVDIRKSLQLEELLAREQLEYTIEEEVAKQTIRMWLKKCLKRIRAKQQQSCSIIHSLRESQQQELSRFLNPPSIETTQPSEDTNANSQDNSMQPETSSQQQLLSPTLSDRGGSRQDAADAGKPQRKFGQWRLPSAPKPISHSVSSVNLRFGGRTTMKSVVCKMNPMTDAASCGSEVKKWWTRQLTVESDESGDDLLDI

SEQ ID NO: 2 - protein, 458 aa, Homo sapiens
  REGION 1..458: note = misc_feature - Human FAM155 UniProt B1AL88
MTRGAWMCRQYDDGLKIWLAAPRENEKPFIDSERAQKWRLSLASLLFFTVLLSDHLWFCAEAKLTRARDKEHQQQQRQQQQQQQQQRQRQQQQQQRRQQEPSWPALLASMGESSPAAQAHRLLSASSSPTLPPSPGDGGGGGGKGNRGKDDRGKALFLGNSAKPVWRLETCYPQGASSGQCFTVENADAVCARNWSRGAAGGDGQEVRSKHPTPLWNLSDFYLSFCNSYTLWELFSGLSSPNTLNCSLDVVLKEGGEMTTCRQCVEAYQDYDHHAQEKYEEFESVLHKYLQSEEYSVKSCPEDCKIVYKAWLCSQYFEVTQFNCRKTIPCKQYCLEVQTRCPFILPDNDEVIYGGLSSFICTGLYETFLTNDEPECCDVRREEKSNNPSKGTVEKSGSCHRTSLTVSSATRLCNSRLKLCVLVLILLHTVLTASAAQNTAGLSFGGINTLEENSTNEE

SEQ ID NO: 3 - protein, 2635 aa, Homo sapiens
  REGION 1..2635: note = misc_feature - Human UNC79 UniProt Q9P2D8
MSTKAEQFASKIRYLQEYHNRVLHNIYPVPSGTDIANTLKYFSQTLLSILSRTGKKENQDASNLTVPMTMCLFPVPFPLTPSLRPQVSSINPTVTRSLLYSVLRDAPSERGPQSRDAQLSDYPSLDYQGLYVTLVTLLDLVPLLQHGQHDLGQSIFYTTTCLLPFLNDDILSTLPYTMISTLATFPPFLHKDIIEYLSTSFLPMAILGSSRREGVPAHVNLSASSMLMIAMQYTSNPVYHCQLLECLMKYKQEVWKDLLYVIAYGPSQVKPPAVQMLFHYWPNLKPPGAISEYRGLQYTAWNPIHCQHIECHNAINKPAVKMCIDPSLSVALGDKPPPLYLCEECSERIAGDHSEWLIDVLLPQAEISAICQKKNCSSHVRRAVVTCFSAGCCGRHGNRPVRYCKRCHSNHHSNEVGAAAETHLYQTSPPPINTRECGAEELVCAVEAVISLLKEAEFHAEQREHELNRRRQLGLSSSHHSLDNADFDNKDDDKHDQRLLSQFGIWFLVSLCTPSENTPTESLARLVAMVFQWFHSTAYMMDDEVGSLVEKLKPQFVTKWLKTVCDVRFDVMVMCLLPKPMEFARVGGYWDKSCSTVTQLKEGLNRILCLIPYNVINQSVWECIMPEWLEAIRTEVPDNQLKEFREVLSKMFDIELCPLPFSMEEMFGFISCRFTGYPSSVQEQALLWLHVLSELDIMVPLQLLISMFSDGVNSVKELANQRKSRVSELAGNLASRRVSVASDPGRRVQHNMLSPFHSPFQSPFRSPLRSPFRSPFKNFGHPGGRTIDFDCEDDEMNLNCFILMFDLLLKQMELQDDGITMGLEHSLSKDIISIINNVFQAPWGGSHTCQKDEKAIECNLCQSSILCYQLACELLERLAPKEESRLVEPTDSLEDSLLSSRPEFIIGPEGEEEENPASKHGENPGNCTEPVEHAAVKNDTERKFCYQQLPVTLRLIYTIFQEMAKFEEPDILFNMLNCLKILCLHGECLYIARKDHPQFLAYIQDHMLIASLWRVVKSEFSQLSSLAVPLLLHALSLPHGADIFWTIINGNFNSKDWKMRFEAVEKVAVICRFLDIHSVTKNHLLKYSLAHAFCCFLTAVEDVNPAVATRAGLLLDTIKRPALQGLCLCLDFQFDTVVKDRPTILSKLLLLHFLKQDIPALSWEFFVNRFETLSLEAQLHLDCNKEFPFPTTITAVRTNVANLSDAALWKIKRARFARNRQKSVRSLRDSVKGPVESKRALSLPETLTSKIRQQSPENDNTIKDLLPEDAGIDHQTVHQLITVLMKFMAKDESSAESDISSAKAFNTVKRHLYVLLGYDQQEGCFMIAPQKMRLSTCFNAFIAGIAQVMDYNINLGKHLLPLVVQVLKYCSCPQLRHYFQQPPRCSLWSLKPHIRQMWLKALLVILYKYPYRDCDISKILLHLIHITVNTLNAQYHSCKPHATAGPLYSDNSNISRYSEKEKGEIELAEYRETGALQDSLLHCVREESIPKKKLRSFKQKSLDIGNADSLLFTLDEHRRKSCIDRCDIEKPPTQAAYIAQRPNDPGRSRQNSATRPDNSEIPENPAMEGFPDARRPVIPEVRLNCMETFEVKVDSPVKPAPKEDLDLIDLSSDSTSGPEKHSILSTSDSDSLVFEPLPPLRIVESDEEEETMNQGDDGPSGKNAASSPSVPSHPSVLSLSTAPLVQVSVEDCSKDFSSKDSGNNQSAGNTDSALITLEDPMDAEGSSKPEELPEFSCGSPLTLKQKRDLLQKSFALPEMSLDDHPDPGTEGEKPGELMPSSGAKTVLLKVPEDAENPTESEKPDTSAESDTEQNPERKVEEDGAEESEFKIQIVPRQRKQRKIAVSAIQREYLDISFNILDKLGEQKDPDPSTKGLSTLEMPRESSSAPTLDAGVPETSSHSSISTQYRQMKRGSLGVLTMSQLMKRQLEHQSSAPHNISNWDTEQIQPGKRQCNVPTCLNPDLEGQPLRMRGATKSSLLSAPSIVSMFVPAPEEFTDEQPTVMTDKCHDCGAILEEYDEETLGLAIVVLSTFIHLSPDLAAPLLLDIMQSVGRLASSTTFSNQAESMMVPGNAAGVAKQFLRCIFHQLAPNGIFPQLFQSTIKDGTFLRTLASSLMDFNELSSIAALSQLLEGLNNKKNLPAGGAMIRCLENIATFMEALPMDSPSSLWTTISNQFQTFFAKLPCVLPLKCSLDSSLRIMICLLKIPSTNATRSLLEPFSKLLSFVIQNAVFTLAYLVELCGLCYRAFTKERDKFYLSRSVVLELLQALKLKSPLPDTNLLLLVQFICADAGTKLAESTILSKQMIASVPGCGTAAMECVRQYINEVLDFMADMHTLTKLKSHMKTCSQPLHEDTFGGHLKVGLAQIAAMDISRGNHRDNKAVIRYLPWLYHPPSAMQQGPKEFIECVSHIRLLSWLLLGSLTHNAVCPNASSPCLPIPLDAGSHVADHLIVILIGFPEQSKTSVLHMCSLFHAFIFAQLWTVYCEQSAVATNLQNQNEFSFTAILTALEFWSRVTPSILQLMAHNKVMVEMVCLHVISLMEALQECNSTIFVKLIPMWLPMIQSNIKHLSAGLQLRLQAIQNHVNHHSLRTLPGSGQSSAGLAALRKWLQCTQFKMAQVEIQSSEAASQFYPL

SEQ ID NO: 4 - protein, 3258 aa, Homo sapiens
  REGION 1..3258: note = misc_feature - Human UNC80 Uniprot Q8N2C7
MVKRKSSEGQEQDGGRGIPLPIQTFLWRQTSAFLRPKLGKQYEASCVSFERVLVENKLHGLSPALSEAIQSISRWELVQAALPHVLHCTATLLSNRNKLGHQDKLGVAETKLLHTLHWMLLEAPQDCNNERFGGTDRGSSWGGSSSAFIHQVENQGSPGQPCQSSSNDEEENNRRKIFQNSMATVELFVFLFAPLVHRIKESDLTFRLASGLVIWQPMWEHRQPGVSGFTALVKPIRNIITAKRSSPINSQSRTCESPNQDARHLEGLQVVCETFQSDSISPKATISGCHRGNSFDGSLSSQTSQERGPSHSRASLVIPPCQRSRYATYFDVAVLRCLLQPHWSEEGTQWSLMYYLQRLRHMLEEKPEKPPEPDIPLLPRPRSSSMVAAAPSLVNTHKTQDLTMKCNEEEKSLSSEAFSKVSLTNLRRSAVPDLSSDLGMNIFKKFKSRKEDRERKGSIPFHHTGKRRPRRMGVPFLLHEDHLDVSPTRSTFSFGSFSGLGEDRRGIEKGGWQTTILGKLTRRGSSDAATEMESLSARHSHSHHTLVSDLPDPSNSHGENTVKEVRSQISTITVATFNTTLASFNVGYADFFNEHMRKLCNQVPIPEMPHEPLACANLPRSLTDSCINYSYLEDTEHIDGTNNFVHKNGMLDLSVVLKAVYLVLNHDISSRICDVALNIVECLLQLGVVPCVEKNRKKSENKENETLEKRPSEGAFQFKGVSGSSTCGFGGPAVSGAGDGGGEEGGGGDGGGGGGDGGGGGGGGGGPYEKNDKNQEKDESTPVSNHRLALTMLIKIVKSLGCAYGCGEGHRGLSGDRLRHQVFRENAQNCLTKLYKLDKMQFRQTMRDYVNKDSLNNVVDFLHALLGFCMEPVTDNKAGFGNNFTTVDNKSTAQNVEGIIVSAMFKSLITRCASTTHELHSPENLGLYCDIRQLVQFIKEAHGNVFRRVALSALLDSAEKLAPGKKVEENEQESKPAGSKRSEAGSIVDKGQVSSAPEECRSFMSGRPSQTPEHDEQMQGANLGRKDFWRKMFKSQSAASDTSSQSEQDTSECTTAHSGTTSDRRARSRSRRISLRKKLKLPIGKRNWLKRSSLSGLADGVEDLLDISSVDRLSFIRQSSKVKFTSAVKLSEGGPGSGMENGRDEEENFFKRLGCHSFDDHLSPNQDGGKSKNVVNLGAIRQGMKRFQFLLNCCEPGTIPDASILAAALDLEAPVVARAALFLECARFVHRCNRGNWPEWMKGHHVNITKKGLSRGRSPIVGNKRNQKLQWNAAKLFYQWGDAIGVRLNELCHGESESPANLLGLIYDEETKRRLRKEDEEEDFLDDSTVNPSKCGCPFALKMAACQLLLEITTFLRETFSCLPRPRTEPLVDLESCRLRLDPELDRHRYERKISFAGVLDENEDSKDSLHSSSHTLKSDAGVEEKKEGSPWSASEPSIEPEGMSNAGAEENYHRNMSWLHVMILLCNQQSFICTHVDYCHPHCYLHHSRSCARLVRAIKLLYGDSVDSLRESSNISSVALRGKKQKECSDKSCLRTPSLKKRVSDANLEGKKDSGMLKYIRLQVMSLSPAPLSLLIKAAPILTEEMYGDIQPAAWELLLSMDEHMAGAAAAMFLLCAVKVPEAVSDMLMSEFHHPETVQRLNAVLKFHTLWRFRYQVWPRMEEGAQQIFKIPPPSINFTLPSPVLGMPSVPMFDPPWVPQCSGSVQDPINEDQSKSFSARAVSRSHQRAEHILKNLQQEEEKKRLGREASLITAIPITQEACYEPTCTPNSEPEEEVEEVTNLASRRLSVSPSCTSSTSHRNYSFRRGSVWSVRSAVSAEDEEHTTEHTPNHHVPQPPQAVFPACICAAVLPIVHLMEDGEVREDGVAVSAVAQQVLWNCLIEDPSTVLRHFLEKLTISNRQDELMYMLRKLLLNIGDFPAQTSHILFNYLVGLIMYFVRTPCEWGMDAISATLTFLWEVVGYVEGLFFKDLKQTMKKEQCEVKLLVTASMPGTKTLVVHGQNECDIPTQLPVHEDTQFEALLKECLEFFNIPESQSTHYFLMDKRWNLIHYNKTYVRDIYPFRRSVSPQLNLVHMHPEKGQELIQKQVFTRKLEEVGRVLFLISLTQKIPTAHKQSHVSMLQEDLLRLPSFPRSAIDAEFSLFSDPQAGKELFGLDTLQKSLWIQLLEEMFLGMPSEFPWGDEIMLFLNVFNGALILHPEDSALLRQYAATVINTAVHFNHLFSLSGYQWILPTMLQVYSDYESNPQLRQAIEFACHQFYILHRKPFVLQLFASVAPLLEFPDAANNGPSKGVSAQCLFDLLQSLEGETTDILDILELVKAEKPLKSLDFCYGNEDLTFSISEAIKLCVTVVAYAPESFRSLQMLMVLEALVPCYLQKLKRQTSQVETVPAAREEIAATAALATSLQALLYSVEVLTRPMTAPQMSRCDQGHKGTTTANHTMSSGVNTRYQEQGAKLHFIRENLHLLEEGQGIPREELDERIAREEFRRPRESLLNICTEFYKHCGPRLKILQNLAGEPRVIALELLDVKSHMRLAEIAHSLLKLAPYDTQTMESRGLRRYIMEMLPITDWTAEAVRPALILILKRLDRMFNKIHKMPTLRRQVEWEPASNLIEGVCLTLQRQPIISFLPHLRSLINVCVNLVMGVVGPSSVADGLPLLHLSPYLSPPLPFSTAVVRLVALQIQALKEDFPLSHVISPFTNQERREGMLLNLLIPFVLTVGSGSKDSPWLEQPEVQLLLQTVINVLLPPRIISTSRSKNFMLESSPAHCSTPGDAGKDLRREGLAESTSQAAYLALKVILVCFERQLGSQWYWLSLQVKEMALRKVGGLALWDFLDFIVRTRIPIFVLLRPFIQCKLLAQPAENHEELSARQHIADQLERRFIPRPLCKSSLIAEFNSELKILKEAVHSGSAYQGKTSISTVGTSTSAYRLSLATMSRSNTGTGTVWEQDSEPSQQASQDTLSRTDEEDEENDSISMPSVVSEQEAYLLSAIGRRRFSSHVSSMSVPQAEVGMLPSQSEPNVLDDSQGLAAEGSLSRVASIQSEPGQQNLLVQQPLGRKRGLRQLRRPLLSRQKTQTEPRNRQGARLSTTRRSIQPKTKPSADQKRSVTFIEAQPEPAAAPTDALPATGQLQGCSPAPSRKPEAMDEPVLTSSPAIVVADLHSVSPKQSENFPTEEGEKEEDTEAQGATAHSPLSAQLSDPDDFTGLETSSLLQHGDTVLHISEENGMENPLLSSQFTFTPTELGKTDAVLDESHV

SEQ ID NO: 5 - protein, 149 aa, Homo sapiens [sequence omitted]

SEQ ID NO: 6 - protein, 1723 aa, synthetic construct
  REGION 1..1723: note = Synthetic: hNALCN_delta-349-363
MLKRKQSSRVEAQPVTDFGPDESLSDNADILWINKPWVHSLLRICAIISVISVCMNTPMTFEHYPPLQYVTFTLDTLLMFLYTAEMIAKMHIRGIVKGDSSYVKDRWCVFDGFMVFCLWVSLVLQVFEIADIVDQMSPWGMLRIPRPLIMIRAFRIYFRFELPRTRITNILKRSGEQIWSVSIFLLFFLLLYGILGVQMFGTFTYHCVVNDTKPGNVTWNSLAIPDTHCSPELEEGYQCPPGFKCMDLEDLGLSRQELGYSGFNEIGTSIFTVYEAASQEGWVFLMYRAIDSFPRWRSYFYFITLIFFLAWLVKNVFIAVIIETFAEIRVQFQQMWGSRSSTTSTATTVDVNKPQGRAPACLQKMMRSSVFHMFILSMVTVDVIVAASNYYKGENFRRQYDEFYLAEVAFTVLFDLEALLKIWCLGFTGYISSSLHKFELLLVIGTTLHVYPDLYHSQFTYFQVLRVVRLIKISPALEDFVYKIFGPGKKLGSLVVFTASLLIVMSAISLQMFCFVEELDRFTTFPRAFMSMFQILTQEGWVDVMDQTLNAVGHMWAPVVAIYFILYHLFATLILLSLFVAVILDNLELDEDLKKLKQLKQSEANADTKEKLPLRLRIFEKFPNRPQMVKISKLPSDFTVPKIRESFMKQFIDRQQQDTCCLLRSLPTTSSSSCDHSKRSAIEDNKYIDQKLRKSVFSIRARNLLEKETAVTKILRACTRQRMLSGSFEGQPAKERSILSVQHHIRQERRSLRHGSNSQRISRGKSLETLTQDHSNTVRYRNAQREDSEIKMIQEKKEQAEMKRKVQEEELRENHPYFDKPLFIVGREHRFRNFCRVVVRARFNASKTDPVTGAVKNTKYHQLYDLLGLVTYLDWVMIIVTICSCISMMFESPFRRVMHAPTLQIAEYVFVIFMSIELNLKIMADGLFFTPTAVIRDFGGVMDIFIYLVSLIFLCWMPQNVPAESGAQLLMVLRCLRPLRIFKLVPQMRKVVRELFSGFKEIFLVSILLLTLMLVFASFGVQLFAGKLAKCNDPNIIRREDCNGIFRINVSVSKNLNLKLRPGEKKPGFWVPRVWANPRNFNFDNVGNAMLALFEVLSLKGWVEVRDVIIHRVGPIHGIYIHVFVFLGCMIGLTLFVGVVIANFNENKGTALLTVDQRRWEDLKSRLKIAQPLHLPPRPDNDGFRAKMYDITQHPFFKRTIALLVLAQSVLLSVKWDVEDPVTVPLATMSVVFTFIFVLEVTMKIIAMSPAGFWQSRRNRYDLLVTSLGVVWVVLHFALLNAYTYMMGACVIVFRFFSICGKHVTLKMLLLTVVVSMYKSFFIIVGMFLLLLCYAFAGVVLFGTVKYGENINRHANFSSAGKAITVLFRIVTGEDWNKIMHDCMVQPPFCTPDEFTYWATDCGNYAGALMYFCSFYVIIAYIMLNLLVAIIVENFSLFYSTEEDQLLSYNDLRHFQIIWNMVDDKREGVIPTFRVKFLLRLLRGRLEVDLDKDKLLFKHMCYEMERLHNGGDVTFHDVLSMLSYRSVDIRKSLQLEELLAREQLEYTIEEEVAKQTIRMWLKKCLKRIRAKQQQSCSIIHSLRESQQQELSRFLNPPSIETTQPSEDTNANSQDNSMQPETSSQQQLLSPTLSDRGGSRQDAADAGKPQRKFGQWRLPSAPKPISHSVSSVNLRFGGRTTMKSVVCKMNPMTDAASCGSEVKKWWTRQLTVESDESGDDLLDI

SEQ ID NO: 7 - protein, 1807 aa, synthetic construct
  REGION 1..1807: note = Synthetic: hNALCN-hNaV1.4 DI-DII linker chimera
MLKRKQSSRVEAQPVTDFGPDESLSDNADILWINKPWVHSLLRICAIISVISVCMNTPMTFEHYPPLQYVTFTLDTLLMFLYTAEMIAKMHIRGIVKGDSSYVKDRWCVFDGFMVFCLWVSLVLQVFEIADIVDQMSPWGMLRIPRPLIMIRAFRIYFRFELPRTRITNILKRSGEQIWSVSIFLLFFLLLYGILGVQMFGTFTYHCVVNDTKPGNVTWNSLAIPDTHCSPELEEGYQCPPGFKCMDLEDLGLSRQELGYSGFNEIGTSIFTVYEAASQEGWVFLMYRAIDSFPRWRSYFYFITLIFFLAWLVKNVFIAVIIETFAEIRVQFQQMDKEKEEEFQQMLEKFKKHQEELEKAKAAQALEGGEADGDPAHGKDCNGSLDTSQGEKGAPRQSSSGDSGISDAMEELEEAHQKCPPWWYKCAHKVLIWNCCAPWLKFKACLQKMMRSSVFHMFILSMVTVDVIVAASNYYKGENFRRQYDEFYLAEVAFTVLFDLEALLKIWCLGFTGYISSSLHKFELLLVIGTTLHVYPDLYHSQFTYFQVLRVVRLIKISPALEDFVYKIFGPGKKLGSLVVFTASLLIVMSAISLQMFCFVEELDRFTTFPRAFMSMFQILTQEGWVDVMDQTLNAVGHMWAPVVAIYFILYHLFATLILLSLFVAVILDNLELDEDLKKLKQLKQSEANADTKEKLPLRLRIFEKFPNRPQMVKISKLPSDFTVPKIRESFMKQFIDRQQQDTCCLLRSLPTTSSSSCDHSKRSAIEDNKYIDQKLRKSVFSIRARNLLEKETAVTKILRACTRQRMLSGSFEGQPAKERSILSVQHHIRQERRSLRHGSNSQRISRGKSLETLTQDHSNTVRYRNAQREDSEIKMIQEKKEQAEMKRKVQEEELRENHPYFDKPLFIVGREHRFRNFCRVVVRARFNASKTDPVTGAVKNTKYHQLYDLLGLVTYLDWVMIIVTICSCISMMFESPFRRVMHAPTLQIAEYVFVIFMSIELNLKIMADGLFFTPTAVIRDFGGVMDIFIYLVSLIFLCWMPQNVPAESGAQLLMVLRCLRPLRIFKLVPQMRKVVRELFSGFKEIFLVSILLLTLMLVFASFGVQLFAGKLAKCNDPNIIRREDCNGIFRINVSVSKNLNLKLRPGEKKPGFWVPRVWANPRNFNFDNVGNAMLALFEVLSLKGWVEVRDVIIHRVGPIHGIYIHVFVFLGCMIGLTLFVGVVIANFNENKGTALLTVDQRRWEDLKSRLKIAQPLHLPPRPDNDGFRAKMYDITQHPFFKRTIALLVLAQSVLLSVKWDVEDPVTVPLATMSVVFTFIFVLEVTMKIIAMSPAGFWQSRRNRYDLLVTSLGVVWVVLHFALLNAYTYMMGACVIVFRFFSICGKHVTLKMLLLTVVVSMYKSFFIIVGMFLLLLCYAFAGVVLFGTVKYGENINRHANFSSAGKAITVLFRIVTGEDWNKIMHDCMVQPPFCTPDEFTYWATDCGNYAGALMYFCSFYVIIAYIMLNLLVAIIVENFSLFYSTEEDQLLSYNDLRHFQIIWNMVDDKREGVIPTFRVKFLLRLLRGRLEVDLDKDKLLFKHMCYEMERLHNGGDVTFHDVLSMLSYRSVDIRKSLQLEELLAREQLEYTIEEEVAKQTIRMWLKKCLKRIRAKQQQSCSIIHSLRESQQQELSRFLNPPSIETTQPSEDTNANSQDNSMQPETSSQQQLLSPTLSDRGGSRQDAADAGKPQRKFGQWRLPSAPKPISHSVSSVNLRFGGRTTMKSVVCKMNPMTDAASCGSEVKKWWTRQLTVESDESGDDLLDI

SEQ ID NO: 8 - protein, 1738 aa, synthetic construct
  REGION 1..1738: note = Synthetic: hNALCN-GGGS DI-DII linker chimera
MLKRKQSSRVEAQPVTDFGPDESLSDNADILWINKPWVHSLLRICAIISVISVCMNTPMTFEHYPPLQYVTFTLDTLLMFLYTAEMIAKMHIRGIVKGDSSYVKDRWCVFDGFMVFCLWVSLVLQVFEIADIVDQMSPWGMLRIPRPLIMIRAFRIYFRFELPRTRITNILKRSGEQIWSVSIFLLFFLLLYGILGVQMFGTFTYHCVVNDTKPGNVTWNSLAIPDTHCSPELEEGYQCPPGFKCMDLEDLGLSRQELGYSGFNEIGTSIFTVYEAASQEGWVFLMYRAIDSFPRWRSYFYFITLIFFLAWLVKNVFIAVIIETFAEIRVQFQQMGGGSGGGSGGGSGGGSGGGSGGGSGGGSGGGSGGGSGGGACLQKMMRSSVFHMFILSMVTVDVIVAASNYYKGENFRRQYDEFYLAEVAFTVLFDLEALLKIWCLGFTGYISSSLHKFELLLVIGTTLHVYPDLYHSQFTYFQVLRVVRLIKISPALEDFVYKIFGPGKKLGSLVVFTASLLIVMSAISLQMFCFVEELDRFTTFPRAFMSMFQILTQEGWVDVMDQTLNAVGHMWAPVVAIYFILYHLFATLILLSLFVAVILDNLELDEDLKKLKQLKQSEANADTKEKLPLRLRIFEKFPNRPQMVKISKLPSDFTVPKIRESFMKQFIDRQQQDTCCLLRSLPTTSSSSCDHSKRSAIEDNKYIDQKLRKSVFSIRARNLLEKETAVTKILRACTRQRMLSGSFEGQPAKERSILSVQHHIRQERRSLRHGSNSQRISRGKSLETLTQDHSNTVRYRNAQREDSEIKMIQEKKEQAEMKRKVQEEELRENHPYFDKPLFIVGREHRFRNFCRVVVRARFNASKTDPVTGAVKNTKYHQLYDLLGLVTYLDWVMIIVTICSCISMMFESPFRRVMHAPTLQIAEYVFVIFMSIELNLKIMADGLFFTPTAVIRDFGGVMDIFIYLVSLIFLCWMPQNVPAESGAQLLMVLRCLRPLRIFKLVPQMRKVVRELFSGFKEIFLVSILLLTLMLVFASFGVQLFAGKLAKCNDPNIIRREDCNGIFRINVSVSKNLNLKLRPGEKKPGFWVPRVWANPRNFNFDNVGNAMLALFEVLSLKGWVEVRDVIIHRVGPIHGIYIHVFVFLGCMIGLTLFVGVVIANFNENKGTALLTVDQRRWEDLKSRLKIAQPLHLPPRPDNDGFRAKMYDITQHPFFKRTIALLVLAQSVLLSVKWDVEDPVTVPLATMSVVFTFIFVLEVTMKIIAMSPAGFWQSRRNRYDLLVTSLGVVWVVLHFALLNAYTYMMGACVIVFRFFSICGKHVTLKMLLLTVVVSMYKSFFIIVGMFLLLLCYAFAGVVLFGTVKYGENINRHANFSSAGKAITVLFRIVTGEDWNKIMHDCMVQPPFCTPDEFTYWATDCGNYAGALMYFCSFYVIIAYIMLNLLVAIIVENFSLFYSTEEDQLLSYNDLRHFQIIWNMVDDKREGVIPTFRVKFLLRLLRGRLEVDLDKDKLLFKHMCYEMERLHNGGDVTFHDVLSMLSYRSVDIRKSLQLEELLAREQLEYTIEEEVAKQTIRMWLKKCLKRIRAKQQQSCSIIHSLRESQQQELSRFLNPPSIETTQPSEDTNANSQDNSMQPETSSQQQLLSPTLSDRGGSRQDAADAGKPQRKFGQWRLPSAPKPISHSVSSVNLRFGGRTTMKSVVCKMNPMTDAASCGSEVKKWWTRQLTVESDESGDDLLDI

SEQ ID NO: 9 - protein, 1705 aa, synthetic construct
  REGION 1..1705: note = Synthetic: hNALCN_delta-638-670
MLKRKQSSRVEAQPVTDFGPDESLSDNADILWINKPWVHSLLRICAIISVISVCMNTPMTFEHYPPLQYVTFTLDTLLMFLYTAEMIAKMHIRGIVKGDSSYVKDRWCVFDGFMVFCLWVSLVLQVFEIADIVDQMSPWGMLRIPRPLIMIRAFRIYFRFELPRTRITNILKRSGEQIWSVSIFLLFFLLLYGILGVQMFGTFTYHCVVNDTKPGNVTWNSLAIPDTHCSPELEEGYQCPPGFKCMDLEDLGLSRQELGYSGFNEIGTSIFTVYEAASQEGWVFLMYRAIDSFPRWRSYFYFITLIFFLAWLVKNVFIAVIIETFAEIRVQFQQMWGSRSSTTSTATTQMFHEDAAGGWQLVAVDVNKPQGRAPACLQKMMRSSVFHMFILSMVTVDVIVAASNYYKGENFRRQYDEFYLAEVAFTVLFDLEALLKIWCLGFTGYISSSLHKFELLLVIGTTLHVYPDLYHSQFTYFQVLRVVRLIKISPALEDFVYKIFGPGKKLGSLVVFTASLLIVMSAISLQMFCFVEELDRFTTFPRAFMSMFQILTQEGWVDVMDQTLNAVGHMWAPVVAIYFILYHLFATLILLSLFVAVILDNLELDEDLKKLKQLKQSEANADTKEKLPLRLRIFEKFQQDTCCLLRSLPTTSSSSCDHSKRSAIEDNKYIDQKLRKSVFSIRARNLLEKETAVTKILRACTRQRMLSGSFEGQPAKERSILSVQHHIRQERRSLRHGSNSQRISRGKSLETLTQDHSNTVRYRNAQREDSEIKMIQEKKEQAEMKRKVQEEELRENHPYFDKPLFIVGREHRFRNFCRVVVRARFNASKTDPVTGAVKNTKYHQLYDLLGLVTYLDWVMIIVTICSCISMMFESPFRRVMHAPTLQIAEYVFVIFMSIELNLKIMADGLFFTPTAVIRDFGGVMDIFIYLVSLIFLCWMPQNVPAESGAQLLMVLRCLRPLRIFKLVPQMRKVVRELFSGFKEIFLVSILLLTLMLVFASFGVQLFAGKLAKCNDPNIIRREDCNGIFRINVSVSKNLNLKLRPGEKKPGFWVPRVWANPRNFNFDNVGNAMLALFEVLSLKGWVEVRDVIIHRVGPIHGIYIHVFVFLGCMIGLTLFVGVVIANFNENKGTALLTVDQRRWEDLKSRLKIAQPLHLPPRPDNDGFRAKMYDITQHPFFKRTIALLVLAQSVLLSVKWDVEDPVTVPLATMSVVFTFIFVLEVTMKIIAMSPAGFWQSRRNRYDLLVTSLGVVWVVLHFALLNAYTYMMGACVIVFRFFSICGKHVTLKMLLLTVVVSMYKSFFIIVGMFLLLLCYAFAGVVLFGTVKYGENINRHANFSSAGKAITVLFRIVTGEDWNKIMHDCMVQPPFCTPDEFTYWATDCGNYAGALMYFCSFYVIIAYIMLNLLVAIIVENFSLFYSTEEDQLLSYNDLRHFQIIWNMVDDKREGVIPTFRVKFLLRLLRGRLEVDLDKDKLLFKHMCYEMERLHNGGDVTFHDVLSMLSYRSVDIRKSLQLEELLAREQLEYTIEEEVAKQTIRMWLKKCLKRIRAKQQQSCSIIHSLRESQQQELSRFLNPPSIETTQPSEDTNANSQDNSMQPETSSQQQLLSPTLSDRGGSRQDAADAGKPQRKFGQWRLPSAPKPISHSVSSVNLRFGGRTTMKSVVCKMNPMTDAASCGSEVKKWWTRQLTVESDESGDDLLDI

SEQ ID NO: 10 - protein, 1703 aa, synthetic construct
  REGION 1..1703: note = Synthetic: hNALCN_delta-794-828
MLKRKQSSRVEAQPVTDFGPDESLSDNADILWINKPWVHSLLRICAIISVISVCMNTPMTFEHYPPLQYVTFTLDTLLMFLYTAEMIAKMHIRGIVKGDSSYVKDRWCVFDGFMVFCLWVSLVLQVFEIADIVDQMSPWGMLRIPRPLIMIRAFRIYFRFELPRTRITNILKRSGEQIWSVSIFLLFFLLLYGILGVQMFGTFTYHCVVNDTKPGNVTWNSLAIPDTHCSPELEEGYQCPPGFKCMDLEDLGLSRQELGYSGFNEIGTSIFTVYEAASQEGWVFLMYRAIDSFPRWRSYFYFITLIFFLAWLVKNVFIAVIIETFAEIRVQFQQMWGSRSSTTSTATTQMFHEDAAGGWQLVAVDVNKPQGRAPACLQKMMRSSVFHMFILSMVTVDVIVAASNYYKGENFRRQYDEFYLAEVAFTVLFDLEALLKIWCLGFTGYISSSLHKFELLLVIGTTLHVYPDLYHSQFTYFQVLRVVRLIKISPALEDFVYKIFGPGKKLGSLVVFTASLLIVMSAISLQMFCFVEELDRFTTFPRAFMSMFQILTQEGWVDVMDQTLNAVGHMWAPVVAIYFILYHLFATLILLSLFVAVILDNLELDEDLKKLKQLKQSEANADTKEKLPLRLRIFEKFPNRPQMVKISKLPSDFTVPKIRESFMKQFIDRQQQDTCCLLRSLPTTSSSSCDHSKRSAIEDNKYIDQKLRKSVFSIRARNLLEKETAVTKILRACTRQRMLSGSFEGQPAKERSILSVQHHIRQERRSLRHGSNSQRISRGKSLETLTQDHSNTVNHPYFDKPLFIVGREHRFRNFCRVVVRARFNASKTDPVTGAVKNTKYHQLYDLLGLVTYLDWVMIIVTICSCISMMFESPFRRVMHAPTLQIAEYVFVIFMSIELNLKIMADGLFFTPTAVIRDFGGVMDIFIYLVSLIFLCWMPQNVPAESGAQLLMVLRCLRPLRIFKLVPQMRKVVRELFSGFKEIFLVSILLLTLMLVFASFGVQLFAGKLAKCNDPNIIRREDCNGIFRINVSVSKNLNLKLRPGEKKPGFWVPRVWANPRNFNFDNVGNAMLALFEVLSLKGWVEVRDVIIHRVGPIHGIYIHVFVFLGCMIGLTLFVGVVIANFNENKGTALLTVDQRRWEDLKSRLKIAQPLHLPPRPDNDGFRAKMYDITQHPFFKRTIALLVLAQSVLLSVKWDVEDPVTVPLATMSVVFTFIFVLEVTMKIIAMSPAGFWQSRRNRYDLLVTSLGVVWVVLHFALLNAYTYMMGACVIVFRFFSICGKHVTLKMLLLTVVVSMYKSFFIIVGMFLLLLCYAFAGVVLFGTVKYGENINRHANFSSAGKAITVLFRIVTGEDWNKIMHDCMVQPPFCTPDEFTYWATDCGNYAGALMYFCSFYVIIAYIMLNLLVAIIVENFSLFYSTEEDQLLSYNDLRHFQIIWNMVDDKREGVIPTFRVKFLLRLLRGRLEVDLDKDKLLFKHMCYEMERLHNGGDVTFHDVLSMLSYRSVDIRKSLQLEELLAREQLEYTIEEEVAKQTIRMWLKKCLKRIRAKQQQSCSIIHSLRESQQQELSRFLNPPSIETTQPSEDTNANSQDNSMQPETSSQQQLLSPTLSDRGGSRQDAADAGKPQRKFGQWRLPSAPKPISHSVSSVNLRFGGRTTMKSVVCKMNPMTDAASCGSEVKKWWTRQLTVESDESGDDLLDI

SEQ ID NO: 11 - protein, 1732 aa, synthetic construct
  REGION 1..1732: note = Synthetic: hNALCN_delta-829-834
MLKRKQSSRVEAQPVTDFGPDESLSDNADILWINKPWVHSLLRICAIISVISVCMNTPMTFEHYPPLQYVTFTLDTLLMFLYTAEMIAKMHIRGIVKGDSSYVKDRWCVFDGFMVFCLWVSLVLQVFEIADIVDQMSPWGMLRIPRPLIMIRAFRIYFRFELPRTRITNILKRSGEQIWSVSIFLLFFLLLYGILGVQMFGTFTYHCVVNDTKPGNVTWNSLAIPDTHCSPELEEGYQCPPGFKCMDLEDLGLSRQELGYSGFNEIGTSIFTVYEAASQEGWVFLMYRAIDSFPRWRSYFYFITLIFFLAWLVKNVFIAVIIETFAEIRVQFQQMWGSRSSTTSTATTQMFHEDAAGGWQLVAVDVNKPQGRAPACLQKMMRSSVFHMFILSMVTVDVIVAASNYYKGENFRRQYDEFYLAEVAFTVLFDLEALLKIWCLGFTGYISSSLHKFELLLVIGTTLHVYPDLYHSQFTYFQVLRVVRLIKISPALEDFVYKIFGPGKKLGSLVVFTASLLIVMSAISLQMFCFVEELDRFTTFPRAFMSMFQILTQEGWVDVMDQTLNAVGHMWAPVVAIYFILYHLFATLILLSLFVAVILDNLELDEDLKKLKQLKQSEANADTKEKLPLRLRIFEKFPNRPQMVKISKLPSDFTVPKIRESFMKQFIDRQQQDTCCLLRSLPTTSSSSCDHSKRSAIEDNKYIDQKLRKSVFSIRARNLLEKETAVTKILRACTRQRMLSGSFEGQPAKERSILSVQHHIRQERRSLRHGSNSQRISRGKSLETLTQDHSNTVRYRNAQREDSEIKMIQEKKEQAEMKRKVQEEELREKPLFIVGREHRFRNFCRVVVRARFNASKTDPVTGAVKNTKYHQLYDLLGLVTYLDWVMIIVTICSCISMMFESPFRRVMHAPTLQIAEYVFVIFMSIELNLKIMADGLFFTPTAVIRDFGGVMDIFIYLVSLIFLCWMPQNVPAESGAQLLMVLRCLRPLRIFKLVPQMRKVVRELFSGFKEIFLVSILLLTLMLVFASFGVQLFAGKLAKCNDPNIIRREDCNGIFRINVSVSKNLNLKLRPGEKKPGFWVPRVWANPRNFNFDNVGNAMLALFEVLSLKGWVEVRDVIIHRVGPIHGIYIHVFVFLGCMIGLTLFVGVVIANFNENKGTALLTVDQRRWEDLKSRLKIAQPLHLPPRPDNDGFRAKMYDITQHPFFKRTIALLVLAQSVLLSVKWDVEDPVTVPLATMSVVFTFIFVLEVTMKIIAMSPAGFWQSRRNRYDLLVTSLGVVWVVLHFALLNAYTYMMGACVIVFRFFSICGKHVTLKMLLLTVVVSMYKSFFIIVGMFLLLLCYAFAGVVLFGTVKYGENINRHANFSSAGKAITVLFRIVTGEDWNKIMHDCMVQPPFCTPDEFTYWATDCGNYAGALMYFCSFYVIIAYIMLNLLVAIIVENFSLFYSTEEDQLLSYNDLRHFQIIWNMVDDKREGVIPTFRVKFLLRLLRGRLEVDLDKDKLLFKHMCYEMERLHNGGDVTFHDVLSMLSYRSVDIRKSLQLEELLAREQLEYTIEEEVAKQTIRMWLKKCLKRIRAKQQQSCSIIHSLRESQQQELSRFLNPPSIETTQPSEDTNANSQDNSMQPETSSQQQLLSPTLSDRGGSRQDAADAGKPQRKFGQWRLPSAPKPISHSVSSVNLRFGGRTTMKSVVCKMNPMTDAASCGSEVKKWWTRQLTVESDESGDDLLDI

SEQ ID NO: 12 - protein, 1709 aa, synthetic construct
  REGION 1..1709: note = Synthetic: hNALCN-hNaV1.4 DII-DIII linker chimera1
MLKRKQSSRVEAQPVTDFGPDESLSDNADILWINKPWVHSLLRICAIISVISVCMNTPMTFEHYPPLQYVTFTLDTLLMFLYTAEMIAKMHIRGIVKGDSSYVKDRWCVFDGFMVFCLWVSLVLQVFEIADIVDQMSPWGMLRIPRPLIMIRAFRIYFRFELPRTRITNILKRSGEQIWSVSIFLLFFLLLYGILGVQMFGTFTYHCVVNDTKPGNVTWNSLAIPDTHCSPELEEGYQCPPGFKCMDLEDLGLSRQELGYSGFNEIGTSIFTVYEAASQEGWVFLMYRAIDSFPRWRSYFYFITLIFFLAWLVKNVFIAVIIETFAEIRVQFQQMWGSRSSTTSTATTQMFHEDAAGGWQLVAVDVNKPQGRAPACLQKMMRSSVFHMFILSMVTVDVIVAASNYYKGENFRRQYDEFYLAEVAFTVLFDLEALLKIWCLGFTGYISSSLHKFELLLVIGTTLHVYPDLYHSQFTYFQVLRVVRLIKISPALEDFVYKIFGPGKKLGSLVVFTASLLIVMSAISLQMFCFVEELDRFTTFPRAFMSMFQILTQEGWVDVMDQTLNAVGHMWAPVVAIYFILYHLFATLILLSLFVAVILDNLELDEDLKKLKQLKQSNLQIAIGRIKLGIGFAKAFLLGLLHGKILSPKDIMLSLGEADGAGEAGEAGETAPEDEKKEPPEEDLKKDNHILNHMGLADGPPSSLELDHLNFINNPYLTIQVPIASEESDLEMPTEEETDTFSEPEDSKKPPQPLYDGNSSVCSTADYKPPEEDPEEQAEENPEGEQPEECFTEACVQRWPCLYVDISQGRGKKWWRFRNFCRVVVRARFNASKTDPVTGAVKNTKYHQLYDLLGLVTYLDWVMIIVTICSCISMMFESPFRRVMHAPTLQIAEYVFVIFMSIELNLKIMADGLFFTPTAVIRDFGGVMDIFIYLVSLIFLCWMPQNVPAESGAQLLMVLRCLRPLRIFKLVPQMRKVVRELFSGFKEIFLVSILLLTLMLVFASFGVQLFAGKLAKCNDPNIIRREDCNGIFRINVSVSKNLNLKLRPGEKKPGFWVPRVWANPRNFNFDNVGNAMLALFEVLSLKGWVEVRDVIIHRVGPIHGIYIHVFVFLGCMIGLTLFVGVVIANFNENKGTALLTVDQRRWEDLKSRLKIAQPLHLPPRPDNDGFRAKMYDITQHPFFKRTIALLVLAQSVLLSVKWDVEDPVTVPLATMSVVFTFIFVLEVTMKIIAMSPAGFWQSRRNRYDLLVTSLGVVWVVLHFALLNAYTYMMGACVIVFRFFSICGKHVTLKMLLLTVVVSMYKSFFIIVGMFLLLLCYAFAGVVLFGTVKYGENINRHANFSSAGKAITVLFRIVTGEDWNKIMHDCMVQPPFCTPDEFTYWATDCGNYAGALMYFCSFYVIIAYIMLNLLVAIIVENFSLFYSTEEDQLLSYNDLRHFQIIWNMVDDKREGVIPTFRVKFLLRLLRGRLEVDLDKDKLLFKHMCYEMERLHNGGDVTFHDVLSMLSYRSVDIRKSLQLEELLAREQLEYTIEEEVAKQTIRMWLKKCLKRIRAKQQQSCSIIHSLRESQQQELSRFLNPPSIETTQPSEDTNANSQDNSMQPETSSQQQLLSPTLSDRGGSRQDAADAGKPQRKFGQWRLPSAPKPISHSVSSVNLRFGGRTTMKSVVCKMNPMTDAASCGSEVKKWWTRQLTVESDESGDDLLDI

SEQ ID NO: 13 - protein, 1709 aa, synthetic construct
  REGION 1..1709: note = Synthetic: hNALCN-hNaV1.4 DII-DIII linker chimera2
MLKRKQSSRVEAQPVTDFGPDESLSDNADILWINKPWVHSLLRICAIISVISVCMNTPMTFEHYPPLQYVTFTLDTLLMFLYTAEMIAKMHIRGIVKGDSSYVKDRWCVFDGFMVFCLWVSLVLQVFEIADIVDQMSPWGMLRIPRPLIMIRAFRIYFRFELPRTRITNILKRSGEQIWSVSIFLLFFLLLYGILGVQMFGTFTYHCVVNDTKPGNVTWNSLAIPDTHCSPELEEGYQCPPGFKCMDLEDLGLSRQELGYSGFNEIGTSIFTVYEAASQEGWVFLMYRAIDSFPRWRSYFYFITLIFFLAWLVKNVFIAVIIETFAEIRVQFQQMWGSRSSTTSTATTQMFHEDAAGGWQLVAVDVNKPQGRAPACLQKMMRSSVFHMFILSMVTVDVIVAASNYYKGENFRRQYDEFYLAEVAFTVLFDLEALLKIWCLGFTGYISSSLHKFELLLVIGTTLHVYPDLYHSQFTYFQVLRVVRLIKISPALEDFVYKIFGPGKKLGSLVVFTASLLIVMSAISLQMFCFVEELDRFTTFPRAFMSMFQILTQEGWVDVMDQTLNAVGHMWAPVVAIYFILYHLFATLILLSLFVAVILDNLELDEDLKKLKQLKQSEANADTKEKLPLRLRIFEKFPNRPQMVKISKLPSDFTVPKIRESFMKQFIDRQQQDDEKKEPPEEDLKKDNHILNHMGLADGPPSSLELDHLNFINNPYLTIQVPIASEESDLEMPTEEETDTFSEPEDSKKPPQPLYDGNSSVCSTADYKPPEEDPEEQAEENPEGEQPEECFTEACVQRWPCLYVDISQGRGKKWWRFRNFCRVVVRARFNASKTDPVTGAVKNTKYHQLYDLLGLVTYLDWVMIIVTICSCISMMFESPFRRVMHAPTLQIAEYVFVIFMSIELNLKIMADGLFFTPTAVIRDFGGVMDIFIYLVSLIFLCWMPQNVPAESGAQLLMVLRCLRPLRIFKLVPQMRKVVRELFSGFKEIFLVSILLLTLMLVFASFGVQLFAGKLAKCNDPNIIRREDCNGIFRINVSVSKNLNLKLRPGEKKPGFWVPRVWANPRNFNFDNVGNAMLALFEVLSLKGWVEVRDVIIHRVGPIHGIYIHVFVFLGCMIGLTLFVGVVIANFNENKGTALLTVDQRRWEDLKSRLKIAQPLHLPPRPDNDGFRAKMYDITQHPFFKRTIALLVLAQSVLLSVKWDVEDPVTVPLATMSVVFTFIFVLEVTMKIIAMSPAGFWQSRRNRYDLLVTSLGVVWVVLHFALLNAYTYMMGACVIVFRFFSICGKHVTLKMLLLTVVVSMYKSFFIIVGMFLLLLCYAFAGVVLFGTVKYGENINRHANFSSAGKAITVLFRIVTGEDWNKIMHDCMVQPPFCTPDEFTYWATDCGNYAGALMYFCSFYVIIAYIMLNLLVAIIVENFSLFYSTEEDQLLSYNDLRHFQIIWNMVDDKREGVIPTFRVKFLLRLLRGRLEVDLDKDKLLFKHMCYEMERLHNGGDVTFHDVLSMLSYRSVDIRKSLQLEELLAREQLEYTIEEEVAKQTIRMWLKKCLKRIRAKQQQSCSIIHSLRESQQQELSRFLNPPSIETTQPSEDTNANSQDNSMQPETSSQQQLLSPTLSDRGGSRQDAADAGKPQRKFGQWRLPSAPKPISHSVSSVNLRFGGRTTMKSVVCKMNPMTDAASCGSEVKKWWTRQLTVESDESGDDLLDI

SEQ ID NO: 14 - protein, 1709 aa, synthetic construct
  REGION 1..1709: note = Synthetic: hNALCN-hNaV1.4 DII-DIII linker chimera3
MLKRKQSSRVEAQPVTDFGPDESLSDNADILWINKPWVHSLLRICAIISVISVCMNTPMTFEHYPPLQYVTFTLDTLLMFLYTAEMIAKMHIRGIVKGDSSYVKDRWCVFDGFMVFCLWVSLVLQVFEIADIVDQMSPWGMLRIPRPLIMIRAFRIYFRFELPRTRITNILKRSGEQIWSVSIFLLFFLLLYGILGVQMFGTFTYHCVVNDTKPGNVTWNSLAIPDTHCSPELEEGYQCPPGFKCMDLEDLGLSRQELGYSGFNEIGTSIFTVYEAASQEGWVFLMYRAIDSFPRWRSYFYFITLIFFLAWLVKNVFIAVIIETFAEIRVQFQQMWGSRSSTTSTATTQMFHEDAAGGWQLVAVDVNKPQGRAPACLQKMMRSSVFHMFILSMVTVDVIVAASNYYKGENFRRQYDEFYLAEVAFTVLFDLEALLKIWCLGFTGYISSSLHKFELLLVIGTTLHVYPDLYHSQFTYFQVLRVVRLIKISPALEDFVYKIFGPGKKLGSLVVFTASLLIVMSAISLQMFCFVEELDRFTTFPRAFMSMFQILTQEGWVDVMDQTLNAVGHMWAPVVAIYFILYHLFATLILLSLFVAVILDNLELDEDLKKLKQLKQSEANADTKEKLPLRLRIFEKFPNRPQMVKISKLPSDFTVPKIRESFMKQFIDRQQQDDEKKEPPEEDLKKDNHILNHMGLADGPPSSLELDHLNFINNPYLTIQVPIASEESDLEMPTEEETDTFSEPEDSKKPPQPLYDGNSSVCSTADYKAQREDSEIKMIQEKKEQAEMKRKVQEEELRENHPYFDKPLFIVGREHRFRNFCRVVVRARFNASKTDPVTGAVKNTKYHQLYDLLGLVTYLDWVMIIVTICSCISMMFESPFRRVMHAPTLQIAEYVFVIFMSIELNLKIMADGLFFTPTAVIRDFGGVMDIFIYLVSLIFLCWMPQNVPAESGAQLLMVLRCLRPLRIFKLVPQMRKVVRELFSGFKEIFLVSILLLTLMLVFASFGVQLFAGKLAKCNDPNIIRREDCNGIFRINVSVSKNLNLKLRPGEKKPGFWVPRVWANPRNFNFDNVGNAMLALFEVLSLKGWVEVRDVIIHRVGPIHGIYIHVFVFLGCMIGLTLFVGVVIANFNENKGTALLTVDQRRWEDLKSRLKIAQPLHLPPRPDNDGFRAKMYDITQHPFFKRTIALLVLAQSVLLSVKWDVEDPVTVPLATMSVVFTFIFVLEVTMKIIAMSPAGFWQSRRNRYDLLVTSLGVVWVVLHFALLNAYTYMMGACVIVFRFFSICGKHVTLKMLLLTVVVSMYKSFFIIVGMFLLLLCYAFAGVVLFGTVKYGENINRHANFSSAGKAITVLFRIVTGEDWNKIMHDCMVQPPFCTPDEFTYWATDCGNYAGALMYFCSFYVIIAYIMLNLLVAIIVENFSLFYSTEEDQLLSYNDLRHFQIIWNMVDDKREGVIPTFRVKFLLRLLRGRLEVDLDKDKLLFKHMCYEMERLHNGGDVTFHDVLSMLSYRSVDIRKSLQLEELLAREQLEYTIEEEVAKQTIRMWLKKCLKRIRAKQQQSCSIIHSLRESQQQELSRFLNPPSIETTQPSEDTNANSQDNSMQPETSSQQQLLSPTLSDRGGSRQDAADAGKPQRKFGQWRLPSAPKPISHSVSSVNLRFGGRTTMKSVVCKMNPMTDAASCGSEVKKWWTRQLTVESDESGDDLLDI

SEQ ID NO: 15 - protein, 1738 aa, synthetic construct
  REGION 1..1738: note = Synthetic: hNALCN-hNaV1.4 DII-DIII linker chimera4
MLKRKQSSRVEAQPVTDFGPDESLSDNADILWINKPWVHSLLRICAIISVISVCMNTPMTFEHYPPLQYVTFTLDTLLMFLYTAEMIAKMHIRGIVKGDSSYVKDRWCVFDGFMVFCLWVSLVLQVFEIADIVDQMSPWGMLRIPRPLIMIRAFRIYFRFELPRTRITNILKRSGEQIWSVSIFLLFFLLLYGILGVQMFGTFTYHCVVNDTKPGNVTWNSLAIPDTHCSPELEEGYQCPPGFKCMDLEDLGLSRQELGYSGFNEIGTSIFTVYEAASQEGWVFLMYRAIDSFPRWRSYFYFITLIFFLAWLVKNVFIAVIIETFAEIRVQFQQMWGSRSSTTSTATTQMFHEDAAGGWQLVAVDVNKPQGRAPACLQKMMRSSVFHMFILSMVTVDVIVAASNYYKGENFRRQYDEFYLAEVAFTVLFDLEALLKIWCLGFTGYISSSLHKFELLLVIGTTLHVYPDLYHSQFTYFQVLRVVRLIKISPALEDFVYKIFGPGKKLGSLVVFTASLLIVMSAISLQMFCFVEELDRFTTFPRAFMSMFQILTQEGWVDVMDQTLNAVGHMWAPVVAIYFILYHLFATLILLSLFVAVILDNLELDEDLKKLKQLKQSEANADTKEKLPLRLRIFEKFPNRPQMVKISKLPSDFTVPKIRESFMKQFIDRQQQDTCCLLRSLPTTSSSSCDHSKRSAIEDNKYIDQKLRKSVFSIRARNLLEKETAVTKILRACTRQRMLSGSFEGQPAKERSILSVQHHIRQERRSLRHGSNSQRISRGKSLETLTQDHSNTVRYRNPPEEDPEEQAEENPEGEQPEECFTEACVQRWPCLYVDISQGRGKKWWRFRNFCRVVVRARFNASKTDPVTGAVKNTKYHQLYDLLGLVTYLDWVMIIVTICSCISMMFESPFRRVMHAPTLQIAEYVFVIFMSIELNLKIMADGLFFTPTAVIRDFGGVMDIFIYLVSLIFLCWMPQNVPAESGAQLLMVLRCLRPLRIFKLVPQMRKVVRELFSGFKEIFLVSILLLTLMLVFASFGVQLFAGKLAKCNDPNIIRREDCNGIFRINVSVSKNLNLKLRPGEKKPGFWVPRVWANPRNFNFDNVGNAMLALFEVLSLKGWVEVRDVIIHRVGPIHGIYIHVFVFLGCMIGLTLFVGVVIANFNENKGTALLTVDQRRWEDLKSRLKIAQPLHLPPRPDNDGFRAKMYDITQHPFFKRTIALLVLAQSVLLSVKWDVEDPVTVPLATMSVVFTFIFVLEVTMKIIAMSPAGFWQSRRNRYDLLVTSLGVVWVVLHFALLNAYTYMMGACVIVFRFFSICGKHVTLKMLLLTVVVSMYKSFFIIVGMFLLLLCYAFAGVVLFGTVKYGENINRHANFSSAGKAITVLFRIVTGEDWNKIMHDCMVQPPFCTPDEFTYWATDCGNYAGALMYFCSFYVIIAYIMLNLLVAIIVENFSLFYSTEEDQLLSYNDLRHFQIIWNMVDDKREGVIPTFRVKFLLRLLRGRLEVDLDKDKLLFKHMCYEMERLHNGGDVTFHDVLSMLSYRSVDIRKSLQLEELLAREQLEYTIEEEVAKQTIRMWLKKCLKRIRAKQQQSCSIIHSLRESQQQELSRFLNPPSIETTQPSEDTNANSQDNSMQPETSSQQQLLSPTLSDRGGSRQDAADAGKPQRKFGQWRLPSAPKPISHSVSSVNLRFGGRTTMKSVVCKMNPMTDAASCGSEVKKWWTRQLTVESDESGDDLLDI

SEQ ID NO: 16 - protein, 619 aa, synthetic construct
  REGION 1..619: note = Synthetic: MBP-modified hNALCN DII-DIII linker
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSNNNNNNNNNNGENLYFQGSSEANADTKEKLPLRLRIFEKFPNRPQMVKISKLPSDFTVPKIRESFMKQFIDRQQQDTCCLLRSLPTTSSSSCDHSKRSAIEDNKYIDQKLRKSVFSIRARNLLEKETAVTKILRACTRQRMLSGSFEGQPAKERSILSVQHHIRQERRSLRHGSNSQRISRGKSLETLTQDHSNTVRYRNAQREDSEIKMIQEKKEQAEMKRKVQEEELRENHPYFDKPLFIVGREHR

SEQ ID NO: 17 - protein, 253 aa, synthetic construct
  REGION 1..253: note = Synthetic: Palmitoyl peptide-modified hNALCN DII-DIII linker
MMLCCMRRTKQGTGGSGGTGGSGGSEANADTKEKLPLRLRIFEKFPNRPQMVKISKLPSDFTVPKIRESFMKQFIDRQQQDTCCLLRSLPTTSSSSCDHSKRSAIEDNKYIDQKLRKSVFSIRARNLLEKETAVTKILRACTRQRMLSGSFEGQPAKERSILSVQHHIRQERRSLRHGSNSQRISRGKSLETLTQDHSNTVRYRNAQREDSEIKMIQEKKEQAEMKRKVQEEELRENHPYFDKPLFIVGREHR

SEQ ID NO: 18 - protein, 229 aa, synthetic construct
  REGION 1..229: note = Synthetic: Unmodified hNALCN DII-DIII linker (residues S617-R845)
SEANADTKEKLPLRLRIFEKFPNRPQMVKISKLPSDFTVPKIRESFMKQFIDRQQQDTCCLLRSLPTTSSSSCDHSKRSAIEDNKYIDQKLRKSVFSIRARNLLEKETAVTKILRACTRQRMLSGSFEGQPAKERSILSVQHHIRQERRSLRHGSNSQRISRGKSLETLTQDHSNTVRYRNAQREDSEIKMIQEKKEQAEMKRKVQEEELRENHPYFDKPLFIVGREHR

SEQ ID NO: 19 - protein, 15 aa, synthetic construct
  REGION 1..15: note = Synthetic: hNALCN DI-DII linker peptide (residues Q349-A363
QMFHEDAAGGWQLVA

SEQ ID NO: 20 - protein, 33 aa, synthetic construct
  REGION 1..33: note = Synthetic: hNALCN DII-DIII linker peptide (residues P638-Q670)
PNRPQMVKISKLPSDFTVPKIRESFMKQFIDRQ

SEQ ID NO: 21 - protein, 35 aa, synthetic construct
  REGION 1..35: note = Synthetic: hNALCN DII-DIII linker peptide (residues R794-E828)
RYRNAQREDSEIKMIQEKKEQAEMKRKVQEEELRE

SEQ ID NO: 22 - protein, 7 aa, synthetic construct
  REGION 1..7: note = Synthetic: hNALCN DII-DIII linker peptide (residues N829-K835)
NHPYFDK

SEQ ID NO: 23 - protein, 124 aa, synthetic construct
  REGION 1..124: note = Synthetic: hNALCN DII-DIII linker peptide (residues S617-S740)
SEANADTKEKLPLRLRIFEKFPNRPQMVKISKLPSDFTVPKIRESFMKQFIDRQQQDTCCLLRSLPTTSSSSCDHSKRSAIEDNKYIDQKLRKSVFSIRARNLLEKETAVTKILRACTRQRMLS

SEQ ID NO: 24 - protein, 116 aa, synthetic construct
  REGION 1..116: note = Synthetic: hNALCN DII-DIII linker peptide (residues R659-Q774)
RESFMKQFIDRQQQDTCCLLRSLPTTSSSSCDHSKRSAIEDNKYIDQKLRKSVFSIRARNLLEKETAVTKILRACTRQRMLSGSFEGQPAKERSILSVQHHIRQERRSLRHGSNSQ

SEQ ID NO: 25 - protein, 105 aa, synthetic construct
  REGION 1..105: note = Synthetic: hNALCN DII-DIII linker peptide (residues G741-R845)
GSFEGQPAKERSILSVQHHIRQERRSLRHGSNSQRISRGKSLETLTQDHSNTVRYRNAQREDSEIKMIQEKKEQAEMKRKVQEEELRENHPYFDKPLFIVGREHR

SEQ ID NO: 26 - protein, 41 aa, synthetic construct
  REGION 1..41: note = Synthetic: hNALCN DI-DII linker peptide (residues M335-A375)
MWGSRSSTTSTATTQMFHEDAAGGWQLVAVDVNKPQGRAPA

SEQ ID NO: 27 - protein, 60 aa, synthetic construct
  REGION 1..60: note = Synthetic: hNALCN DII-DIII linker peptide (residues S617-C676)
SEANADTKEKLPLRLRIFEKFPNRPQMVKISKLPSDFTVPKIRESFMKQFIDRQQQDTCC

SEQ ID NO: 28 - protein, 64 aa, synthetic construct
  REGION 1..64: note = Synthetic: hNALCN DII-DIII linker peptide (residues L677-S740)
LLRSLPTTSSSSCDHSKRSAIEDNKYIDQKLRKSVFSIRARNLLEKETAVTKILRACTRQRMLS

SEQ ID NO: 29 - protein, 49 aa, synthetic construct
  REGION 1..49: note = Synthetic: hNALCN DII-DIII linker peptide (residues G741-H789)
GSFEGQPAKERSILSVQHHIRQERRSLRHGSNSQRISRGKSLETLTQDH

SEQ ID NO: 30 - protein, 56 aa, synthetic construct
  REGION 1..56: note = Synthetic: hNALCN DII-DIII linker peptide (residues S790-R845)
SNTVRYRNAQREDSEIKMIQEKKEQAEMKRKVQEEELRENHPYFDKPLFIVGREHR

SEQ ID NO: 31 - protein, 285 aa, synthetic construct
  REGION 1..285: note = Synthetic: hNALCN residues Y1454-I1738
YSTEEDQLLSYNDLRHFQIIWNMVDDKREGVIPTFRVKFLLRLLRGRLEVDLDKDKLLFKHMCYEMERLHNGGDVTFHDVLSMLSYRSVDIRKSLQLEELLAREQLEYTIEEEVAKQTIRMWLKKCLKRIRAKQQQSCSIIHSLRESQQQELSRFLNPPSIETTQPSEDTNANSQDNSMQPETSSQQQLLSPTLSDRGGSRQDAADAGKPQRKFGQWRLPSAPKPISHSVSSVNLRFGGRTTMKSVVCKMNPMTDAASCGSEVKKWWTRQLTVESDESGDDLLDI

SEQ ID NO: 32 - protein, 2007 aa, synthetic construct
  REGION 1..2007: note = Synthetic: hNALCN eGFP-2X Flag
MLKRKQSSRVEAQPVTDFGPDESLSDNADILWINKPWVHSLLRICAIISVISVCMNTPMTFEHYPPLQYVTFTLDTLLMFLYTAEMIAKMHIRGIVKGDSSYVKDRWCVFDGFMVFCLWVSLVLQVFEIADIVDQMSPWGMLRIPRPLIMIRAFRIYFRFELPRTRITNILKRSGEQIWSVSIFLLFFLLLYGILGVQMFGTFTYHCVVNDTKPGNVTWNSLAIPDTHCSPELEEGYQCPPGFKCMDLEDLGLSRQELGYSGFNEIGTSIFTVYEAASQEGWVFLMYRAIDSFPRWRSYFYFITLIFFLAWLVKNVFIAVIIETFAEIRVQFQQMWGSRSSTTSTATTQMFHEDAAGGWQLVAVDVNKPQGRAPACLQKMMRSSVFHMFILSMVTVDVIVAASNYYKGENFRRQYDEFYLAEVAFTVLFDLEALLKIWCLGFTGYISSSLHKFELLLVIGTTLHVYPDLYHSQFTYFQVLRVVRLIKISPALEDFVYKIFGPGKKLGSLVVFTASLLIVMSAISLQMFCFVEELDRFTTFPRAFMSMFQILTQEGWVDVMDQTLNAVGHMWAPVVAIYFILYHLFATLILLSLFVAVILDNLELDEDLKKLKQLKQSEANADTKEKLPLRLRIFEKFPNRPQMVKISKLPSDFTVPKIRESFMKQFIDRQQQDTCCLLRSLPTTSSSSCDHSKRSAIEDNKYIDQKLRKSVFSIRARNLLEKETAVTKILRACTRQRMLSGSFEGQPAKERSILSVQHHIRQERRSLRHGSNSQRISRGKSLETLTQDHSNTVRYRNAQREDSEIKMIQEKKEQAEMKRKVQEEELRENHPYFDKPLFIVGREHRFRNFCRVVVRARFNASKTDPVTGAVKNTKYHQLYDLLGLVTYLDWVMIIVTICSCISMMFESPFRRVMHAPTLQIAEYVFVIFMSIELNLKIMADGLFFTPTAVIRDFGGVMDIFIYLVSLIFLCWMPQNVPAESGAQLLMVLRCLRPLRIFKLVPQMRKVVRELFSGFKEIFLVSILLLTLMLVFASFGVQLFAGKLAKCNDPNIIRREDCNGIFRINVSVSKNLNLKLRPGEKKPGFWVPRVWANPRNFNFDNVGNAMLALFEVLSLKGWVEVRDVIIHRVGPIHGIYIHVFVFLGCMIGLTLFVGVVIANFNENKGTALLTVDQRRWEDLKSRLKIAQPLHLPPRPDNDGFRAKMYDITQHPFFKRTIALLVLAQSVLLSVKWDVEDPVTVPLATMSVVFTFIFVLEVTMKIIAMSPAGFWQSRRNRYDLLVTSLGVVWVVLHFALLNAYTYMMGACVIVFRFFSICGKHVTLKMLLLTVVVSMYKSFFIIVGMFLLLLCYAFAGVVLFGTVKYGENINRHANFSSAGKAITVLFRIVTGEDWNKIMHDCMVQPPFCTPDEFTYWATDCGNYAGALMYFCSFYVIIAYIMLNLLVAIIVENFSLFYSTEEDQLLSYNDLRHFQIIWNMVDDKREGVIPTFRVKFLLRLLRGRLEVDLDKDKLLFKHMCYEMERLHNGGDVTFHDVLSMLSYRSVDIRKSLQLEELLAREQLEYTIEEEVAKQTIRMWLKKCLKRIRAKQQQSCSIIHSLRESQQQELSRFLNPPSIETTQPSEDTNANSQDNSMQPETSSQQQLLSPTLSDRGGSRQDAADAGKPQRKFGQWRLPSAPKPISHSVSSVNLRFGGRTTMKSVVCKMNPMTDAASCGSEVKKWWTRQLTVESDESGDDLLDIGGSGGSVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKGGSGGSDYKDDDDKGSGDYKDDDDK

SEQ ID NO: 33 - protein, 1737 aa, synthetic construct
  REGION 1..1737: note = Synthetic: hNALCN delta-351
MLKRKQSSRVEAQPVTDFGPDESLSDNADILWINKPWVHSLLRICAIISVISVCMNTPMTFEHYPPLQYVTFTLDTLLMFLYTAEMIAKMHIRGIVKGDSSYVKDRWCVFDGFMVFCLWVSLVLQVFEIADIVDQMSPWGMLRIPRPLIMIRAFRIYFRFELPRTRITNILKRSGEQIWSVSIFLLFFLLLYGILGVQMFGTFTYHCVVNDTKPGNVTWNSLAIPDTHCSPELEEGYQCPPGFKCMDLEDLGLSRQELGYSGFNEIGTSIFTVYEAASQEGWVFLMYRAIDSFPRWRSYFYFITLIFFLAWLVKNVFIAVIIETFAEIRVQFQQMWGSRSSTTSTATTQMHEDAAGGWQLVAVDVNKPQGRAPACLQKMMRSSVFHMFILSMVTVDVIVAASNYYKGENFRRQYDEFYLAEVAFTVLFDLEALLKIWCLGFTGYISSSLHKFELLLVIGTTLHVYPDLYHSQFTYFQVLRVVRLIKISPALEDFVYKIFGPGKKLGSLVVFTASLLIVMSAISLQMFCFVEELDRFTTFPRAFMSMFQILTQEGWVDVMDQTLNAVGHMWAPVVAIYFILYHLFATLILLSLFVAVILDNLELDEDLKKLKQLKQSEANADTKEKLPLRLRIFEKFPNRPQMVKISKLPSDFTVPKIRESFMKQFIDRQQQDTCCLLRSLPTTSSSSCDHSKRSAIEDNKYIDQKLRKSVFSIRARNLLEKETAVTKILRACTRQRMLSGSFEGQPAKERSILSVQHHIRQERRSLRHGSNSQRISRGKSLETLTQDHSNTVRYRNAQREDSEIKMIQEKKEQAEMKRKVQEEELRENHPYFDKPLFIVGREHRFRNFCRVVVRARFNASKTDPVTGAVKNTKYHQLYDLLGLVTYLDWVMIIVTICSCISMMFESPFRRVMHAPTLQIAEYVFVIFMSIELNLKIMADGLFFTPTAVIRDFGGVMDIFIYLVSLIFLCWMPQNVPAESGAQLLMVLRCLRPLRIFKLVPQMRKVVRELFSGFKEIFLVSILLLTLMLVFASFGVQLFAGKLAKCNDPNIIRREDCNGIFRINVSVSKNLNLKLRPGEKKPGFWVPRVWANPRNFNFDNVGNAMLALFEVLSLKGWVEVRDVIIHRVGPIHGIYIHVFVFLGCMIGLTLFVGVVIANFNENKGTALLTVDQRRWEDLKSRLKIAQPLHLPPRPDNDGFRAKMYDITQHPFFKRTIALLVLAQSVLLSVKWDVEDPVTVPLATMSVVFTFIFVLEVTMKIIAMSPAGFWQSRRNRYDLLVTSLGVVWVVLHFALLNAYTYMMGACVIVFRFFSICGKHVTLKMLLLTVVVSMYKSFFIIVGMFLLLLCYAFAGVVLFGTVKYGENINRHANFSSAGKAITVLFRIVTGEDWNKIMHDCMVQPPFCTPDEFTYWATDCGNYAGALMYFCSFYVIIAYIMLNLLVAIIVENFSLFYSTEEDQLLSYNDLRHFQIIWNMVDDKREGVIPTFRVKFLLRLLRGRLEVDLDKDKLLFKHMCYEMERLHNGGDVTFHDVLSMLSYRSVDIRKSLQLEELLAREQLEYTIEEEVAKQTIRMWLKKCLKRIRAKQQQSCSIIHSLRESQQQELSRFLNPPSIETTQPSEDTNANSQDNSMQPETSSQQQLLSPTLSDRGGSRQDAADAGKPQRKFGQWRLPSAPKPISHSVSSVNLRFGGRTTMKSVVCKMNPMTDAASCGSEVKKWWTRQLTVESDESGDDLLDI

SEQ ID NO: 34 - protein, 1737 aa, synthetic construct
  REGION 1..1737: note = Synthetic: hNALCN_delta-359
MLKRKQSSRVEAQPVTDFGPDESLSDNADILWINKPWVHSLLRICAIISVISVCMNTPMTFEHYPPLQYVTFTLDTLLMFLYTAEMIAKMHIRGIVKGDSSYVKDRWCVFDGFMVFCLWVSLVLQVFEIADIVDQMSPWGMLRIPRPLIMIRAFRIYFRFELPRTRITNILKRSGEQIWSVSIFLLFFLLLYGILGVQMFGTFTYHCVVNDTKPGNVTWNSLAIPDTHCSPELEEGYQCPPGFKCMDLEDLGLSRQELGYSGFNEIGTSIFTVYEAASQEGWVFLMYRAIDSFPRWRSYFYFITLIFFLAWLVKNVFIAVIIETFAEIRVQFQQMWGSRSSTTSTATTQMFHEDAAGGQLVAVDVNKPQGRAPACLQKMMRSSVFHMFILSMVTVDVIVAASNYYKGENFRRQYDEFYLAEVAFTVLFDLEALLKIWCLGFTGYISSSLHKFELLLVIGTTLHVYPDLYHSQFTYFQVLRVVRLIKISPALEDFVYKIFGPGKKLGSLVVFTASLLIVMSAISLQMFCFVEELDRFTTFPRAFMSMFQILTQEGWVDVMDQTLNAVGHMWAPVVAIYFILYHLFATLILLSLFVAVILDNLELDEDLKKLKQLKQSEANADTKEKLPLRLRIFEKFPNRPQMVKISKLPSDFTVPKIRESFMKQFIDRQQQDTCCLLRSLPTTSSSSCDHSKRSAIEDNKYIDQKLRKSVFSIRARNLLEKETAVTKILRACTRQRMLSGSFEGQPAKERSILSVQHHIRQERRSLRHGSNSQRISRGKSLETLTQDHSNTVRYRNAQREDSEIKMIQEKKEQAEMKRKVQEEELRENHPYFDKPLFIVGREHRFRNFCRVVVRARFNASKTDPVTGAVKNTKYHQLYDLLGLVTYLDWVMIIVTICSCISMMFESPFRRVMHAPTLQIAEYVFVIFMSIELNLKIMADGLFFTPTAVIRDFGGVMDIFIYLVSLIFLCWMPQNVPAESGAQLLMVLRCLRPLRIFKLVPQMRKVVRELFSGFKEIFLVSILLLTLMLVFASFGVQLFAGKLAKCNDPNIIRREDCNGIFRINVSVSKNLNLKLRPGEKKPGFWVPRVWANPRNFNFDNVGNAMLALFEVLSLKGWVEVRDVIIHRVGPIHGIYIHVFVFLGCMIGLTLFVGVVIANFNENKGTALLTVDQRRWEDLKSRLKIAQPLHLPPRPDNDGFRAKMYDITQHPFFKRTIALLVLAQSVLLSVKWDVEDPVTVPLATMSVVFTFIFVLEVTMKIIAMSPAGFWQSRRNRYDLLVTSLGVVWVVLHFALLNAYTYMMGACVIVFRFFSICGKHVTLKMLLLTVVVSMYKSFFIIVGMFLLLLCYAFAGVVLFGTVKYGENINRHANFSSAGKAITVLFRIVTGEDWNKIMHDCMVQPPFCTPDEFTYWATDCGNYAGALMYFCSFYVIIAYIMLNLLVAIIVENFSLFYSTEEDQLLSYNDLRHFQIIWNMVDDKREGVIPTFRVKFLLRLLRGRLEVDLDKDKLLFKHMCYEMERLHNGGDVTFHDVLSMLSYRSVDIRKSLQLEELLAREQLEYTIEEEVAKQTIRMWLKKCLKRIRAKQQQSCSIIHSLRESQQQELSRFLNPPSIETTQPSEDTNANSQDNSMQPETSSQQQLLSPTLSDRGGSRQDAADAGKPQRKFGQWRLPSAPKPISHSVSSVNLRFGGRTTMKSVVCKMNPMTDAASCGSEVKKWWTRQLTVESDESGDDLLDI

SEQ ID NO: 35 - protein, 1737 aa, synthetic construct
  REGION 1..1737: note = Synthetic: hNALCN_delta-361
MLKRKQSSRVEAQPVTDFGPDESLSDNADILWINKPWVHSLLRICAIISVISVCMNTPMTFEHYPPLQYVTFTLDTLLMFLYTAEMIAKMHIRGIVKGDSSYVKDRWCVFDGFMVFCLWVSLVLQVFEIADIVDQMSPWGMLRIPRPLIMIRAFRIYFRFELPRTRITNILKRSGEQIWSVSIFLLFFLLLYGILGVQMFGTFTYHCVVNDTKPGNVTWNSLAIPDTHCSPELEEGYQCPPGFKCMDLEDLGLSRQELGYSGFNEIGTSIFTVYEAASQEGWVFLMYRAIDSFPRWRSYFYFITLIFFLAWLVKNVFIAVIIETFAEIRVQFQQMWGSRSSTTSTATTQMFHEDAAGGWQVAVDVNKPQGRAPACLQKMMRSSVFHMFILSMVTVDVIVAASNYYKGENFRRQYDEFYLAEVAFTVLFDLEALLKIWCLGFTGYISSSLHKFELLLVIGTTLHVYPDLYHSQFTYFQVLRVVRLIKISPALEDFVYKIFGPGKKLGSLVVFTASLLIVMSAISLQMFCFVEELDRFTTFPRAFMSMFQILTQEGWVDVMDQTLNAVGHMWAPVVAIYFILYHLFATLILLSLFVAVILDNLELDEDLKKLKQLKQSEANADTKEKLPLRLRIFEKFPNRPQMVKISKLPSDFTVPKIRESFMKQFIDRQQQDTCCLLRSLPTTSSSSCDHSKRSAIEDNKYIDQKLRKSVFSIRARNLLEKETAVTKILRACTRQRMLSGSFEGQPAKERSILSVQHHIRQERRSLRHGSNSQRISRGKSLETLTQDHSNTVRYRNAQREDSEIKMIQEKKEQAEMKRKVQEEELRENHPYFDKPLFIVGREHRFRNFCRVVVRARFNASKTDPVTGAVKNTKYHQLYDLLGLVTYLDWVMIIVTICSCISMMFESPFRRVMHAPTLQIAEYVFVIFMSIELNLKIMADGLFFTPTAVIRDFGGVMDIFIYLVSLIFLCWMPQNVPAESGAQLLMVLRCLRPLRIFKLVPQMRKVVRELFSGFKEIFLVSILLLTLMLVFASFGVQLFAGKLAKCNDPNIIRREDCNGIFRINVSVSKNLNLKLRPGEKKPGFWVPRVWANPRNFNFDNVGNAMLALFEVLSLKGWVEVRDVIIHRVGPIHGIYIHVFVFLGCMIGLTLFVGVVIANFNENKGTALLTVDQRRWEDLKSRLKIAQPLHLPPRPDNDGFRAKMYDITQHPFFKRTIALLVLAQSVLLSVKWDVEDPVTVPLATMSVVFTFIFVLEVTMKIIAMSPAGFWQSRRNRYDLLVTSLGVVWVVLHFALLNAYTYMMGACVIVFRFFSICGKHVTLKMLLLTVVVSMYKSFFIIVGMFLLLLCYAFAGVVLFGTVKYGENINRHANFSSAGKAITVLFRIVTGEDWNKIMHDCMVQPPFCTPDEFTYWATDCGNYAGALMYFCSFYVIIAYIMLNLLVAIIVENFSLFYSTEEDQLLSYNDLRHFQIIWNMVDDKREGVIPTFRVKFLLRLLRGRLEVDLDKDKLLFKHMCYEMERLHNGGDVTFHDVLSMLSYRSVDIRKSLQLEELLAREQLEYTIEEEVAKQTIRMWLKKCLKRIRAKQQQSCSIIHSLRESQQQELSRFLNPPSIETTQPSEDTNANSQDNSMQPETSSQQQLLSPTLSDRGGSRQDAADAGKPQRKFGQWRLPSAPKPISHSVSSVNLRFGGRTTMKSVVCKMNPMTDAASCGSEVKKWWTRQLTVESDESGDDLLDI

SEQ ID NO: 36 - protein, 1727 aa, synthetic construct
  REGION 1..1727: note = Synthetic: hNALCN_delta-364-374
MLKRKQSSRVEAQPVTDFGPDESLSDNADILWINKPWVHSLLRICAIISVISVCMNTPMTFEHYPPLQYVTFTLDTLLMFLYTAEMIAKMHIRGIVKGDSSYVKDRWCVFDGFMVFCLWVSLVLQVFEIADIVDQMSPWGMLRIPRPLIMIRAFRIYFRFELPRTRITNILKRSGEQIWSVSIFLLFFLLLYGILGVQMFGTFTYHCVVNDTKPGNVTWNSLAIPDTHCSPELEEGYQCPPGFKCMDLEDLGLSRQELGYSGFNEIGTSIFTVYEAASQEGWVFLMYRAIDSFPRWRSYFYFITLIFFLAWLVKNVFIAVIIETFAEIRVQFQQMWGSRSSTTSTATTQMFHEDAAGGWQLVAACLQKMMRSSVFHMFILSMVTVDVIVAASNYYKGENFRRQYDEFYLAEVAFTVLFDLEALLKIWCLGFTGYISSSLHKFELLLVIGTTLHVYPDLYHSQFTYFQVLRVVRLIKISPALEDFVYKIFGPGKKLGSLVVFTASLLIVMSAISLQMFCFVEELDRFTTFPRAFMSMFQILTQEGWVDVMDQTLNAVGHMWAPVVAIYFILYHLFATLILLSLFVAVILDNLELDEDLKKLKQLKQSEANADTKEKLPLRLRIFEKFPNRPQMVKISKLPSDFTVPKIRESFMKQFIDRQQQDTCCLLRSLPTTSSSSCDHSKRSAIEDNKYIDQKLRKSVFSIRARNLLEKETAVTKILRACTRQRMLSGSFEGQPAKERSILSVQHHIRQERRSLRHGSNSQRISRGKSLETLTQDHSNTVRYRNAQREDSEIKMIQEKKEQAEMKRKVQEEELRENHPYFDKPLFIVGREHRFRNFCRVVVRARFNASKTDPVTGAVKNTKYHQLYDLLGLVTYLDWVMIIVTICSCISMMFESPFRRVMHAPTLQIAEYVFVIFMSIELNLKIMADGLFFTPTAVIRDFGGVMDIFIYLVSLIFLCWMPQNVPAESGAQLLMVLRCLRPLRIFKLVPQMRKVVRELFSGFKEIFLVSILLLTLMLVFASFGVQLFAGKLAKCNDPNIIRREDCNGIFRINVSVSKNLNLKLRPGEKKPGFWVPRVWANPRNFNFDNVGNAMLALFEVLSLKGWVEVRDVIIHRVGPIHGIYIHVFVFLGCMIGLTLFVGVVIANFNENKGTALLTVDQRRWEDLKSRLKIAQPLHLPPRPDNDGFRAKMYDITQHPFFKRTIALLVLAQSVLLSVKWDVEDPVTVPLATMSVVFTFIFVLEVTMKIIAMSPAGFWQSRRNRYDLLVTSLGVVWVVLHFALLNAYTYMMGACVIVFRFFSICGKHVTLKMLLLTVVVSMYKSFFIIVGMFLLLLCYAFAGVVLFGTVKYGENINRHANFSSAGKAITVLFRIVTGEDWNKIMHDCMVQPPFCTPDEFTYWATDCGNYAGALMYFCSFYVIIAYIMLNLLVAIIVENFSLFYSTEEDQLLSYNDLRHFQIIWNMVDDKREGVIPTFRVKFLLRLLRGRLEVDLDKDKLLFKHMCYEMERLHNGGDVTFHDVLSMLSYRSVDIRKSLQLEELLAREQLEYTIEEEVAKQTIRMWLKKCLKRIRAKQQQSCSIIHSLRESQQQELSRFLNPPSIETTQPSEDTNANSQDNSMQPETSSQQQLLSPTLSDRGGSRQDAADAGKPQRKFGQWRLPSAPKPISHSVSSVNLRFGGRTTMKSVVCKMNPMTDAASCGSEVKKWWTRQLTVESDESGDDLLDI

SEQ ID NO: 37 - protein, 1732 aa, synthetic construct
  REGION 1..1732: note = Synthetic: hNALCN_delta-662-667
MLKRKQSSRVEAQPVTDFGPDESLSDNADILWINKPWVHSLLRICAIISVISVCMNTPMTFEHYPPLQYVTFTLDTLLMFLYTAEMIAKMHIRGIVKGDSSYVKDRWCVFDGFMVFCLWVSLVLQVFEIADIVDQMSPWGMLRIPRPLIMIRAFRIYFRFELPRTRITNILKRSGEQIWSVSIFLLFFLLLYGILGVQMFGTFTYHCVVNDTKPGNVTWNSLAIPDTHCSPELEEGYQCPPGFKCMDLEDLGLSRQELGYSGFNEIGTSIFTVYEAASQEGWVFLMYRAIDSFPRWRSYFYFITLIFFLAWLVKNVFIAVIIETFAEIRVQFQQMWGSRSSTTSTATTQMFHEDAAGGWQLVAVDVNKPQGRAPACLQKMMRSSVFHMFILSMVTVDVIVAASNYYKGENFRRQYDEFYLAEVAFTVLFDLEALLKIWCLGFTGYISSSLHKFELLLVIGTTLHVYPDLYHSQFTYFQVLRVVRLIKISPALEDFVYKIFGPGKKLGSLVVFTASLLIVMSAISLQMFCFVEELDRFTTFPRAFMSMFQILTQEGWVDVMDQTLNAVGHMWAPVVAIYFILYHLFATLILLSLFVAVILDNLELDEDLKKLKQLKQSEANADTKEKLPLRLRIFEKFPNRPQMVKISKLPSDFTVPKIRESDRQQQDTCCLLRSLPTTSSSSCDHSKRSAIEDNKYIDQKLRKSVFSIRARNLLEKETAVTKILRACTRQRMLSGSFEGQPAKERSILSVQHHIRQERRSLRHGSNSQRISRGKSLETLTQDHSNTVRYRNAQREDSEIKMIQEKKEQAEMKRKVQEEELRENHPYFDKPLFIVGREHRFRNFCRVVVRARFNASKTDPVTGAVKNTKYHQLYDLLGLVTYLDWVMIIVTICSCISMMFESPFRRVMHAPTLQIAEYVFVIFMSIELNLKIMADGLFFTPTAVIRDFGGVMDIFIYLVSLIFLCWMPQNVPAESGAQLLMVLRCLRPLRIFKLVPQMRKVVRELFSGFKEIFLVSILLLTLMLVFASFGVQLFAGKLAKCNDPNIIRREDCNGIFRINVSVSKNLNLKLRPGEKKPGFWVPRVWANPRNFNFDNVGNAMLALFEVLSLKGWVEVRDVIIHRVGPIHGIYIHVFVFLGCMIGLTLFVGVVIANFNENKGTALLTVDQRRWEDLKSRLKIAQPLHLPPRPDNDGFRAKMYDITQHPFFKRTIALLVLAQSVLLSVKWDVEDPVTVPLATMSVVFTFIFVLEVTMKIIAMSPAGFWQSRRNRYDLLVTSLGVVWVVLHFALLNAYTYMMGACVIVFRFFSICGKHVTLKMLLLTVVVSMYKSFFIIVGMFLLLLCYAFAGVVLFGTVKYGENINRHANFSSAGKAITVLFRIVTGEDWNKIMHDCMVQPPFCTPDEFTYWATDCGNYAGALMYFCSFYVIIAYIMLNLLVAIIVENFSLFYSTEEDQLLSYNDLRHFQIIWNMVDDKREGVIPTFRVKFLLRLLRGRLEVDLDKDKLLFKHMCYEMERLHNGGDVTFHDVLSMLSYRSVDIRKSLQLEELLAREQLEYTIEEEVAKQTIRMWLKKCLKRIRAKQQQSCSIIHSLRESQQQELSRFLNPPSIETTQPSEDTNANSQDNSMQPETSSQQQLLSPTLSDRGGSRQDAADAGKPQRKFGQWRLPSAPKPISHSVSSVNLRFGGRTTMKSVVCKMNPMTDAASCGSEVKKWWTRQLTVESDESGDDLLDI

SEQ ID NO: 38 - protein, 1569 aa, synthetic construct
  REGION 1..1569: note = Synthetic: hNALCN_delta-1570-1738
MLKRKQSSRVEAQPVTDFGPDESLSDNADILWINKPWVHSLLRICAIISVISVCMNTPMTFEHYPPLQYVTFTLDTLLMFLYTAEMIAKMHIRGIVKGDSSYVKDRWCVFDGFMVFCLWVSLVLQVFEIADIVDQMSPWGMLRIPRPLIMIRAFRIYFRFELPRTRITNILKRSGEQIWSVSIFLLFFLLLYGILGVQMFGTFTYHCVVNDTKPGNVTWNSLAIPDTHCSPELEEGYQCPPGFKCMDLEDLGLSRQELGYSGFNEIGTSIFTVYEAASQEGWVFLMYRAIDSFPRWRSYFYFITLIFFLAWLVKNVFIAVIIETFAEIRVQFQQMWGSRSSTTSTATTQMFHEDAAGGWQLVAVDVNKPQGRAPACLQKMMRSSVFHMFILSMVTVDVIVAASNYYKGENFRRQYDEFYLAEVAFTVLFDLEALLKIWCLGFTGYISSSLHKFELLLVIGTTLHVYPDLYHSQFTYFQVLRVVRLIKISPALEDFVYKIFGPGKKLGSLVVFTASLLIVMSAISLQMFCFVEELDRFTTFPRAFMSMFQILTQEGWVDVMDQTLNAVGHMWAPVVAIYFILYHLFATLILLSLFVAVILDNLELDEDLKKLKQLKQSEANADTKEKLPLRLRIFEKFPNRPQMVKISKLPSDFTVPKIRESFMKQFIDRQQQDTCCLLRSLPTTSSSSCDHSKRSAIEDNKYIDQKLRKSVFSIRARNLLEKETAVTKILRACTRQRMLSGSFEGQPAKERSILSVQHHIRQERRSLRHGSNSQRISRGKSLETLTQDHSNTVRYRNAQREDSEIKMIQEKKEQAEMKRKVQEEELRENHPYFDKPLFIVGREHRFRNFCRVVVRARFNASKTDPVTGAVKNTKYHQLYDLLGLVTYLDWVMIIVTICSCISMMFESPFRRVMHAPTLQIAEYVFVIFMSIELNLKIMADGLFFTPTAVIRDFGGVMDIFIYLVSLIFLCWMPQNVPAESGAQLLMVLRCLRPLRIFKLVPQMRKVVRELFSGFKEIFLVSILLLTLMLVFASFGVQLFAGKLAKCNDPNIIRREDCNGIFRINVSVSKNLNLKLRPGEKKPGFWVPRVWANPRNFNFDNVGNAMLALFEVLSLKGWVEVRDVIIHRVGPIHGIYIHVFVFLGCMIGLTLFVGVVIANFNENKGTALLTVDQRRWEDLKSRLKIAQPLHLPPRPDNDGFRAKMYDITQHPFFKRTIALLVLAQSVLLSVKWDVEDPVTVPLATMSVVFTFIFVLEVTMKIIAMSPAGFWQSRRNRYDLLVTSLGVVWVVLHFALLNAYTYMMGACVIVFRFFSICGKHVTLKMLLLTVVVSMYKSFFIIVGMFLLLLCYAFAGVVLFGTVKYGENINRHANFSSAGKAITVLFRIVTGEDWNKIMHDCMVQPPFCTPDEFTYWATDCGNYAGALMYFCSFYVIIAYIMLNLLVAIIVENFSLFYSTEEDQLLSYNDLRHFQIIWNMVDDKREGVIPTFRVKFLLRLLRGRLEVDLDKDKLLFKHMCYEMERLHNGGDVTFHDVLSMLSYRSVDIRKSLQLEELLAREQLEYTIEEEVAK

SEQ ID NO: 39 - protein, 1637 aa, synthetic construct
  REGION 1..1637: note = Synthetic: hNALCN_delta-1638-1738
MLKRKQSSRVEAQPVTDFGPDESLSDNADILWINKPWVHSLLRICAIISVISVCMNTPMTFEHYPPLQYVTFTLDTLLMFLYTAEMIAKMHIRGIVKGDSSYVKDRWCVFDGFMVFCLWVSLVLQVFEIADIVDQMSPWGMLRIPRPLIMIRAFRIYFRFELPRTRITNILKRSGEQIWSVSIFLLFFLLLYGILGVQMFGTFTYHCVVNDTKPGNVTWNSLAIPDTHCSPELEEGYQCPPGFKCMDLEDLGLSRQELGYSGFNEIGTSIFTVYEAASQEGWVFLMYRAIDSFPRWRSYFYFITLIFFLAWLVKNVFIAVIIETFAEIRVQFQQMWGSRSSTTSTATTQMFHEDAAGGWQLVAVDVNKPQGRAPACLQKMMRSSVFHMFILSMVTVDVIVAASNYYKGENFRRQYDEFYLAEVAFTVLFDLEALLKIWCLGFTGYISSSLHKFELLLVIGTTLHVYPDLYHSQFTYFQVLRVVRLIKISPALEDFVYKIFGPGKKLGSLVVFTASLLIVMSAISLQMFCFVEELDRFTTFPRAFMSMFQILTQEGWVDVMDQTLNAVGHMWAPVVAIYFILYHLFATLILLSLFVAVILDNLELDEDLKKLKQLKQSEANADTKEKLPLRLRIFEKFPNRPQMVKISKLPSDFTVPKIRESFMKQFIDRQQQDTCCLLRSLPTTSSSSCDHSKRSAIEDNKYIDQKLRKSVFSIRARNLLEKETAVTKILRACTRQRMLSGSFEGQPAKERSILSVQHHIRQERRSLRHGSNSQRISRGKSLETLTQDHSNTVRYRNAQREDSEIKMIQEKKEQAEMKRKVQEEELRENHPYFDKPLFIVGREHRFRNFCRVVVRARFNASKTDPVTGAVKNTKYHQLYDLLGLVTYLDWVMIIVTICSCISMMFESPFRRVMHAPTLQIAEYVFVIFMSIELNLKIMADGLFFTPTAVIRDFGGVMDIFIYLVSLIFLCWMPQNVPAESGAQLLMVLRCLRPLRIFKLVPQMRKVVRELFSGFKEIFLVSILLLTLMLVFASFGVQLFAGKLAKCNDPNIIRREDCNGIFRINVSVSKNLNLKLRPGEKKPGFWVPRVWANPRNFNFDNVGNAMLALFEVLSLKGWVEVRDVIIHRVGPIHGIYIHVFVFLGCMIGLTLFVGVVIANFNENKGTALLTVDQRRWEDLKSRLKIAQPLHLPPRPDNDGFRAKMYDITQHPFFKRTIALLVLAQSVLLSVKWDVEDPVTVPLATMSVVFTFIFVLEVTMKIIAMSPAGFWQSRRNRYDLLVTSLGVVWVVLHFALLNAYTYMMGACVIVFRFFSICGKHVTLKMLLLTVVVSMYKSFFIIVGMFLLLLCYAFAGVVLFGTVKYGENINRHANFSSAGKAITVLFRIVTGEDWNKIMHDCMVQPPFCTPDEFTYWATDCGNYAGALMYFCSFYVIIAYIMLNLLVAIIVENFSLFYSTEEDQLLSYNDLRHFQIIWNMVDDKREGVIPTFRVKFLLRLLRGRLEVDLDKDKLLFKHMCYEMERLHNGGDVTFHDVLSMLSYRSVDIRKSLQLEELLAREQLEYTIEEEVAKQTIRMWLKKCLKRIRAKQQQSCSIIHSLRESQQQELSRFLNPPSIETTQPSEDTNANSQDNSMQPETS

SEQ ID NO: 40 - protein, 1738 aa, synthetic construct
  REGION 1..1738: note = Synthetic: hNALCN_W359A
MLKRKQSSRVEAQPVTDFGPDESLSDNADILWINKPWVHSLLRICAIISVISVCMNTPMTFEHYPPLQYVTFTLDTLLMFLYTAEMIAKMHIRGIVKGDSSYVKDRWCVFDGFMVFCLWVSLVLQVFEIADIVDQMSPWGMLRIPRPLIMIRAFRIYFRFELPRTRITNILKRSGEQIWSVSIFLLFFLLLYGILGVQMFGTFTYHCVVNDTKPGNVTWNSLAIPDTHCSPELEEGYQCPPGFKCMDLEDLGLSRQELGYSGFNEIGTSIFTVYEAASQEGWVFLMYRAIDSFPRWRSYFYFITLIFFLAWLVKNVFIAVIIETFAEIRVQFQQMWGSRSSTTSTATTQMFHEDAAGGAQLVAVDVNKPQGRAPACLQKMMRSSVFHMFILSMVTVDVIVAASNYYKGENFRRQYDEFYLAEVAFTVLFDLEALLKIWCLGFTGYISSSLHKFELLLVIGTTLHVYPDLYHSQFTYFQVLRVVRLIKISPALEDFVYKIFGPGKKLGSLVVFTASLLIVMSAISLQMFCFVEELDRFTTFPRAFMSMFQILTQEGWVDVMDQTLNAVGHMWAPVVAIYFILYHLFATLILLSLFVAVILDNLELDEDLKKLKQLKQSEANADTKEKLPLRLRIFEKFPNRPQMVKISKLPSDFTVPKIRESFMKQFIDRQQQDTCCLLRSLPTTSSSSCDHSKRSAIEDNKYIDQKLRKSVFSIRARNLLEKETAVTKILRACTRQRMLSGSFEGQPAKERSILSVQHHIRQERRSLRHGSNSQRISRGKSLETLTQDHSNTVRYRNAQREDSEIKMIQEKKEQAEMKRKVQEEELRENHPYFDKPLFIVGREHRFRNFCRVVVRARFNASKTDPVTGAVKNTKYHQLYDLLGLVTYLDWVMIIVTICSCISMMFESPFRRVMHAPTLQIAEYVFVIFMSIELNLKIMADGLFFTPTAVIRDFGGVMDIFIYLVSLIFLCWMPQNVPAESGAQLLMVLRCLRPLRIFKLVPQMRKVVRELFSGFKEIFLVSILLLTLMLVFASFGVQLFAGKLAKCNDPNIIRREDCNGIFRINVSVSKNLNLKLRPGEKKPGFWVPRVWANPRNFNFDNVGNAMLALFEVLSLKGWVEVRDVIIHRVGPIHGIYIHVFVFLGCMIGLTLFVGVVIANFNENKGTALLTVDQRRWEDLKSRLKIAQPLHLPPRPDNDGFRAKMYDITQHPFFKRTIALLVLAQSVLLSVKWDVEDPVTVPLATMSVVFTFIFVLEVTMKIIAMSPAGFWQSRRNRYDLLVTSLGVVWVVLHFALLNAYTYMMGACVIVFRFFSICGKHVTLKMLLLTVVVSMYKSFFIIVGMFLLLLCYAFAGVVLFGTVKYGENINRHANFSSAGKAITVLFRIVTGEDWNKIMHDCMVQPPFCTPDEFTYWATDCGNYAGALMYFCSFYVIIAYIMLNLLVAIIVENFSLFYSTEEDQLLSYNDLRHFQIIWNMVDDKREGVIPTFRVKFLLRLLRGRLEVDLDKDKLLFKHMCYEMERLHNGGDVTFHDVLSMLSYRSVDIRKSLQLEELLAREQLEYTIEEEVAKQTIRMWLKKCLKRIRAKQQQSCSIIHSLRESQQQELSRFLNPPSIETTQPSEDTNANSQDNSMQPETSSQQQLLSPTLSDRGGSRQDAADAGKPQRKFGQWRLPSAPKPISHSVSSVNLRFGGRTTMKSVVCKMNPMTDAASCGSEVKKWWTRQLTVESDESGDDLLDI

SEQ ID NO: 41 - protein, 1738 aa, synthetic construct
  REGION 1..1738: note = Synthetic: hNALCN_W359E
MLKRKQSSRVEAQPVTDFGPDESLSDNADILWINKPWVHSLLRICAIISVISVCMNTPMTFEHYPPLQYVTFTLDTLLMFLYTAEMIAKMHIRGIVKGDSSYVKDRWCVFDGFMVFCLWVSLVLQVFEIADIVDQMSPWGMLRIPRPLIMIRAFRIYFRFELPRTRITNILKRSGEQIWSVSIFLLFFLLLYGILGVQMFGTFTYHCVVNDTKPGNVTWNSLAIPDTHCSPELEEGYQCPPGFKCMDLEDLGLSRQELGYSGFNEIGTSIFTVYEAASQEGWVFLMYRAIDSFPRWRSYFYFITLIFFLAWLVKNVFIAVIIETFAEIRVQFQQMWGSRSSTTSTATTQMFHEDAAGGEQLVAVDVNKPQGRAPACLQKMMRSSVFHMFILSMVTVDVIVAASNYYKGENFRRQYDEFYLAEVAFTVLFDLEALLKIWCLGFTGYISSSLHKFELLLVIGTTLHVYPDLYHSQFTYFQVLRVVRLIKISPALEDFVYKIFGPGKKLGSLVVFTASLLIVMSAISLQMFCFVEELDRFTTFPRAFMSMFQILTQEGWVDVMDQTLNAVGHMWAPVVAIYFILYHLFATLILLSLFVAVILDNLELDEDLKKLKQLKQSEANADTKEKLPLRLRIFEKFPNRPQMVKISKLPSDFTVPKIRESFMKQFIDRQQQDTCCLLRSLPTTSSSSCDHSKRSAIEDNKYIDQKLRKSVFSIRARNLLEKETAVTKILRACTRQRMLSGSFEGQPAKERSILSVQHHIRQERRSLRHGSNSQRISRGKSLETLTQDHSNTVRYRNAQREDSEIKMIQEKKEQAEMKRKVQEEELRENHPYFDKPLFIVGREHRFRNFCRVVVRARFNASKTDPVTGAVKNTKYHQLYDLLGLVTYLDWVMIIVTICSCISMMFESPFRRVMHAPTLQIAEYVFVIFMSIELNLKIMADGLFFTPTAVIRDFGGVMDIFIYLVSLIFLCWMPQNVPAESGAQLLMVLRCLRPLRIFKLVPQMRKVVRELFSGFKEIFLVSILLLTLMLVFASFGVQLFAGKLAKCNDPNIIRREDCNGIFRINVSVSKNLNLKLRPGEKKPGFWVPRVWANPRNFNFDNVGNAMLALFEVLSLKGWVEVRDVIIHRVGPIHGIYIHVFVFLGCMIGLTLFVGVVIANFNENKGTALLTVDQRRWEDLKSRLKIAQPLHLPPRPDNDGFRAKMYDITQHPFFKRTIALLVLAQSVLLSVKWDVEDPVTVPLATMSVVFTFIFVLEVTMKIIAMSPAGFWQSRRNRYDLLVTSLGVVWVVLHFALLNAYTYMMGACVIVFRFFSICGKHVTLKMLLLTVVVSMYKSFFIIVGMFLLLLCYAFAGVVLFGTVKYGENINRHANFSSAGKAITVLFRIVTGEDWNKIMHDCMVQPPFCTPDEFTYWATDCGNYAGALMYFCSFYVIIAYIMLNLLVAIIVENFSLFYSTEEDQLLSYNDLRHFQIIWNMVDDKREGVIPTFRVKFLLRLLRGRLEVDLDKDKLLFKHMCYEMERLHNGGDVTFHDVLSMLSYRSVDIRKSLQLEELLAREQLEYTIEEEVAKQTIRMWLKKCLKRIRAKQQQSCSIIHSLRESQQQELSRFLNPPSIETTQPSEDTNANSQDNSMQPETSSQQQLLSPTLSDRGGSRQDAADAGKPQRKFGQWRLPSAPKPISHSVSSVNLRFGGRTTMKSVVCKMNPMTDAASCGSEVKKWWTRQLTVESDESGDDLLDI

SEQ ID NO: 42 - protein, 1738 aa, synthetic construct
  REGION 1..1738: note = Synthetic: hNALCN-P340-P345 (S340P/S341P/T342P/T343P/S344P/T345P)
MLKRKQSSRVEAQPVTDFGPDESLSDNADILWINKPWVHSLLRICAIISVISVCMNTPMTFEHYPPLQYVTFTLDTLLMFLYTAEMIAKMHIRGIVKGDSSYVKDRWCVFDGFMVFCLWVSLVLQVFEIADIVDQMSPWGMLRIPRPLIMIRAFRIYFRFELPRTRITNILKRSGEQIWSVSIFLLFFLLLYGILGVQMFGTFTYHCVVNDTKPGNVTWNSLAIPDTHCSPELEEGYQCPPGFKCMDLEDLGLSRQELGYSGFNEIGTSIFTVYEAASQEGWVFLMYRAIDSFPRWRSYFYFITLIFFLAWLVKNVFIAVIIETFAEIRVQFQQMWGSRPPPPPPATTQMFHEDAAGGWQLVAVDVNKPQGRAPACLQKMMRSSVFHMFILSMVTVDVIVAASNYYKGENFRRQYDEFYLAEVAFTVLFDLEALLKIWCLGFTGYISSSLHKFELLLVIGTTLHVYPDLYHSQFTYFQVLRVVRLIKISPALEDFVYKIFGPGKKLGSLVVFTASLLIVMSAISLQMFCFVEELDRFTTFPRAFMSMFQILTQEGWVDVMDQTLNAVGHMWAPVVAIYFILYHLFATLILLSLFVAVILDNLELDEDLKKLKQLKQSEANADTKEKLPLRLRIFEKFPNRPQMVKISKLPSDFTVPKIRESFMKQFIDRQQQDTCCLLRSLPTTSSSSCDHSKRSAIEDNKYIDQKLRKSVFSIRARNLLEKETAVTKILRACTRQRMLSGSFEGQPAKERSILSVQHHIRQERRSLRHGSNSQRISRGKSLETLTQDHSNTVRYRNAQREDSEIKMIQEKKEQAEMKRKVQEEELRENHPYFDKPLFIVGREHRFRNFCRVVVRARFNASKTDPVTGAVKNTKYHQLYDLLGLVTYLDWVMIIVTICSCISMMFESPFRRVMHAPTLQIAEYVFVIFMSIELNLKIMADGLFFTPTAVIRDFGGVMDIFIYLVSLIFLCWMPQNVPAESGAQLLMVLRCLRPLRIFKLVPQMRKVVRELFSGFKEIFLVSILLLTLMLVFASFGVQLFAGKLAKCNDPNIIRREDCNGIFRINVSVSKNLNLKLRPGEKKPGFWVPRVWANPRNFNFDNVGNAMLALFEVLSLKGWVEVRDVIIHRVGPIHGIYIHVFVFLGCMIGLTLFVGVVIANFNENKGTALLTVDQRRWEDLKSRLKIAQPLHLPPRPDNDGFRAKMYDITQHPFFKRTIALLVLAQSVLLSVKWDVEDPVTVPLATMSVVFTFIFVLEVTMKIIAMSPAGFWQSRRNRYDLLVTSLGVVWVVLHFALLNAYTYMMGACVIVFRFFSICGKHVTLKMLLLTVVVSMYKSFFIIVGMFLLLLCYAFAGVVLFGTVKYGENINRHANFSSAGKAITVLFRIVTGEDWNKIMHDCMVQPPFCTPDEFTYWATDCGNYAGALMYFCSFYVIIAYIMLNLLVAIIVENFSLFYSTEEDQLLSYNDLRHFQIIWNMVDDKREGVIPTFRVKFLLRLLRGRLEVDLDKDKLLFKHMCYEMERLHNGGDVTFHDVLSMLSYRSVDIRKSLQLEELLAREQLEYTIEEEVAKQTIRMWLKKCLKRIRAKQQQSCSIIHSLRESQQQELSRFLNPPSIETTQPSEDTNANSQDNSMQPETSSQQQLLSPTLSDRGGSRQDAADAGKPQRKFGQWRLPSAPKPISHSVSSVNLRFGGRTTMKSVVCKMNPMTDAASCGSEVKKWWTRQLTVESDESGDDLLDI

SEQ ID NO: 43 - protein, 1732 aa, synthetic construct
  REGION 1..1732: note = Synthetic: hNALCN_delta-S340-T345
MLKRKQSSRVEAQPVTDFGPDESLSDNADILWINKPWVHSLLRICAIISVISVCMNTPMTFEHYPPLQYVTFTLDTLLMFLYTAEMIAKMHIRGIVKGDSSYVKDRWCVFDGFMVFCLWVSLVLQVFEIADIVDQMSPWGMLRIPRPLIMIRAFRIYFRFELPRTRITNILKRSGEQIWSVSIFLLFFLLLYGILGVQMFGTFTYHCVVNDTKPGNVTWNSLAIPDTHCSPELEEGYQCPPGFKCMDLEDLGLSRQELGYSGFNEIGTSIFTVYEAASQEGWVFLMYRAIDSFPRWRSYFYFITLIFFLAWLVKNVFIAVIIETFAEIRVQFQQMWGSRATTQMFHEDAAGGWQLVAVDVNKPQGRAPACLQKMMRSSVFHMFILSMVTVDVIVAASNYYKGENFRRQYDEFYLAEVAFTVLFDLEALLKIWCLGFTGYISSSLHKFELLLVIGTTLHVYPDLYHSQFTYFQVLRVVRLIKISPALEDFVYKIFGPGKKLGSLVVFTASLLIVMSAISLQMFCFVEELDRFTTFPRAFMSMFQILTQEGWVDVMDQTLNAVGHMWAPVVAIYFILYHLFATLILLSLFVAVILDNLELDEDLKKLKQLKQSEANADTKEKLPLRLRIFEKFPNRPQMVKISKLPSDFTVPKIRESFMKQFIDRQQQDTCCLLRSLPTTSSSSCDHSKRSAIEDNKYIDQKLRKSVFSIRARNLLEKETAVTKILRACTRQRMLSGSFEGQPAKERSILSVQHHIRQERRSLRHGSNSQRISRGKSLETLTQDHSNTVRYRNAQREDSEIKMIQEKKEQAEMKRKVQEEELRENHPYFDKPLFIVGREHRFRNFCRVVVRARFNASKTDPVTGAVKNTKYHQLYDLLGLVTYLDWVMIIVTICSCISMMFESPFRRVMHAPTLQIAEYVFVIFMSIELNLKIMADGLFFTPTAVIRDFGGVMDIFIYLVSLIFLCWMPQNVPAESGAQLLMVLRCLRPLRIFKLVPQMRKVVRELFSGFKEIFLVSILLLTLMLVFASFGVQLFAGKLAKCNDPNIIRREDCNGIFRINVSVSKNLNLKLRPGEKKPGFWVPRVWANPRNFNFDNVGNAMLALFEVLSLKGWVEVRDVIIHRVGPIHGIYIHVFVFLGCMIGLTLFVGVVIANFNENKGTALLTVDQRRWEDLKSRLKIAQPLHLPPRPDNDGFRAKMYDITQHPFFKRTIALLVLAQSVLLSVKWDVEDPVTVPLATMSVVFTFIFVLEVTMKIIAMSPAGFWQSRRNRYDLLVTSLGVVWVVLHFALLNAYTYMMGACVIVFRFFSICGKHVTLKMLLLTVVVSMYKSFFIIVGMFLLLLCYAFAGVVLFGTVKYGENINRHANFSSAGKAITVLFRIVTGEDWNKIMHDCMVQPPFCTPDEFTYWATDCGNYAGALMYFCSFYVIIAYIMLNLLVAIIVENFSLFYSTEEDQLLSYNDLRHFQIIWNMVDDKREGVIPTFRVKFLLRLLRGRLEVDLDKDKLLFKHMCYEMERLHNGGDVTFHDVLSMLSYRSVDIRKSLQLEELLAREQLEYTIEEEVAKQTIRMWLKKCLKRIRAKQQQSCSIIHSLRESQQQELSRFLNPPSIETTQPSEDTNANSQDNSMQPETSSQQQLLSPTLSDRGGSRQDAADAGKPQRKFGQWRLPSAPKPISHSVSSVNLRFGGRTTMKSVVCKMNPMTDAASCGSEVKKWWTRQLTVESDESGDDLLDI

SEQ ID NO: 44 - protein, 1750 aa, synthetic construct
  REGION 1..1750: note = Synthetic: hNALCN_T345-[GGGS]3-A346
MLKRKQSSRVEAQPVTDFGPDESLSDNADILWINKPWVHSLLRICAIISVISVCMNTPMTFEHYPPLQYVTFTLDTLLMFLYTAEMIAKMHIRGIVKGDSSYVKDRWCVFDGFMVFCLWVSLVLQVFEIADIVDQMSPWGMLRIPRPLIMIRAFRIYFRFELPRTRITNILKRSGEQIWSVSIFLLFFLLLYGILGVQMFGTFTYHCVVNDTKPGNVTWNSLAIPDTHCSPELEEGYQCPPGFKCMDLEDLGLSRQELGYSGFNEIGTSIFTVYEAASQEGWVFLMYRAIDSFPRWRSYFYFITLIFFLAWLVKNVFIAVIIETFAEIRVQFQQMWGSRSSTTSTGGGSGGGSGGGSATTQMFHEDAAGGWQLVAVDVNKPQGRAPACLQKMMRSSVFHMFILSMVTVDVIVAASNYYKGENFRRQYDEFYLAEVAFTVLFDLEALLKIWCLGFTGYISSSLHKFELLLVIGTTLHVYPDLYHSQFTYFQVLRVVRLIKISPALEDFVYKIFGPGKKLGSLVVFTASLLIVMSAISLQMFCFVEELDRFTTFPRAFMSMFQILTQEGWVDVMDQTLNAVGHMWAPVVAIYFILYHLFATLILLSLFVAVILDNLELDEDLKKLKQLKQSEANADTKEKLPLRLRIFEKFPNRPQMVKISKLPSDFTVPKIRESFMKQFIDRQQQDTCCLLRSLPTTSSSSCDHSKRSAIEDNKYIDQKLRKSVFSIRARNLLEKETAVTKILRACTRQRMLSGSFEGQPAKERSILSVQHHIRQERRSLRHGSNSQRISRGKSLETLTQDHSNTVRYRNAQREDSEIKMIQEKKEQAEMKRKVQEEELRENHPYFDKPLFIVGREHRFRNFCRVVVRARFNASKTDPVTGAVKNTKYHQLYDLLGLVTYLDWVMIIVTICSCISMMFESPFRRVMHAPTLQIAEYVFVIFMSIELNLKIMADGLFFTPTAVIRDFGGVMDIFIYLVSLIFLCWMPQNVPAESGAQLLMVLRCLRPLRIFKLVPQMRKVVRELFSGFKEIFLVSILLLTLMLVFASFGVQLFAGKLAKCNDPNIIRREDCNGIFRINVSVSKNLNLKLRPGEKKPGFWVPRVWANPRNFNFDNVGNAMLALFEVLSLKGWVEVRDVIIHRVGPIHGIYIHVFVFLGCMIGLTLFVGVVIANFNENKGTALLTVDQRRWEDLKSRLKIAQPLHLPPRPDNDGFRAKMYDITQHPFFKRTIALLVLAQSVLLSVKWDVEDPVTVPLATMSVVFTFIFVLEVTMKIIAMSPAGFWQSRRNRYDLLVTSLGVVWVVLHFALLNAYTYMMGACVIVFRFFSICGKHVTLKMLLLTVVVSMYKSFFIIVGMFLLLLCYAFAGVVLFGTVKYGENINRHANFSSAGKAITVLFRIVTGEDWNKIMHDCMVQPPFCTPDEFTYWATDCGNYAGALMYFCSFYVIIAYIMLNLLVAIIVENFSLFYSTEEDQLLSYNDLRHFQIIWNMVDDKREGVIPTFRVKFLLRLLRGRLEVDLDKDKLLFKHMCYEMERLHNGGDVTFHDVLSMLSYRSVDIRKSLQLEELLAREQLEYTIEEEVAKQTIRMWLKKCLKRIRAKQQQSCSIIHSLRESQQQELSRFLNPPSIETTQPSEDTNANSQDNSMQPETSSQQQLLSPTLSDRGGSRQDAADAGKPQRKFGQWRLPSAPKPISHSVSSVNLRFGGRTTMKSVVCKMNPMTDAASCGSEVKKWWTRQLTVESDESGDDLLDI

SEQ ID NO: 45 - protein, 1750 aa, synthetic construct
  REGION 1..1750: note = Synthetic: hNALCN_Q370-[GGGS]3-G371
MLKRKQSSRVEAQPVTDFGPDESLSDNADILWINKPWVHSLLRICAIISVISVCMNTPMTFEHYPPLQYVTFTLDTLLMFLYTAEMIAKMHIRGIVKGDSSYVKDRWCVFDGFMVFCLWVSLVLQVFEIADIVDQMSPWGMLRIPRPLIMIRAFRIYFRFELPRTRITNILKRSGEQIWSVSIFLLFFLLLYGILGVQMFGTFTYHCVVNDTKPGNVTWNSLAIPDTHCSPELEEGYQCPPGFKCMDLEDLGLSRQELGYSGFNEIGTSIFTVYEAASQEGWVFLMYRAIDSFPRWRSYFYFITLIFFLAWLVKNVFIAVIIETFAEIRVQFQQMWGSRSSTTSTATTQMFHEDAAGGWQLVAVDVNKPQGGGSGGGSGGGSGRAPACLQKMMRSSVFHMFILSMVTVDVIVAASNYYKGENFRRQYDEFYLAEVAFTVLFDLEALLKIWCLGFTGYISSSLHKFELLLVIGTTLHVYPDLYHSQFTYFQVLRVVRLIKISPALEDFVYKIFGPGKKLGSLVVFTASLLIVMSAISLQMFCFVEELDRFTTFPRAFMSMFQILTQEGWVDVMDQTLNAVGHMWAPVVAIYFILYHLFATLILLSLFVAVILDNLELDEDLKKLKQLKQSEANADTKEKLPLRLRIFEKFPNRPQMVKISKLPSDFTVPKIRESFMKQFIDRQQQDTCCLLRSLPTTSSSSCDHSKRSAIEDNKYIDQKLRKSVFSIRARNLLEKETAVTKILRACTRQRMLSGSFEGQPAKERSILSVQHHIRQERRSLRHGSNSQRISRGKSLETLTQDHSNTVRYRNAQREDSEIKMIQEKKEQAEMKRKVQEEELRENHPYFDKPLFIVGREHRFRNFCRVVVRARFNASKTDPVTGAVKNTKYHQLYDLLGLVTYLDWVMIIVTICSCISMMFESPFRRVMHAPTLQIAEYVFVIFMSIELNLKIMADGLFFTPTAVIRDFGGVMDIFIYLVSLIFLCWMPQNVPAESGAQLLMVLRCLRPLRIFKLVPQMRKVVRELFSGFKEIFLVSILLLTLMLVFASFGVQLFAGKLAKCNDPNIIRREDCNGIFRINVSVSKNLNLKLRPGEKKPGFWVPRVWANPRNFNFDNVGNAMLALFEVLSLKGWVEVRDVIIHRVGPIHGIYIHVFVFLGCMIGLTLFVGVVIANFNENKGTALLTVDQRRWEDLKSRLKIAQPLHLPPRPDNDGFRAKMYDITQHPFFKRTIALLVLAQSVLLSVKWDVEDPVTVPLATMSVVFTFIFVLEVTMKIIAMSPAGFWQSRRNRYDLLVTSLGVVWVVLHFALLNAYTYMMGACVIVFRFFSICGKHVTLKMLLLTVVVSMYKSFFIIVGMFLLLLCYAFAGVVLFGTVKYGENINRHANFSSAGKAITVLFRIVTGEDWNKIMHDCMVQPPFCTPDEFTYWATDCGNYAGALMYFCSFYVIIAYIMLNLLVAIIVENFSLFYSTEEDQLLSYNDLRHFQIIWNMVDDKREGVIPTFRVKFLLRLLRGRLEVDLDKDKLLFKHMCYEMERLHNGGDVTFHDVLSMLSYRSVDIRKSLQLEELLAREQLEYTIEEEVAKQTIRMWLKKCLKRIRAKQQQSCSIIHSLRESQQQELSRFLNPPSIETTQPSEDTNANSQDNSMQPETSSQQQLLSPTLSDRGGSRQDAADAGKPQRKFGQWRLPSAPKPISHSVSSVNLRFGGRTTMKSVVCKMNPMTDAASCGSEVKKWWTRQLTVESDESGDDLLDI

SEQ ID NO: 46 - protein, 1762 aa, synthetic construct
  REGION 1..1762: note = Synthetic: hNALCN_T345-[GGGS]3-A346+Q370-[GGGS]3-G371
MLKRKQSSRVEAQPVTDFGPDESLSDNADILWINKPWVHSLLRICAIISVISVCMNTPMTFEHYPPLQYVTFTLDTLLMFLYTAEMIAKMHIRGIVKGDSSYVKDRWCVFDGFMVFCLWVSLVLQVFEIADIVDQMSPWGMLRIPRPLIMIRAFRIYFRFELPRTRITNILKRSGEQIWSVSIFLLFFLLLYGILGVQMFGTFTYHCVVNDTKPGNVTWNSLAIPDTHCSPELEEGYQCPPGFKCMDLEDLGLSRQELGYSGFNEIGTSIFTVYEAASQEGWVFLMYRAIDSFPRWRSYFYFITLIFFLAWLVKNVFIAVIIETFAEIRVQFQQMWGSRSSTTSTGGGSGGGSGGGSATTQMFHEDAAGGWQLVAVDVNKPQGGGSGGGSGGGSGRAPACLQKMMRSSVFHMFILSMVTVDVIVAASNYYKGENFRRQYDEFYLAEVAFTVLFDLEALLKIWCLGFTGYISSSLHKFELLLVIGTTLHVYPDLYHSQFTYFQVLRVVRLIKISPALEDFVYKIFGPGKKLGSLVVFTASLLIVMSAISLQMFCFVEELDRFTTFPRAFMSMFQILTQEGWVDVMDQTLNAVGHMWAPVVAIYFILYHLFATLILLSLFVAVILDNLELDEDLKKLKQLKQSEANADTKEKLPLRLRIFEKFPNRPQMVKISKLPSDFTVPKIRESFMKQFIDRQQQDTCCLLRSLPTTSSSSCDHSKRSAIEDNKYIDQKLRKSVFSIRARNLLEKETAVTKILRACTRQRMLSGSFEGQPAKERSILSVQHHIRQERRSLRHGSNSQRISRGKSLETLTQDHSNTVRYRNAQREDSEIKMIQEKKEQAEMKRKVQEEELRENHPYFDKPLFIVGREHRFRNFCRVVVRARFNASKTDPVTGAVKNTKYHQLYDLLGLVTYLDWVMIIVTICSCISMMFESPFRRVMHAPTLQIAEYVFVIFMSIELNLKIMADGLFFTPTAVIRDFGGVMDIFIYLVSLIFLCWMPQNVPAESGAQLLMVLRCLRPLRIFKLVPQMRKVVRELFSGFKEIFLVSILLLTLMLVFASFGVQLFAGKLAKCNDPNIIRREDCNGIFRINVSVSKNLNLKLRPGEKKPGFWVPRVWANPRNFNFDNVGNAMLALFEVLSLKGWVEVRDVIIHRVGPIHGIYIHVFVFLGCMIGLTLFVGVVIANFNENKGTALLTVDQRRWEDLKSRLKIAQPLHLPPRPDNDGFRAKMYDITQHPFFKRTIALLVLAQSVLLSVKWDVEDPVTVPLATMSVVFTFIFVLEVTMKIIAMSPAGFWQSRRNRYDLLVTSLGVVWVVLHFALLNAYTYMMGACVIVFRFFSICGKHVTLKMLLLTVVVSMYKSFFIIVGMFLLLLCYAFAGVVLFGTVKYGENINRHANFSSAGKAITVLFRIVTGEDWNKIMHDCMVQPPFCTPDEFTYWATDCGNYAGALMYFCSFYVIIAYIMLNLLVAIIVENFSLFYSTEEDQLLSYNDLRHFQIIWNMVDDKREGVIPTFRVKFLLRLLRGRLEVDLDKDKLLFKHMCYEMERLHNGGDVTFHDVLSMLSYRSVDIRKSLQLEELLAREQLEYTIEEEVAKQTIRMWLKKCLKRIRAKQQQSCSIIHSLRESQQQELSRFLNPPSIETTQPSEDTNANSQDNSMQPETSSQQQLLSPTLSDRGGSRQDAADAGKPQRKFGQWRLPSAPKPISHSVSSVNLRFGGRTTMKSVVCKMNPMTDAASCGSEVKKWWTRQLTVESDESGDDLLDI

SEQ ID NO: 47 - protein, 1738 aa, synthetic construct
  REGION 1..1738: note = Synthetic: hNALCN_P621-626 (A621P/D622P/T623P/K624P/E625P/K626P)
MLKRKQSSRVEAQPVTDFGPDESLSDNADILWINKPWVHSLLRICAIISVISVCMNTPMTFEHYPPLQYVTFTLDTLLMFLYTAEMIAKMHIRGIVKGDSSYVKDRWCVFDGFMVFCLWVSLVLQVFEIADIVDQMSPWGMLRIPRPLIMIRAFRIYFRFELPRTRITNILKRSGEQIWSVSIFLLFFLLLYGILGVQMFGTFTYHCVVNDTKPGNVTWNSLAIPDTHCSPELEEGYQCPPGFKCMDLEDLGLSRQELGYSGFNEIGTSIFTVYEAASQEGWVFLMYRAIDSFPRWRSYFYFITLIFFLAWLVKNVFIAVIIETFAEIRVQFQQMWGSRSSTTSTATTQMFHEDAAGGWQLVAVDVNKPQGRAPACLQKMMRSSVFHMFILSMVTVDVIVAASNYYKGENFRRQYDEFYLAEVAFTVLFDLEALLKIWCLGFTGYISSSLHKFELLLVIGTTLHVYPDLYHSQFTYFQVLRVVRLIKISPALEDFVYKIFGPGKKLGSLVVFTASLLIVMSAISLQMFCFVEELDRFTTFPRAFMSMFQILTQEGWVDVMDQTLNAVGHMWAPVVAIYFILYHLFATLILLSLFVAVILDNLELDEDLKKLKQLKQSEANPPPPPPLPLRLRIFEKFPNRPQMVKISKLPSDFTVPKIRESFMKQFIDRQQQDTCCLLRSLPTTSSSSCDHSKRSAIEDNKYIDQKLRKSVFSIRARNLLEKETAVTKILRACTRQRMLSGSFEGQPAKERSILSVQHHIRQERRSLRHGSNSQRISRGKSLETLTQDHSNTVRYRNAQREDSEIKMIQEKKEQAEMKRKVQEEELRENHPYFDKPLFIVGREHRFRNFCRVVVRARFNASKTDPVTGAVKNTKYHQLYDLLGLVTYLDWVMIIVTICSCISMMFESPFRRVMHAPTLQIAEYVFVIFMSIELNLKIMADGLFFTPTAVIRDFGGVMDIFIYLVSLIFLCWMPQNVPAESGAQLLMVLRCLRPLRIFKLVPQMRKVVRELFSGFKEIFLVSILLLTLMLVFASFGVQLFAGKLAKCNDPNIIRREDCNGIFRINVSVSKNLNLKLRPGEKKPGFWVPRVWANPRNFNFDNVGNAMLALFEVLSLKGWVEVRDVIIHRVGPIHGIYIHVFVFLGCMIGLTLFVGVVIANFNENKGTALLTVDQRRWEDLKSRLKIAQPLHLPPRPDNDGFRAKMYDITQHPFFKRTIALLVLAQSVLLSVKWDVEDPVTVPLATMSVVFTFIFVLEVTMKIIAMSPAGFWQSRRNRYDLLVTSLGVVWVVLHFALLNAYTYMMGACVIVFRFFSICGKHVTLKMLLLTVVVSMYKSFFIIVGMFLLLLCYAFAGVVLFGTVKYGENINRHANFSSAGKAITVLFRIVTGEDWNKIMHDCMVQPPFCTPDEFTYWATDCGNYAGALMYFCSFYVIIAYIMLNLLVAIIVENFSLFYSTEEDQLLSYNDLRHFQIIWNMVDDKREGVIPTFRVKFLLRLLRGRLEVDLDKDKLLFKHMCYEMERLHNGGDVTFHDVLSMLSYRSVDIRKSLQLEELLAREQLEYTIEEEVAKQTIRMWLKKCLKRIRAKQQQSCSIIHSLRESQQQELSRFLNPPSIETTQPSEDTNANSQDNSMQPETSSQQQLLSPTLSDRGGSRQDAADAGKPQRKFGQWRLPSAPKPISHSVSSVNLRFGGRTTMKSVVCKMNPMTDAASCGSEVKKWWTRQLTVESDESGDDLLDI

SEQ ID NO: 48 - protein, 1732 aa, synthetic construct
  REGION 1..1732: note = Synthetic: hNALCN_delta-A621-K626
MLKRKQSSRVEAQPVTDFGPDESLSDNADILWINKPWVHSLLRICAIISVISVCMNTPMTFEHYPPLQYVTFTLDTLLMFLYTAEMIAKMHIRGIVKGDSSYVKDRWCVFDGFMVFCLWVSLVLQVFEIADIVDQMSPWGMLRIPRPLIMIRAFRIYFRFELPRTRITNILKRSGEQIWSVSIFLLFFLLLYGILGVQMFGTFTYHCVVNDTKPGNVTWNSLAIPDTHCSPELEEGYQCPPGFKCMDLEDLGLSRQELGYSGFNEIGTSIFTVYEAASQEGWVFLMYRAIDSFPRWRSYFYFITLIFFLAWLVKNVFIAVIIETFAEIRVQFQQMWGSRSSTTSTATTQMFHEDAAGGWQLVAVDVNKPQGRAPACLQKMMRSSVFHMFILSMVTVDVIVAASNYYKGENFRRQYDEFYLAEVAFTVLFDLEALLKIWCLGFTGYISSSLHKFELLLVIGTTLHVYPDLYHSQFTYFQVLRVVRLIKISPALEDFVYKIFGPGKKLGSLVVFTASLLIVMSAISLQMFCFVEELDRFTTFPRAFMSMFQILTQEGWVDVMDQTLNAVGHMWAPVVAIYFILYHLFATLILLSLFVAVILDNLELDEDLKKLKQLKQSEANLPLRLRIFEKFPNRPQMVKISKLPSDFTVPKIRESFMKQFIDRQQQDTCCLLRSLPTTSSSSCDHSKRSAIEDNKYIDQKLRKSVFSIRARNLLEKETAVTKILRACTRQRMLSGSFEGQPAKERSILSVQHHIRQERRSLRHGSNSQRISRGKSLETLTQDHSNTVRYRNAQREDSEIKMIQEKKEQAEMKRKVQEEELRENHPYFDKPLFIVGREHRFRNFCRVVVRARFNASKTDPVTGAVKNTKYHQLYDLLGLVTYLDWVMIIVTICSCISMMFESPFRRVMHAPTLQIAEYVFVIFMSIELNLKIMADGLFFTPTAVIRDFGGVMDIFIYLVSLIFLCWMPQNVPAESGAQLLMVLRCLRPLRIFKLVPQMRKVVRELFSGFKEIFLVSILLLTLMLVFASFGVQLFAGKLAKCNDPNIIRREDCNGIFRINVSVSKNLNLKLRPGEKKPGFWVPRVWANPRNFNFDNVGNAMLALFEVLSLKGWVEVRDVIIHRVGPIHGIYIHVFVFLGCMIGLTLFVGVVIANFNENKGTALLTVDQRRWEDLKSRLKIAQPLHLPPRPDNDGFRAKMYDITQHPFFKRTIALLVLAQSVLLSVKWDVEDPVTVPLATMSVVFTFIFVLEVTMKIIAMSPAGFWQSRRNRYDLLVTSLGVVWVVLHFALLNAYTYMMGACVIVFRFFSICGKHVTLKMLLLTVVVSMYKSFFIIVGMFLLLLCYAFAGVVLFGTVKYGENINRHANFSSAGKAITVLFRIVTGEDWNKIMHDCMVQPPFCTPDEFTYWATDCGNYAGALMYFCSFYVIIAYIMLNLLVAIIVENFSLFYSTEEDQLLSYNDLRHFQIIWNMVDDKREGVIPTFRVKFLLRLLRGRLEVDLDKDKLLFKHMCYEMERLHNGGDVTFHDVLSMLSYRSVDIRKSLQLEELLAREQLEYTIEEEVAKQTIRMWLKKCLKRIRAKQQQSCSIIHSLRESQQQELSRFLNPPSIETTQPSEDTNANSQDNSMQPETSSQQQLLSPTLSDRGGSRQDAADAGKPQRKFGQWRLPSAPKPISHSVSSVNLRFGGRTTMKSVVCKMNPMTDAASCGSEVKKWWTRQLTVESDESGDDLLDI

SEQ ID NO: 49 - protein, 1750 aa, synthetic construct
  REGION 1..1750: note = Synthetic: hNALCN_K626-[GGGS]3-L627
MLKRKQSSRVEAQPVTDFGPDESLSDNADILWINKPWVHSLLRICAIISVISVCMNTPMTFEHYPPLQYVTFTLDTLLMFLYTAEMIAKMHIRGIVKGDSSYVKDRWCVFDGFMVFCLWVSLVLQVFEIADIVDQMSPWGMLRIPRPLIMIRAFRIYFRFELPRTRITNILKRSGEQIWSVSIFLLFFLLLYGILGVQMFGTFTYHCVVNDTKPGNVTWNSLAIPDTHCSPELEEGYQCPPGFKCMDLEDLGLSRQELGYSGFNEIGTSIFTVYEAASQEGWVFLMYRAIDSFPRWRSYFYFITLIFFLAWLVKNVFIAVIIETFAEIRVQFQQMWGSRSSTTSTATTQMFHEDAAGGWQLVAVDVNKPQGRAPACLQKMMRSSVFHMFILSMVTVDVIVAASNYYKGENFRRQYDEFYLAEVAFTVLFDLEALLKIWCLGFTGYISSSLHKFELLLVIGTTLHVYPDLYHSQFTYFQVLRVVRLIKISPALEDFVYKIFGPGKKLGSLVVFTASLLIVMSAISLQMFCFVEELDRFTTFPRAFMSMFQILTQEGWVDVMDQTLNAVGHMWAPVVAIYFILYHLFATLILLSLFVAVILDNLELDEDLKKLKQLKQSEANADTKEKGGGSGGGSGGGSLPLRLRIFEKFPNRPQMVKISKLPSDFTVPKIRESFMKQFIDRQQQDTCCLLRSLPTTSSSSCDHSKRSAIEDNKYIDQKLRKSVFSIRARNLLEKETAVTKILRACTRQRMLSGSFEGQPAKERSILSVQHHIRQERRSLRHGSNSQRISRGKSLETLTQDHSNTVRYRNAQREDSEIKMIQEKKEQAEMKRKVQEEELRENHPYFDKPLFIVGREHRFRNFCRVVVRARFNASKTDPVTGAVKNTKYHQLYDLLGLVTYLDWVMIIVTICSCISMMFESPFRRVMHAPTLQIAEYVFVIFMSIELNLKIMADGLFFTPTAVIRDFGGVMDIFIYLVSLIFLCWMPQNVPAESGAQLLMVLRCLRPLRIFKLVPQMRKVVRELFSGFKEIFLVSILLLTLMLVFASFGVQLFAGKLAKCNDPNIIRREDCNGIFRINVSVSKNLNLKLRPGEKKPGFWVPRVWANPRNFNFDNVGNAMLALFEVLSLKGWVEVRDVIIHRVGPIHGIYIHVFVFLGCMIGLTLFVGVVIANFNENKGTALLTVDQRRWEDLKSRLKIAQPLHLPPRPDNDGFRAKMYDITQHPFFKRTIALLVLAQSVLLSVKWDVEDPVTVPLATMSVVFTFIFVLEVTMKIIAMSPAGFWQSRRNRYDLLVTSLGVVWVVLHFALLNAYTYMMGACVIVFRFFSICGKHVTLKMLLLTVVVSMYKSFFIIVGMFLLLLCYAFAGVVLFGTVKYGENINRHANFSSAGKAITVLFRIVTGEDWNKIMHDCMVQPPFCTPDEFTYWATDCGNYAGALMYFCSFYVIIAYIMLNLLVAIIVENFSLFYSTEEDQLLSYNDLRHFQIIWNMVDDKREGVIPTFRVKFLLRLLRGRLEVDLDKDKLLFKHMCYEMERLHNGGDVTFHDVLSMLSYRSVDIRKSLQLEELLAREQLEYTIEEEVAKQTIRMWLKKCLKRIRAKQQQSCSIIHSLRESQQQELSRFLNPPSIETTQPSEDTNANSQDNSMQPETSSQQQLLSPTLSDRGGSRQDAADAGKPQRKFGQWRLPSAPKPISHSVSSVNLRFGGRTTMKSVVCKMNPMTDAASCGSEVKKWWTRQLTVESDESGDDLLDI

SEQ ID NO: 50 - protein, 1750 aa, synthetic construct
  REGION 1..1750: note = Synthetic: hNALCN_K835-[GGGS]3-P836
MLKRKQSSRVEAQPVTDFGPDESLSDNADILWINKPWVHSLLRICAIISVISVCMNTPMTFEHYPPLQYVTFTLDTLLMFLYTAEMIAKMHIRGIVKGDSSYVKDRWCVFDGFMVFCLWVSLVLQVFEIADIVDQMSPWGMLRIPRPLIMIRAFRIYFRFELPRTRITNILKRSGEQIWSVSIFLLFFLLLYGILGVQMFGTFTYHCVVNDTKPGNVTWNSLAIPDTHCSPELEEGYQCPPGFKCMDLEDLGLSRQELGYSGFNEIGTSIFTVYEAASQEGWVFLMYRAIDSFPRWRSYFYFITLIFFLAWLVKNVFIAVIIETFAEIRVQFQQMWGSRSSTTSTATTQMFHEDAAGGWQLVAVDVNKPQGRAPACLQKMMRSSVFHMFILSMVTVDVIVAASNYYKGENFRRQYDEFYLAEVAFTVLFDLEALLKIWCLGFTGYISSSLHKFELLLVIGTTLHVYPDLYHSQFTYFQVLRVVRLIKISPALEDFVYKIFGPGKKLGSLVVFTASLLIVMSAISLQMFCFVEELDRFTTFPRAFMSMFQILTQEGWVDVMDQTLNAVGHMWAPVVAIYFILYHLFATLILLSLFVAVILDNLELDEDLKKLKQLKQSEANADTKEKLPLRLRIFEKFPNRPQMVKISKLPSDFTVPKIRESFMKQFIDRQQQDTCCLLRSLPTTSSSSCDHSKRSAIEDNKYIDQKLRKSVFSIRARNLLEKETAVTKILRACTRQRMLSGSFEGQPAKERSILSVQHHIRQERRSLRHGSNSQRISRGKSLETLTQDHSNTVRYRNAQREDSEIKMIQEKKEQAEMKRKVQEEELRENHPYFDKGGGSGGGSGGGSPLFIVGREHRFRNFCRVVVRARFNASKTDPVTGAVKNTKYHQLYDLLGLVTYLDWVMIIVTICSCISMMFESPFRRVMHAPTLQIAEYVFVIFMSIELNLKIMADGLFFTPTAVIRDFGGVMDIFIYLVSLIFLCWMPQNVPAESGAQLLMVLRCLRPLRIFKLVPQMRKVVRELFSGFKEIFLVSILLLTLMLVFASFGVQLFAGKLAKCNDPNIIRREDCNGIFRINVSVSKNLNLKLRPGEKKPGFWVPRVWANPRNFNFDNVGNAMLALFEVLSLKGWVEVRDVIIHRVGPIHGIYIHVFVFLGCMIGLTLFVGVVIANFNENKGTALLTVDQRRWEDLKSRLKIAQPLHLPPRPDNDGFRAKMYDITQHPFFKRTIALLVLAQSVLLSVKWDVEDPVTVPLATMSVVFTFIFVLEVTMKIIAMSPAGFWQSRRNRYDLLVTSLGVVWVVLHFALLNAYTYMMGACVIVFRFFSICGKHVTLKMLLLTVVVSMYKSFFIIVGMFLLLLCYAFAGVVLFGTVKYGENINRHANFSSAGKAITVLFRIVTGEDWNKIMHDCMVQPPFCTPDEFTYWATDCGNYAGALMYFCSFYVIIAYIMLNLLVAIIVENFSLFYSTEEDQLLSYNDLRHFQIIWNMVDDKREGVIPTFRVKFLLRLLRGRLEVDLDKDKLLFKHMCYEMERLHNGGDVTFHDVLSMLSYRSVDIRKSLQLEELLAREQLEYTIEEEVAKQTIRMWLKKCLKRIRAKQQQSCSIIHSLRESQQQELSRFLNPPSIETTQPSEDTNANSQDNSMQPETSSQQQLLSPTLSDRGGSRQDAADAGKPQRKFGQWRLPSAPKPISHSVSSVNLRFGGRTTMKSVVCKMNPMTDAASCGSEVKKWWTRQLTVESDESGDDLLDI

SEQ ID NO: 51 - protein, 1762 aa, synthetic construct
  REGION 1..1762: note = Synthetic: hNALCN_K626-[GGGS]3-L627+K835-[GGGS]3-P836
MLKRKQSSRVEAQPVTDFGPDESLSDNADILWINKPWVHSLLRICAIISVISVCMNTPMTFEHYPPLQYVTFTLDTLLMFLYTAEMIAKMHIRGIVKGDSSYVKDRWCVFDGFMVFCLWVSLVLQVFEIADIVDQMSPWGMLRIPRPLIMIRAFRIYFRFELPRTRITNILKRSGEQIWSVSIFLLFFLLLYGILGVQMFGTFTYHCVVNDTKPGNVTWNSLAIPDTHCSPELEEGYQCPPGFKCMDLEDLGLSRQELGYSGFNEIGTSIFTVYEAASQEGWVFLMYRAIDSFPRWRSYFYFITLIFFLAWLVKNVFIAVIIETFAEIRVQFQQMWGSRSSTTSTATTQMFHEDAAGGWQLVAVDVNKPQGRAPACLQKMMRSSVFHMFILSMVTVDVIVAASNYYKGENFRRQYDEFYLAEVAFTVLFDLEALLKIWCLGFTGYISSSLHKFELLLVIGTTLHVYPDLYHSQFTYFQVLRVVRLIKISPALEDFVYKIFGPGKKLGSLVVFTASLLIVMSAISLQMFCFVEELDRFTTFPRAFMSMFQILTQEGWVDVMDQTLNAVGHMWAPVVAIYFILYHLFATLILLSLFVAVILDNLELDEDLKKLKQLKQSEANADTKEKGGGSGGGSGGGSLPLRLRIFEKFPNRPQMVKISKLPSDFTVPKIRESFMKQFIDRQQQDTCCLLRSLPTTSSSSCDHSKRSAIEDNKYIDQKLRKSVFSIRARNLLEKETAVTKILRACTRQRMLSGSFEGQPAKERSILSVQHHIRQERRSLRHGSNSQRISRGKSLETLTQDHSNTVRYRNAQREDSEIKMIQEKKEQAEMKRKVQEEELRENHPYFDKGGGSGGGSGGGSPLFIVGREHRFRNFCRVVVRARFNASKTDPVTGAVKNTKYHQLYDLLGLVTYLDWVMIIVTICSCISMMFESPFRRVMHAPTLQIAEYVFVIFMSIELNLKIMADGLFFTPTAVIRDFGGVMDIFIYLVSLIFLCWMPQNVPAESGAQLLMVLRCLRPLRIFKLVPQMRKVVRELFSGFKEIFLVSILLLTLMLVFASFGVQLFAGKLAKCNDPNIIRREDCNGIFRINVSVSKNLNLKLRPGEKKPGFWVPRVWANPRNFNFDNVGNAMLALFEVLSLKGWVEVRDVIIHRVGPIHGIYIHVFVFLGCMIGLTLFVGVVIANFNENKGTALLTVDQRRWEDLKSRLKIAQPLHLPPRPDNDGFRAKMYDITQHPFFKRTIALLVLAQSVLLSVKWDVEDPVTVPLATMSVVFTFIFVLEVTMKIIAMSPAGFWQSRRNRYDLLVTSLGVVWVVLHFALLNAYTYMMGACVIVFRFFSICGKHVTLKMLLLTVVVSMYKSFFIIVGMFLLLLCYAFAGVVLFGTVKYGENINRHANFSSAGKAITVLFRIVTGEDWNKIMHDCMVQPPFCTPDEFTYWATDCGNYAGALMYFCSFYVIIAYIMLNLLVAIIVENFSLFYSTEEDQLLSYNDLRHFQIIWNMVDDKREGVIPTFRVKFLLRLLRGRLEVDLDKDKLLFKHMCYEMERLHNGGDVTFHDVLSMLSYRSVDIRKSLQLEELLAREQLEYTIEEEVAKQTIRMWLKKCLKRIRAKQQQSCSIIHSLRESQQQELSRFLNPPSIETTQPSEDTNANSQDNSMQPETSSQQQLLSPTLSDRGGSRQDAADAGKPQRKFGQWRLPSAPKPISHSVSSVNLRFGGRTTMKSVVCKMNPMTDAASCGSEVKKWWTRQLTVESDESGDDLLDI

SEQ ID NO: 52 - protein, 2669 aa, synthetic construct
  REGION 1..2669: note = Synthetic: hUNC79_1-2400*
MSTKAEQFASKIRYLQEYHNRVLHNIYPVPSGTDIANTLKYFSQTLLSILSRTGKKENQDASNLTVPMTMCLFPVPFPLTPSLRPQVSSINPTVTRSLLYSVLRDAPSERGPQSRDAQLSDYPSLDYQGLYVTLVTLLDLVPLLQHGQHDLGQSIFYTTTCLLPFLNDDILSTLPYTMISTLATFPPFLHKDIIEYLSTSFLPMAILGSSRREGVPAHVNLSASSMLMIAMQYTSNPVYHCQLLECLMKYKQEVWKDLLYVIAYGPSQVKPPAVQMLFHYWPNLKPPGAISEYRGLQYTAWNPIHCQHIECHNAINKPAVKMCIDPSLSVALGDKPPPLYLCEECSERIAGDHSEWLIDVLLPQAEISAICQKKNCSSHVRRAVVTCFSAGCCGRHGNRPVRYCKRCHSNHHSNEVGAAAETHLYQTSPPPINTRECGAEELVCAVEAVISLLKEAEFHAEQREHELNRRRQLGLSSSHHSLDNADFDNKDDDKHDQRLLSQFGIWFLVSLCTPSENTPTESLARLVAMVFQWFHSTAYMMDDEVGSLVEKLKPQFVTKWLKTVCDVRFDVMVMCLLPKPMEFARVGGYWDKSCSTVTQLKEGLNRILCLIPYNVINQSVWECIMPEWLEAIRTEVPDNQLKEFREVLSKMFDIELCPLPFSMEEMFGFISCRFTGYPSSVQEQALLWLHVLSELDIMVPLQLLISMFSDGVNSVKELANQRKSRVSELAGNLASRRVSVASDPGRRVQHNMLSPFHSPFQSPFRSPLRSPFRSPFKNFGHPGGRTIDFDCEDDEMNLNCFILMFDLLLKQMELQDDGITMGLEHSLSKDIISIINNVFQAPWGGSHTCQKDEKAIECNLCQSSILCYQLACELLERLAPKEESRLVEPTDSLEDSLLSSRPEFIIGPEGEEEENPASKHGENPGNCTEPVEHAAVKNDTERKFCYQQLPVTLRLIYTIFQEMAKFEEPDILFNMLNCLKILCLHGECLYIARKDHPQFLAYIQDHMLIASLWRVVKSEFSQLSSLAVPLLLHALSLPHGADIFWTIINGNFNSKDWKMRFEAVEKVAVICRFLDIHSVTKNHLLKYSLAHAFCCFLTAVEDVNPAVATRAGLLLDTIKRPALQGLCLCLDFQFDTVVKDRPTILSKLLLLHFLKQDIPALSWEFFVNRFETLSLEAQLHLDCNKEFPFPTTITAVRTNVANLSDAALWKIKRARFARNRQKSVRSLRDSVKGPVESKRALSLPETLTSKIRQQSPENDNTIKDLLPEDAGIDHQTVHQLITVLMKFMAKDESSAESDISSAKAFNTVKRHLYVLLGYDQQEGCFMIAPQKMRLSTCFNAFIAGIAQVMDYNINLGKHLLPLVVQVLKYCSCPQLRHYFQQPPRCSLWSLKPHIRQMWLKALLVILYKYPYRDCDISKILLHLIHITVNTLNAQYHSCKPHATAGPLYSDNSNISRYSEKEKGEIELAEYRETGALQDSLLHCVREESIPKKKLRSFKQKSLDIGNADSLLFTLDEHRRKSCIDRCDIEKPPTQAAYIAQRPNDPGRSRQNSATRPDNSEIPENPAMEGFPDARRPVIPEVRLNCMETFEVKVDSPVKPAPKEDLDLIDLSSDSTSGPEKHSILSTSDSDSLVFEPLPPLRIVESDEEEETMNQGDDGPSGKNAASSPSVPSHPSVLSLSTAPLVQVSVEDCSKDFSSKDSGNNQSAGNTDSALITLEDPMDAEGSSKPEELPEFSCGSPLTLKQKRDLLQKSFALPEMSLDDHPDPGTEGEKPGELMPSSGAKTVLLKVPEDAENPTESEKPDTSAESDTEQNPERKVEEDGAEESEFKIQIVPRQRKQRKIAVSAIQREYLDISFNILDKLGEQKDPDPSTKGLSTLEMPRESSSAPTLDAGVPETSSHSSISTQYRQMKRGSLGVLTMSQLMKRQLEHQSSAPHNISNWDTEQIQPGKRQCNVPTCLNPDLEGQPLRMRGATKSSLLSAPSIVSMFVPAPEEFTDEQPTVMTDKCHDCGAILEEYDEETLGLAIVVLSTFIHLSPDLAAPLLLDIMQSVGRLASSTTFSNQAESMMVPGNAAGVAKQFLRCIFHQLAPNGIFPQLFQSTIKDGTFLRTLASSLMDFNELSSIAALSQLLEGLNNKKNLPAGGAMIRCLENIATFMEALPMDSPSSLWTTISNQFQTFFAKLPCVLPLKCSLDSSLRIMICLLKIPSTNATRSLLEPFSKLLSFVIQNAVFTLAYLVELCGLCYRAFTKERDKFYLSRSVVLELLQALKLKSPLPDTNLLLLVQFICADAGTKLAESTILSKQMIASVPGCGTAAMECVRQYINEVLDFMADMHTLTKLKSHMKTCSQPLHEDTFGGHLKVGLAQIAAMDISRGNHRDNKAVIRYLPWLYHPPSAMQQGGSGGSVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKGGSGGSDYKDDDDKGSGDYKDDDDK

SEQ ID NO: 53 - protein, 736 aa, synthetic construct
  REGION 1..736: note = Synthetic: hUNC79_1-467*
MSTKAEQFASKIRYLQEYHNRVLHNIYPVPSGTDIANTLKYFSQTLLSILSRTGKKENQDASNLTVPMTMCLFPVPFPLTPSLRPQVSSINPTVTRSLLYSVLRDAPSERGPQSRDAQLSDYPSLDYQGLYVTLVTLLDLVPLLQHGQHDLGQSIFYTTTCLLPFLNDDILSTLPYTMISTLATFPPFLHKDIIEYLSTSFLPMAILGSSRREGVPAHVNLSASSMLMIAMQYTSNPVYHCQLLECLMKYKQEVWKDLLYVIAYGPSQVKPPAVQMLFHYWPNLKPPGAISEYRGLQYTAWNPIHCQHIECHNAINKPAVKMCIDPSLSVALGDKPPPLYLCEECSERIAGDHSEWLIDVLLPQAEISAICQKKNCSSHVRRAVVTCFSAGCCGRHGNRPVRYCKRCHSNHHSNEVGAAAETHLYQTSPPPINTRECGAEELVCAVEAVISLLKEAEFHAEQREHELGGSGGSVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKGGSGGSDYKDDDDKGSGDYKDDDDK

SEQ ID NO: 54 - protein, 2438 aa, synthetic construct
  REGION 1..2438: note = Synthetic: hUNC79_468-2635*
MNRRRQLGLSSSHHSLDNADFDNKDDDKHDQRLLSQFGIWFLVSLCTPSENTPTESLARLVAMVFQWFHSTAYMMDDEVGSLVEKLKPQFVTKWLKTVCDVRFDVMVMCLLPKPMEFARVGGYWDKSCSTVTQLKEGLNRILCLIPYNVINQSVWECIMPEWLEAIRTEVPDNQLKEFREVLSKMFDIELCPLPFSMEEMFGFISCRFTGYPSSVQEQALLWLHVLSELDIMVPLQLLISMFSDGVNSVKELANQRKSRVSELAGNLASRRVSVASDPGRRVQHNMLSPFHSPFQSPFRSPLRSPFRSPFKNFGHPGGRTIDFDCEDDEMNLNCFILMFDLLLKQMELQDDGITMGLEHSLSKDIISIINNVFQAPWGGSHTCQKDEKAIECNLCQSSILCYQLACELLERLAPKEESRLVEPTDSLEDSLLSSRPEFIIGPEGEEEENPASKHGENPGNCTEPVEHAAVKNDTERKFCYQQLPVTLRLIYTIFQEMAKFEEPDILFNMLNCLKILCLHGECLYIARKDHPQFLAYIQDHMLIASLWRVVKSEFSQLSSLAVPLLLHALSLPHGADIFWTIINGNFNSKDWKMRFEAVEKVAVICRFLDIHSVTKNHLLKYSLAHAFCCFLTAVEDVNPAVATRAGLLLDTIKRPALQGLCLCLDFQFDTVVKDRPTILSKLLLLHFLKQDIPALSWEFFVNRFETLSLEAQLHLDCNKEFPFPTTITAVRTNVANLSDAALWKIKRARFARNRQKSVRSLRDSVKGPVESKRALSLPETLTSKIRQQSPENDNTIKDLLPEDAGIDHQTVHQLITVLMKFMAKDESSAESDISSAKAFNTVKRHLYVLLGYDQQEGCFMIAPQKMRLSTCFNAFIAGIAQVMDYNINLGKHLLPLVVQVLKYCSCPQLRHYFQQPPRCSLWSLKPHIRQMWLKALLVILYKYPYRDCDISKILLHLIHITVNTLNAQYHSCKPHATAGPLYSDNSNISRYSEKEKGEIELAEYRETGALQDSLLHCVREESIPKKKLRSFKQKSLDIGNADSLLFTLDEHRRKSCIDRCDIEKPPTQAAYIAQRPNDPGRSRQNSATRPDNSEIPENPAMEGFPDARRPVIPEVRLNCMETFEVKVDSPVKPAPKEDLDLIDLSSDSTSGPEKHSILSTSDSDSLVFEPLPPLRIVESDEEEETMNQGDDGPSGKNAASSPSVPSHPSVLSLSTAPLVQVSVEDCSKDFSSKDSGNNQSAGNTDSALITLEDPMDAEGSSKPEELPEFSCGSPLTLKQKRDLLQKSFALPEMSLDDHPDPGTEGEKPGELMPSSGAKTVLLKVPEDAENPTESEKPDTSAESDTEQNPERKVEEDGAEESEFKIQIVPRQRKQRKIAVSAIQREYLDISFNILDKLGEQKDPDPSTKGLSTLEMPRESSSAPTLDAGVPETSSHSSISTQYRQMKRGSLGVLTMSQLMKRQLEHQSSAPHNISNWDTEQIQPGKRQCNVPTCLNPDLEGQPLRMRGATKSSLLSAPSIVSMFVPAPEEFTDEQPTVMTDKCHDCGAILEEYDEETLGLAIVVLSTFIHLSPDLAAPLLLDIMQSVGRLASSTTFSNQAESMMVPGNAAGVAKQFLRCIFHQLAPNGIFPQLFQSTIKDGTFLRTLASSLMDFNELSSIAALSQLLEGLNNKKNLPAGGAMIRCLENIATFMEALPMDSPSSLWTTISNQFQTFFAKLPCVLPLKCSLDSSLRIMICLLKIPSTNATRSLLEPFSKLLSFVIQNAVFTLAYLVELCGLCYRAFTKERDKFYLSRSVVLELLQALKLKSPLPDTNLLLLVQFICADAGTKLAESTILSKQMIASVPGCGTAAMECVRQYINEVLDFMADMHTLTKLKSHMKTCSQPLHEDTFGGHLKVGLAQIAAMDISRGNHRDNKAVIRYLPWLYHPPSAMQQGPKEFIECVSHIRLLSWLLLGSLTHNAVCPNASSPCLPIPLDAGSHVADHLIVILIGFPEQSKTSVLHMCSLFHAFIFAQLWTVYCEQSAVATNLQNQNEFSFTAILTALEFWSRVTPSILQLMAHNKVMVEMVCLHVISLMEALQECNSTIFVKLIPMWLPMIQSNIKHLSAGLQLRLQAIQNHVNHHSLRTLPGSGQSSAGLAALRKWLQCTQFKMAQVEIQSSEAASQFYPLGGSGGSVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKGGSGGSDYKDDDDKGSGDYKDDDDK

SEQ ID NO: 55 - protein, 1069 aa, synthetic construct
  REGION 1..1069: note = Synthetic: hUNC79_1-800*
MSTKAEQFASKIRYLQEYHNRVLHNIYPVPSGTDIANTLKYFSQTLLSILSRTGKKENQDASNLTVPMTMCLFPVPFPLTPSLRPQVSSINPTVTRSLLYSVLRDAPSERGPQSRDAQLSDYPSLDYQGLYVTLVTLLDLVPLLQHGQHDLGQSIFYTTTCLLPFLNDDILSTLPYTMISTLATFPPFLHKDIIEYLSTSFLPMAILGSSRREGVPAHVNLSASSMLMIAMQYTSNPVYHCQLLECLMKYKQEVWKDLLYVIAYGPSQVKPPAVQMLFHYWPNLKPPGAISEYRGLQYTAWNPIHCQHIECHNAINKPAVKMCIDPSLSVALGDKPPPLYLCEECSERIAGDHSEWLIDVLLPQAEISAICQKKNCSSHVRRAVVTCFSAGCCGRHGNRPVRYCKRCHSNHHSNEVGAAAETHLYQTSPPPINTRECGAEELVCAVEAVISLLKEAEFHAEQREHELNRRRQLGLSSSHHSLDNADFDNKDDDKHDQRLLSQFGIWFLVSLCTPSENTPTESLARLVAMVFQWFHSTAYMMDDEVGSLVEKLKPQFVTKWLKTVCDVRFDVMVMCLLPKPMEFARVGGYWDKSCSTVTQLKEGLNRILCLIPYNVINQSVWECIMPEWLEAIRTEVPDNQLKEFREVLSKMFDIELCPLPFSMEEMFGFISCRFTGYPSSVQEQALLWLHVLSELDIMVPLQLLISMFSDGVNSVKELANQRKSRVSELAGNLASRRVSVASDPGRRVQHNMLSPFHSPFQSPFRSPLRSPFRSPFKNFGHPGGRTIDFDCEDDEMNLNCGGSGGSVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKGGSGGSDYKDDDDKGSGDYKDDDDK

SEQ ID NO: 56 - protein, 2105 aa, synthetic construct
  REGION 1..2105: note = Synthetic: hUNC79_801-2635*
MFILMFDLLLKQMELQDDGITMGLEHSLSKDIISIINNVFQAPWGGSHTCQKDEKAIECNLCQSSILCYQLACELLERLAPKEESRLVEPTDSLEDSLLSSRPEFIIGPEGEEEENPASKHGENPGNCTEPVEHAAVKNDTERKFCYQQLPVTLRLIYTIFQEMAKFEEPDILFNMLNCLKILCLHGECLYIARKDHPQFLAYIQDHMLIASLWRVVKSEFSQLSSLAVPLLLHALSLPHGADIFWTIINGNFNSKDWKMRFEAVEKVAVICRFLDIHSVTKNHLLKYSLAHAFCCFLTAVEDVNPAVATRAGLLLDTIKRPALQGLCLCLDFQFDTVVKDRPTILSKLLLLHFLKQDIPALSWEFFVNRFETLSLEAQLHLDCNKEFPFPTTITAVRTNVANLSDAALWKIKRARFARNRQKSVRSLRDSVKGPVESKRALSLPETLTSKIRQQSPENDNTIKDLLPEDAGIDHQTVHQLITVLMKFMAKDESSAESDISSAKAFNTVKRHLYVLLGYDQQEGCFMIAPQKMRLSTCFNAFIAGIAQVMDYNINLGKHLLPLVVQVLKYCSCPQLRHYFQQPPRCSLWSLKPHIRQMWLKALLVILYKYPYRDCDISKILLHLIHITVNTLNAQYHSCKPHATAGPLYSDNSNISRYSEKEKGEIELAEYRETGALQDSLLHCVREESIPKKKLRSFKQKSLDIGNADSLLFTLDEHRRKSCIDRCDIEKPPTQAAYIAQRPNDPGRSRQNSATRPDNSEIPENPAMEGFPDARRPVIPEVRLNCMETFEVKVDSPVKPAPKEDLDLIDLSSDSTSGPEKHSILSTSDSDSLVFEPLPPLRIVESDEEEETMNQGDDGPSGKNAASSPSVPSHPSVLSLSTAPLVQVSVEDCSKDFSSKDSGNNQSAGNTDSALITLEDPMDAEGSSKPEELPEFSCGSPLTLKQKRDLLQKSFALPEMSLDDHPDPGTEGEKPGELMPSSGAKTVLLKVPEDAENPTESEKPDTSAESDTEQNPERKVEEDGAEESEFKIQIVPRQRKQRKIAVSAIQREYLDISFNILDKLGEQKDPDPSTKGLSTLEMPRESSSAPTLDAGVPETSSHSSISTQYRQMKRGSLGVLTMSQLMKRQLEHQSSAPHNISNWDTEQIQPGKRQCNVPTCLNPDLEGQPLRMRGATKSSLLSAPSIVSMFVPAPEEFTDEQPTVMTDKCHDCGAILEEYDEETLGLAIVVLSTFIHLSPDLAAPLLLDIMQSVGRLASSTTFSNQAESMMVPGNAAGVAKQFLRCIFHQLAPNGIFPQLFQSTIKDGTFLRTLASSLMDFNELSSIAALSQLLEGLNNKKNLPAGGAMIRCLENIATFMEALPMDSPSSLWTTISNQFQTFFAKLPCVLPLKCSLDSSLRIMICLLKIPSTNATRSLLEPFSKLLSFVIQNAVFTLAYLVELCGLCYRAFTKERDKFYLSRSVVLELLQALKLKSPLPDTNLLLLVQFICADAGTKLAESTILSKQMIASVPGCGTAAMECVRQYINEVLDFMADMHTLTKLKSHMKTCSQPLHEDTFGGHLKVGLAQIAAMDISRGNHRDNKAVIRYLPWLYHPPSAMQQGPKEFIECVSHIRLLSWLLLGSLTHNAVCPNASSPCLPIPLDAGSHVADHLIVILIGFPEQSKTSVLHMCSLFHAFIFAQLWTVYCEQSAVATNLQNQNEFSFTAILTALEFWSRVTPSILQLMAHNKVMVEMVCLHVISLMEALQECNSTIFVKLIPMWLPMIQSNIKHLSAGLQLRLQAIQNHVNHHSLRTLPGSGQSSAGLAALRKWLQCTQFKMAQVEIQSSEAASQFYPLGGSGGSVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKGGSGGSDYKDDDDKGSGDYKDDDDK

SEQ ID NO: 57 - protein, 1669 aa, synthetic construct
  REGION 1..1669: note = Synthetic: hUNC79_1-1400*
MSTKAEQFASKIRYLQEYHNRVLHNIYPVPSGTDIANTLKYFSQTLLSILSRTGKKENQDASNLTVPMTMCLFPVPFPLTPSLRPQVSSINPTVTRSLLYSVLRDAPSERGPQSRDAQLSDYPSLDYQGLYVTLVTLLDLVPLLQHGQHDLGQSIFYTTTCLLPFLNDDILSTLPYTMISTLATFPPFLHKDIIEYLSTSFLPMAILGSSRREGVPAHVNLSASSMLMIAMQYTSNPVYHCQLLECLMKYKQEVWKDLLYVIAYGPSQVKPPAVQMLFHYWPNLKPPGAISEYRGLQYTAWNPIHCQHIECHNAINKPAVKMCIDPSLSVALGDKPPPLYLCEECSERIAGDHSEWLIDVLLPQAEISAICQKKNCSSHVRRAVVTCFSAGCCGRHGNRPVRYCKRCHSNHHSNEVGAAAETHLYQTSPPPINTRECGAEELVCAVEAVISLLKEAEFHAEQREHELNRRRQLGLSSSHHSLDNADFDNKDDDKHDQRLLSQFGIWFLVSLCTPSENTPTESLARLVAMVFQWFHSTAYMMDDEVGSLVEKLKPQFVTKWLKTVCDVRFDVMVMCLLPKPMEFARVGGYWDKSCSTVTQLKEGLNRILCLIPYNVINQSVWECIMPEWLEAIRTEVPDNQLKEFREVLSKMFDIELCPLPFSMEEMFGFISCRFTGYPSSVQEQALLWLHVLSELDIMVPLQLLISMFSDGVNSVKELANQRKSRVSELAGNLASRRVSVASDPGRRVQHNMLSPFHSPFQSPFRSPLRSPFRSPFKNFGHPGGRTIDFDCEDDEMNLNCFILMFDLLLKQMELQDDGITMGLEHSLSKDIISIINNVFQAPWGGSHTCQKDEKAIECNLCQSSILCYQLACELLERLAPKEESRLVEPTDSLEDSLLSSRPEFIIGPEGEEEENPASKHGENPGNCTEPVEHAAVKNDTERKFCYQQLPVTLRLIYTIFQEMAKFEEPDILFNMLNCLKILCLHGECLYIARKDHPQFLAYIQDHMLIASLWRVVKSEFSQLSSLAVPLLLHALSLPHGADIFWTIINGNFNSKDWKMRFEAVEKVAVICRFLDIHSVTKNHLLKYSLAHAFCCFLTAVEDVNPAVATRAGLLLDTIKRPALQGLCLCLDFQFDTVVKDRPTILSKLLLLHFLKQDIPALSWEFFVNRFETLSLEAQLHLDCNKEFPFPTTITAVRTNVANLSDAALWKIKRARFARNRQKSVRSLRDSVKGPVESKRALSLPETLTSKIRQQSPENDNTIKDLLPEDAGIDHQTVHQLITVLMKFMAKDESSAESDISSAKAFNTVKRHLYVLLGYDQQEGCFMIAPQKMRLSTCFNAFIAGIAQVMDYNINLGKHLLPLVVQVLKYCSCPQLRHYFQQPPRCSLWSLKPHIRQMWLKGGSGGSVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKGGSGGSDYKDDDDKGSGDYKDDDDK

SEQ ID NO: 58 - protein, 1236 aa, synthetic construct
  REGION 1..1236: note = Synthetic: hUNC79_1401-2635
MALLVILYKYPYRDCDISKILLHLIHITVNTLNAQYHSCKPHATAGPLYSDNSNISRYSEKEKGEIELAEYRETGALQDSLLHCVREESIPKKKLRSFKQKSLDIGNADSLLFTLDEHRRKSCIDRCDIEKPPTQAAYIAQRPNDPGRSRQNSATRPDNSEIPENPAMEGFPDARRPVIPEVRLNCMETFEVKVDSPVKPAPKEDLDLIDLSSDSTSGPEKHSILSTSDSDSLVFEPLPPLRIVESDEEEETMNQGDDGPSGKNAASSPSVPSHPSVLSLSTAPLVQVSVEDCSKDFSSKDSGNNQSAGNTDSALITLEDPMDAEGSSKPEELPEFSCGSPLTLKQKRDLLQKSFALPEMSLDDHPDPGTEGEKPGELMPSSGAKTVLLKVPEDAENPTESEKPDTSAESDTEQNPERKVEEDGAEESEFKIQIVPRQRKQRKIAVSAIQREYLDISFNILDKLGEQKDPDPSTKGLSTLEMPRESSSAPTLDAGVPETSSHSSISTQYRQMKRGSLGVLTMSQLMKRQLEHQSSAPHNISNWDTEQIQPGKRQCNVPTCLNPDLEGQPLRMRGATKSSLLSAPSIVSMFVPAPEEFTDEQPTVMTDKCHDCGAILEEYDEETLGLAIVVLSTFIHLSPDLAAPLLLDIMQSVGRLASSTTFSNQAESMMVPGNAAGVAKQFLRCIFHQLAPNGIFPQLFQSTIKDGTFLRTLASSLMDFNELSSIAALSQLLEGLNNKKNLPAGGAMIRCLENIATFMEALPMDSPSSLWTTISNQFQTFFAKLPCVLPLKCSLDSSLRIMICLLKIPSTNATRSLLEPFSKLLSFVIQNAVFTLAYLVELCGLCYRAFTKERDKFYLSRSVVLELLQALKLKSPLPDTNLLLLVQFICADAGTKLAESTILSKQMIASVPGCGTAAMECVRQYINEVLDFMADMHTLTKLKSHMKTCSQPLHEDTFGGHLKVGLAQIAAMDISRGNHRDNKAVIRYLPWLYHPPSAMQQGPKEFIECVSHIRLLSWLLLGSLTHNAVCPNASSPCLPIPLDAGSHVADHLIVILIGFPEQSKTSVLHMCSLFHAFIFAQLWTVYCEQSAVATNLQNQNEFSFTAILTALEFWSRVTPSILQLMAHNKVMVEMVCLHVISLMEALQECNSTIFVKLIPMWLPMIQSNIKHLSAGLQLRLQAIQNHVNHHSLRTLPGSGQSSAGLAALRKWLQCTQFKMAQVEIQSSEAASQFYPL

SEQ ID NO: 59 - protein, 1817 aa, synthetic construct
  REGION 1..1817: note = Synthetic: hNALCN-rNav1.4_N-term_chimera
MASSSLPNLVPPGPHCLRPFTPESLAAIEQRAVEEEARLQRNKQMEIEEPERKPRSDLEAGKNLPLIYGDPPPEVIGIPLEDLDPYYSDKKTFIVLNKGKAIFRFSATPALYLLSPFSILLRICAIISVISVCMNTPMTFEHYPPLQYVTFTLDTLLMFLYTAEMIAKMHIRGIVKGDSSYVKDRWCVFDGFMVFCLWVSLVLQVFEIADIVDQMSPWGMLRIPRPLIMIRAFRIYFRFELPRTRITNILKRSGEQIWSVSIFLLFFLLLYGILGVQMFGTFTYHCVVNDTKPGNVTWNSLAIPDTHCSPELEEGYQCPPGFKCMDLEDLGLSRQELGYSGFNEIGTSIFTVYEAASQEGWVFLMYRAIDSFPRWRSYFYFITLIFFLAWLVKNVFIAVIIETFAEIRVQFQQMWGSRSSTTSTATTQMFHEDAAGGWQLVAVDVNKPQGRAPACLQKMMRSSVFHMFILSMVTVDVIVAASNYYKGENFRRQYDEFYLAEVAFTVLFDLEALLKIWCLGFTGYISSSLHKFELLLVIGTTLHVYPDLYHSQFTYFQVLRVVRLIKISPALEDFVYKIFGPGKKLGSLVVFTASLLIVMSAISLQMFCFVEELDRFTTFPRAFMSMFQILTQEGWVDVMDQTLNAVGHMWAPVVAIYFILYHLFATLILLSLFVAVILDNLELDEDLKKLKQLKQSEANADTKEKLPLRLRIFEKFPNRPQMVKISKLPSDFTVPKIRESFMKQFIDRQQQDTCCLLRSLPTTSSSSCDHSKRSAIEDNKYIDQKLRKSVFSIRARNLLEKETAVTKILRACTRQRMLSGSFEGQPAKERSILSVQHHIRQERRSLRHGSNSQRISRGKSLETLTQDHSNTVRYRNAQREDSEIKMIQEKKEQAEMKRKVQEEELRENHPYFDKPLFIVGREHRFRNFCRVVVRARFNASKTDPVTGAVKNTKYHQLYDLLGLVTYLDWVMIIVTICSCISMMFESPFRRVMHAPTLQIAEYVFVIFMSIELNLKIMADGLFFTPTAVIRDFGGVMDIFIYLVSLIFLCWMPQNVPAESGAQLLMVLRCLRPLRIFKLVPQMRKVVRELFSGFKEIFLVSILLLTLMLVFASFGVQLFAGKLAKCNDPNIIRREDCNGIFRINVSVSKNLNLKLRPGEKKPGFWVPRVWANPRNFNFDNVGNAMLALFEVLSLKGWVEVRDVIIHRVGPIHGIYIHVFVFLGCMIGLTLFVGVVIANFNENKGTALLTVDQRRWEDLKSRLKIAQPLHLPPRPDNDGFRAKMYDITQHPFFKRTIALLVLAQSVLLSVKWDVEDPVTVPLATMSVVFTFIFVLEVTMKIIAMSPAGFWQSRRNRYDLLVTSLGVVWVVLHFALLNAYTYMMGACVIVFRFFSICGKHVTLKMLLLTVVVSMYKSFFIIVGMFLLLLCYAFAGVVLFGTVKYGENINRHANFSSAGKAITVLFRIVTGEDWNKIMHDCMVQPPFCTPDEFTYWATDCGNYAGALMYFCSFYVIIAYIMLNLLVAIIVENFSLFYSTEEDQLLSYNDLRHFQIIWNMVDDKREGVIPTFRVKFLLRLLRGRLEVDLDKDKLLFKHMCYEMERLHNGGDVTFHDVLSMLSYRSVDIRKSLQLEELLAREQLEYTIEEEVAKQTIRMWLKKCLKRIRAKQQQSCSIIHSLRESQQQELSRFLNPPSIETTQPSEDTNANSQDNSMQPETSSQQQLLSPTLSDRGGSRQDAADAGKPQRKFGQWRLPSAPKPISHSVSSVNLRFGGRTTMKSVVCKMNPMTDAASCGSEVKKWWTRQLTVESDESGDDLLDI

SEQ ID NO: 60 - protein, 1742 aa, synthetic construct
  REGION 1..1742: note = Synthetic: hNALCN-rNav1.4_DIII-DIV_linker_chimera
MLKRKQSSRVEAQPVTDFGPDESLSDNADILWINKPWVHSLLRICAIISVISVCMNTPMTFEHYPPLQYVTFTLDTLLMFLYTAEMIAKMHIRGIVKGDSSYVKDRWCVFDGFMVFCLWVSLVLQVFEIADIVDQMSPWGMLRIPRPLIMIRAFRIYFRFELPRTRITNILKRSGEQIWSVSIFLLFFLLLYGILGVQMFGTFTYHCVVNDTKPGNVTWNSLAIPDTHCSPELEEGYQCPPGFKCMDLEDLGLSRQELGYSGFNEIGTSIFTVYEAASQEGWVFLMYRAIDSFPRWRSYFYFITLIFFLAWLVKNVFIAVIIETFAEIRVQFQQMWGSRSSTTSTATTQMFHEDAAGGWQLVAVDVNKPQGRAPACLQKMMRSSVFHMFILSMVTVDVIVAASNYYKGENFRRQYDEFYLAEVAFTVLFDLEALLKIWCLGFTGYISSSLHKFELLLVIGTTLHVYPDLYHSQFTYFQVLRVVRLIKISPALEDFVYKIFGPGKKLGSLVVFTASLLIVMSAISLQMFCFVEELDRFTTFPRAFMSMFQILTQEGWVDVMDQTLNAVGHMWAPVVAIYFILYHLFATLILLSLFVAVILDNLELDEDLKKLKQLKQSEANADTKEKLPLRLRIFEKFPNRPQMVKISKLPSDFTVPKIRESFMKQFIDRQQQDTCCLLRSLPTTSSSSCDHSKRSAIEDNKYIDQKLRKSVFSIRARNLLEKETAVTKILRACTRQRMLSGSFEGQPAKERSILSVQHHIRQERRSLRHGSNSQRISRGKSLETLTQDHSNTVRYRNAQREDSEIKMIQEKKEQAEMKRKVQEEELRENHPYFDKPLFIVGREHRFRNFCRVVVRARFNASKTDPVTGAVKNTKYHQLYDLLGLVTYLDWVMIIVTICSCISMMFESPFRRVMHAPTLQIAEYVFVIFMSIELNLKIMADGLFFTPTAVIRDFGGVMDIFIYLVSLIFLCWMPQNVPAESGAQLLMVLRCLRPLRIFKLVPQMRKVVRELFSGFKEIFLVSILLLTLMLVFASFGVQLFAGKLAKCNDPNIIRREDCNGIFRINVSVSKNLNLKLRPGEKKPGFWVPRVWANPRNFNFDNVGNAMLALFEVLSLKGWVEVRDVIIHRVGPIHGIYIHVFVFLGCMIGLTLFVGVVIANFNENKGTFGGKDIFMTEEQKKYYNAMKKLGSKKPQKPIPRPQNGFRAKMYDITQHPFFKRTIALLVLAQSVLLSVKWDVEDPVTVPLATMSVVFTFIFVLEVTMKIIAMSPAGFWQSRRNRYDLLVTSLGVVWVVLHFALLNAYTYMMGACVIVFRFFSICGKHVTLKMLLLTVVVSMYKSFFIIVGMFLLLLCYAFAGVVLFGTVKYGENINRHANFSSAGKAITVLFRIVTGEDWNKIMHDCMVQPPFCTPDEFTYWATDCGNYAGALMYFCSFYVIIAYIMLNLLVAIIVENFSLFYSTEEDQLLSYNDLRHFQIIWNMVDDKREGVIPTFRVKFLLRLLRGRLEVDLDKDKLLFKHMCYEMERLHNGGDVTFHDVLSMLSYRSVDIRKSLQLEELLAREQLEYTIEEEVAKQTIRMWLKKCLKRIRAKQQQSCSIIHSLRESQQQELSRFLNPPSIETTQPSEDTNANSQDNSMQPETSSQQQLLSPTLSDRGGSRQDAADAGKPQRKFGQWRLPSAPKPISHSVSSVNLRFGGRTTMKSVVCKMNPMTDAASCGSEVKKWWTRQLTVESDESGDDLLDI

SEQ ID NO: 61 - protein, 1697 aa, synthetic construct
  REGION 1..1697: note = Synthetic: hNALCN-rNav1.4_C-term_chimera
MLKRKQSSRVEAQPVTDFGPDESLSDNADILWINKPWVHSLLRICAIISVISVCMNTPMTFEHYPPLQYVTFTLDTLLMFLYTAEMIAKMHIRGIVKGDSSYVKDRWCVFDGFMVFCLWVSLVLQVFEIADIVDQMSPWGMLRIPRPLIMIRAFRIYFRFELPRTRITNILKRSGEQIWSVSIFLLFFLLLYGILGVQMFGTFTYHCVVNDTKPGNVTWNSLAIPDTHCSPELEEGYQCPPGFKCMDLEDLGLSRQELGYSGFNEIGTSIFTVYEAASQEGWVFLMYRAIDSFPRWRSYFYFITLIFFLAWLVKNVFIAVIIETFAEIRVQFQQMWGSRSSTTSTATTQMFHEDAAGGWQLVAVDVNKPQGRAPACLQKMMRSSVFHMFILSMVTVDVIVAASNYYKGENFRRQYDEFYLAEVAFTVLFDLEALLKIWCLGFTGYISSSLHKFELLLVIGTTLHVYPDLYHSQFTYFQVLRVVRLIKISPALEDFVYKIFGPGKKLGSLVVFTASLLIVMSAISLQMFCFVEELDRFTTFPRAFMSMFQILTQEGWVDVMDQTLNAVGHMWAPVVAIYFILYHLFATLILLSLFVAVILDNLELDEDLKKLKQLKQSEANADTKEKLPLRLRIFEKFPNRPQMVKISKLPSDFTVPKIRESFMKQFIDRQQQDTCCLLRSLPTTSSSSCDHSKRSAIEDNKYIDQKLRKSVFSIRARNLLEKETAVTKILRACTRQRMLSGSFEGQPAKERSILSVQHHIRQERRSLRHGSNSQRISRGKSLETLTQDHSNTVRYRNAQREDSEIKMIQEKKEQAEMKRKVQEEELRENHPYFDKPLFIVGREHRFRNFCRVVVRARFNASKTDPVTGAVKNTKYHQLYDLLGLVTYLDWVMIIVTICSCISMMFESPFRRVMHAPTLQIAEYVFVIFMSIELNLKIMADGLFFTPTAVIRDFGGVMDIFIYLVSLIFLCWMPQNVPAESGAQLLMVLRCLRPLRIFKLVPQMRKVVRELFSGFKEIFLVSILLLTLMLVFASFGVQLFAGKLAKCNDPNIIRREDCNGIFRINVSVSKNLNLKLRPGEKKPGFWVPRVWANPRNFNFDNVGNAMLALFEVLSLKGWVEVRDVIIHRVGPIHGIYIHVFVFLGCMIGLTLFVGVVIANFNENKGTALLTVDQRRWEDLKSRLKIAQPLHLPPRPDNDGFRAKMYDITQHPFFKRTIALLVLAQSVLLSVKWDVEDPVTVPLATMSVVFTFIFVLEVTMKIIAMSPAGFWQSRRNRYDLLVTSLGVVWVVLHFALLNAYTYMMGACVIVFRFFSICGKHVTLKMLLLTVVVSMYKSFFIIVGMFLLLLCYAFAGVVLFGTVKYGENINRHANFSSAGKAITVLFRIVTGEDWNKIMHDCMVQPPFCTPDEFTYWATDCGNYAGALMYFCSFYVIIAYIMLNLLVAIIVLENFNVATEESSEPLSEDDFEMFYETWEKFDPDATQFIDYSRLSDFVDTLQEPLKIAKPNKIKLITLDLPMVPGDKIHCLDILFALTKEVLGDSGEMDALKQTMEEKFMAANPSKVSYEPITTTLKRKQEEVCAIKIQRAYRRHLLQRSVKQASYMYRHSQDGNGDGAPEKEGLLANTMNKMYGHEKEGDGVQSQGEEEKASTEDAGPTVEPKPTSSSDTALTPSPPPLPPSSSPPQGQTVRPGVKESLV

SEQ ID NO: 62 - protein, 1738 aa, synthetic construct
  REGION 1..1738: note = Synthetic: hNALCN_R1573E/K1577E/K1578G
MLKRKQSSRVEAQPVTDFGPDESLSDNADILWINKPWVHSLLRICAIISVISVCMNTPMTFEHYPPLQYVTFTLDTLLMFLYTAEMIAKMHIRGIVKGDSSYVKDRWCVFDGFMVFCLWVSLVLQVFEIADIVDQMSPWGMLRIPRPLIMIRAFRIYFRFELPRTRITNILKRSGEQIWSVSIFLLFFLLLYGILGVQMFGTFTYHCVVNDTKPGNVTWNSLAIPDTHCSPELEEGYQCPPGFKCMDLEDLGLSRQELGYSGFNEIGTSIFTVYEAASQEGWVFLMYRAIDSFPRWRSYFYFITLIFFLAWLVKNVFIAVIIETFAEIRVQFQQMWGSRSSTTSTATTQMFHEDAAGGWQLVAVDVNKPQGRAPACLQKMMRSSVFHMFILSMVTVDVIVAASNYYKGENFRRQYDEFYLAEVAFTVLFDLEALLKIWCLGFTGYISSSLHKFELLLVIGTTLHVYPDLYHSQFTYFQVLRVVRLIKISPALEDFVYKIFGPGKKLGSLVVFTASLLIVMSAISLQMFCFVEELDRFTTFPRAFMSMFQILTQEGWVDVMDQTLNAVGHMWAPVVAIYFILYHLFATLILLSLFVAVILDNLELDEDLKKLKQLKQSEANADTKEKLPLRLRIFEKFPNRPQMVKISKLPSDFTVPKIRESFMKQFIDRQQQDTCCLLRSLPTTSSSSCDHSKRSAIEDNKYIDQKLRKSVFSIRARNLLEKETAVTKILRACTRQRMLSGSFEGQPAKERSILSVQHHIRQERRSLRHGSNSQRISRGKSLETLTQDHSNTVRYRNAQREDSEIKMIQEKKEQAEMKRKVQEEELRENHPYFDKPLFIVGREHRFRNFCRVVVRARFNASKTDPVTGAVKNTKYHQLYDLLGLVTYLDWVMIIVTICSCISMMFESPFRRVMHAPTLQIAEYVFVIFMSIELNLKIMADGLFFTPTAVIRDFGGVMDIFIYLVSLIFLCWMPQNVPAESGAQLLMVLRCLRPLRIFKLVPQMRKVVRELFSGFKEIFLVSILLLTLMLVFASFGVQLFAGKLAKCNDPNIIRREDCNGIFRINVSVSKNLNLKLRPGEKKPGFWVPRVWANPRNFNFDNVGNAMLALFEVLSLKGWVEVRDVIIHRVGPIHGIYIHVFVFLGCMIGLTLFVGVVIANFNENKGTALLTVDQRRWEDLKSRLKIAQPLHLPPRPDNDGFRAKMYDITQHPFFKRTIALLVLAQSVLLSVKWDVEDPVTVPLATMSVVFTFIFVLEVTMKIIAMSPAGFWQSRRNRYDLLVTSLGVVWVVLHFALLNAYTYMMGACVIVFRFFSICGKHVTLKMLLLTVVVSMYKSFFIIVGMFLLLLCYAFAGVVLFGTVKYGENINRHANFSSAGKAITVLFRIVTGEDWNKIMHDCMVQPPFCTPDEFTYWATDCGNYAGALMYFCSFYVIIAYIMLNLLVAIIVENFSLFYSTEEDQLLSYNDLRHFQIIWNMVDDKREGVIPTFRVKFLLRLLRGRLEVDLDKDKLLFKHMCYEMERLHNGGDVTFHDVLSMLSYRSVDIRKSLQLEELLAREQLEYTIEEEVAKQTIEMWLEGCLKRIRAKQQQSCSIIHSLRESQQQELSRFLNPPSIETTQPSEDTNANSQDNSMQPETSSQQQLLSPTLSDRGGSRQDAADAGKPQRKFGQWRLPSAPKPISHSVSSVNLRFGGRTTMKSVVCKMNPMTDAASCGSEVKKWWTRQLTVESDESGDDLLDI

SEQ ID NO: 63 - protein, 1738 aa, synthetic construct
  REGION 1..1738: note = Synthetic: hNALCN_W1575E/L1576E
MLKRKQSSRVEAQPVTDFGPDESLSDNADILWINKPWVHSLLRICAIISVISVCMNTPMTFEHYPPLQYVTFTLDTLLMFLYTAEMIAKMHIRGIVKGDSSYVKDRWCVFDGFMVFCLWVSLVLQVFEIADIVDQMSPWGMLRIPRPLIMIRAFRIYFRFELPRTRITNILKRSGEQIWSVSIFLLFFLLLYGILGVQMFGTFTYHCVVNDTKPGNVTWNSLAIPDTHCSPELEEGYQCPPGFKCMDLEDLGLSRQELGYSGFNEIGTSIFTVYEAASQEGWVFLMYRAIDSFPRWRSYFYFITLIFFLAWLVKNVFIAVIIETFAEIRVQFQQMWGSRSSTTSTATTQMFHEDAAGGWQLVAVDVNKPQGRAPACLQKMMRSSVFHMFILSMVTVDVIVAASNYYKGENFRRQYDEFYLAEVAFTVLFDLEALLKIWCLGFTGYISSSLHKFELLLVIGTTLHVYPDLYHSQFTYFQVLRVVRLIKISPALEDFVYKIFGPGKKLGSLVVFTASLLIVMSAISLQMFCFVEELDRFTTFPRAFMSMFQILTQEGWVDVMDQTLNAVGHMWAPVVAIYFILYHLFATLILLSLFVAVILDNLELDEDLKKLKQLKQSEANADTKEKLPLRLRIFEKFPNRPQMVKISKLPSDFTVPKIRESFMKQFIDRQQQDTCCLLRSLPTTSSSSCDHSKRSAIEDNKYIDQKLRKSVFSIRARNLLEKETAVTKILRACTRQRMLSGSFEGQPAKERSILSVQHHIRQERRSLRHGSNSQRISRGKSLETLTQDHSNTVRYRNAQREDSEIKMIQEKKEQAEMKRKVQEEELRENHPYFDKPLFIVGREHRFRNFCRVVVRARFNASKTDPVTGAVKNTKYHQLYDLLGLVTYLDWVMIIVTICSCISMMFESPFRRVMHAPTLQIAEYVFVIFMSIELNLKIMADGLFFTPTAVIRDFGGVMDIFIYLVSLIFLCWMPQNVPAESGAQLLMVLRCLRPLRIFKLVPQMRKVVRELFSGFKEIFLVSILLLTLMLVFASFGVQLFAGKLAKCNDPNIIRREDCNGIFRINVSVSKNLNLKLRPGEKKPGFWVPRVWANPRNFNFDNVGNAMLALFEVLSLKGWVEVRDVIIHRVGPIHGIYIHVFVFLGCMIGLTLFVGVVIANFNENKGTALLTVDQRRWEDLKSRLKIAQPLHLPPRPDNDGFRAKMYDITQHPFFKRTIALLVLAQSVLLSVKWDVEDPVTVPLATMSVVFTFIFVLEVTMKIIAMSPAGFWQSRRNRYDLLVTSLGVVWVVLHFALLNAYTYMMGACVIVFRFFSICGKHVTLKMLLLTVVVSMYKSFFIIVGMFLLLLCYAFAGVVLFGTVKYGENINRHANFSSAGKAITVLFRIVTGEDWNKIMHDCMVQPPFCTPDEFTYWATDCGNYAGALMYFCSFYVIIAYIMLNLLVAIIVENFSLFYSTEEDQLLSYNDLRHFQIIWNMVDDKREGVIPTFRVKFLLRLLRGRLEVDLDKDKLLFKHMCYEMERLHNGGDVTFHDVLSMLSYRSVDIRKSLQLEELLAREQLEYTIEEEVAKQTIRMEEKKCLKRIRAKQQQSCSIIHSLRESQQQELSRFLNPPSIETTQPSEDTNANSQDNSMQPETSSQQQLLSPTLSDRGGSRQDAADAGKPQRKFGQWRLPSAPKPISHSVSSVNLRFGGRTTMKSVVCKMNPMTDAASCGSEVKKWWTRQLTVESDESGDDLLDI

SEQ ID NO: 64 - protein, 500 aa, synthetic construct
  REGION 1..500: note = Synthetic: UNC79_frag1
MSTKAEQFASKIRYLQEYHNRVLHNIYPVPSGTDIANTLKYFSQTLLSILSRTGKKENQDASNLTVPMTMCLFPVPFPLTPSLRPQVSSINPTVTRSLLYSVLRDAPSERGPQSRDAQLSDYPSLDYQGLYVTLVTLLDLVPLLQHGQHDLGQSIFYTTTCLLPFLNDDILSTLPYTMISTLATFPPFLHKDIIEYLSTSFLPMAILGSSRREGVPAHVNLSASSMLMIAMQYTSNPVYHCQLLECLMKYKQEVWKDLLYVIAYGPSQVKPPAVQMLFHYWPNLKPPGAISEYRGLQYTAWNPIHCQHIECHNAINKPAVKMCIDPSLSVALGDKPPPLYLCEECSERIAGDHSEWLIDVLLPQAEISAICQKKNCSSHVRRAVVTCFSAGCCGRHGNRPVRYCKRCHSNHHSNEVGAAAETHLYQTSPPPINTRECGAEELVCAVEAVISLLKEAEFHAEQREHELNRRRQLGLSSSHHSLDNADFDNKDDDKHDQRLL

SEQ ID NO: 65 - protein, 502 aa, synthetic construct
  REGION 1..502: note = Synthetic: UNC79_frag2
MYKQEVWKDLLYVIAYGPSQVKPPAVQMLFHYWPNLKPPGAISEYRGLQYTAWNPIHCQHIECHNAINKPAVKMCIDPSLSVALGDKPPPLYLCEECSERIAGDHSEWLIDVLLPQAEISAICQKKNCSSHVRRAVVTCFSAGCCGRHGNRPVRYCKRCHSNHHSNEVGAAAETHLYQTSPPPINTRECGAEELVCAVEAVISLLKEAEFHAEQREHELNRRRQLGLSSSHHSLDNADFDNKDDDKHDQRLLSQFGIWFLVSLCTPSENTPTESLARLVAMVFQWFHSTAYMMDDEVGSLVEKLKPQFVTKWLKTVCDVRFDVMVMCLLPKPMEFARVGGYWDKSCSTVTQLKEGLNRILCLIPYNVINQSVWECIMPEWLEAIRTEVPDNQLKEFREVLSKMFDIELCPLPFSMEEMFGFISCRFTGYPSSVQEQALLWLHVLSELDIMVPLQLLISMFSDGVNSVKELANQRKSRVSELAGNLASRRVSVASDPGRRVQH

SEQ ID NO: 66 - protein, 502 aa, synthetic construct
  REGION 1..502: note = Synthetic: UNC79_frag3
MLSQFGIWFLVSLCTPSENTPTESLARLVAMVFQWFHSTAYMMDDEVGSLVEKLKPQFVTKWLKTVCDVRFDVMVMCLLPKPMEFARVGGYWDKSCSTVTQLKEGLNRILCLIPYNVINQSVWECIMPEWLEAIRTEVPDNQLKEFREVLSKMFDIELCPLPFSMEEMFGFISCRFTGYPSSVQEQALLWLHVLSELDIMVPLQLLISMFSDGVNSVKELANQRKSRVSELAGNLASRRVSVASDPGRRVQHNMLSPFHSPFQSPFRSPLRSPFRSPFKNFGHPGGRTIDFDCEDDEMNLNCFILMFDLLLKQMELQDDGITMGLEHSLSKDIISIINNVFQAPWGGSHTCQKDEKAIECNLCQSSILCYQLACELLERLAPKEESRLVEPTDSLEDSLLSSRPEFIIGPEGEEEENPASKHGENPGNCTEPVEHAAVKNDTERKFCYQQLPVTLRLIYTIFQEMAKFEEPDILFNMLNCLKILCLHGECLYIARKDHPQFL

SEQ ID NO: 67 - protein, 502 aa, synthetic construct
  REGION 1..502: note = Synthetic: UNC79_frag4
MHNMLSPFHSPFQSPFRSPLRSPFRSPFKNFGHPGGRTIDFDCEDDEMNLNCFILMFDLLLKQMELQDDGITMGLEHSLSKDIISIINNVFQAPWGGSHTCQKDEKAIECNLCQSSILCYQLACELLERLAPKEESRLVEPTDSLEDSLLSSRPEFIIGPEGEEEENPASKHGENPGNCTEPVEHAAVKNDTERKFCYQQLPVTLRLIYTIFQEMAKFEEPDILFNMLNCLKILCLHGECLYIARKDHPQFLAYIQDHMLIASLWRVVKSEFSQLSSLAVPLLLHALSLPHGADIFWTIINGNFNSKDWKMRFEAVEKVAVICRFLDIHSVTKNHLLKYSLAHAFCCFLTAVEDVNPAVATRAGLLLDTIKRPALQGLCLCLDFQFDTVVKDRPTILSKLLLLHFLKQDIPALSWEFFVNRFETLSLEAQLHLDCNKEFPFPTTITAVRTNVANLSDAALWKIKRARFARNRQKSVRSLRDSVKGPVESKRALSLPETLTSK

SEQ ID NO: 68 - protein, 502 aa, synthetic construct
  REGION 1..502: note = Synthetic: UNC79_frag5
MLAYIQDHMLIASLWRVVKSEFSQLSSLAVPLLLHALSLPHGADIFWTIINGNFNSKDWKMRFEAVEKVAVICRFLDIHSVTKNHLLKYSLAHAFCCFLTAVEDVNPAVATRAGLLLDTIKRPALQGLCLCLDFQFDTVVKDRPTILSKLLLLHFLKQDIPALSWEFFVNRFETLSLEAQLHLDCNKEFPFPTTITAVRTNVANLSDAALWKIKRARFARNRQKSVRSLRDSVKGPVESKRALSLPETLTSKIRQQSPENDNTIKDLLPEDAGIDHQTVHQLITVLMKFMAKDESSAESDISSAKAFNTVKRHLYVLLGYDQQEGCFMIAPQKMRLSTCFNAFIAGIAQVMDYNINLGKHLLPLVVQVLKYCSCPQLRHYFQQPPRCSLWSLKPHIRQMWLKALLVILYKYPYRDCDISKILLHLIHITVNTLNAQYHSCKPHATAGPLYSDNSNISRYSEKEKGEIELAEYRETGALQDSLLHCVREESIPKKKLRSFKQK

SEQ ID NO: 69 - protein, 502 aa, synthetic construct
  REGION 1..502: note = Synthetic: UNC79_frag6
MKIRQQSPENDNTIKDLLPEDAGIDHQTVHQLITVLMKFMAKDESSAESDISSAKAFNTVKRHLYVLLGYDQQEGCFMIAPQKMRLSTCFNAFIAGIAQVMDYNINLGKHLLPLVVQVLKYCSCPQLRHYFQQPPRCSLWSLKPHIRQMWLKALLVILYKYPYRDCDISKILLHLIHITVNTLNAQYHSCKPHATAGPLYSDNSNISRYSEKEKGEIELAEYRETGALQDSLLHCVREESIPKKKLRSFKQKSLDIGNADSLLFTLDEHRRKSCIDRCDIEKPPTQAAYIAQRPNDPGRSRQNSATRPDNSEIPENPAMEGFPDARRPVIPEVRLNCMETFEVKVDSPVKPAPKEDLDLIDLSSDSTSGPEKHSILSTSDSDSLVFEPLPPLRIVESDEEEETMNQGDDGPSGKNAASSPSVPSHPSVLSLSTAPLVQVSVEDCSKDFSSKDSGNNQSAGNTDSALITLEDPMDAEGSSKPEELPEFSCGSPLTLKQKRDLL

SEQ ID NO: 70 - protein, 502 aa, synthetic construct
  REGION 1..502: note = Synthetic: UNC79_frag7
MKSLDIGNADSLLFTLDEHRRKSCIDRCDIEKPPTQAAYIAQRPNDPGRSRQNSATRPDNSEIPENPAMEGFPDARRPVIPEVRLNCMETFEVKVDSPVKPAPKEDLDLIDLSSDSTSGPEKHSILSTSDSDSLVFEPLPPLRIVESDEEEETMNQGDDGPSGKNAASSPSVPSHPSVLSLSTAPLVQVSVEDCSKDFSSKDSGNNQSAGNTDSALITLEDPMDAEGSSKPEELPEFSCGSPLTLKQKRDLLQKSFALPEMSLDDHPDPGTEGEKPGELMPSSGAKTVLLKVPEDAENPTESEKPDTSAESDTEQNPERKVEEDGAEESEFKIQIVPRQRKQRKIAVSAIQREYLDISFNILDKLGEQKDPDPSTKGLSTLEMPRESSSAPTLDAGVPETSSHSSISTQYRQMKRGSLGVLTMSQLMKRQLEHQSSAPHNISNWDTEQIQPGKRQCNVPTCLNPDLEGQPLRMRGATKSSLLSAPSIVSMFVPAPEEFTDEQ

SEQ ID NO: 71 - protein, 502 aa, synthetic construct
  REGION 1..502: note = Synthetic: UNC79_frag8
MLQKSFALPEMSLDDHPDPGTEGEKPGELMPSSGAKTVLLKVPEDAENPTESEKPDTSAESDTEQNPERKVEEDGAEESEFKIQIVPRQRKQRKIAVSAIQREYLDISFNILDKLGEQKDPDPSTKGLSTLEMPRESSSAPTLDAGVPETSSHSSISTQYRQMKRGSLGVLTMSQLMKRQLEHQSSAPHNISNWDTEQIQPGKRQCNVPTCLNPDLEGQPLRMRGATKSSLLSAPSIVSMFVPAPEEFTDEQPTVMTDKCHDCGAILEEYDEETLGLAIVVLSTFIHLSPDLAAPLLLDIMQSVGRLASSTTFSNQAESMMVPGNAAGVAKQFLRCIFHQLAPNGIFPQLFQSTIKDGTFLRTLASSLMDFNELSSIAALSQLLEGLNNKKNLPAGGAMIRCLENIATFMEALPMDSPSSLWTTISNQFQTFFAKLPCVLPLKCSLDSSLRIMICLLKIPSTNATRSLLEPFSKLLSFVIQNAVFTLAYLVELCGLCYRAFT

SEQ ID NO: 72 - protein, 502 aa, synthetic construct
  REGION 1..502: note = Synthetic: UNC79_frag9
MQPTVMTDKCHDCGAILEEYDEETLGLAIVVLSTFIHLSPDLAAPLLLDIMQSVGRLASSTTFSNQAESMMVPGNAAGVAKQFLRCIFHQLAPNGIFPQLFQSTIKDGTFLRTLASSLMDFNELSSIAALSQLLEGLNNKKNLPAGGAMIRCLENIATFMEALPMDSPSSLWTTISNQFQTFFAKLPCVLPLKCSLDSSLRIMICLLKIPSTNATRSLLEPFSKLLSFVIQNAVFTLAYLVELCGLCYRAFTKERDKFYLSRSVVLELLQALKLKSPLPDTNLLLLVQFICADAGTKLAESTILSKQMIASVPGCGTAAMECVRQYINEVLDFMADMHTLTKLKSHMKTCSQPLHEDTFGGHLKVGLAQIAAMDISRGNHRDNKAVIRYLPWLYHPPSAMQQGPKEFIECVSHIRLLSWLLLGSLTHNAVCPNASSPCLPIPLDAGSHVADHLIVILIGFPEQSKTSVLHMCSLFHAFIFAQLWTVYCEQSAVATNLQNQNE

SEQ ID NO: 73 - protein, 387 aa, synthetic construct
  REGION 1..387: note = Synthetic: UNC79_frag10
MTKERDKFYLSRSVVLELLQALKLKSPLPDTNLLLLVQFICADAGTKLAESTILSKQMIASVPGCGTAAMECVRQYINEVLDFMADMHTLTKLKSHMKTCSQPLHEDTFGGHLKVGLAQIAAMDISRGNHRDNKAVIRYLPWLYHPPSAMQQGPKEFIECVSHIRLLSWLLLGSLTHNAVCPNASSPCLPIPLDAGSHVADHLIVILIGFPEQSKTSVLHMCSLFHAFIFAQLWTVYCEQSAVATNLQNQNEFSFTAILTALEFWSRVTPSILQLMAHNKVMVEMVCLHVISLMEALQECNSTIFVKLIPMWLPMIQSNIKHLSAGLQLRLQAIQNHVNHHSLRTLPGSGQSSAGLAALRKWLQCTQFKMAQVEIQSSEAASQFYPL

SEQ ID NO: 74 - protein, 500 aa, synthetic construct
  REGION 1..500: note = Synthetic: UNC80_frag1
MVKRKSSEGQEQDGGRGIPLPIQTFLWRQTSAFLRPKLGKQYEASCVSFERVLVENKLHGLSPALSEAIQSISRWELVQAALPHVLHCTATLLSNRNKLGHQDKLGVAETKLLHTLHWMLLEAPQDCNNERFGGTDRGSSWGGSSSAFIHQVENQGSPGQPCQSSSNDEEENNRRKIFQNSMATVELFVFLFAPLVHRIKESDLTFRLASGLVIWQPMWEHRQPGVSGFTALVKPIRNIITAKRSSPINSQSRTCESPNQDARHLEGLQVVCETFQSDSISPKATISGCHRGNSFDGSLSSQTSQERGPSHSRASLVIPPCQRSRYATYFDVAVLRCLLQPHWSEEGTQWSLMYYLQRLRHMLEEKPEKPPEPDIPLLPRPRSSSMVAAAPSLVNTHKTQDLTMKCNEEEKSLSSEAFSKVSLTNLRRSAVPDLSSDLGMNIFKKFKSRKEDRERKGSIPFHHTGKRRPRRMGVPFLLHEDHLDVSPTRSTFSFGSFSGL

SEQ ID NO: 75 - protein, 502 aa, synthetic construct
  REGION 1..502: note = Synthetic: UNC80_frag2
MSQSRTCESPNQDARHLEGLQVVCETFQSDSISPKATISGCHRGNSFDGSLSSQTSQERGPSHSRASLVIPPCQRSRYATYFDVAVLRCLLQPHWSEEGTQWSLMYYLQRLRHMLEEKPEKPPEPDIPLLPRPRSSSMVAAAPSLVNTHKTQDLTMKCNEEEKSLSSEAFSKVSLTNLRRSAVPDLSSDLGMNIFKKFKSRKEDRERKGSIPFHHTGKRRPRRMGVPFLLHEDHLDVSPTRSTFSFGSFSGLGEDRRGIEKGGWQTTILGKLTRRGSSDAATEMESLSARHSHSHHTLVSDLPDPSNSHGENTVKEVRSQISTITVATFNTTLASFNVGYADFFNEHMRKLCNQVPIPEMPHEPLACANLPRSLTDSCINYSYLEDTEHIDGTNNFVHKNGMLDLSVVLKAVYLVLNHDISSRICDVALNIVECLLQLGVVPCVEKNRKKSENKENETLEKRPSEGAFQFKGVSGSSTCGFGGPAVSGAGDGGGEEGGGGDG

SEQ ID NO: 76 - protein, 502 aa, synthetic construct
  REGION 1..502: note = Synthetic: UNC80_frag3
MLGEDRRGIEKGGWQTTILGKLTRRGSSDAATEMESLSARHSHSHHTLVSDLPDPSNSHGENTVKEVRSQISTITVATFNTTLASFNVGYADFFNEHMRKLCNQVPIPEMPHEPLACANLPRSLTDSCINYSYLEDTEHIDGTNNFVHKNGMLDLSVVLKAVYLVLNHDISSRICDVALNIVECLLQLGVVPCVEKNRKKSENKENETLEKRPSEGAFQFKGVSGSSTCGFGGPAVSGAGDGGGEEGGGGDGGGGGGDGGGGGGGGGGPYEKNDKNQEKDESTPVSNHRLALTMLIKIVKSLGCAYGCGEGHRGLSGDRLRHQVFRENAQNCLTKLYKLDKMQFRQTMRDYVNKDSLNNVVDFLHALLGFCMEPVTDNKAGFGNNFTTVDNKSTAQNVEGIIVSAMFKSLITRCASTTHELHSPENLGLYCDIRQLVQFIKEAHGNVFRRVALSALLDSAEKLAPGKKVEENEQESKPAGSKRSEAGSIVDKGQVSSAPEEC

SEQ ID NO: 77 - protein, 502 aa, synthetic construct
  REGION 1..502: note = Synthetic: UNC80_frag4
MGGGGGGDGGGGGGGGGGPYEKNDKNQEKDESTPVSNHRLALTMLIKIVKSLGCAYGCGEGHRGLSGDRLRHQVFRENAQNCLTKLYKLDKMQFRQTMRDYVNKDSLNNVVDFLHALLGFCMEPVTDNKAGFGNNFTTVDNKSTAQNVEGIIVSAMFKSLITRCASTTHELHSPENLGLYCDIRQLVQFIKEAHGNVFRRVALSALLDSAEKLAPGKKVEENEQESKPAGSKRSEAGSIVDKGQVSSAPEECRSFMSGRPSQTPEHDEQMQGANLGRKDFWRKMFKSQSAASDTSSQSEQDTSECTTAHSGTTSDRRARSRSRRISLRKKLKLPIGKRNWLKRSSLSGLADGVEDLLDISSVDRLSFIRQSSKVKFTSAVKLSEGGPGSGMENGRDEEENFFKRLGCHSFDDHLSPNQDGGKSKNVVNLGAIRQGMKRFQFLLNCCEPGTIPDASILAAALDLEAPVVARAALFLECARFVHRCNRGNWPEWMKGHHVNITK

SEQ ID NO: 78 - protein, 502 aa, synthetic construct
  REGION 1..502: note = Synthetic: UNC80_frag5
MCRSFMSGRPSQTPEHDEQMQGANLGRKDFWRKMFKSQSAASDTSSQSEQDTSECTTAHSGTTSDRRARSRSRRISLRKKLKLPIGKRNWLKRSSLSGLADGVEDLLDISSVDRLSFIRQSSKVKFTSAVKLSEGGPGSGMENGRDEEENFFKRLGCHSFDDHLSPNQDGGKSKNVVNLGAIRQGMKRFQFLLNCCEPGTIPDASILAAALDLEAPVVARAALFLECARFVHRCNRGNWPEWMKGHHVNITKKGLSRGRSPIVGNKRNQKLQWNAAKLFYQWGDAIGVRLNELCHGESESPANLLGLIYDEETKRRLRKEDEEEDFLDDSTVNPSKCGCPFALKMAACQLLLEITTFLRETFSCLPRPRTEPLVDLESCRLRLDPELDRHRYERKISFAGVLDENEDSKDSLHSSSHTLKSDAGVEEKKEGSPWSASEPSIEPEGMSNAGAEENYHRNMSWLHVMILLCNQQSFICTHVDYCHPHCYLHHSRSCARLVRAIK

SEQ ID NO: 79 - protein, 502 aa, synthetic construct
  REGION 1..502: note = Synthetic: UNC80_frag6
MKKGLSRGRSPIVGNKRNQKLQWNAAKLFYQWGDAIGVRLNELCHGESESPANLLGLIYDEETKRRLRKEDEEEDFLDDSTVNPSKCGCPFALKMAACQLLLEITTFLRETFSCLPRPRTEPLVDLESCRLRLDPELDRHRYERKISFAGVLDENEDSKDSLHSSSHTLKSDAGVEEKKEGSPWSASEPSIEPEGMSNAGAEENYHRNMSWLHVMILLCNQQSFICTHVDYCHPHCYLHHSRSCARLVRAIKLLYGDSVDSLRESSNISSVALRGKKQKECSDKSCLRTPSLKKRVSDANLEGKKDSGMLKYIRLQVMSLSPAPLSLLIKAAPILTEEMYGDIQPAAWELLLSMDEHMAGAAAAMFLLCAVKVPEAVSDMLMSEFHHPETVQRLNAVLKFHTLWRFRYQVWPRMEEGAQQIFKIPPPSINFTLPSPVLGMPSVPMFDPPWVPQCSGSVQDPINEDQSKSFSARAVSRSHQRAEHILKNLQQEEEKKRLGREA

SEQ ID NO: 80 - protein, 502 aa, synthetic construct
  REGION 1..502: note = Synthetic: UNC80_frag7
MKLLYGDSVDSLRESSNISSVALRGKKQKECSDKSCLRTPSLKKRVSDANLEGKKDSGMLKYIRLQVMSLSPAPLSLLIKAAPILTEEMYGDIQPAAWELLLSMDEHMAGAAAAMFLLCAVKVPEAVSDMLMSEFHHPETVQRLNAVLKFHTLWRFRYQVWPRMEEGAQQIFKIPPPSINFTLPSPVLGMPSVPMFDPPWVPQCSGSVQDPINEDQSKSFSARAVSRSHQRAEHILKNLQQEEEKKRLGREASLITAIPITQEACYEPTCTPNSEPEEEVEEVTNLASRRLSVSPSCTSSTSHRNYSFRRGSVWSVRSAVSAEDEEHTTEHTPNHHVPQPPQAVFPACICAAVLPIVHLMEDGEVREDGVAVSAVAQQVLWNCLIEDPSTVLRHFLEKLTISNRQDELMYMLRKLLLNIGDFPAQTSHILFNYLVGLIMYFVRTPCEWGMDAISATLTFLWEVVGYVEGLFFKDLKQTMKKEQCEVKLLVTASMPGTKTLVV

SEQ ID NO: 81 - protein, 502 aa, synthetic construct
  REGION 1..502: note = Synthetic: UNC80_frag8
MASLITAIPITQEACYEPTCTPNSEPEEEVEEVTNLASRRLSVSPSCTSSTSHRNYSFRRGSVWSVRSAVSAEDEEHTTEHTPNHHVPQPPQAVFPACICAAVLPIVHLMEDGEVREDGVAVSAVAQQVLWNCLIEDPSTVLRHFLEKLTISNRQDELMYMLRKLLLNIGDFPAQTSHILFNYLVGLIMYFVRTPCEWGMDAISATLTFLWEVVGYVEGLFFKDLKQTMKKEQCEVKLLVTASMPGTKTLVVHGQNECDIPTQLPVHEDTQFEALLKECLEFFNIPESQSTHYFLMDKRWNLIHYNKTYVRDIYPFRRSVSPQLNLVHMHPEKGQELIQKQVFTRKLEEVGRVLFLISLTQKIPTAHKQSHVSMLQEDLLRLPSFPRSAIDAEFSLFSDPQAGKELFGLDTLQKSLWIQLLEEMFLGMPSEFPWGDEIMLFLNVFNGALILHPEDSALLRQYAATVINTAVHFNHLFSLSGYQWILPTMLQVYSDYESNPQL

SEQ ID NO: 82 - protein, 502 aa, synthetic construct
  REGION 1..502: note = Synthetic: UNC80_frag9
MVHGQNECDIPTQLPVHEDTQFEALLKECLEFFNIPESQSTHYFLMDKRWNLIHYNKTYVRDIYPFRRSVSPQLNLVHMHPEKGQELIQKQVFTRKLEEVGRVLFLISLTQKIPTAHKQSHVSMLQEDLLRLPSFPRSAIDAEFSLFSDPQAGKELFGLDTLQKSLWIQLLEEMFLGMPSEFPWGDEIMLFLNVFNGALILHPEDSALLRQYAATVINTAVHFNHLFSLSGYQWILPTMLQVYSDYESNPQLRQAIEFACHQFYILHRKPFVLQLFASVAPLLEFPDAANNGPSKGVSAQCLFDLLQSLEGETTDILDILELVKAEKPLKSLDFCYGNEDLTFSISEAIKLCVTVVAYAPESFRSLQMLMVLEALVPCYLQKLKRQTSQVETVPAAREEIAATAALATSLQALLYSVEVLTRPMTAPQMSRCDQGHKGTTTANHTMSSGVNTRYQEQGAKLHFIRENLHLLEEGQGIPREELDERIAREEFRRPRESLLNIC

SEQ ID NO: 83 - protein, 502 aa, synthetic construct
  REGION 1..502: note = Synthetic: UNC80_frag10
MLRQAIEFACHQFYILHRKPFVLQLFASVAPLLEFPDAANNGPSKGVSAQCLFDLLQSLEGETTDILDILELVKAEKPLKSLDFCYGNEDLTFSISEAIKLCVTVVAYAPESFRSLQMLMVLEALVPCYLQKLKRQTSQVETVPAAREEIAATAALATSLQALLYSVEVLTRPMTAPQMSRCDQGHKGTTTANHTMSSGVNTRYQEQGAKLHFIRENLHLLEEGQGIPREELDERIAREEFRRPRESLLNICTEFYKHCGPRLKILQNLAGEPRVIALELLDVKSHMRLAEIAHSLLKLAPYDTQTMESRGLRRYIMEMLPITDWTAEAVRPALILILKRLDRMFNKIHKMPTLRRQVEWEPASNLIEGVCLTLQRQPIISFLPHLRSLINVCVNLVMGVVGPSSVADGLPLLHLSPYLSPPLPFSTAVVRLVALQIQALKEDFPLSHVISPFTNQERREGMLLNLLIPFVLTVGSGSKDSPWLEQPEVQLLLQTVINVLLP

SEQ ID NO: 84 - protein, 502 aa, synthetic construct
  REGION 1..502: note = Synthetic: UNC80_frag11
MCTEFYKHCGPRLKILQNLAGEPRVIALELLDVKSHMRLAEIAHSLLKLAPYDTQTMESRGLRRYIMEMLPITDWTAEAVRPALILILKRLDRMFNKIHKMPTLRRQVEWEPASNLIEGVCLTLQRQPIISFLPHLRSLINVCVNLVMGVVGPSSVADGLPLLHLSPYLSPPLPFSTAVVRLVALQIQALKEDFPLSHVISPFTNQERREGMLLNLLIPFVLTVGSGSKDSPWLEQPEVQLLLQTVINVLLPPRIISTSRSKNFMLESSPAHCSTPGDAGKDLRREGLAESTSQAAYLALKVILVCFERQLGSQWYWLSLQVKEMALRKVGGLALWDFLDFIVRTRIPIFVLLRPFIQCKLLAQPAENHEELSARQHIADQLERRFIPRPLCKSSLIAEFNSELKILKEAVHSGSAYQGKTSISTVGTSTSAYRLSLATMSRSNTGTGTVWEQDSEPSQQASQDTLSRTDEEDEENDSISMPSVVSEQEAYLLSAIGRRRFS

SEQ ID NO: 85 - protein, 510 aa, synthetic construct
  REGION 1..510: note = Synthetic: UNC80_frag12
MPPRIISTSRSKNFMLESSPAHCSTPGDAGKDLRREGLAESTSQAAYLALKVILVCFERQLGSQWYWLSLQVKEMALRKVGGLALWDFLDFIVRTRIPIFVLLRPFIQCKLLAQPAENHEELSARQHIADQLERRFIPRPLCKSSLIAEFNSELKILKEAVHSGSAYQGKTSISTVGTSTSAYRLSLATMSRSNTGTGTVWEQDSEPSQQASQDTLSRTDEEDEENDSISMPSVVSEQEAYLLSAIGRRRFSSHVSSMSVPQAEVGMLPSQSEPNVLDDSQGLAAEGSLSRVASIQSEPGQQNLLVQQPLGRKRGLRQLRRPLLSRQKTQTEPRNRQGARLSTTRRSIQPKTKPSADQKRSVTFIEAQPEPAAAPTDALPATGQLQGCSPAPSRKPEAMDEPVLTSSPAIVVADLHSVSPKQSENFPTEEGEKEEDTEAQGATAHSPLSAQLSDPDDFTGLETSSLLQHGDTVLHISEENGMENPLLSSQFTFTPTELGKTDAVLDESHV

SEQ ID NO: 86 - protein, 4 aa, synthetic construct
  REGION 1..4: note = Synthetic: linker
GGGS

SEQ ID NO: 87 - protein, 12 aa, synthetic construct
  REGION 1..12: note = Synthetic: linker
GGGSGGGSGGGS

(19) United States
(12) Patent Application Publication
(10) Pub. No.: US 2024/0295565 A1
(43) Pub. Date: Sep. 5, 2024

(54) NALCN Channel Protein and Modulators Thereof

(71) Applicants: Genentech Inc., South San Francisco, CA (US); University of Copenhagen, København (DK)

(72) Inventors: Jian Mehr-Dean Payandeh, Belmont, CA (US); Stephan Alexander Pless, Copenhagen (DK); Han Chow Chua, Copenhagen (DK); Marc Kschonsak, South San Francisco, CA (US)

(73) Assignees: Genentech Inc., South San Francisco, CA (US); University of Copenhagen, København (DK)

(21) Appl. No.: 18/405,256

(22) Filed: Jan. 5, 2024

Related U.S. Application Data
(63) Continuation of application No. PCT/US22/73449, filed on Jul. 6, 2022.
(60) Provisional application No. 63/219,776, filed on Jul. 8, 2021.

(51) Int. Cl. G01N 33/68; C07K 14/47; G01N 33/557

(52) U.S. Cl. CPC G01N 33/6872; C07K 14/47; G01N 33/557

(57) ABSTRACT

The present disclosure relates, inter alia, to complexes of the Na+ leak channel non-selective protein (NALCN) with FAM155A (also called FAM155; Family with sequence similarity 155 member A), UNC79 (uncoordinated 79), and UNC80 (uncoordinated 80), methods of screening for molecules that modulate the activity of the complex, and identified modulators thereof.

### CROSS-REFERENCE TO RELATED APPLICATIONS

This application is a continuation of International Patent Application No. PCT/US2022/073449, filed Jul. 6, 2022, which claims the benefit of priority of U.S. Provisional Application No. 63/219,776, filed Jul. 8, 2021, the contents of each of which are incorporated by reference herein in their entireties for any purpose.

### SEQUENCE LISTING

The instant application contains a Sequence Listing which has been submitted electronically in XML format and is hereby incorporated by reference in its entirety. Said XML copy, created on Dec. 18, 2023, is named “01164-0018-00US_Sequence Listing.xml” and is 204,232 bytes in size.

### FIELD

The present disclosure relates, inter alia, to complexes of the Na+ leak channel non-selective protein (NALCN) with FAM155A (also called FAM155; Family with sequence similarity 155 member A), UNC79 (uncoordinated 79), and UNC80 (uncoordinated 80), methods of screening for molecules that modulate the activity of the complex, and identified modulators thereof.

### BACKGROUND

In many neurons, the NALCN (Na+ leak channel non-selective) channelosome contributes to basal Na+ leak currents that regulate resting membrane potential, spontaneous firing, and pace-making activity. NALCN represents a distinct branch of the four-domain ion channel superfamily and is an orphan gene in humans. Unlike distantly related voltage-gated sodium (Nav) and calcium (Cav) channels, NALCN is not gated by changes in membrane potential. Robust activity in heterologous systems requires the co-expression of three other proteins: FAM155A (also called FAM155; Family with sequence similarity 155 member A), UNC79 (uncoordinated 79), and UNC80 (uncoordinated 80). Mutations in NALCN and UNC80 have been found, for example, to cause a range of neurodevelopmental disorders, such as motor, mental, and visual defects. FAM155A, UNC79, and UNC80 bear no sequence resemblance to canonical Nav or Cav channel auxiliary subunits, suggesting they serve specialized scaffolding or functional roles.

Recently, the basic architecture of the NALCN-FAM155A pore-forming subcomplex was determined, and revealed that FAM155A forms an integrated dome-like structure over the NALCN selectivity filter, while the S6-gate was closed in the absence of UNC79 and UNC80. (M. Kschonsak et al., Nature 587: 313-318 (2020); Y. Kang et al., Nature Comm. 11:6199 [doi.org/10.1038/s41467-020-20002-9] (2000); J. Xie et al., Nature Comm. 11:5831 [doi.org/10.1038/s41467-020-19667-z] (2000).) UNC79 and UNC80 have been proposed to physically interact with NALCN, and UNC79 has been suggested to bind UNC80 to promote dendritic localization of NALCN. Previous attempts to form a complex of NALCN and FAM155A further comprising UNC79 and UNC80 were not successful, however, and resulted only in an NALCN-FAM155A subcomplex. (See Id.)

### SUMMARY

The present inventors have now successfully prepared complexes of all four of NALCN, FAM155A, UNC79, and UNC80, in some embodiments, further with calmodulin (CaM), as described in more detail in the Examples section below. And the instant inventors have determined an overall structure of this complex at 3.3-3.1 Angstrom resolution by cryo-electron microscopy. The ability to obtain a complex of these proteins suitable for functional assays such as ion channel assays and binding affinity assays, for example, allows, among other things, for screening molecules to identify modulators of the complex and molecules that bind to the complex.

Accordingly, the application includes, for example, a method of identifying a modulator of a complex of human NALCN (Na+ leak channel non-selective), FAM155 (Family with sequence similarity 155 member A), UNC79 (uncoordinated 79), and UNC80 (uncoordinated 80) (a human NALCN-FAM155-UNC79-UNC80 complex), comprising: (a) providing a human NALCN-FAM155-UNC79-UNC80 complex in vitro; (b) contacting the complex with a potential modulator of the complex; (c) performing an ion channel assay of the complex in the presence of the potential modulator; and (d) identifying the potential modulator as a modulator of the complex if the activity of the complex in the assay in the presence of the potential modulator is higher or lower than the activity of the complex in the assay in the absence of the potential modulator. In some cases, the ion channel assay is a patch clamp or an automated patch clamp assay, an ion flux assay, or an ion- or voltage-sensitive dye assay. In some cases, the activity of the complex in the assay in the presence of the potential modulator is lower than the activity of the complex in the assay in the absence of the potential modulator (i.e., the potential modulator reduces the activity of the complex in the assay). In some cases, the potential modulator identified in part (c) reduces the activity of the complex in the assay by at least 25%, at least 30%, at least 40%, at least 50%, at least 60%, at least 70%, at least 80%, or at least 90%. In some aspects, the activity of the complex in the assay in the presence of the potential modulator is higher than the activity of the complex in the assay in the absence of the potential modulator (i.e., the potential modulator increases the activity of the complex in the assay). In some embodiments, the method further comprises determining the binding affinity of the potential modulator identified in part (d) to the human NALCN-FAM155-UNC79-UNC80 complex, a human NALCN-FAM155 complex, a UNC79-UNC80 complex, or to one or more of human NALCN, FAM155, UNC79, or UNC80. In some cases, the potential modulator binds to the human NALCN-FAM155-UNC79-UNC80 complex with an EC50 or IC50 of 10 uM or less, 10 uM to 50 nM, 10 uM to 500 nM, 1 uM or less, 1 uM to 50 nM, or 100 nM or less. In some aspects, the method is performed in the presence of an NALCN-DII-DIII linker peptide or an NALCN-DI-DII linker peptide or an identified modulator of the human NALCN-FAM155-UNC79-UNC80 complex. In some aspects, the linker peptide comprises an amino acid sequence of any one of SEQ ID Nos: 16-24, or comprises an amino acid sequence of any one of SEQ ID Nos: 16-18 or 23-24. In some cases, the method further comprises determining whether the potential modulator modulates activity of a human NALCN-FAM155-UNC79-UNC80 complex, wherein the complex comprises a mutant human NALCN. In some cases, the mutant human NALCN comprises a substitution, insertion, or deletion in one or both of the DI-DII linker or the DII-DIII linker. In some cases, the DI-DII linker or the DII-DIII linker is replaced by a corresponding region from a human or mammalian Nav or Cav protein, such as from human Nav1.4; comprise a deletion; or comprise an insertion of a GGGS element. In some cases, the mutant human NALCN comprises an amino acid sequence of any one of SEQ ID Nos: 7, 8, 12-15, 32-52, or 59-63. In some aspects, the method further comprises determining whether the potential modulator modulates activity of a human NALCN-FAM155-UNC79-UNC80 complex, wherein the complex comprises a mutant human UNC79, for example comprising an amino acid sequence of any one of SEQ ID Nos: 53, 54, 55, 58, or 64-73. In some aspects, the method further comprises determining whether the potential modulator modulates activity of a human NALCN-FAM155-UNC79-UNC80 complex, wherein the complex comprises a mutant human UNC80, for example comprising an amino acid sequence of any one of SEQ ID Nos: 74-85.

The present disclosure also includes, for example, a method of identifying a molecule that binds to a complex of human NALCN (Na+ leak channel non-selective), human FAM155 (Family with sequence similarity 155 member A), human UNC79 (uncoordinated 79), and human UNC80 (uncoordinated 80): (a human NALCN-FAM155-UNC79-UNC80 complex), the method comprising: (a) providing a human NALCN-FAM155-UNC79-UNC80 complex in vitro; (b) contacting the complex with one or more test molecules, and separating bound from unbound test molecules; and (c) identifying a test molecule as binding to the human NALCN-FAM155-UNC79-UNC80 complex if the test molecule remains bound to the complex after separating bound from unbound test molecules.

The present disclosure also includes, for example, a method of identifying a molecule that binds to a complex of human NALCN (Na+ leak channel non-selective), human FAM155 (Family with sequence similarity 155 member A), human UNC79 (uncoordinated 79), and human UNC80 (uncoordinated 80): (a human NALCN-FAM155-UNC79-UNC80 complex), the method comprising: (a) providing a human NALCN-FAM155-UNC79-UNC80 complex in vitro; (b) contacting the complex with an NALCN-DII-DIII linker peptide or an NALCN-DI-DII linker peptide, and further with a test molecule; and (c) identifying the test molecule as binding to the human NALCN-FAM155-UNC79-UNC80 complex if the test molecule competes with the NALCN-DII-DIII linker peptide and/or the NALCN-DI-DII linker peptide for binding to the complex.

The present disclosure also includes, for example, a method of identifying a molecule that modulates formation of or stability of a complex of human NALCN (Na+ leak channel non-selective), human FAM155 (Family with sequence similarity 155 member A), human UNC79 (uncoordinated 79), and human UNC80 (uncoordinated 80): (a human NALCN-FAM155-UNC79-UNC80 complex) in vitro, the method comprising: (a) providing human NALCN, human FAM155, human UNC79, and human UNC80 under conditions in which human NALCN-FAM155-UNC79-UNC80 complex formation is promoted in vitro; (b) contacting the complex with a test molecule; and (c) identifying the test molecule as modulating formation of or stability of the human NALCN-FAM155-UNC79-UNC80 complex if the degree of complex formation in the presence of the test molecule is increased or reduced compared to in the absence of the test molecule.

In some aspects of these methods, the potential modulator binds to the human NALCN-FAM155-UNC79-UNC80 complex with an EC50 or IC50 of 10 UM or less, 10 uM to 50 nM, 10 uM to 500 nM, 1 uM or less, 1 uM to 50 nM, or 100 nM or less. In some aspects, binding affinity is determined by an ELISA, AlphaLISA, or FRET assay.

In some cases, for any of the methods herein, at least one of the human NALCN, human FAM155, human UNC79, or human UNC80 of the human NALCN-FAM155-UNC79-UNC80 complex is labeled. In some cases, at least one of the human NALCN, human FAM155, human UNC79, or human UNC80 of the human NALCN-FAM155-UNC79-UNC80 complex is attached to a matrix, such as a bead, chip, or plate. In some cases, the human NALCN-FAM155-UNC79-UNC80 complex is solubilized in a lipid bilayer, detergent, or lipid nanodisc. In some cases, the method further comprises determining whether the molecule binds to a human NALCN-FAM155 complex, a human UNC79-UNC80 complex, or to one or more of human NALCN, FAM155, UNC79, or UNC80.

In some aspects, in any of the methods herein, the method further comprises determining whether the test molecule binds to a human NALCN-FAM155-UNC79-UNC80 complex wherein the complex comprises a mutant human NALCN. In some such cases, the mutant human NALCN comprises a substitution, insertion, or deletion in one or both of the DI-DII linker or the DII-DIII linker. In some cases, the DI-DII linker or the DII-DIII linker are replaced by a corresponding loop from a human or mammalian Nav or Cav protein, such as from human Nav1.4; comprise a deletion; or comprise an insertion of a GGGS element (SEQ ID NO: 86). In some cases, the mutant human NALCN comprises an amino acid sequence of any one of SEQ ID Nos: 7, 8, 12-15, 32-52, or 59-63. In some cases, in any of the methods herein, the human NALCN-FAM155-UNC79-UNC80 complex further comprises human calmodulin.

In some cases, in any of the methods herein, the potential modulator or test molecule is a peptide, macrocycle, or antibody. In some cases, the potential modulator or test molecule is a small molecule.

The present disclosure also encompasses, for example, molecules identified by the methods herein. In some cases, such a molecule is a peptide, macrocycle, or antibody. In other cases, it is a small molecule. The present disclosure also includes an identified modulator of the complex, as described herein, such as an identified peptide modulator of human NALCN, for instance, a peptide comprising the amino acid sequence of any one of SEQ ID Nos: 16-18 or 23-24.

The present disclosure also includes, for example, use of a molecule or identified modulator as described herein in treating a neurodevelopmental disorder, a circadian rhythm disorder, or pain in a subject, as well as, for example, a method of treating a subject with neurodevelopmental disorder, a circadian rhythm disorder, or pain, comprising administering an effective amount of a molecule or identified peptide modulator as described herein.

The disclosure further includes, for example, a kit comprising at least one of: an identified modulator of the human NALCN-FAM155-UNC79-UNC80 complex, one or more reagents for conducting an ion channel assay, labeled NALCN, UNC79, UNC80, or FAM155 optionally attached to a matrix such as beads, chips or plates, and instructions for use.

The disclosure also includes, for instance, an isolated complex of human NALCN, UNC79, UNC80, and FAM155, optionally wherein the complex is comprised within a lipid bilayer or lipid nanodisc, and optionally wherein at least one of the human NALCN, UNC79, UNC80, and FAM155 includes a label or is attached to a matrix, such as a bead, plate, or chip. In some cases, a complex further comprises a modulator of human NALCN or a molecule identified by any of the methods herein.

Additional objects and advantages will be set forth in part in the description which follows, and in part will be understood from the description, or may be learned by practice. The objects and advantages will be realized and attained by means of the elements and combinations particularly pointed out in the appended claims. It is to be understood that both the foregoing general description and the following detailed description are exemplary and explanatory only and are not restrictive of the claims. The accompanying drawings, which are incorporated in and constitute a part of this specification, illustrate one (several) embodiment(s) and together with the description, serve to explain the principles described herein.

### BRIEF DESCRIPTION OF THE DRAWINGS

FIGS. 1A-F show overall structure of the human NALCN channelosome. FIG. 1A shows current traces from Xenopus oocytes expressing NALCN alone or with indicated combinations of FAM155A, UNC79 and/or UNC80. NALCN subunit is wild-type or contains a C-terminal GFP-Flag tag fusion, as indicated. Steps from +80 to −100 mV, 20 mV increments, in ND96 recording solution. FIG. 1B shows an overall cryo-EM map. FIG. 1C shows a cartoon representation of the overall NALCN channelsome model. FIG. 1D-F show alternative views of the NALCN channelosome.

FIGS. 2A-T show overall structure of the UNC79-UNC80 subcomplex. FIG. 2A shows cartoon cylinder representations of UNC79 and UNC80. FIG. 2B shows a close-in view of the N—C interface. FIG. 2C shows a close-in view of the C—N interface. FIG. 2D shows a close-in view of the crossover interface. FIG. 2E shows mapping of deletion and disease mutations. FIGS. 2F-H show contact (FIG. 2F), electrostatic (FIG. 2G), and conservation surface (FIG. 2H) representations of the crossover interface. FIG. 2I-J show structural superposition of UNC79 and UNC80. FIG. 2K shows Ub-like insert domain of UNC80, shown in pink. FIG. 2L shows superposition of UNC80 Ub-like domain with Ub. FIGS. 2M-N show superposition of importin-α3 onto UNC79. FIG. 2O-P show overlay of importin-ß (FIG. 2O) and PP2A (FIG. 2P) onto UNC79. FIG. 2Q shows current traces from Xenopus oocytes expressing NALCN, FAM155A, UNC79 and UNC80, where subunits are wild-type or truncated as indicated. Star (*) indicates a construct containing a C-terminal GFP-Flag tag fusion. Steps from +80 to −100 mV, 20 mV increments, in ND96 recording solution. FIGS. 2R-T provides similar traces as in FIG. 2Q, but with full-length, N-terminally truncated (UNC79 468-2635* and UNC80 734-3258*) or C-terminally truncated (UNC79 1-2400* and UNC80 1-2794) constructs expressed as is or in indicated combinations. Right summary shows mean current amplitudes elicited at +80 mV from a holding potential of 0 mV for indicated construct combinations.

FIGS. 3A-J show NALCN interactions with the UNC79-UNC80 subcomplex. FIG. 3A shows NALCN in cartoon representation and UNC79 and UNC80 in surface representations. FIG. 3B shows an overall view of the NALCN DII-DIII linker structure. FIG. 3C shows a close-in view of the DI-DII linker-UNC79 interaction interface. FIG. 3D shows a view of the hinge module. FIG. 3E shows a close-in view of the DII-DIII linker lasso loop interaction with UNC80. FIG. 3F shows a close-in view of the DII-DIII linker a2-helix interaction with UNC80. FIG. 3G shows a view of DII-DIII linker Arg717 interaction with UNC79 and UNC80. FIG. 3H shows a view of CaM interaction with the NALCN CTD and UNC80. FIG. 3I-J show current traces from Xenopus oocytes expressing NALCN, FAM155A, UNC79 and UNC80, where the NALCN subunit is wild-type or has indicated deletions. Steps from +80 to −100 mV, 20 mV increments, in ND96 recording solution. Right summary shows mean current amplitudes elicited at +80 mV from a holding potential of 0 mV for indicated construct combinations.

FIGS. 4A-HF show the NALCN channelosome pore structure and gating model. FIG. 4A shows the pore radius of the ion conduction pathway in the NALCN channelosome or NALCN-FAM155A subcomplex structures. FIG. 4B shows the superposition highlighting the S6-gate region in the NALCN channelosome and NALCN-FAM155A subcomplex structures, intracellular view. FIG. 4C shows superposition of the two conformations of the NALCN channelosome, top view, with the NALCN subunit removed for clarity. Insets highlight the DI-DII and DII-DIII linkers which are both colored cyan or pink in respective conformations. FIG. 4D shows a schematic of NALCN illustrating where linker manipulations occurred. FIG. 4E shows current traces (top left panel) and mean current amplitudes (bottom left panel) for constructs WT and A-J (right panel). FIG. 4E, top left panel, shows exemplary current traces from Xenopus oocytes expressing NALCN, FAM155A, UNC79 and UNC80, where NALCN is wild-type or has indicated manipulations. FIG. 4E, bottom left panel, shows a summary of mean current amplitudes elicited at +80 mV (top bar graph) or −80 mV (bottom bar graph) from a holding potential of 0 mV expressing indicated constructs combinations. FIG. 4F shows current traces from Xenopus oocytes expressing NALCN, FAM155A, UNC79 and UNC80, where control (H₂O) or indicated isolated DII-DIII linker constructs are expressed in trans. FIG. 4G shows a schematic of the DII-DIII linker (top panel) along with summary showing mean current amplitudes elicited at +80 mV from a holding potential of 0 mV for indicated construct combinations (bottom panel). FIG. 4H shows a schematic of a potential gating model shown at for the entire NALCN channelosome (top) or at the level of the S6-gate.

FIGS. 5A-J illustrate the purification and structure determination of the NALCN channelosome. FIG. 5A shows a NALCN channelosome protein expression and purification scheme. FIGS. 5B-C shows an example size exclusion chromatograph (FIG. 5B) and SDS-PAGE (FIG. 5C) of nanodisc-reconstituted NALCN channelosome sample. FIG. 5D shows an example cryo-EM micrograph image of the NALCN channelosome-MSP1E3D1 complex. FIG. 5E shows representative 2D-class averages after 2 rounds of 2D classification from 200 classes and approximately 720,000 particles. FIG. 5F shows data collection and processing workflow. FIG. 5G shows conformation 1, FSC between two half datasets yields a global resolution estimate of approximately 3.1 Å resolution. FIG. 5H shows conformation 1, heat map representation of the distribution of assigned particle orientations. FIG. 5I shows conformation 2, FSC between two half datasets yields a global resolution estimate of approximately 3.1 Å resolution. FIG. 5J shows conformation 2, heat map representation of the distribution of assigned particle orientations.

FIGS. 6A-D show select cryo-EM map regions of the NALCN channelosome. FIG. 6A-D Example 3D map overlay is shown for DI-DII linker (FIG. 6A), DII-DIII linker (FIG. 6B), NALCN-CTD (FIG. 6C) and CaM (FIG. 6D).

FIGS. 7A-B show NALCN and FAM155A structures in the NALCN channelosome and NALCN-FAM155A subcomplex. FIG. 7A shows a comparison of the NALCN subunit in the NALCN-FAM155A subcomplex and NALCN channelosome (with UNC79 and UNC80 removed for clarity). FIG. 7B shows a superposition of the FAM155A subunit in the NALCN-FAM155A subcomplex and NALCN channelosome.

FIGS. 8A-B show that UNC79 and UNC80 are HEAT-repeat proteins. FIG. 8A shows side- and top-views of UNC79. Position of disordered loops >50 residues in length are indicated. FIG. 8B shows side- and top-views of UNC80. Position of disordered loops >50 residues in length are indicated.

FIG. 9 shows a multi-sequence alignment of UNC79.

FIG. 10 shows a multi-sequence alignment of UNC80.

FIGS. 11A-O show structure-function analysis of UNC79 and UNC80. FIGS. 11A and 11D shows a schematic of UNC79 (FIG. 11A) and a schematic of UNC80 (FIG. 11D), each with corresponding amino acid residues of fragments. FIGS. 11C and 11E show exemplary current traces from Xenopus oocytes expressing NALCN, FAM155A, UNC79 and UNC80, where various isolated ˜500 residue fragments of either UNC79 or UNC80 are co-expressed in trans, with steps from +80 to −80 mV, 40 mV increments, in ND96 recording solution. FIGS. 11C and 11F show summaries of mean current amplitudes elicited at +80 mV (top bar graphs of FIGS. 11C and 11F) or −80 mV (bottom bar graphs of FIGS. 11C and 11F) from a holding potential of 0 mV for indicated construct combinations. FIG. 11G shows a schematic of UNC80 with corresponding truncated constructs indicated. FIG. 11H show exemplary current traces from Xenopus oocytes expressing NALCN, FAM155A, UNC79 and UNC80, where wild-type UNC80 or truncated constructs are expressed, with steps from +80 to −80 mV, 20 mV increments, in ND96 recording solution. Star (*) indicates constructs that contain fusion of a C-terminal GFP-Flag tag. FIG. 11I shows a summary of mean current amplitudes elicited at +80 mV (top bar graph) or −80 mV (bottom bar graph) from a holding potential of 0 mV for indicated construct combinations. FIG. 11J shows a schematic of UNC80 with corresponding nonsense mutation constructs indicated. FIG. 11K shows exemplary current traces from Xenopus oocytes expressing NALCN, FAM155A, UNC79 and UNC80, where wild-type UNC80 or nonsense mutation constructs are expressed, with steps from +80 to −80 mV, 20 mV increments, in ND96 recording solution. FIG. 11L shows summary of mean current amplitudes elicited at +80 mV (top bar graph) or −80 mV (bottom bar graph) from a holding potential of 0 mV for indicated construct combinations. FIG. 11M shows exemplary current traces from Xenopus oocytes expressing NALCN, FAM155A, UNC79 and UNC80, where wild-type UNC80 or missense mutation constructs are expressed, with steps from +80 to −80 mV, 20 mV increments, in ND96 recording solution. FIG. 11N shows a summary of mean current amplitudes elicited at +80 mV (top bar graph) or −80 mV (bottom bar graph) from a holding potential of 0 mV for indicated construct combinations. FIG. 11O shows close-in views of select missense mutations previously identified in UNC80 mapped onto the UNC79-UNC80 subcomplex structure.

FIGS. 12A-G show characterization of NALCN-Nav1.4 chimeras. FIGS. 12A-F, top panels) show schematics of human NALCN and human NALCN-rat Nav1.4 chimeric constructs. FIGS. 12A-F, bottom panels show exemplary current traces from Xenopus oocytes expressing NALCN, FAM155A, UNC79 and UNC80, where wild-type or chimeric truncated NALCN constructs are expressed, with steps from +80 to −80 mV, 20 mV increments, in ND96 recording solution. FIG. 12G shows a summary of mean current amplitudes elicited at +80 mV (top bar graph) or −80 mV (bottom bar graph) from a holding potential of 0 mV for indicated constructs.

FIGS. 13A-C show structure-function of the NALCN C-terminal domain. FIG. 13A shows current traces from Xenopus oocytes expressing NALCN, FAM155A, UNC79 and UNC80, where the NALCN subunit is wild-type or has indicated CTD-deletions or mutations. Steps from +80 to −100 mV, 20 mV increments, in ND96 recording solution. FIG. 13B shows a summary of mean current amplitudes elicited at +80 mV (top bar graph) or −80 mV (bottom bar graph) from a holding potential of 0 mV for indicated constructs. FIG. 13C show current traces from Xenopus oocytes expressing NALCN, FAM155A, UNC79, UNC80 and control (H₂O) or an isolated NALCN-CTD construct in trans. Steps from +80 to −100 mV, 20 mV increments, in ND96 recording solution. FIG. 13D shows a summary of mean current amplitudes elicited at +80 mV from a holding potential of 0 mV in for indicated conditions. FIG. 13E shows current traces from Xenopus oocytes expressing wild-type NALCN (1-1738) or indicated C-terminal truncation constructs recorded in ND96 (top) and divalent cation (X²⁺)-free buffer (bottom). Steps from +80 to −100 mV, 20 mV increments. FIG. 13Ft shows fold-increase in inward current elicited at −100 mV for wild-type NALCN and indicated truncation mutants in response to removal of divalent cations.

FIGS. 14A-D show structure-function of the NALCN DI-DII and DII-DIII linkers. FIG. 14A shows current traces from Xenopus oocytes expressing NALCN, FAM155A, UNC79 and UNC80, where the NALCN subunit is wild-type or has indicated insertions, deletions or mutations in the DI-DII linker. Steps from +80 to −100 mV, 20 mV increments, in ND96 recording solution. FIG. 14B shows a summary of mean current amplitudes elicited at +80 m V (top bar graph) or −80 mV (bottom bar graph) from a holding potential of 0 mV for indicated constructs. FIG. 14C shows current traces from Xenopus oocytes expressing NALCN, FAM155A, UNC79 and UNC80, where the NALCN subunit is wild-type or has indicated insertions, deletions or mutations in the DII-DIII linker. Steps from +80 to −100 mV, 20 mV increments, in ND96 recording solution. FIG. 14D shows a summary of mean current amplitudes elicited at +80 mV (top bar graph) or −80 mV (bottom bar graph) from a holding potential of 0 mV for indicated constructs.

### DETAILED DESCRIPTION OF CERTAIN EMBODIMENTS

### Definitions

As used herein, the term about refers to a numeric value, including, for example, whole numbers, fractions, and percentages, whether or not explicitly indicated. The term about generally refers to a range of numerical values (e.g., +/−5-10% of the recited range) that one of ordinary skill in the art would consider equivalent to the recited value (e.g., having the same function or result). When terms such as at least and about precede a list of numerical values or ranges, the terms modify all of the values or ranges provided in the list. In some instances, the term about may include numerical values that are rounded to the nearest significant figure.

Unless otherwise defined, scientific and technical terms used in connection with the present invention shall have the meanings that are commonly understood by those of ordinary skill in the art. As utilized in accordance with the present disclosure, the following terms, unless otherwise indicated, shall be understood to have the following meanings:

In this application, the use of “or” means “and/or” unless stated otherwise. In the context of a multiple dependent claim, the use of “or” refers back to more than one preceding independent or dependent claim in the alternative only. Also, terms such as “element” or “component” encompass both elements and components comprising one unit and elements and components that comprise more than one subunit unless specifically stated otherwise.

As used herein, the transition term “consisting essentially of,” when referring to steps of a claimed process signifies that the process comprises no additional steps beyond those specified that would materially affect the basic and novel characteristics of the process. As used herein, the transition term “consisting essentially of,” when referring to a composition or product, such as a kit, signifies that it comprises no additional components beyond those specified that would materially affect its basic and novel characteristics.

As used herein, the singular forms “a,” “and,” and “the” include plural referents unless the context clearly dictates otherwise.

As described herein, any concentration range, percentage range, ratio range or integer range is to be understood to include the value of any integer within the recited range and, when appropriate, fractions thereof (such as one tenth and one hundredth of an integer), unless otherwise indicated.

Units, prefixes, and symbols are denoted in their Système International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

All proteins described herein are human proteins unless expressly described otherwise, as in “mammalian NALCN” or “murine NALCN” or the like phrases.

The term “NALCN” refers to the human Na+ leak channel non-selective protein. An exemplary amino acid sequence of the protein is provided in SEQ ID NO: 1, and may be found in the UniProt database under accession number Q8IZF0. The protein is also referred to as VGCNL1 (voltage-gated channel-like protein 1).

The “FAM155” or “FAM155A” protein refers to the human Family with sequence similarity 155 member A protein, which is a transmembrane protein that interacts with NALCN. An exemplary amino acid sequence of the protein may be found in SEQ ID NO: 2, or in the UniProt database under accession number B1AL88.

The “UNC79” protein refers to the human “uncoordinated 79” protein or the human “protein unc-79 homolog.” This protein is produced through transcription of the human UNC79 or KIAA1409 gene. An exemplary amino acid sequence may be found in SEQ ID NO: 3, or in the UniProt database under accession number Q9P2D8.

The “UNC80” protein refers to the human “uncoordinated 80” protein or the human “protein unc-80 homolog.” This protein is produced through transcription of the human UNC80 or KIAA1843 or C2orf21 gene. An exemplary amino acid sequence may be found in SEQ ID NO: 4, or in the UniProt database under accession number Q8N2C7.

The term “CaM” or “calmodulin” refers to human calmodulin protein, which is also abbreviated CALM, CALM1, or CAM1. An exemplary amino acid sequence is provided in SEQ ID NO: 5, or may be found in the UniProt database under accession number P0DP23.

A “mutant NALCN” as used herein refers to a human NALCN protein that comprises at least one engineered amino acid substitution, insertion or deletion compared to the protein of SEQ ID NO: 1. In some cases a “mutant NALCN” comprises at least one amino acid substitution in which at least one equivalent/corresponding residue of a different ion channel protein, such as a Nav or Cav family protein. In some cases, a region or a complete domain of a different ion channel protein is substituted for the equivalent region or domain of a different ion channel protein, such as a Nav or Cav family protein, e.g., some or all of the DI-DII or DII-DIII linker. In such cases, the mutant NALCN protein may alternatively be referred to herein as a “chimeric NALCN” or “chimera,” as it contains amino acid sequence segments from more than one native protein.

The term “chimeric” herein, when referring to a protein, means that the protein is made up of amino acid sequences from more than one native protein. For instance, as described above, a “chimeric NALCN” as used herein refers to a type of mutant NALCN in which at least one region of the NALCN protein has been exchanged with a corresponding region from a different ion channel protein, such as a human Nav or Cav family member protein. In some cases, the exchanged regions may be complete domains, or one or more individual transmembrane alpha helices or linker regions or loops within a domain, or a portion of an alpha helix and/or loop within a domain.

As used herein, the terms “corresponding” or “equivalent” are used interchangeably when referring to a residue or region from one protein that replaces a residue or region deleted from a different protein. As used herein, “corresponding” or “equivalent” residues or regions are those residues or regions that are in the same location within the two proteins when properly folded. In some cases, such corresponding or equivalent regions or amino acid residues may be identified using sequence alignments and structural information for the two proteins.

A “modulator” as used herein refers to a molecule that is capable of altering the behavior of a protein, or a complex of proteins, such as NALCN alone or in a complex with FAM155, UNC79, and UNC80. For example, in some cases a modulator may alter the behavior of a target protein or complex through binding to the protein. A modulator herein may act to either increase or decrease the activity of the protein, such as, for example, the degree to which the protein regulates the flow of ions across a cellular membrane. A modulator that decreases the activity of NALCN, for example, is an “inhibitor” of NALCN. A modulator that increases the activity of NALCN, for example, is an “activator” of NALCN. In some embodiments, a modulator may only modulate the activity of NALCN under certain conditions, such as in the presence of certain ions or when NALCN is complexed with other proteins such as FAM155, UNC79, and UNC80. A molecule that is a “modulator” of the NALCN-FAM155-UNC79-UNC80 complex, increases or decreases the activity of the complex, for instance in an ion channel assay. Such molecules may affect function of the complex through a variety of mechanisms, such as by affecting the binding between members of the complex, the folding or stability, or the trafficking or localization of one of the members of the complex, or impacting the folding or stability of the interfaces between the proteins, or blocking entry or exit of molecules from the central channel of the complex.

For purposes herein, the “NALCN-FAM155-UNC79-UNC80 complex” and the “NALCN complex” are used interchangeably unless it is made clear otherwise that they differ.

As used herein, a “potential modulator” of the NALCN complex is a molecule that is to be tested to determine if it acts as a modulator of the NALCN complex.

An “ion channel assay” herein refers to an assay that is used to measure the activity of an ion channel protein, such as a voltage-gated sodium channel. A variety of assays and assay formats are regularly used in the art, and many are provided in automated formats for high-throughput screening (HTS) analysis. Examples include ion flux assays, for example, using radioactive ions such as radioactive Na+ ions, an ion- or voltage-sensitive dye assay such as fluorescence assays for example using fluorescent indicator molecules whose fluorescence signal increases or decreases with changes in ion concentration, and various types of patch clamp assays. Such assays may directly or indirectly measure changes in ionic currents across a membrane comprising an ion channel protein in a variety of conditions. In some cases, such assays are used to assess the “activity” of the ion channel protein in the presence or absence of a potential or identified modulator. The term “activity” in this sense is meant in the broadest sense, given that these different assays measure the activity of the protein either directly or indirectly and through the measurement of different parameters, such as changes in fluorescence, radioactivity, or ionic current.

A “patch clamp assay” is used herein in the broadest sense to refer to an assay that is used to assesses changes in the movement of ions across a small patch of cell membrane containing an ion channel protein under different solution conditions, for example.

The term “peptide” as used herein refers to a chain of fifty amino acids or less linked by peptide bonds, including amino acid chains of 2 to 50, 2 to 15, 2 to 10, 2 to 8, or 6 to 14 amino acids.

The term “small molecule” as used herein refers to an organic molecule having a molecular weight of 50 Daltons to 2500 Daltons.

The term “macrocycle” or “macrocylic molecule” as used herein refers to a cyclic macromolecule or a macromolecular cyclic portion of a macromolecule. Macrocycles range in size from 500 Daltons to 7500 Daltons. In some cases herein, macrocycles are cyclic peptides or peptide derivatives.

The term “binding fragment” as used herein refers to a portion of a larger molecule, such as a small molecule, peptide, or antibody, that is expected to directly contact a target protein. Binding fragments may be used in high-throughput screens.

In this disclosure, “binds” or “binding” or “specific binding” and similar terms, when referring to a molecule that “binds” to a protein such as a NALCN, for example, means that the binding affinity is sufficiently strong that the interaction between the members of the binding pair cannot be due to random molecular associations (i.e. “nonspecific binding”). Thus, the binding is selective or specific.

The term “competition assay” as used herein refers to an assay in which a molecule being tested prevents or inhibits specific binding of a reference molecule to a common target.

Other definitions are included in the sections below, as appropriate.

As used herein, a “subject” refers to a human. As used herein, “treatment” refers clinical intervention, for example, to alleviate at least one symptom associated with a disease or disorder, slow progression of one or more symptoms, or prevent or delay onset of at least one symptom.

### An Isolated Human NALCN-FAM155-UNC79-UNC80 Complex

Some embodiments herein involve the formation of an isolated human NALCN-FAM155-UNC79-UNC80 complex in vitro. In some embodiments, the complex is formed by expressing each protein on an isolated vector, which may be expressed in a host cell, such as a HEK293 cell in vitro.

In some embodiments, at least one of the four proteins contains a label. In some embodiments, a label is used to assist in attaching the protein or protein complex to a matrix, such as beads (e.g., streptavidin-coated beads or the like). Thus, for instance, in some embodiments, a label is attached to either or both of the NALCN or FAM155 protein, e.g., at the C terminus. In some embodiments, a label may be attached to each of the complex proteins. An example of such a label is a molecule that allows for attachment of the protein or complex to another molecule or to a matrix, such as a streptavidin or flag tag, comprising a peptide segment recognized by streptavidin, for example, on the surface of a matrix. Another type of label, for instance, includes a label that allows for detection of the protein or complex by a color change, fluorescence or phosphorescence, or the like, such as a conjugated small molecule fluorescent label or a co-expressed label such as green fluorescent protein or the like.

In some embodiments, after expression and formation of the complex in a host cell, the complex may be isolated and resuspended, for example, in a lipid bilayer or lipid nanodisc or solubilized in detergent. In some embodiments, the complex is resuspended in lipid nanodiscs comprising a mixture of POPC (1-palmitoyl-2-oleoyl-sn-glycero-3-phosphocholine), POPE (1-palmitoyl-2-oleoyl-sn-glycero-3-phosphoethanolamine), and POPG (1-palmitoyl-2-oleoyl-sn-glycero-3-phosphatidylglycerol). In other embodiments, nanodiscs may comprise other lipids such as POPS (10% palmitoyl-oleoyl phostatidylserine) or POPA (1-palmitoyl-2-oleoyl-sn-glycero-3-phosphate). In some embodiments, these are mixed in a 3:1:1 ratio. In some embodiments, the complex may be solubilized in detergent, such as n-dodecyl-B-D-maltoside (DDM), lauryl maltose neopentyl glycol (LMNG), or glycol-diosgenin (GDN) or a mixture of such detergents. In some embodiments, the complex, for example, resuspended in a lipid bilayer or detergent or lipid nanodisc, may be bound to a matrix, such as a bead, plate, or chip.

Similar methods may also be used to obtain a UNC79-UNC80 subcomplex or an NALCN-FAM155 subcomplex.

### Exemplary Screening Methods to Identify Human NALCN-FAM155-UNC79-UNC80 Complex Modulators and Binders

An isolated human NALCN-FAM155-UNC79-UNC80 complex may be used in a variety of screening experiments, among other potential uses. For example, the complex may be used to screen for modulators of the human NALCN complex. In some embodiments, such methods may comprise: (a) providing a complex of human NALCN with human FAM155 (Family with sequence similarity 155 member A), UNC79 (uncoordinated 79), and UNC80 (uncoordinated 80) (a human NALCN-FAM155-UNC79-UNC80 complex) in vitro; (b) contacting the complex with a potential modulator of human NALCN-FAM155-UNC79-UNC80 complex; (c) performing an ion channel assay of the complex in the presence of the potential modulator; and (d) identifying the potential modulator as a modulator of human NALCN-FAM155-UNC79-UNC80 complex if the activity of the complex in the assay in the presence of the potential modulator is higher or lower than the activity of the complex in the assay in the absence of the potential modulator.

In some embodiments, the activity of the complex in the assay in the presence of the potential modulator is lower than the activity of the complex in the assay in the absence of the potential modulator (i.e., the potential modulator reduces the activity of the complex in the assay). For example, in some embodiments, the potential modulator identified in part (c) reduces the activity of the complex in the assay by at least 25%, at least 30%, at least 40%, at least 50%, at least 60%, at least 70%, at least 80%, or at least 90%. In some cases, the activity the complex in the presence of the modulator may be, for example, 1-90%, 10-90%, 1-10%, 1-20%, 25-90%, 50-90%, 25-75%, 40-80%, or 50-75% of the activity of the complex in the absence of the modulator. In some cases, the potential modulator identified in the assay reduces the activity of the complex in the assay with a half-maximal concentration of 10 nM to 500 μM, 50 nM to 500 μM, 10 nM to 50 μM, 100 nM to 500 μM, 100 nM to 50 μM, 1-500 μM, 1-50 μM, 10-500 μM, or 50-250 μM.

In other embodiments, the activity of the complex in the assay in the presence of the potential modulator is higher than the activity of the complex in the assay in the absence of the potential modulator (i.e., the potential modulator increases the activity of the complex in the assay). In such cases, the potential modulator identified in the assay increases the activity of the complex in the assay by at least 10%, at least 20%, at least 25%, at least 30%, at least 40%, at least 50%, at least 60%, at least 70%, at least 75%, at least 80%, or at least 100% (i.e. two-fold), up to 100%, or up to three-fold. In some cases, the activity the complex in the presence of the modulator, may be, for example, 10-100%, 10-80%, 20-80%, 25-100%, 25-75%, 50-100%, two to three fold, or 100-200% higher than the activity of the complex in the absence of the modulator.

In any of the above experiments, the activity of the complex may be measured by any of a variety of ion channel assays, including a patch clamp or an automated patch clamp assay, an ion flux assay, or an ion- or voltage-sensitive dye assay. Exemplary assays are described in more detail below.

In some embodiments, a screening method may further involve determining the binding affinity of a potential modulator or a modulator identified in the assay to the human NALCN-FAM155-UNC79-UNC80 complex and/or to subcomplexes of NALCN-FAM155 or UNC79-UNC80 or to one or more of the individual proteins. For instance, an ELISA or AlphaLISA or FRET assay may be used to determine a binding affinity. In some embodiments, the potential modulator binds to the human NALCN-FAM155-UNC79-UNC80 complex, or to a related subcomplex or to one of the individual proteins of the complex with an EC50 or IC50 of 10 μM or less, 10 μM to 50 nM, 10 μM to 500 nM, 1 μM or less, 1 μM to 50 nM, or 100 nM or less.

In additional embodiments, a screening method may be used to identify a molecule that binds to the human NALCN-FAM155-UNC79-UNC80 complex. For example, such a method may comprise (a) providing a human NALCN-FAM155-UNC79-UNC80 complex in vitro; (b) contacting the complex with one or more test molecules, and separating bound from unbound test molecules; and (c) identifying a test molecule as binding to the human NALCN-FAM155-UNC79-UNC80 complex if the test molecule remains bound to the complex after separating bound from unbound test molecules. In a further example, such a method may comprise (a) providing a human NALCN-FAM155-UNC79-UNC80 complex in vitro; (b) contacting the complex with an NALCN-DII-DIII linker peptide or NALCN-DI-DII linker peptide; and identifying the test molecule as binding to the human NALCN-FAM155-UNC79-UNC80 complex if the test molecule competes with the NALCN-DII-DIII linker peptide and/or the NALCN-DI-DII linker peptide for binding to the complex. For example, as described in the examples below, certain NALCN-DII-DIII peptides were found to modulate, and significantly reduce activity of the complex in ion channel assays. In some cases, both an ion channel assay screen and a binding screen may be performed. And in some cases, a binding assay to the complex may also be performed along with a binding assay to an NALCN-FAM155 and/or UNC79-UNC80 subcomplex or one of the individual proteins of the complex. In some embodiments, the NALCN-DII-DIII linker peptide or NALCN-DI-DII linker peptide may be labeled.

Screening methods herein also include methods of identifying a molecule that modulates formation of or stability of the complex. An example of such an assay may comprise (a) providing a human NALCN-FAM155-UNC79-UNC80 complex in vitro; (b) contacting the complex with one or more test molecules, and (c) identifying the test molecule as modulating formation of or stability of the human NALCN-FAM155-UNC79-UNC80 complex if the degree of complex formation in the presence of the test molecule is increased or reduced compared to in the absence of the test molecule. Such an assay may be conducted, for example, using FRET or similar labels or AlphaLISA to detect the proximity of different components of the complex in the presence and absence of the test molecule, for example. In some embodiments, such a screening method may further comprise additional assays to determine whether the test molecule affects formation or stability of a subcomplex such as NALCN-FAM155 or UNC79-UNC80.

In any of the methods herein, one or more of the components of the complex or the potential modulator may be labeled, either with a label to detect its presence (e.g., via color change, fluorescence, etc.) or to detect its proximity to other complex members (e.g., FRET), or to attach the molecule to a matrix (e.g., a streptavidin recognition element or similar). In any of the methods herein, one or more components of the complex or the potential modulator may also be attached to a matrix, e.g. a bead, chip, or plate. In any of the assays above, the complex may further comprise other elements, such as those that adhere to the complex after its expression in vivo in a host cell. In some embodiments, the complex, for instance, further comprises calmodulin.

In any of the methods above, the methods may be conducted using a NALCN-FAM155-UNC79-UNC80 complex or a related subcomplex comprising a mutant version of one of the proteins, such as a mutant human NALCN. Exemplary mutant human NALCN polypeptides are described in the working examples and sequence table herein. In some cases, a mutant human NALCN comprises a substitution, insertion, or deletion in one or both of the DI-DII or DII-DIII linker elements. These may include, for instance, a shortening of the linker by deletion of residues, insertion of a flexible element such as a GGGS element (SEQ ID NO: 86) into the linker, or a point mutation in the linker, or replacement of some or all of the linker sequence with corresponding sequence from another ion channel protein such as a human Nav or Cav protein, such as Nav1.4. Examples of human NALCN mutants tested herein include those comprising a sequence selected from SEQ ID Nos: 6-15 and 32-52 and 59-63. In other embodiments, a truncated UNC79 or UNC80 polypeptide or a mutated FAM155A molecule might be included in the complex. Examples of human UNC79 mutants include those comprising SEQ ID Nos: 53, 54, 55, and 58, or comprising a UNC79 fragment such as comprising a sequence selected from SEQ ID Nos: 64-73. UNC80 fragments could also be used, such as comprising a sequence selected from SEQ ID Nos: 74-85. For instance, if a test molecule binds to the wild-type complex but does not bind to one or more such mutant complexes, this may provide information about how the test molecule binds to the complex.

In some embodiments, a screening method is performed in the presence of an NALCN-DII-DIII linker peptide or an NALCN-DI-DII linker peptide or another identified modulator of the human NALCN-FAM155-UNC79-UNC80 complex. Thus, for instance, one may determine whether a potential modulator competes with an identified modulator of the activity of the complex by including both the potential modulator and the identified modulator in the assay. As described in the examples below, certain linker peptides, comprising sequences from the DII-DIII linker of human NALCN were found herein to reduce or inhibit the function of the human NALCN-FAM155-UNC79-UNC80 complex in ion channel assays. Thus, these peptides were identified as modulators of the complex in assays described herein. Examples include peptides comprising SEQ ID Nos: 16-18 and 23-24, for instance, comprising residues 617-740, 659-774, and 617-845 of the DII-DIII linker, for example, optionally with an N-terminal modification such as MBP or palmitoyl, or with an N-terminal methionine residue. In some cases, the peptides may be expressed in a host cell in vivo along with the members of the protein complex. In other cases, the peptides may be added separately to the complex in vitro.

### Exemplary Functional Assays

In any of the above methods, the ion channel assay may be, for example, any suitable assay used to detect the activity of the human NALCN-FAM155-UNC79-UNC80 complex as an ion channel. Examples include, but are not limited to, a patch clamp assay, including an automated patch clamp assay, an ion flux assay, and an ion- or voltage-sensitive dye assay. Exemplary assays are described, for example, in H. Yu et al, “high throughput screening technologies for ion channels,” Acta Pharm. Sinica 37: 34-43 (2016), and materials are available from commercial manufacturers. In an ion flux assay, for example, radioactive isotopes, such as sodium 22 (²²Na⁺), can be used to trace the cellular influx and efflux of sodium ions or other ions. Another type of assay is a voltage-sensitive or ion-sensitive dye assay. In a voltage-sensitive dye assay, voltage changes across a membrane comprising the ion channel protein are measured using fluorescence resonance energy transfer (FRET), for example, using dyes such as oxonol derivatives such as bis-(1,3-dibutylbarbituric acid) trimethine oxonol (DiBAC₄) or FMP. In some cases, a FRET dye may be localized or tethered to the membrane. Ion-sensitive dye assays may use dyes that show a difference in signal depending on ion concentration. An example is the sodium indicator dye SBFI. In other embodiments, a patch-clamp assay may be used in the screening methods. In some embodiments, an automated patch-clamp assay may be used. Exemplary platforms and instrumentation for performing patch-clamp assays are sold by several manufacturers. Examples platform assays include IonWorks™ platform assays, PatchXpress™ and IonFlux™ (Molecular Devices), Qpatch™ HT/HTX (Sophion), and Patchliner™ and SynchroPatch™ (Nanion Technologies).

In some embodiments, a two-electrode voltage-clamp assay is performed, for example, in Xenopus laevis oocytes. In this assay, a 1:1:1:1 mixture of mRNAs or cDNAs (or the like) for each member of the complex is injected into the oocytes in order to express the complex proteins. In some embodiments, an identified modulator of the complex, such as a soluble linker peptide, such as a DI-DII or DII-DIII linker or portion thereof, may further be expressed. A two-electrode voltage-clamp assay may then be performed, for instance, as described in the working examples below.

In some embodiments, a patch-clamp assay may be performed in HEK-293T cells, for example. In some embodiments, the complex is formed from a 2:1:1:1 transfected cDNA ratio (or the like) of NALCN-eGFP-2×FLAG-labeled NALCN, UNC79, UNC80, and FAM155A, for example. Further details are provided in the working examples.

In any of the above methods, the potential modulator identified in the method modulates the activity of the human NALCN-FAM155-UNC79-UNC80 complex, e.g., in the presence of the modulator the complex has an activity that is lower or higher than without the modulator. In some cases, the potential modulator reduces the activity of the complex. In some cases, the activity may be reduced by at least 25%, at least 30%, at least 40%, at least 50%, at least 60%, at least 70%, at least 75%, at least 80%, or at least 90%.

In any of the above methods, the ion channel assay may also be performed in the presence of both a potential modulator and an identified modulator of the human NALCN-FAM155-UNC79-UNC80 complex. For example, in such a case, one might determine if the identified modulator competes with the potential modulator for altering the activity of the protein. Examples include peptides comprising the amino acid sequences of SEQ ID Nos: 16-18 and 23-24.

Any of the above methods may also further comprise determining the binding affinity of the potential modulator identified in the assay to human NALCN-FAM155-UNC79-UNC80 complex or its subcomplexes or individual proteins and mutants thereof. For example, such assays may help to understand the mechanism by which a molecule modulates activity of the complex. For instance, an ELISA assay may be used to determine binding affinity, for example, using the human NALCN-FAM155-UNC79-UNC80 complex in a lipid-stabilized form on a matrix, such as a solid surface, such as beads, plates or the like. Beads can have any shape, such as flakes or chips, spheres, pellets, etc. In some embodiments, such beads are streptavidin-coated beads, avidin-coated beads, or deglycosylated-avidin-coated beads. In some embodiments, such beads are magnetic beads. In some cases, the potential modulator binds to human NALCN-FAM155-UNC79-UNC80 complex or to a subcomplex or individual protein thereof with an EC50 or IC50 of 10 μM or less, 10 μM to 50 nM, 10 μM to 500 nM, 1 μM or less, 1 μM to 50 nM, or 100 nM or less.

In some embodiments, further experiments may be performed on molecules selected in the above screens, for example, to determine other biological activities of the molecules. For instance, further assays may be used to determine if the molecules also bind to other ion channel proteins, or to mutants of the human NALCN-FAM155-UNC79-UNC80 complex proteins, thus determining the specificity of the molecules as ion channel modulators or binders.

### Test Molecules for Screening Methods

In any of the above methods, the potential modulator (i.e., the test molecule) may be a peptide or macrocycle or antibody. For example, in some cases, the potential modulator is a small molecule. The present disclosure also relates to modulators of human NALCN or the human NALCN-FAM155-UNC79-UNC80 complex identified by the methods described herein, which may be optionally peptides, macrocycles, small molecules, or antibodies.

In some embodiments, the potential modulator molecule to be tested is a peptide. In some embodiments, the peptide is a 3-40-mer, a 3-20-mer, a 4-16-mer, a 4-14-mer, or a 6-14-mer, such as a 3-mer, 4-mer, 5-mer, 6-mer, 7-mer, 8-mer, 9-mer, 10-mer, 11-mer, 12-mer, 13-mer, 14-mer, 15-mer, 16-mer, 17-mer, 18-mer, 19-mer, 20-mer, 21-mer, 22-mer, 23-mer, 24-mer, 25-mer, 26-mer, 27-mer, 28-mer, 29-mer, 30-mer, 31-mer, 32-mer, 33-mer, 34-mer, 35-mer, 36-mer, 37-mer, 38-mer, 39-mer, or 40-mer.

In some embodiments, the peptide is a macrocycle. In some embodiments, the macrocycle is a 3-40-mer, a 3-20-mer, a 4-16-mer, a 4-14-mer, or a 6-14-mer, such as a 3-mer, 4-mer, 5-mer, 6-mer, 7-mer, 8-mer, 9-mer, 10-mer, 11-mer, 12-mer, 13-mer, 14-mer, 15-mer, 16-mer, 17-mer, 18-mer, 19-mer, 20-mer, 21-mer, 22-mer, 23-mer, 24-mer, 25-mer, 26-mer, 27-mer, 28-mer, 29-mer, 30-mer, 31-mer, 32-mer, 33-mer, 34-mer, 35-mer, 36-mer, 37-mer, 38-mer, 39-mer, or 40-mer macrocycle.

In some embodiments, the molecule to be tested in a screen herein is a small molecule. In some embodiments, the molecule to be tested is an antibody, which may include not only full length antibodies of any of IgG, IgM, IgA, IgD, and IgE, but also an antigen binding fragment of an antibody, such as an Fv, Fab′, (Fab′)2, scFv, or the like, a nanobody, single-chain antibody, bispecific or multispecific antibody.

In some embodiments, the molecule to be tested is a binding fragment of a peptide, a binding fragment of a small molecule, or a binding fragment of an antibody (e.g., an antigen binding fragment).

In some embodiments, test molecules that are identified as modulators of the NALCN-FAM155-UNC79-UNC80 complex, for example, may be used in methods of treating disorders related to disfunction in the NALCN-FAM155-UNC79-UNC80 complex. In some embodiments, a subject with a disfunction in the NALCN-FAM155-UNC79-UNC80 complex may be treated for, for instance, a channelopathy, a neurodevelopmental disorder, a circadian rhythm disorder, or pain. Alternatively, in other embodiments, a molecule identified herein may be useful in treating, for example, a channelopathy, a neurodevelopmental disorder, a circadian rhythm disorder, or pain in a subject who does not have a disfunction in the NALCN-FAM155-UNC79-UNC80 complex. For instance, a molecule identified herein may, in some embodiments, modulate the function of the complex in a subject. A channelopathy, as defined herein, includes a disease or disorder characterized by impaired function in one of the members of the NALCN complex or in a different potassium or calcium channel protein.

### Molecular Complexes

In some embodiments, the disclosure comprises a molecular complex comprising human NALCN-FAM155-UNC79-UNC80, human NALCN-FAM155, human UNC79-UNC80, or human NALCN as described herein bound to a molecule, such as a potential modulator molecule, such as a peptide, small molecule, antibody, or binding fragment of a peptide, small molecule, or antibody.

In some embodiments, the molecule is a peptide. In some embodiments, the peptide is a 3-40-mer, a 3-20-mer, a 4-16-mer, a 4-14-mer, or a 6-14-mer, such as a 3-mer, 4-mer, 5-mer, 6-mer, 7-mer, 8-mer, 9-mer, 10-mer, 11-mer, 12-mer, 13-mer, 14-mer, 15-mer, 16-mer, 17-mer, 18-mer, 19-mer, 20-mer, 21-mer, 22-mer, 23-mer, 24-mer, 25-mer, 26-mer, 27-mer, 28-mer, 29-mer, 30-mer, 31-mer, 32-mer, 33-mer, 34-mer, 35-mer, 36-mer, 37-mer, 38-mer, 39-mer, or 40-mer.

In some embodiments, the peptide is a macrocycle. In some embodiments, the macrocycle is a 3-40-mer, a 3-20-mer, a 4-16-mer, a 4-14-mer, or a 6-14-mer, such as a 3-mer, 4-mer, 5-mer, 6-mer, 7-mer, 8-mer, 9-mer, 10-mer, 11-mer, 12-mer, 13-mer, 14-mer, 15-mer, 16-mer, 17-mer, 18-mer, 19-mer, 20-mer, 21-mer, 22-mer, 23-mer, 24-mer, 25-mer, 26-mer, 27-mer, 28-mer, 29-mer, 30-mer, 31-mer, 32-mer, 33-mer, 34-mer, 35-mer, 36-mer, 37-mer, 38-mer, 39-mer, or 40-mer macrocycle.

In some embodiments, the molecule in the complex is a small molecule. In some embodiments, the molecule is an antibody, which may include not only full length antibodies of any of IgG, IgM, IgA, IgD, and IgE, but also an antigen binding fragment of an antibody, such as an Fv, Fab′, (Fab′)2, scFv, or the like, a nanobody, single-chain antibody, bispecific or multispecific antibody.

In some embodiments, the molecule is a binding fragment of a peptide, a binding fragment of a small molecule, or a binding fragment of an antibody (e.g., an antigen binding fragment).

### Kits

The present disclosure also includes kits comprising reagents associated with screening methods herein. In some cases, kits comprise one or more of the human NALCN, FAM155, UNC79, and UNC80 proteins as described herein, or alternatively an isolated nucleic acid, vector, or host cell for use in expressing the proteins for purification of a complex of the four proteins. In some cases, a kit may comprise one or more mutants of the proteins, such as proteins attached to a label, either for detection or for attaching to a matrix. In some cases, kits comprise reagents used in screening methods herein, either with or without the protein complex components. In some cases, kits comprise more than one type of NALCN, FAM155, UNC79, or UNC80 protein, such as a mutant and wild-type protein.

In some embodiments, kits herein may comprise one or more reagents for performing an ion channel assay. In some cases, kits may comprise particular, identified modulators of the human NALCN-FAM155-UNC79-UNC80 complex, for instance, as positive controls. Kits herein may also include negative controls that are identified as not modulating a human NALCN-FAM155-UNC79-UNC80 complex. The kits may also one or more of NALCN, FAM155, UNC79, or UNC80, or their complex attached to matrix particles such as beads or to another type of matrix such as a plate. Beads can have any shape, such as flakes or chips, spheres, pellets, etc. In some embodiments, such beads are streptavidin-coated beads, avidin-coated beads, or deglycosylated-avidin-coated beads. In some embodiments, such beads are magnetic beads. The protein complex may or may not be pre-attached to a matrix. In some embodiments, reagents are included to facilitate attachment of the protein to a matrix, such as through biotin-streptavidin or a similar system.

In some embodiments, kits may comprise reagents associated with screening methods herein, such as some or all of the reagents needed to perform an ion channel assay as described in the methods herein. In some cases, one or more control reagents, such as modulators of the protein, may also be included.

In some embodiments, kits may comprise test molecules or libraries of test molecules, such as peptides, small molecules, and/or antibodies. In some embodiments, peptides in the kit may be macrocycles. In some embodiments, the kit may comprise test molecules that are a binding fragment of a peptide, small molecule, or antibody.

In some embodiments, kits may also comprise directions for use.

### EXAMPLES

### Example 1. General Methods

### A. Expression and Purification of the NALCN-FAM155A-UNC79-UNC80-CaM Complex

An optimized coding DNA for human NALCN, FAM155A, UNC80 and UNC79 were each cloned into a pRK vector behind a cytomegalovirus (CMV) promoter. A twin-Strep-2×Flag tag was added to the N-terminus of NALCN and 2×Flag tag was added to the C-terminus of FAM155A, UNC80 and UNC79. Expi293 cells in suspension were cultured in SMM 293T-I medium under 5% CO₂ at 37° C. and transfected using polyethylenimine (PEI) with DNAs at a 1:1:1:1 ratio when the cell density reached 4×10⁶ cells per mL. Transfected cells were cultured for 48 hours before harvesting.

Sixty grams of harvested cell pellet was resuspended in 300 mL of 25 mM HEPES pH 7.5, 200 mM NaCl, 1 μg/mL benzonase, 1 mM PMSF and Roche protease inhibitor tablets. Cells were lysed by dounce homogenization and the NALCN complex was subsequently solubilized by addition of 2% (w/v) glycol-diosgenin GDN supplemented with 0.1% (w/v) cholesteryl hemisuccinate and 0.2 mg/mL porcine brain polar lipid extract (Avanti) for 2 hr at 4° C. under gentle agitation. Insoluble debris was pelleted by ultracentrifugation at 125,000×gₘₐₓ for 1 hr, and the supernatant containing the solubilized protein was collected for affinity purification by batch-binding to 5 mL of M2-agarose FLAG resin (Sigma) for 1 hr at 4° C. Unbound proteins were washed with 6 column volumes (CV) of purification buffer (6 CV 25 mM HEPES pH 7.5, 200 mM NaCl, 0.04% (w/v) GDN followed by 10 CV supplemented with 500 mM NaCl, and 10 CV buffer supplemented with 5 mM ATP and 10 mM MgCl₂. NALCN was eluted with 5 CV of purification buffer supplemented with 300 μg/mL FLAG peptide (Sigma). The eluent was collected and applied to 3 mL Strep-Tactin XT high affinity resin (IBA) and bound in batch for 3 hr. Unbound proteins were washed with 10 CV of purification buffer and eluted with 5 CV of purification buffer supplemented with 50 mM biotin. The NALCN complex was then concentrated with an Amicon® Ultra centrifugal filter device (100 kDa MWCO) concentrators to 4 mg/mL.

B. Reconstitution of NALCN Channelosome into Nanodiscs

A 200-fold molar excess of a 3:1:1 lipid mix of 1-palmitoyl-2-oleoyl-sn-glycero-3-phosphocholine (POPC), 1-palmitoyl-2-oleoyl-sn-glycero-3-phosphoethanolamine (POPE), and 1-palmitoyl-2-oleoyl-sn-glycero-3-phosphatidylglycerol (POPG) (pre-solubilized by sonication at 10 mg/mL in a buffer containing 50 mM HEPES pH 7.5, 100 mM NaCl, 5 mM MgCl₂, and 1% CHAPS) was added to aliquots of 0.35 nmol NALCN in 2 mL tubes and incubated at 4° C. for 30 minutes. A 4-fold molar excess of the membrane scaffold protein MSP1E3D1 (Sigma) was then added and incubated for an additional 30 minutes at 4° C. before dilution to 1.5 mL in Purification Buffer. Bio-Beads were added to a concentration of 0.25 mg/mL and the samples were incubated overnight at 4° C. with rotation. Reactions were then combined and the Bio-Beads were removed prior to concentrating the reactions to 100 μL. The concentrated reactions were applied to a Superose® 6 3.2/300 column that was pre-equilibrated in Gel Filtration Buffer (25 mM HEPES pH 7.5, 200 mM NaCl). Peak fractions were pooled and concentrated to 2.4 mg/mL.

### C. Cryo-EM Sample Preparation and Data Acquisition

The NALNC-FAM155A-UNC79-UNC80-CaM complex grids were prepared in the following manner. Holey carbon grids (Ultrafoil™ 25 nm Au R 0.6/1 300 mesh; Quantifoil) were glow-discharged for 30 seconds using the Solarus® plasma cleaner (Gatan). The NALNC-FAM155A-UNC79-UNC80-CaM complex was gently cross-linked with 0.05% EM-grade glutaraldehyde for 10 min at room temperature. Cross-linking was quenched with 9 mM Tris pH 7.5 and 3 μL of the sample was applied to the grid. Grids were blotted with a Leica Microsystems automatic plunge freezer (EM GP2 Leica Microsystems) using 3.5 seconds blotting time with 100% humidity, and plunge-frozen in liquid ethane cooled by liquid nitrogen. Movie stacks were collected from two grids using SerialEM¹ on a Titan Krios™ operated at 300 keV with BioQuantum™ energy filter equipped with a K3™ Summit direct electron detector camera (Gatan). Images were recorded at 105,000× magnification corresponding to 0.838 Å/pixel, using a 20 eV energy slit. Each image stack contains 60 frames recorded every 0.05 seconds for an accumulated dose of ˜60e/Å2 and a total exposure time of 3 seconds. Images were recorded with a set defocus range of 0.5 to 1.5 μm.

### D. Cryo-EM Data Processing

Cryo-EM data were processed using a combination of RELION™³⁴ and cisTEM™³⁵ software packages. 15,080 movies were corrected for frame motion using relion's MotionCor2³⁶ implementation and contrast-transfer function (CTF) parameters were fit using the 30-4.5 Å band of the spectrum with CTFFIND-4™³⁷. Images were filtered based on the detected fit resolution better than 8 Å. 1,778,009 particles were picked using WARP™³⁸ with the re-trained deep learning-based picking algorithm. To obtain a first 3D reconstruction, particles were sorted stringently in three rounds of reference-free 2D classification using 100-200 classes for each classification to select the best aligning particles. 446,099 particles were afterwards subjected to a 3D classification in RELION™. A resulting 3D class was subjected as a 20 Å LPF reference for a second 3D classification run with 233,310 selected particles (2 of 4 classes). To improve the quality of the 3D reconstruction, the 3D map and 76,673 selected particles from a single 3D class were exported from RELION™ and imported to cisTEM™ for iterative rounds of auto-refine without mask and manual refinement with continuously adjusted masks by applying LPF outside the mask (filter resolution 20 Å, outside weight 0.8). The resulting 3D reconstruction was subjected as reference map for an additional round of RELION™ 3D classification using the 446,099 particles after 2D classification followed by cisTEM™ auto and manual refinements. These iterations between RELION™ 3D classification and cisTEM™ refinement were repeated until resolution and quality for the resulting EM reconstructions converged. The 3D reconstruction was afterwards subjected as a 3D reference for RELION™ 3D classification with a broad selection of 1,718,298 particles after a single round of reference-free 2D classification with 300 classes to only separate NALCN complex particles from debris and other false positives. 1,007,024 particles from 4 of 6 selected classes were exported from RELION™ and imported to cisTEM™ for iterative rounds of auto-refine without mask and manual refinement with masks by applying LPF outside the mask (filter resolution 20 Å, outside weight 0.8). Refinements were repeated until the resolution and quality for the resulting EM reconstructions converged. After 3D classification and multi-model manual refinement in cisTEM™ (separation in 6 classes), 365,512 particles from a single class (˜38%) were extracted and refined with cisTEM™ auto-refine without mask followed by manual refinements with masks by applying LPF outside the mask (filter resolution 20 Å, outside weight 0.8) and a score threshold of 0.25. The 3D reconstructions converged to a map resolution of 2.8 Å (FSC=0.143, determined in cisTEM™). For model building and figure preparation, the map was sharpened in cisTEM™ with the following parameters: flattening from a resolution of 8 Å, applying a pre-cut-off B-factor of −90 Å² from the origin of reciprocal space and applying a figure-of-merit filter³⁹. Local resolution was determined in cisTEM™ using an in-house re-implementation of the blocres algorithm⁴⁰.

### E. Model Building and Structure Analysis

Structures of NALCN-FAM155A (PDB: 6XIW) and CaM (PDB: 4DCK) were used as a template for modeling the NALCN complex, and a rigid body fit into the cryo-EM map. Structures of UNC79 and UNC80 as well as the NALCN DI-DII and DII-DIII linker regions were built de novo into the map. After extensive (re)building and manual adjustments, multiple rounds of real space refinement using the phenix.real_space_refinement⁴¹ tool was used to correct global structural differences between the initial model and the map. The model was further manually adjusted in Coot⁴² and UCSF ChimeraX⁴³ with ISOLDE™⁴⁴ through iterative rounds of model building and real space refinements in phenix. The model was validated using phenix.validation_cryoem⁴⁵ with built-in MolProbity scoring⁴⁶. Figures were made using PyMOL™ (The PyMOL Molecular Graphics System, Version 2.07 Schrödinger, LLC), UCSF Chimera⁴⁷ or UCSF ChimeraX⁴³. 3D homology structural analysis was performed using the DALI server³². Sequences were aligned using Clustal Omega™⁴⁸ within JalView™⁴⁹ and illustrated with ESPript 3.0™⁵⁰ followed by manual adjustment based on considerations from the NALCN-FAM155A-UNC79-UNC80-CaM model.

### F. Two-Electrode Voltage-Clamp Electrophysiology

Xenopas laevis frog oocytes were prepared as previously described¹⁰. Healthy-looking stage V-VI oocytes were isolated and injected with 30 to 40 ng of RNA in a volume of 32 to 41 nl using a Napoliter 2010 injector (World Precision Instruments). The NALCN (or NALON truncated mutants or NALON-Nav1.4 chimeras), UNC-79, UNC-80, and FAM155A RNAs were mixed in a ratio of 1:1:1.1. When expressing NALCN, UNC79, UNC80, FAM15SA with an isolated linker, the RNAs were mixed in a ratio of 1:1:1:1:1. For the control combination (i.e., NALCN+UNC79+UNC80+FAM155A without linker), an equivolume of nuclease-free water was added to keep the concentration of each RNA constant. Injected cells were incubated in ND96 storage solution (96 mM NaCl, 2 mM KCl, 1 mM MgCl₂, 1.8 mM CaCl2, 5 mM HEPES, 2.5 mM pyruvate, 0.5 mM theophylline; pH 7.4 with NaOH) supplemented with 50 μg/mL gentamycin and tetracycline at 18° C., 140 rpm. Four to five days after RNA injection, two-electrode voltage-clamp measurements were performed on oocytes continuously perfused in ND96 recording solution (96 mM NaCl, 2 mM KCl, 1 mM MgCl₂, 1.8 mM CaCl₂, and S mM Hepes (pH 7.4) with NaOH) at room temperature using a Warner OC-725C Oocyte Clamp amplifier (Warner Instrument Corp, USA). Data were acquired using the pCLAMP™ 10 software (Molecular Devices) and a Digidata® 1550 digitizer (Molecular devices), sampled at 10 kHz. Electrical powerline interference was filtered with a Hum Bug 50/60 Hz Noise Eliminator (Quest Scientific). Recording microelectrodes with resistances around 0.2 to 1.0 MΩ were pulled from borosilicate glass capillaries (Harvard Apparatus) using a P-1000 Flaming/Brown Micropipette Puller System (Sutter Instrument) and were filled with 3 M KCl

### G. Patch-Clamp Electrophysiology

HEK-293T cells were maintained as previously described (M. Kschonsak et al., Nature 587: 313-318 (2020).) Cells between passages 6 and 20 were used for experiments and were tested for mycoplasma (Eurofins Genomics). For patch-clamp experiments, cells reaching 40 to 60% confluency in 35-mm cell culture dishes were transiently transfected with constructs of interest using LipoD293 ver. II (tebu-bio) 20 to 24 hours before recording. A total of 2.5 μg (for LipoD293) of cDNAs was used. The NALCN-eGFP-2×FLAG, UNC79, UNC80, and FAM155A cDNAs were mixed at the mass ratios of 2:1:1:1.

On the day of the experiment, transfected HEK-293T cells were seeded on poly-L-lysine-coated glass coverslips at least 3 hours before recording. Cells were voltage-clamped at room temperature in the whole-cell configuration using an Axopatch™ 200B amplifier (Molecular Devices). Data were acquired using the pCLAMP™ 10 software (Molecular Devices) and an Axon™ Digidata® 1550A digitizer (Molecular Devices) at 10 kHz. Patch pipettes were pulled from Kwik-Fil 1.5/1.12 [outer diameter (OD)/inner diameter (ID), in millimeters] borosilicate glass capillaries (World Precision Instruments) and fire-polished to resistances around 3.0 to 5.5 MΩ. A custom-built glass perfusion tool with four adjacent barrels (OD/ID 0.45/1.60, in millimeters; CM Scientific) controlled by an MXPZT-300R solution switcher (Siskiyou) was used to rapidly exchange extracellular solutions.

For symmetrical Na conditions, (1) the extracellular solution contained 150 mM NaCl, 10 mM HEPES, and 30 mM D-(+)-glucose (pH 7.4) with NaOH, ˜325 mOsm/L, and (2) intracellular solution contained 136 mM NaCl, 10 mM NaF, 5 mM EGTA, 10 mM HEPES, and 2 mM Na₂ATP (pH 7.2) with NaOH, ˜309 mOsm/L. For a condition that more closely resembles physiological condition, (1) the extracellular solution contained 150 mM NaCl, 5 mM KCl, 0.5 mM CaCl₂), 1.2 mM MgCl₂, 10 mM HEPES, and 13 mM D-(+)-glucose (pH 7.4) with NaOH, ˜320 mOsm/L, and (2) intracellular solution contained 140 mM CsCl, 10 mM CsF, 5 mM EGTA, 10 mM HEPES, and 2 mM Na₂ATP (pH 7.2) with CsOH, ˜304 mOsm/L. To prevent nonspecific leaks from affecting the accuracy of our results, we routinely checked for loose seals by exposing cells to NMDG-only extracellular solution before and/or after experiments. Cells that showed steady-state inward current >10 pA at −80 mV in the absence of permeable ions were discarded.

### Example 2. UNC79/UNC80-NALCN-Linker Based Fluorescence-Based Polarization Assay to Screen for Channel Modulators

A recombinant UNC79-UNC80 complex is expressed and purified as described in Example 1A. In particular, full-length human UNC79 is cloned into a pRK (or similar) vector behind a CMV promotor (or similar) with a carboxy-terminal Flag-tag (or similar) added; full-length human UNC80 is cloned as UNC80, but with a carboxy-terminal Streptavidin-tag (or similar) added. Both plasmids are co-transfected into Expi293 cells using standard protocols and expressed at 37 degrees for 48 hours. Cell pellets are harvested and lysed in standard non-denaturing buffers with added protease inhibitors added. The UNC79-UNC80 complex is purified using standard protocols for anti-Flag affinity purification followed by anti-Streptavidin tag affinity purification. The recovered UNC79-Flag-tag: UNC80-Strepatavdin-tag complex is further purified over a Superose® 6 column and the peak monodisperse fractions is collected. Fractions of purified protein are saved. The concentrations of purified protein are measured and adjusted. Purified protein is divided into aliquots and frozen until further use.

Synthetic peptides comprising targeted intracellular loop regions from human NALCN are synthesized using standard peptide chemistry methods with appropriate fluorescent dyes (e.g., Alexa647, Alex488, TAMRA, and Cy5) conjugated to either the N- or C-terminus of the peptide, either directly or through a PEG6 linker, or the like. These synthetic peptides contain residues from the DI-DII loop (Gln349-Ala363; SEQ ID NO: 19) and/or the DII-DIII loop (Ser617-Arg845; SEQ ID NO: 18) of human NALCN. Shorter synthetic peptides from either of these intracellular regions may also be considered (e.g., 638-670, 794-828 and/or 829-835; SEQ ID NO: 20, 21, and 22, respectively) for conjugation to an appropriate fluorescent dye. Non-labeled versions of these same peptides, and random scrabbled peptides are also generated as assay reagents.

Using purified UNC79: UNC80 complex and synthetic, fluorescent NALCN-based peptides, assay development and optimization using a standard fluorescence-based plate reader in 384-well or 1586-well format is performed. Fluorescent NALCN-based peptides are used to measure binding of the NALCN linker peptide to the UNC79-UNC80 complex. Unlabeled NALCN peptides or scrambled peptides are used with fluorescent NALCN-peptides. Unlabeled peptides compete with fluorescent peptides for binding to the UNC79-UNC80 complex and may even displace fluorescent peptides that are bound to UNC79-UNC80 complex. Scrambled peptides have no impact. Examples of assay parameters include protein and peptide concentrations, the types of dye used and dye-conjugation sites, buffer conditions, and assay temperature are optimized to maximize the sensitivity and robustness of the signal and assay.

Once appropriate assay parameters are determined, the UNC79-UNC80 complex and fluorescent NALCN-based peptides are screened against diverse chemical libraries to identify molecules that can compete with (and displace) the peptides binding to the UNC79-UNC80 complex. Molecules identified from the screen are further characterized by dose-response assays and are subsequently tested for modulation of the NALCN channel activity using standard electrophysiological assays.

### Example 3. Characterization of the Recombinant NALCN Channelosome

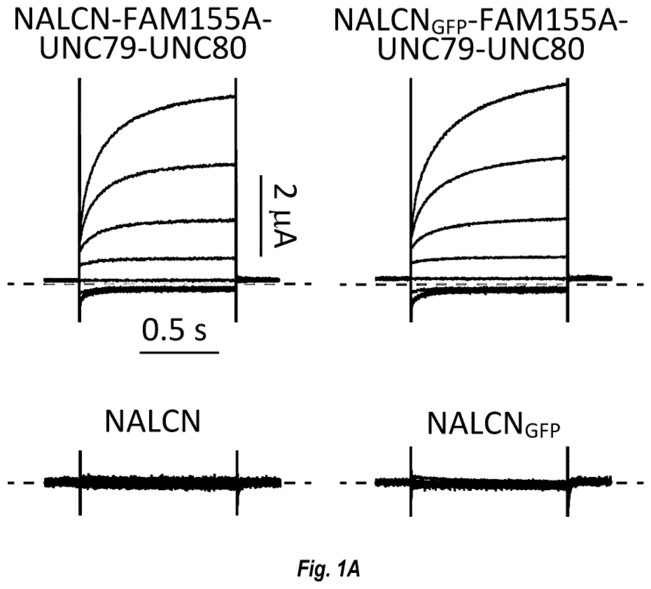

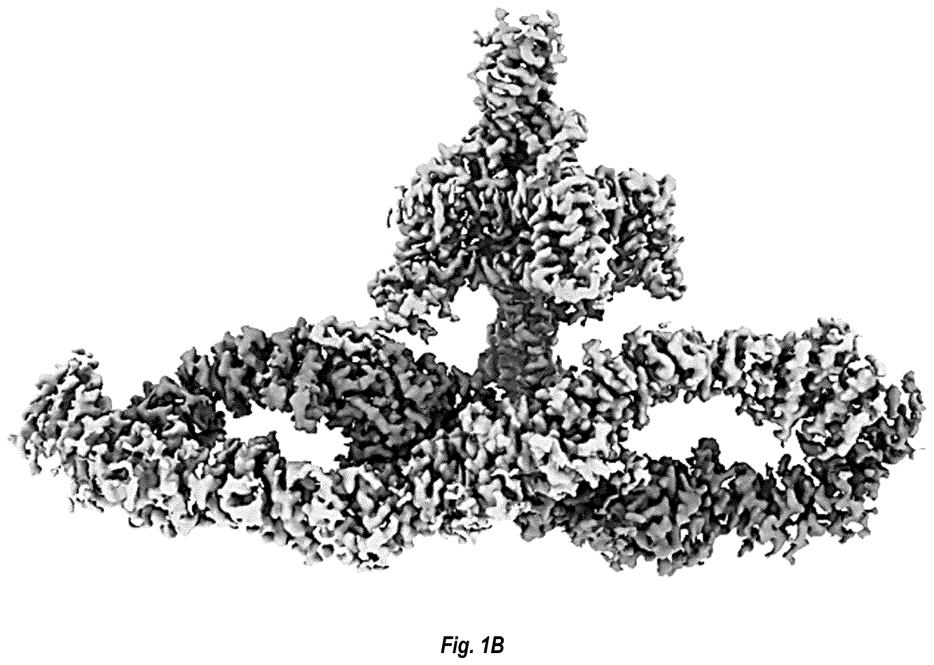

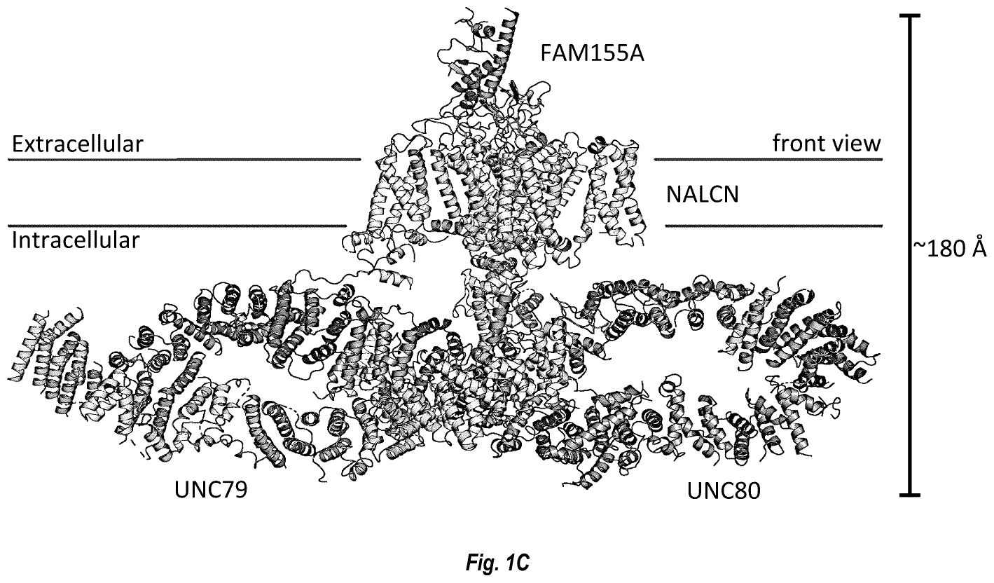

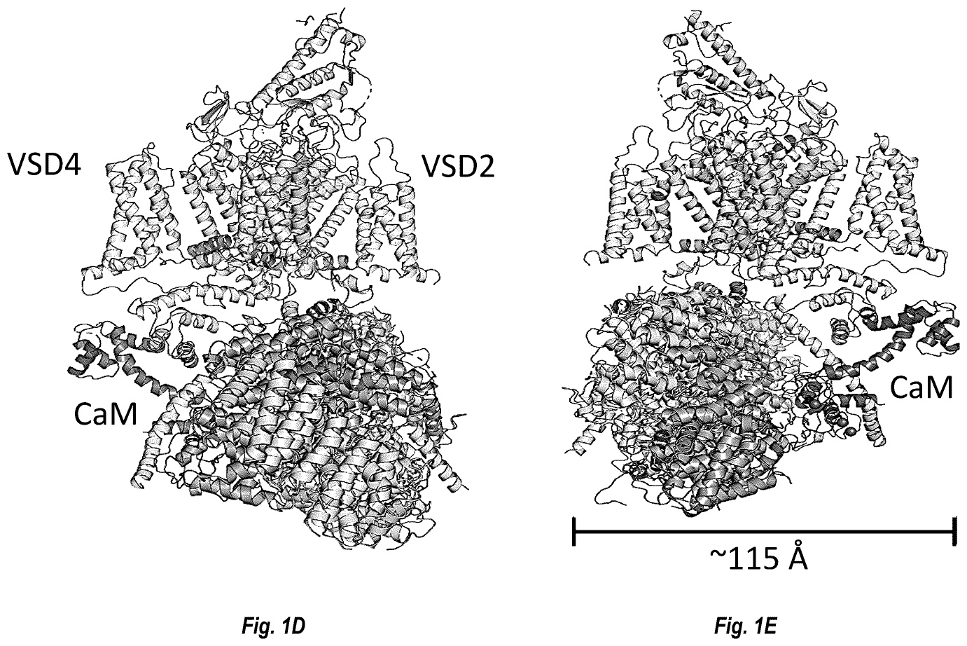

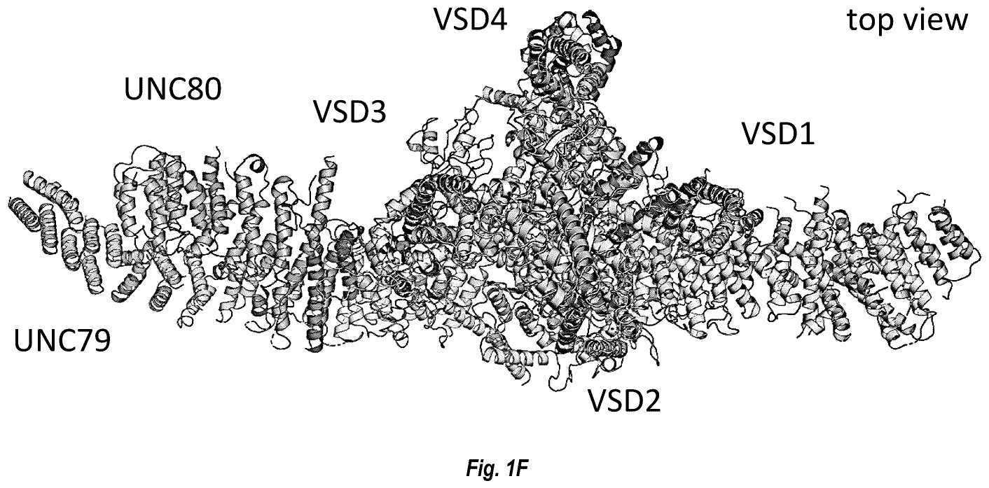

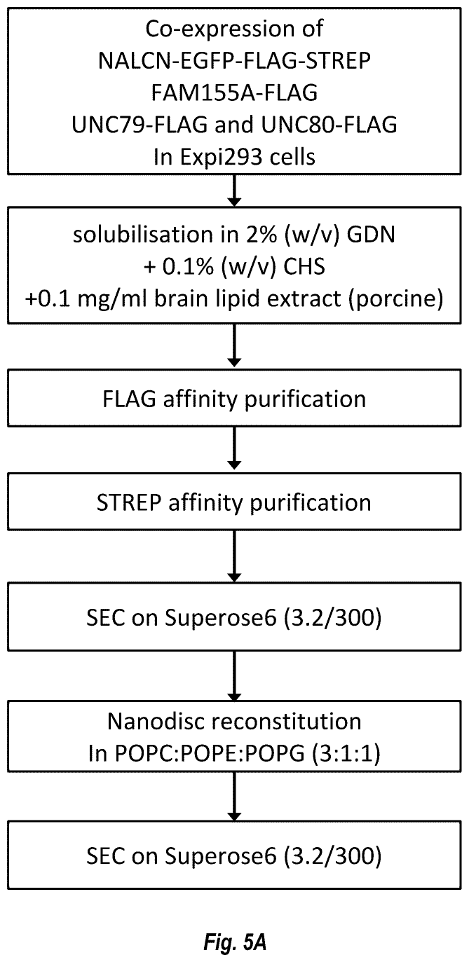

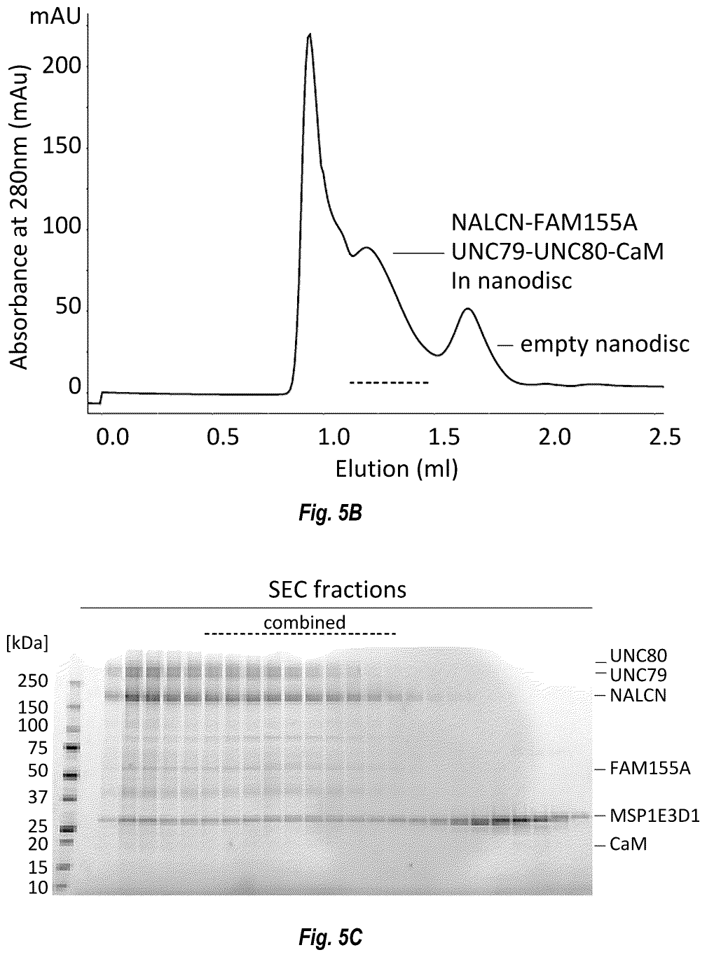

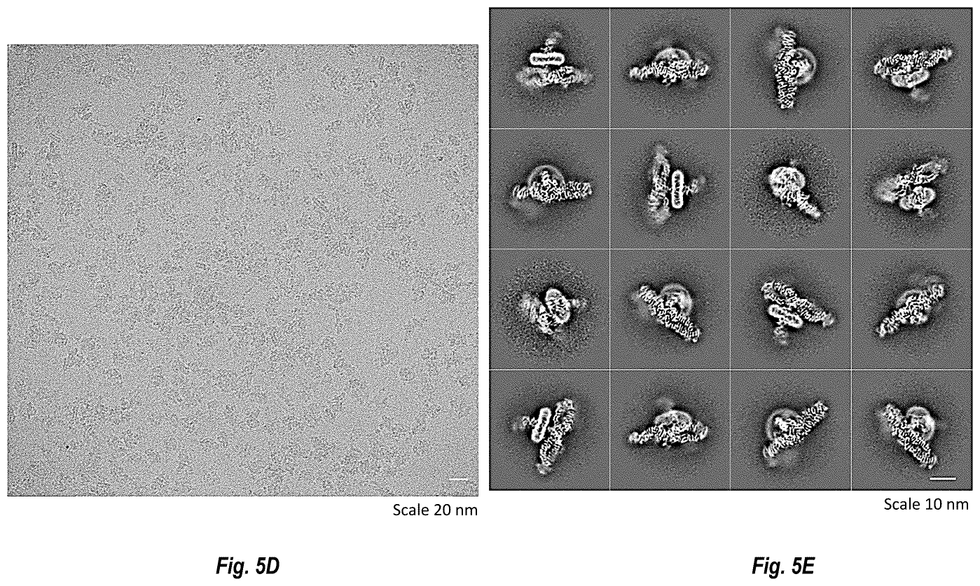

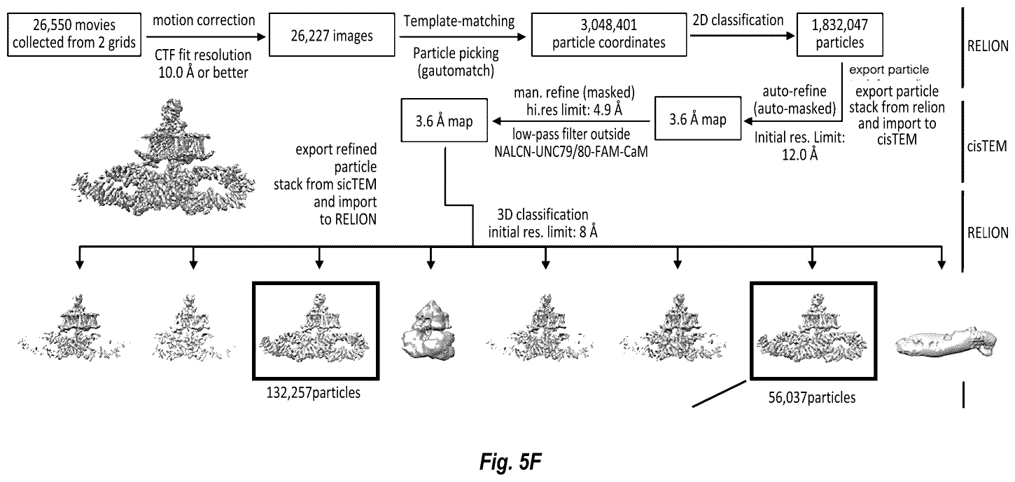

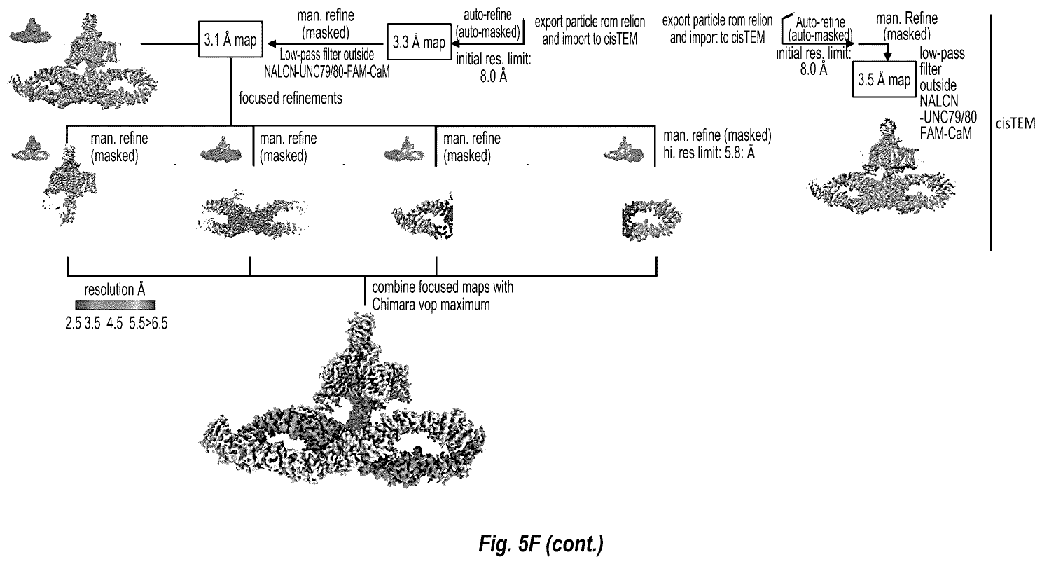

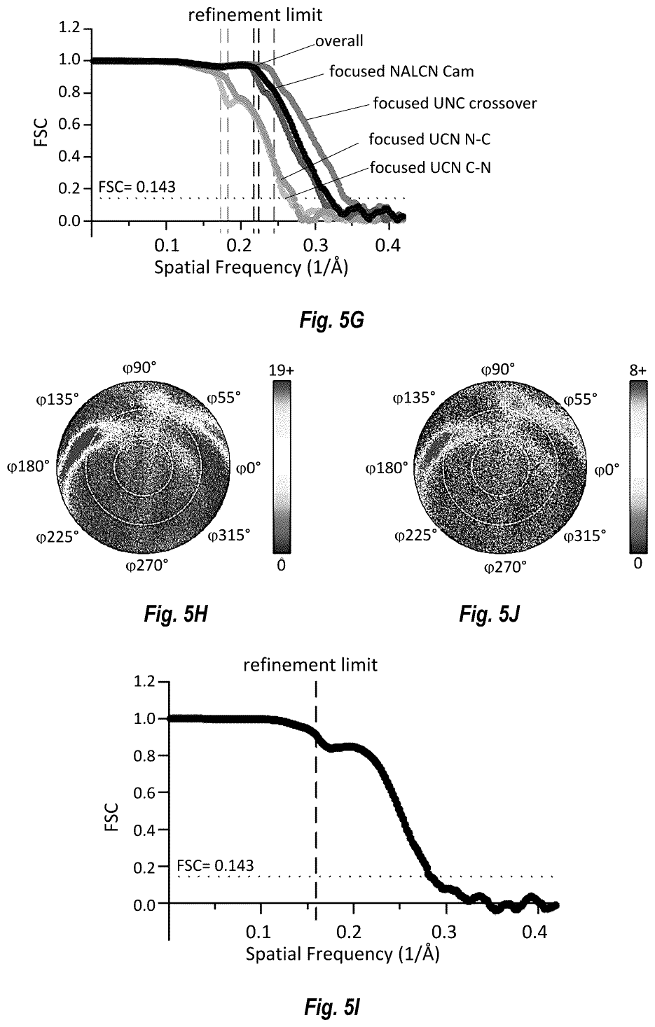

Previous efforts to purify an intact NALCN-FAM155A-UNC79-UNC80 complex resulted only in the isolation of a NALCN-FAM155A subcomplex²⁴⁻²⁶. We noted that fusion of a tagged green fluorescent protein (GFP-Flag) to the carboxy-terminus of the NALCN subunit does not appreciably impact functional properties (FIG. 1A), which allowed isolation of a stable NALCN_{GFP}-FAM155A-UNC79-UNC80 complex (FIG. 5A-C).

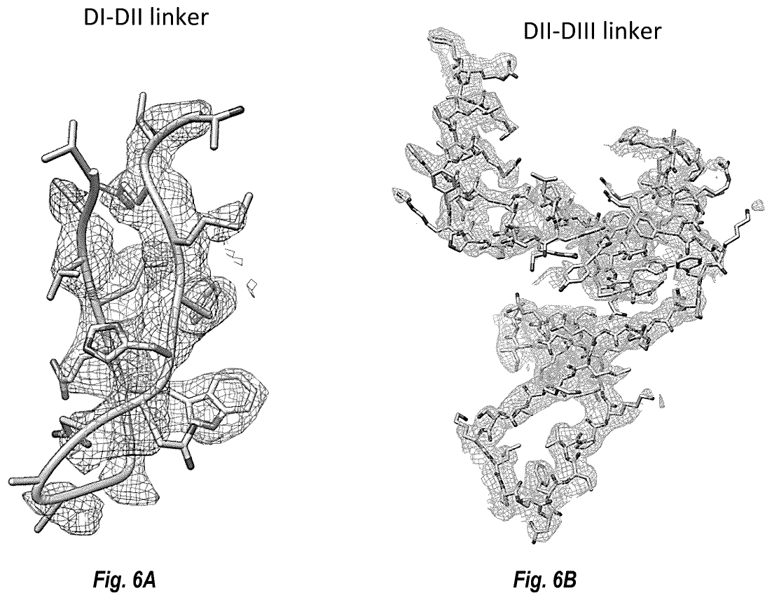

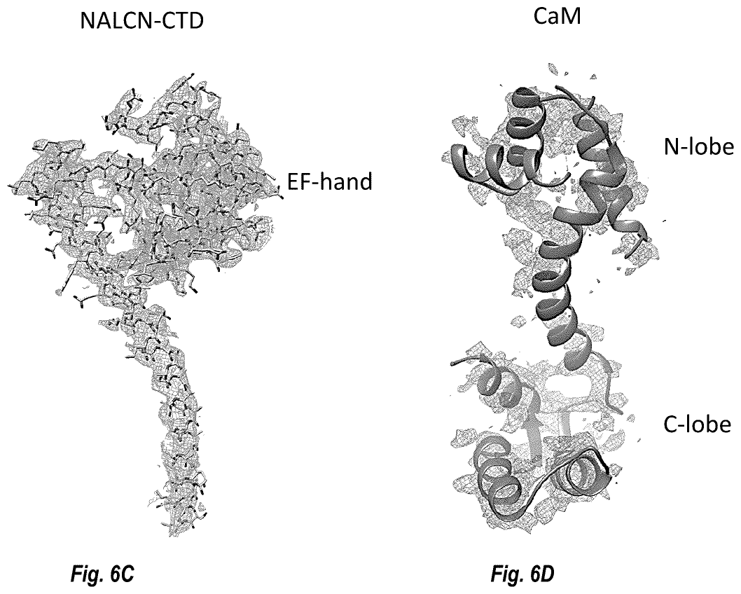

The NALCN_{GFP}-FAM155A-UNC79-UNC80-CaM complex was reconstituted into lipid nanodiscs and vitrified for cryo-electron microscopy (cryo-EM) studies without Ca²⁺ supplementation (FIG. 5C-D). Our 3D reconstruction extended to approximately 3.1 Å resolution and revealed all four components of the NALCN-FAM155A-UNC79-UNC80 channelosome, as well as CaM (FIGS. 1B-F and 5E-H; Table 1). It was not possible to assign a full atomic model in every region of the map due to inherent characteristics of the sample (FIGS. 5F and 6A-B).

TABLE 1
Cryo-EM data collection, refinement and validation statistics

| | NALCN- | NALCN- |
|---|---|---|
| | FAM155A- | FAM155A- |
| | UNC79- | UNC79- |
| | UNC80-CaM | UNC80-CaM |
| | conformation 1 | conformation 2 |
| Data collection and processing | | |
| Magnification | 105,000 | 105,000 |
| Voltage (kV) | 300 | 300 |
| Electron exposure (e−/Å²) | 64.009 | 64.009 |
| Defocus range (μm) | 0.5-1.5 | 0.5-1.5 |
| Pixel size (Å) | 0.838 | 0.838 |
| Symmetry imposed | C1 | C1 |
| Initial particle images (no.) | 3,048,401 | 3,048,401 |
| Final particle images (no.) | 132,257 | 56,037 |
| Map resolution (Å) | 3.1 | 3.5 |
| FSC threshold | 0.143 | 0.143 |
| Map resolution range (Å) | 2.6-46.9 | 3.2-46.9 |
| Refinement | | |
| Initial model used (PDB code) | 6XIW + 4DCK | |
| Model resolution (Å) | 3.2 | 4.1 |
| FSC threshold | 0.5 | 0.5 |
| Model resolution range (Å) | 2.6-46.9 | 3.2-46.9 |
| Map sharpening B factor (Å²) | −90 | −90 |
| Model composition | | |
| Non-hydrogen atoms | 37959 | 37505 |
| Protein residues | 4608 | 4567 |
| Ligands | 11 | 7 |
| Water | 0 | 0 |
| B factors (Å²) | | |
| Protein | 88.22 | 124.97 |
| Ligand | 102.51 | 173.38 |
| Water | — | |
| R.m.s. deviations | | |
| Bond lengths (Å) | 0.004 | 0.004 |
| Bond angles (°) | 0.943 | 0.959 |
| Validation | | |
| MolProbity score | 1.26 | 1.25 |
| Clashscore | 3.21 | 4.10 |
| Poor rotamers (%) | 0.00 | 0.00 |
| Ramachandran plot | | |
| Favored (%) | 97.22 | 97.76 |
| Allowed (%) | 2.78 | 2.24 |
| Disallowed (%) | 0.00 | 0.00 |

### Example 4. Overall Structure of the NALCN Channelosome

Unprecedented among ion channels, the architecture of the NALCN channelosome complex is reminiscent of a bicycle when viewed perpendicular to the membrane (FIG. 1B-C). The distinctive architecture of the complex is contributed by UNC79 and UNC80 which form a massive, intertwined, intracellular assembly that hangs beneath voltage-sensor domain 1 (VSD1), VSD2 and VSD3 on NALCN (FIG. 1B-F-). Below VSD4, the C-terminal domain (CTD) of NALCN is embraced by CaM, which itself is docked against UNC80 near the midpoint of the intracellular assembly (FIG. 1D-E).

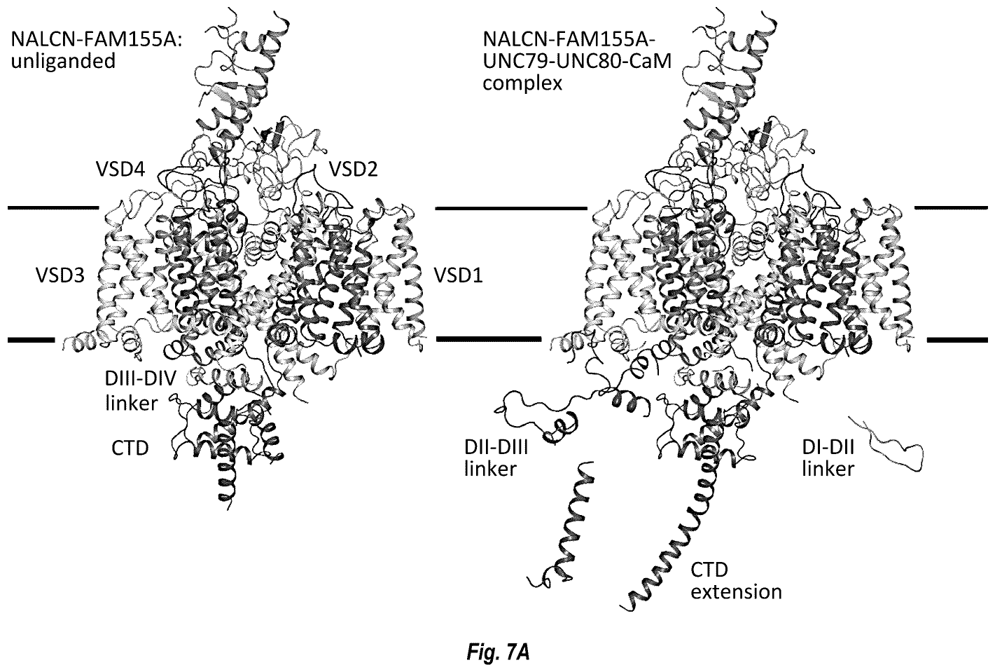

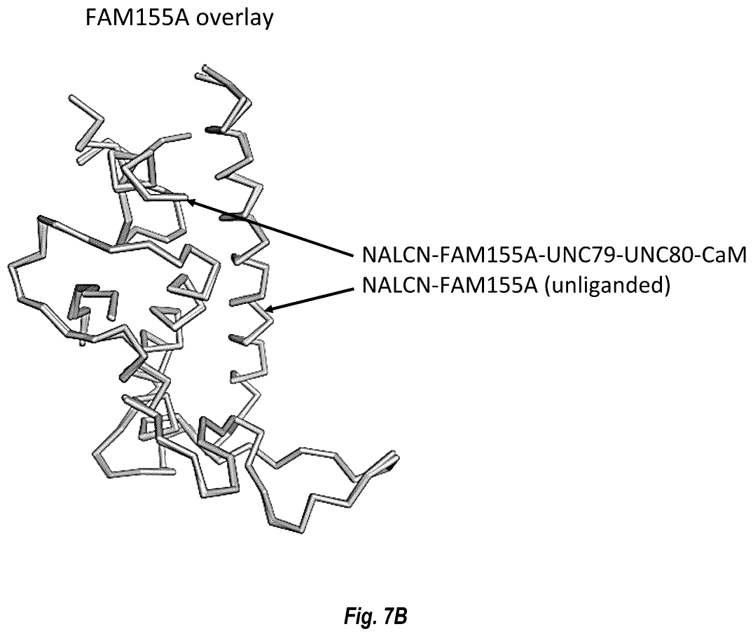

The global conformation of the NALCN-FAM155A pore-forming subcomplex is largely unchanged within the channelosome relative to the unliganded subcomplex (FIG. 7A)²⁴⁻²⁶. FAM155A is unaltered in structure (FIG. 7B), supporting conclusions that it primarily functions to stabilize the NALCN subunit, while shielding the selectivity filter from neurotoxin attack²⁵. Moreover, the two predicted transmembrane helices of FAM155A remain unresolved (FIG. 5F), indicating that these act as simple membrane anchors for the extracellular domain¹⁰. Critically, in the presence of UNC79, UNC80 and CaM, the NALCN pore-forming subunit showed extensive ordering of intracellular linker regions (FIGS. 1B and 7A). These key structural features have important consequences for NALCN channelosome assembly and gating, as discussed below.

### Example 5. UNC79 and UNC80 are Atypical HEAT-Repeat Proteins

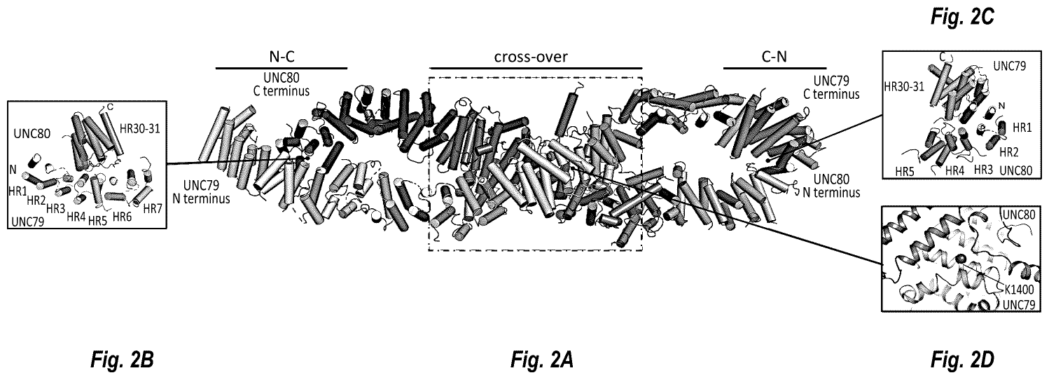

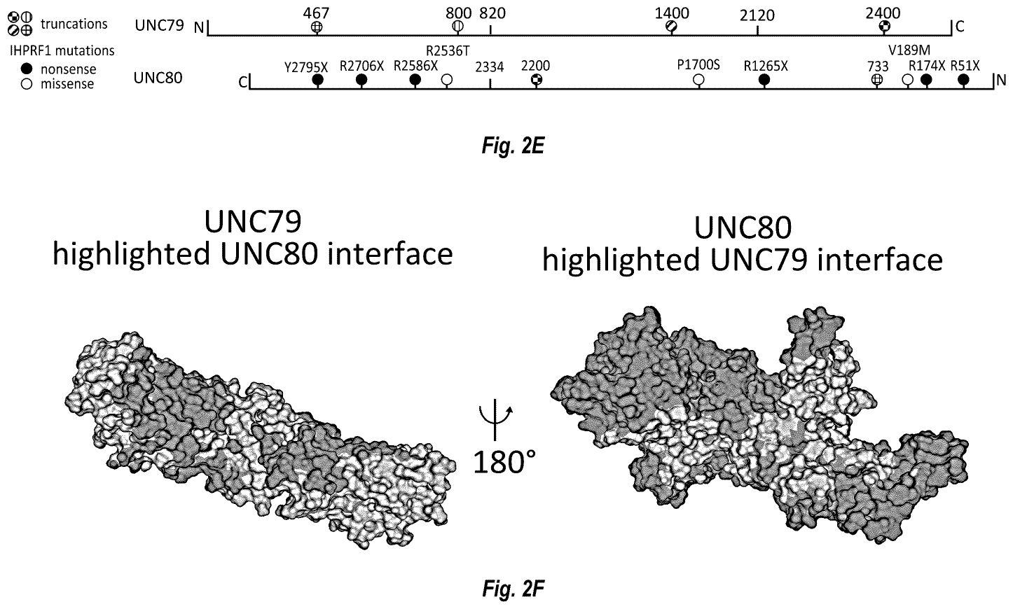

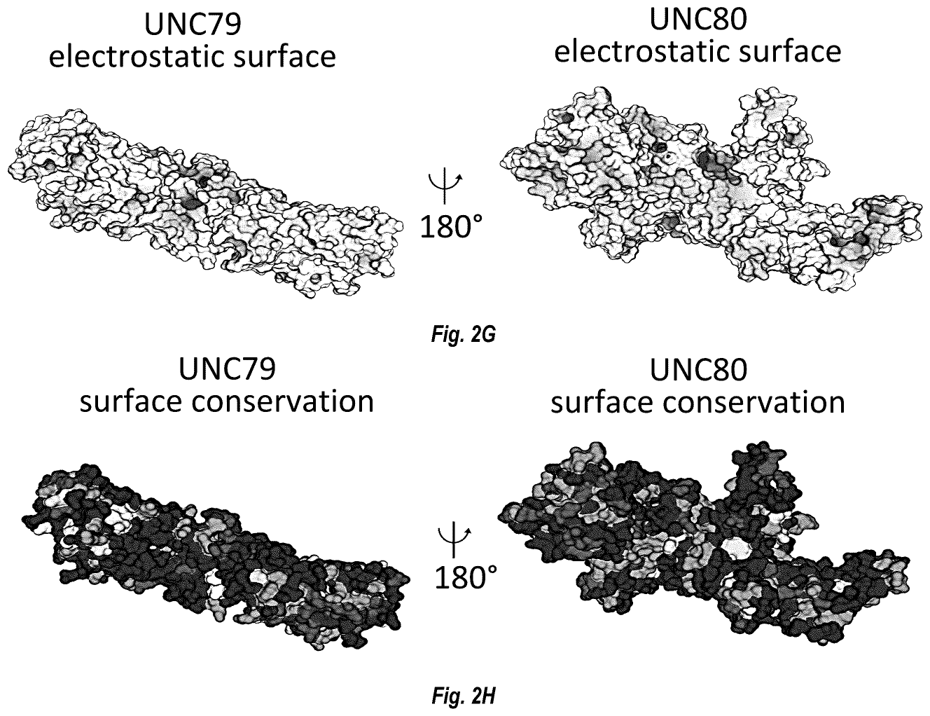

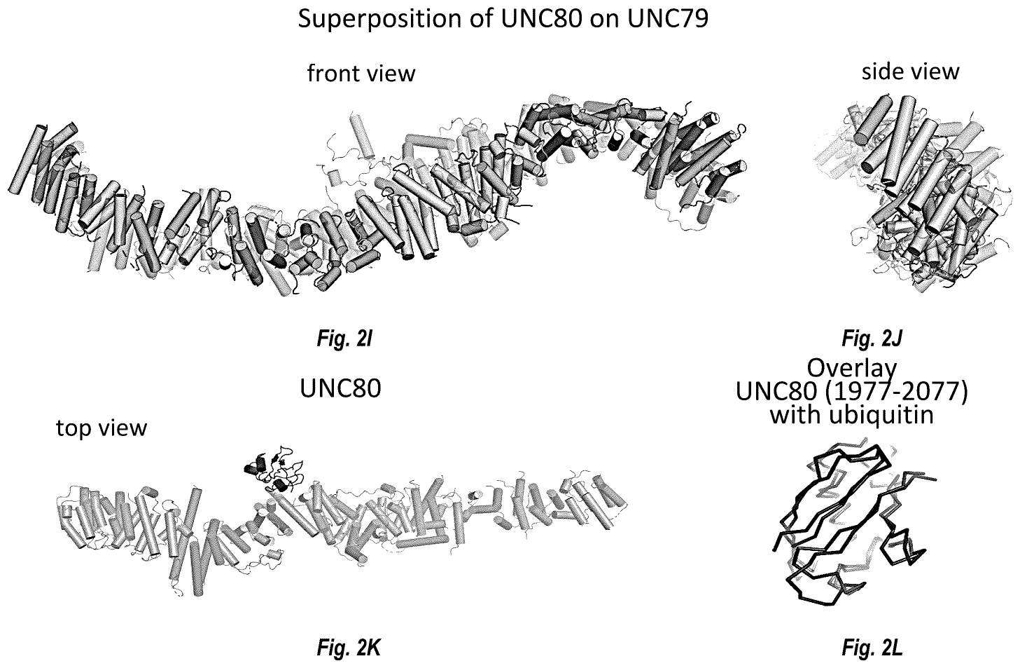

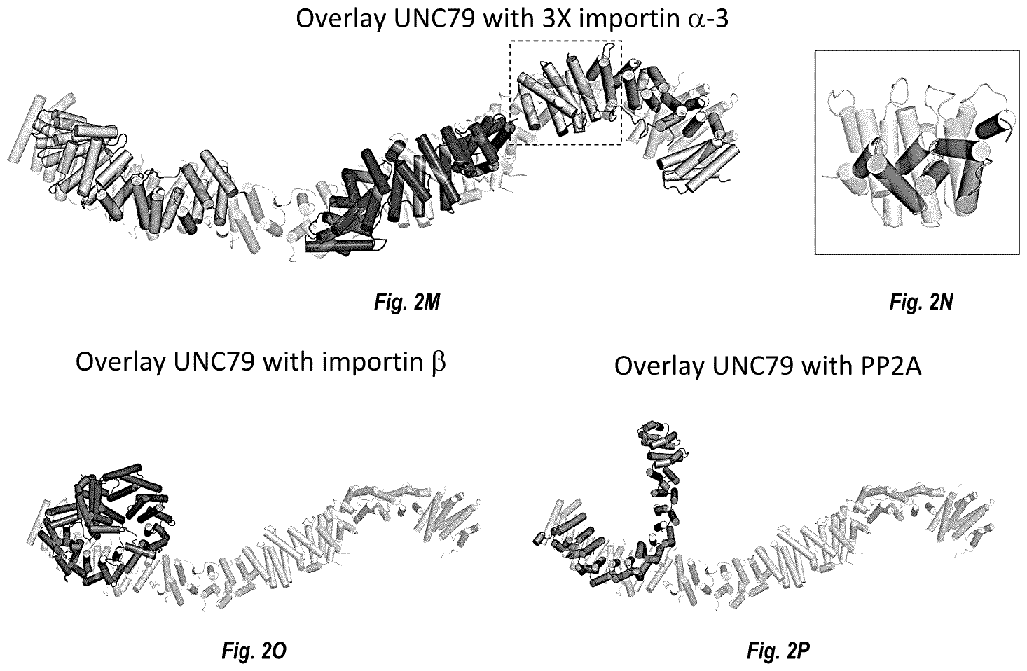

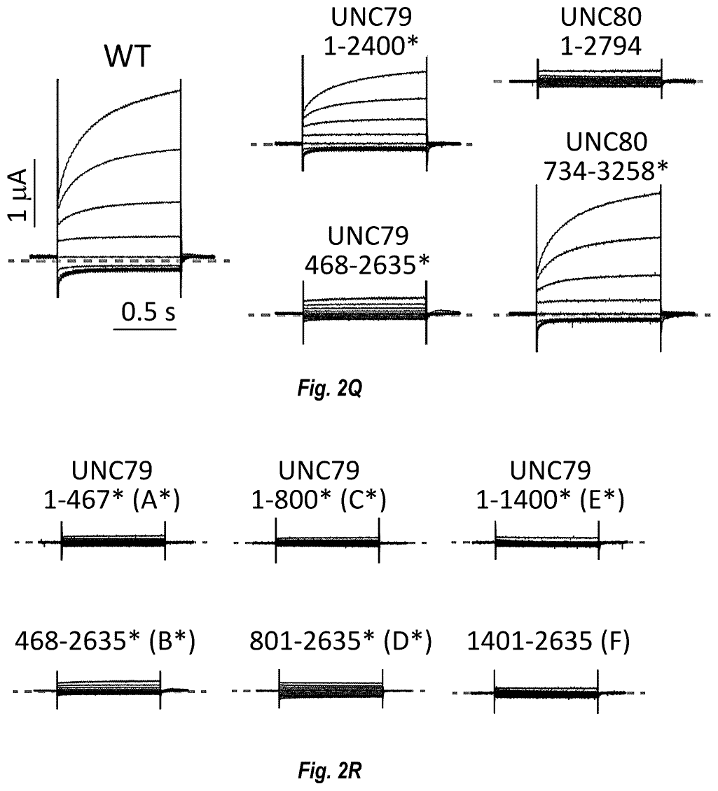

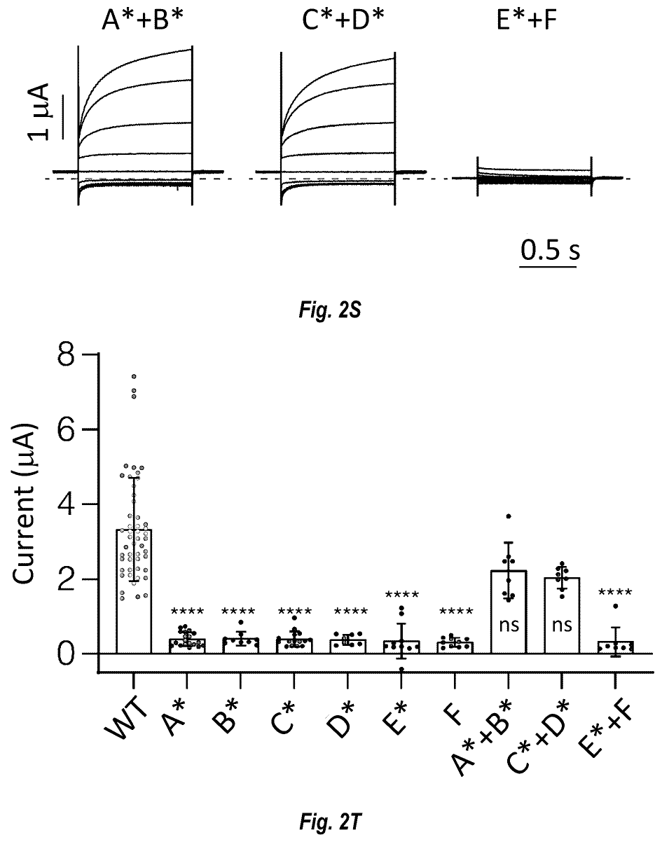

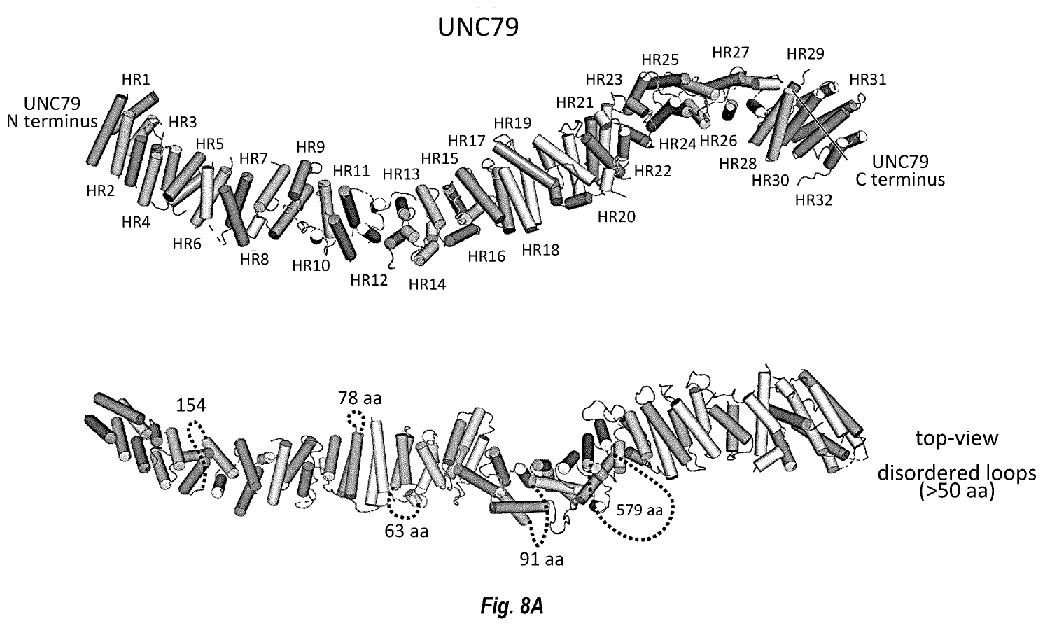

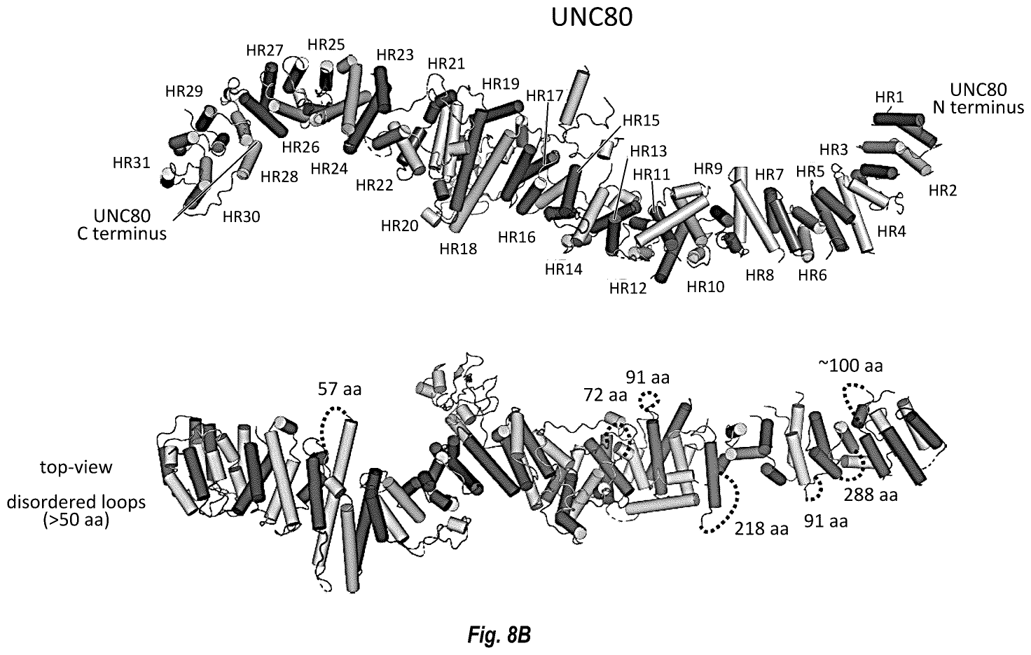

Violating all predictions, UNC79 and UNC80 are well-folded globular proteins (FIGS. 1B-F, 2A-P, and 8A-B). UNC79 and UNC80 form an intimate head-to-tail superhelical assembly reminiscent of an infinity sign that stretches ˜300 Å underneath the membrane bilayer (FIGS. 1C and 2A). Transmembrane spanning helices or membrane interactions are not evident (FIGS. 5F and 8A-B), indicating that UNC79 and UNC80 may strictly associate with the NALCN pore-forming subunit through intracellular contacts (FIG. 1C-F). Three large interaction interfaces are formed between UNC79 and UNC80: the N_{UNC79}-C_{UNC80} (N—C) interface, the crossover interface, and C_{UNC79}-N_{UNC80} (C—N) interface (FIG. 2A-D).

UNC79 and UNC80 are immediately assigned to the HEAT repeat superfamily fold (FIG. 2A), defined by a repeating motif of two linked α-helices, where some HEAT repeats are irregular and present more of a three α-helix armadillo repeat-like character (FIG. 2A and Tables 2 and 3).

TABLE 2
DALI search of the Protein Data Bank using full-length UNC79 as a query.

| PDB ID | Z-score | RMSD | [symbol] | % ID | Molecule |
|---|---|---|---|---|---|
| [symbol] -E | 13.0 | 14.6 | 293 | 8 | IMPORTIN SUBUNIT ALPHA-3 |
| [symbol] -D | 12.8 | 9.7 | 282 | 13 | IMPORTIN SUBUNIT ALPHA-4 |
| [symbol] -A | 12.7 | 7.7 | 303 | 10 | CATENIN BETA-1 |
| [symbol] -F | 12.6 | 3.5 | 199 | 14 | ARMADILLO REPEAT PROTEIN |
| | | | | | ARM00027 |
| [symbol] -E | 12.5 | 3.0 | 177 | 15 | Y [symbol] M3AIII |
| [symbol] -P | 12.4 | 7.4 | 279 | 8 | V-TYPE PROTON ATPASE |
| | | | | | CATALYTIC SUBUNIT A |
| [symbol] -C | 12.3 | 24.9 | 535 | 10 | CULLIN HOMOLOG 1 |
| [symbol] -E | 12.3 | 9.1 | 234 | 12 | Y [symbol] M [symbol] 6A [symbol] |
| [symbol] -A | 12.2 | 16.2 | 400 | 9 | AP-2 COMPLEX SUBUNIT |
| | | | | | ALPHA-2 |
| [symbol] -A | 12.2 | [symbol] | 207 | 14 | [symbol] ( [symbol] 2)4C [symbol] |
| [symbol] -B | 12.2 | [symbol] | 208 | 10 | PROTEIN DIAPHANOUS |
| | | | | | HOMOLOG 1 |
| [symbol] -S | 12.2 | 12.0 | 286 | 9 | IMPORTIN BETA-1 SUBUNIT |
| [symbol] -B | 12.2 | 4.3 | 223 | 15 | [symbol] M5A [symbol] |
| [symbol] -C | 12.1 | 15.3 | 334 | 8 | GTP-BINDING NUCLEAR |
| | | | | | PROTEIN RAN |
| [symbol] -B | 12.1 | 10.7 | 218 | 10 | CELL DIVISION CONTROL |
| | | | | | PROTEIN 42 HOMOLOG |
| [symbol] -B | 12.1 | 5.1 | 265 | 9 | GTPASE ACTIVATOR-LIKE |
| | | | | | PROTEIN |
| [symbol] -F | 12.1 | 3.7 | 261 | 11 | DESIGNED ARMADILLO |
| | | | | | REPEAT PROTEIN WITH |
| | | | | | INTERNAL [symbol] |
| 5mfm-C | 12.0 | 4.0 | 267 | 13 | Y [symbol] M6A [symbol] _GS11_(KR) [symbol] |
| [symbol] -D | 12.0 | 4.6 | 217 | [symbol] | LOCK2_ [symbol] |
| 3wpt-B | 12.0 | 9.6 | 313 | 7 | IMPORTIN SUBUNIT ALPHA-1 |
| [symbol] -D | 11.9 | [symbol] | 184 | 9 | RHO-RELATED GTP-BINDING |
| | | | | | PROTEIN RHOC |
| [symbol] -B | 11.9 | 20.3 | 349 | 9 | CULLIN-ASSOCIATED NEOD8- |
| | | | | | DISSOCIATED PROTEIN 1 |
| [symbol] -A | 11.9 | 15.1 | [symbol] | 9 | TRANSPORTIN-1 |
| [symbol] -A | 11.9 | 7.2 | 275 | 12 | PLAKOGLOBIN |
| [symbol] -B | 11.9 | 5.1 | 356 | 8 | SYS-1 PROTEIN |

[symbol]

indicates data missing or illegible when filed

TABLE 3
DALI search of the Protein Data Bank using full-length UNC80 as a query.

| PDB ID | Z-score | RMSD | [symbol] | % ID | Molecule |
|---|---|---|---|---|---|
| [symbol] -A | 11.1 | 19.1 | [symbol] | 4 | SERINE/THREONINE-PROTEIN KINASE MTOR |
| [symbol] -A | 10.6 | 3.7 | 227 | 9 | CCR4-NOT TRANSCRIPTION COMPLEX SUBUNIT 9 |
| [symbol] -A | 10.5 | 4.0 | 201 | 6 | CLIP-ASSOCIATING PROTEIN 2 |
| [symbol] -C | 10.2 | 4.6 | 204 | 6 | CULLIN HOMOLOG 1 |
| [symbol] -A | 10.2 | 3.8 | 226 | 9 | RCO1 REQUIRED FOR DIFFERENTIATION1 HOMOLOG |
| [symbol] -D | 10.1 | 10.8 | [symbol] | 8 | IMPORTIN-9 |
| [symbol] -B | 10.0 | 4.4 | 267 | 6 | CULLIN-ASSOCIATED NEODE-DISSOCIATED PROTEIN 1 |
| [symbol] -A | 9.8 | 3.8 | 166 | 8 | 253 [symbol] LONG HYPOTHETICAL PROTEIN |
| [symbol] -B | 9.7 | 5.6 | 184 | 9 | IMPORTIN SUBUNIT ALPHA-1A |
| [symbol] -D | 9.6 | 3.9 | 202 | 7 | TRANSPORTIN-3 |
| [symbol] -D | 9.5 | 2.6 | 92 | 12 | RAS-RELATED PROTEIN RAP-1A |
| [symbol] -B | 9.5 | 3.0 | 87 | 11 | PROTEIN 4.1 |
| [symbol] -B | 9.3 | 10.9 | 230 | 8 | IMPORTIN SUBUNIT ALPHA-2 |
| [symbol] -C | 9.3 | 5.1 | 174 | 14 | DESIGNED ARMADILLO REPEAT PROTEIN, Y [symbol] M3A [symbol] |
| [symbol] -A | 9.2 | 3.1 | 87 | 10 | FERM, [symbol] GEF (ARHGEF) AND PLECKSTRIN DOMAIN |
| | | | | | PROTEIN |
| [symbol] -D | 9.2 | 3.3 | 78 | 10 | 2 [symbol] PROTEASOME REGULATORY SUBUNIT N11-LIKE |
| | | | | | PROTEIN |
| [symbol] -A | 9.2 | 4.1 | 251 | 12 | Y [symbol] M [symbol] 6A [symbol] |
| [symbol] -G | 9.2 | 4.2 | 197 | 10 | GTP-BINDING NUCLEAR PROTEIN RAN |
| [symbol] -D | 9.1 | 4.6 | 189 | [symbol] | Y [symbol] _M5_A [symbol] |
| [symbol] -A | 9.1 | 3.5 | 205 | 7 | CLIP-ASSOCIATING PROTEIN |
| [symbol] -A | 9.1 | 6.3 | 90 | 9 | BAND 4.1-LIKE PROTEIN 3 |
| [symbol] -B | 9.1 | 2.8 | 92 | 7 | MOESIN |
| [symbol] -B | 9.1 | 4.3 | 188 | [symbol] | ARM0000 [symbol] |
| [symbol] -B | 9.1 | 7.4 | 170 | 13 | DESIGNED ARMADILLO REPEAT PROTEIN, Y [symbol] M3A [symbol] |
| [symbol] -A | 9.0 | 2.2 | 77 | 9 | SMALL [symbol] -RELATED MODIFIER 1 |

[symbol]

indicates data missing or illegible when filed

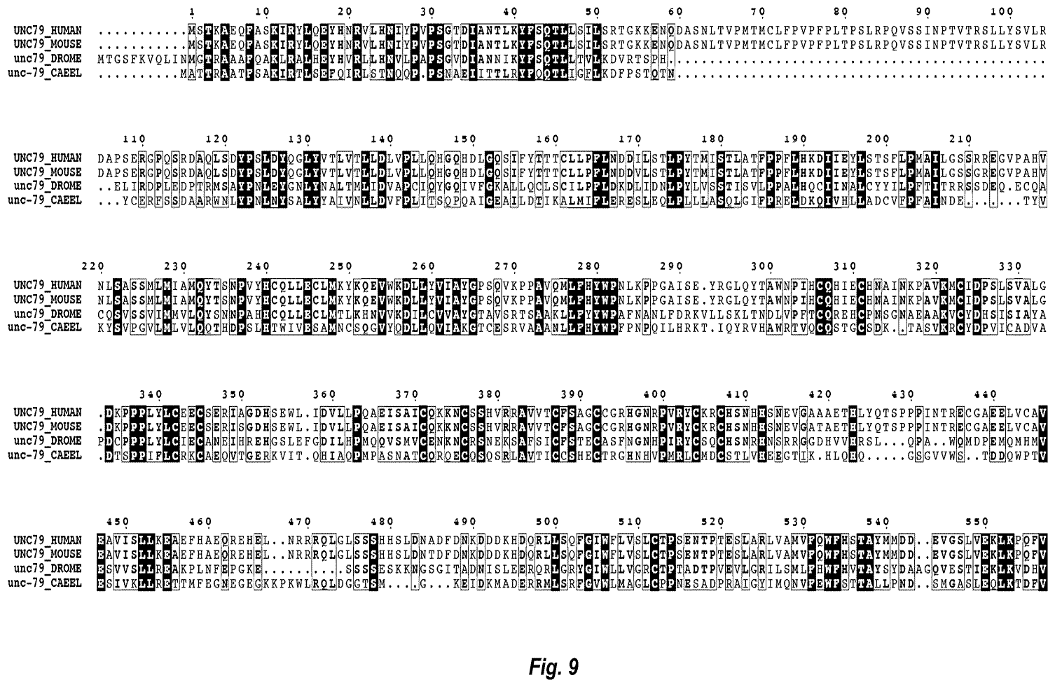

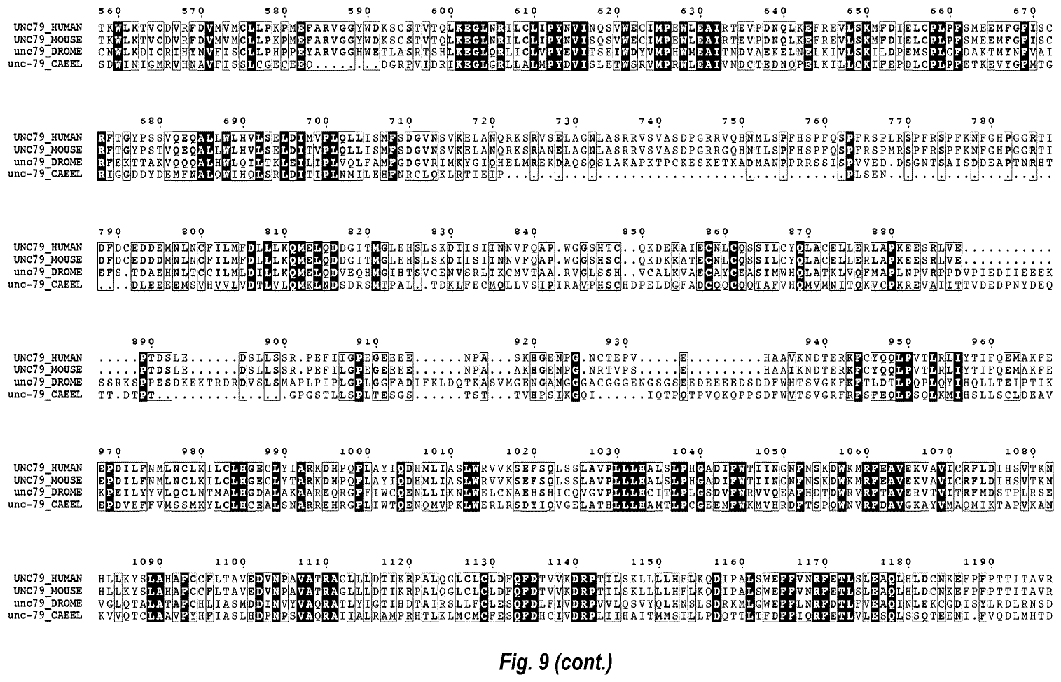

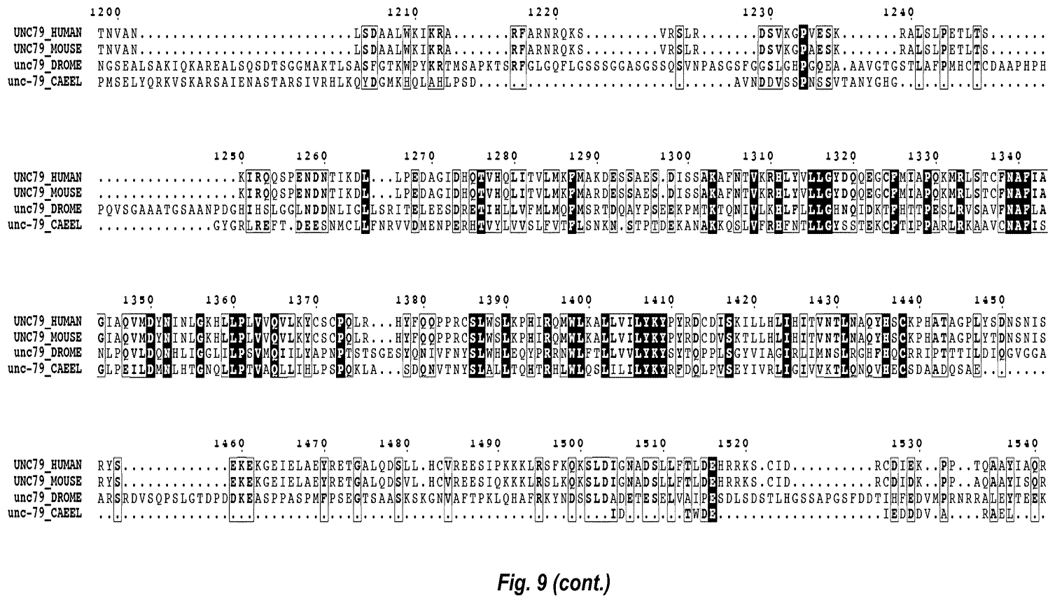

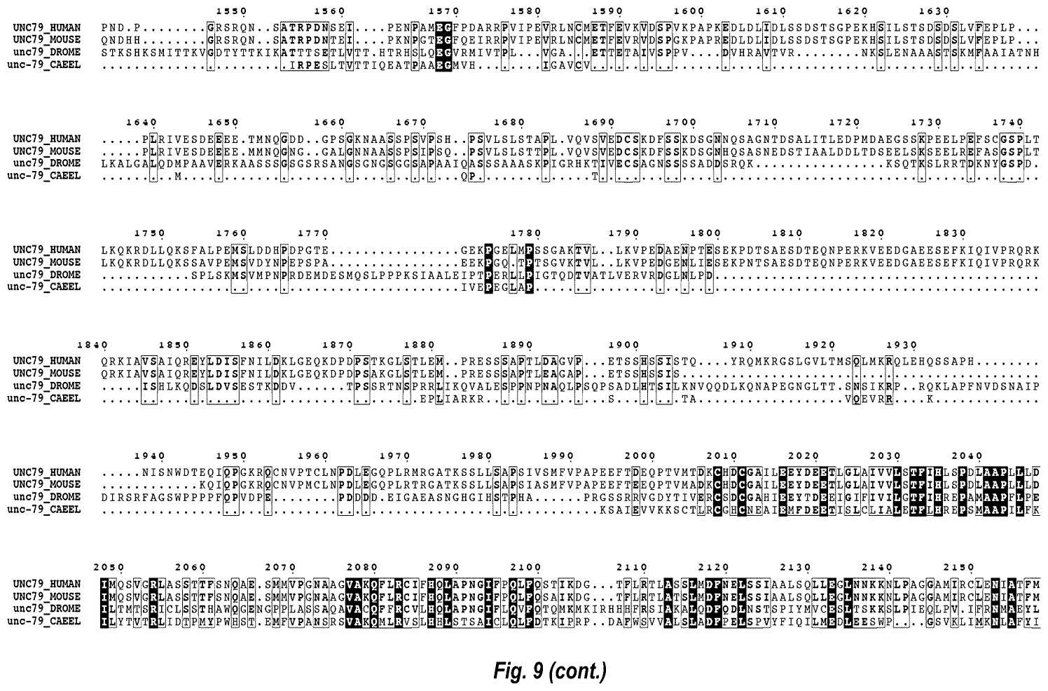

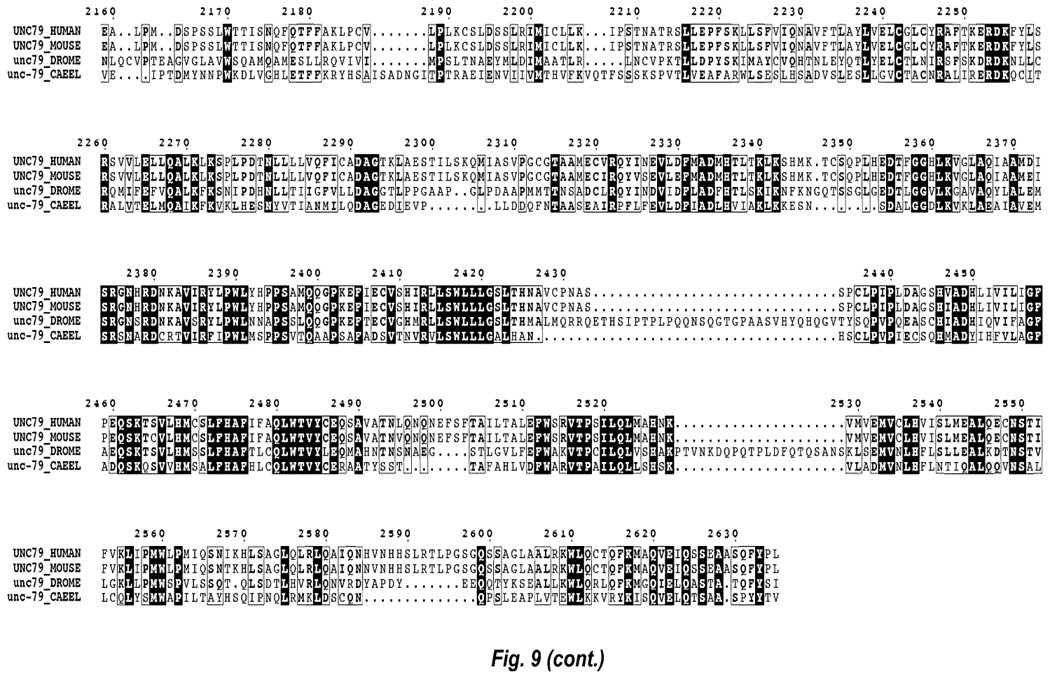

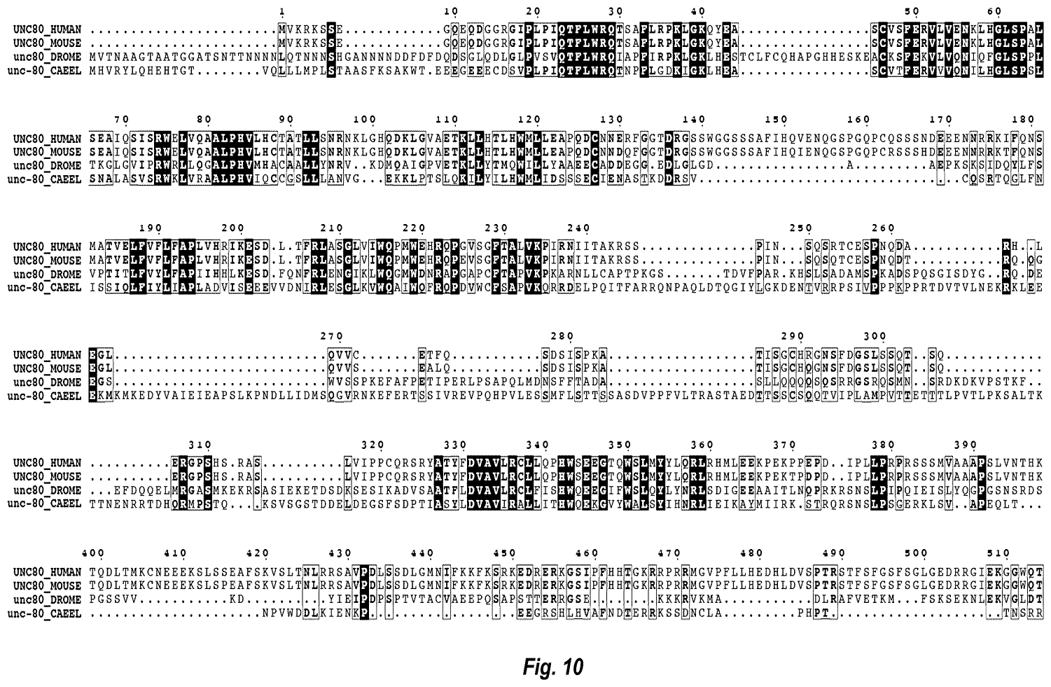

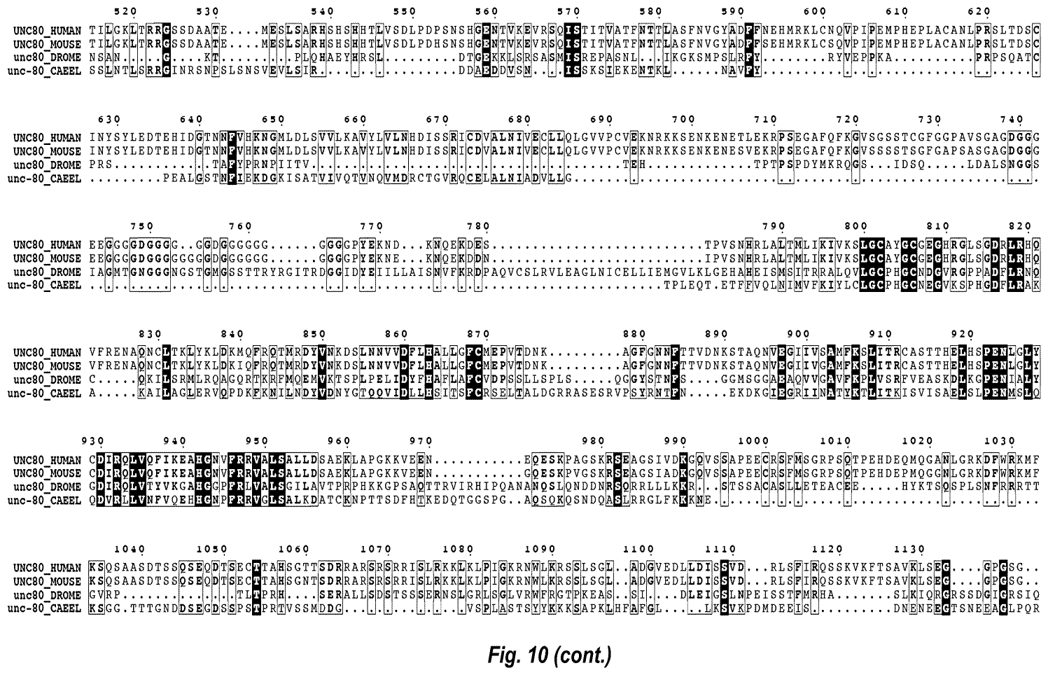

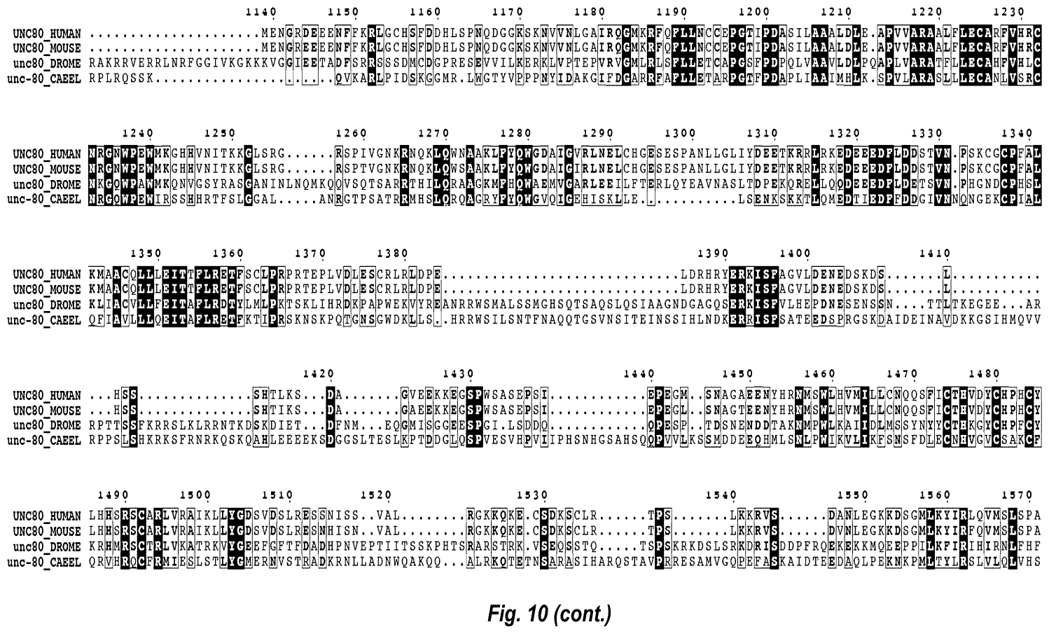

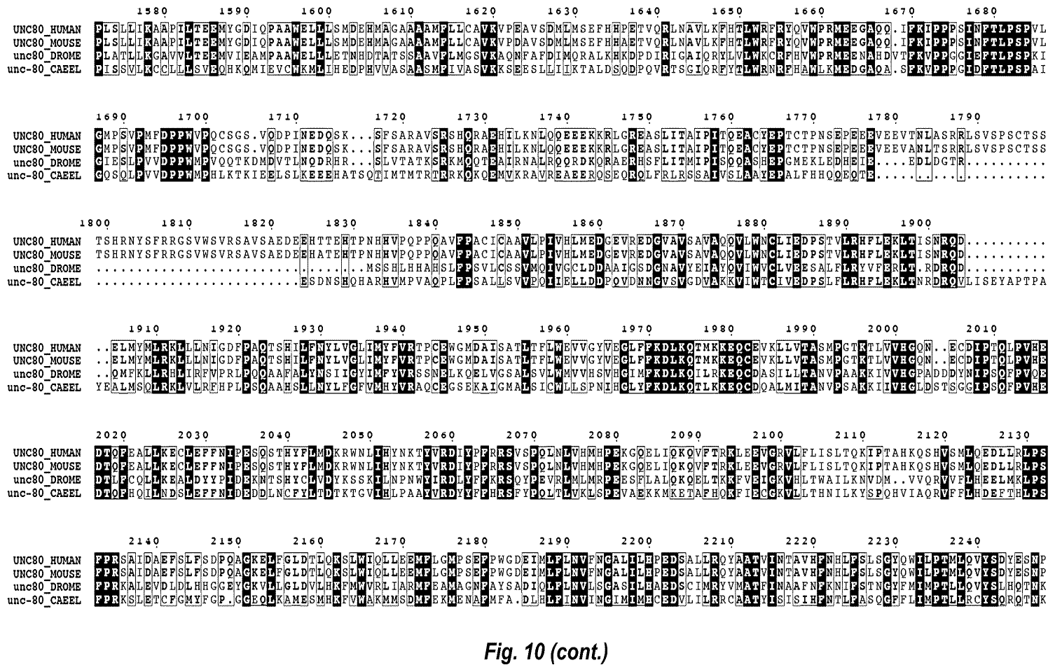

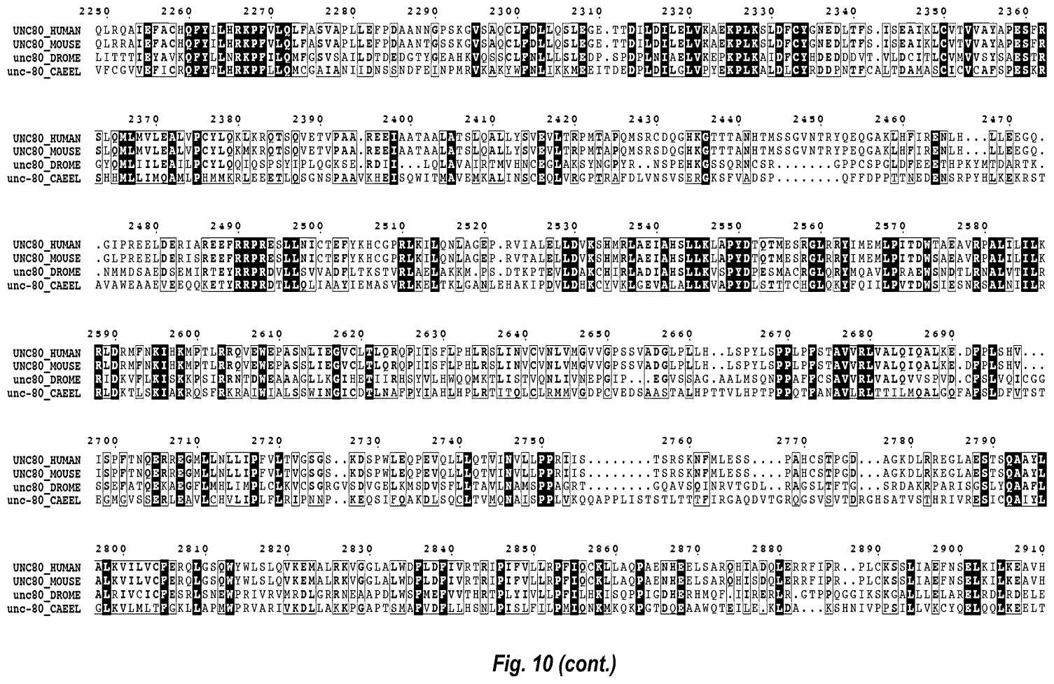

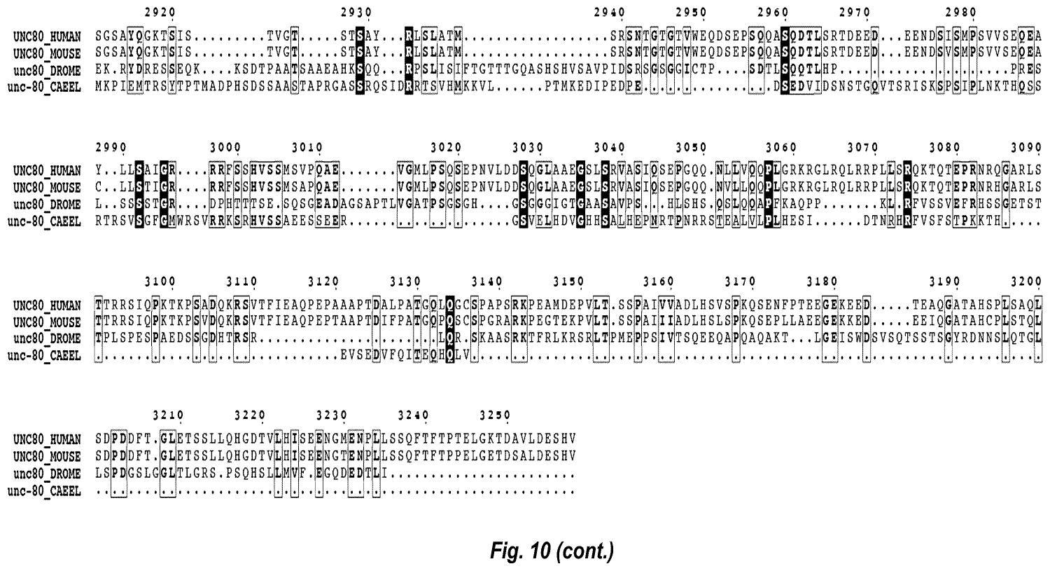

UNC79 (2,635 residues) contains 70 modeled α-helices representing 32 HEAT repeats, whereas UNC80 (3,258 residues) contains 72 modeled α-helices representing 31 HEAT repeats (FIGS. 2A and 8A-B). The folded and highly intertwined structures of UNC79 and UNC80 can finally explain their unusually high sequence conservation among mammals (FIG. 9-10). Compared to UNC80, UNC79 is more regular in shape and dimensions with fewer deviations in α-helical length across its structure, rationalizing why this smaller protein contains more HEAT repeats (FIGS. 2A and 2F-J). UNC80 has many exceptional HEAT repeat units with α-helices extending up to 60 residues in length, as well as elaborations with regular and irregular substructures, including the integration of a ubiquitin (Ub)-like fold near the NALCN-CTD and CaM interaction site (FIGS. 2A and 2F-L). UNC79 and UNC80 both also contain numerous unresolved loops along their respective scaffolds, including a ˜600 residue disordered region spanning 1437-2014 in UNC79 (FIG. 8A-B). Overall, UNC80 appears thicker and less uniform when compared to UNC79, but despite unrecognizable sequence identity, structural superposition reveals a highly similar architecture is shared between UNC79 and UNC80 (FIG. 2F-J).

HEAT repeat proteins are ubiquitous, so it is surprising that UNC79 and UNC80 are the first examples integrated into any ion channel (Tables 2 and 3). A DALI search³² of the Protein Data Bank returned importin-x3 as a top hit, which can be superimposed end-to-end three times over the length of UNC79 and UNC80 (FIG. 2I-J). Beyond this crude relation to importin-α3, UNC79 and UNC80 differ substantially from canonical HEAT-repeat proteins in terms of alpha-solenoid structure pitch (FIG. 2O-P-). These striking architectural differences may explain the lack of strong sequence identity to other HEAT repeat proteins (Tables 2 and 3), and suggest that the UNC79 and UNC80 subunits have evolved highly specialized roles in the assembly and function of the NALCN channelosome complex.

### Example 6. UNC79 and UNC80 Form an Integrated Structural Module

Three extensive and conserved interaction interfaces are observed between UNC79 and UNC80, providing the impression that these subunits form a stable subcomplex (FIG. 2A-H). UNC79 and UNC80 are both S-shaped and dock against each other in a complementary fashion along an apparent degenerate pseudo-twofold axis (FIGS. 2A and 2I-J). Recognizable structural features are found at each end of the complex, where the N—C domain is stabilized by C-terminal helices contributed by UNC80, the C—N domain is similarly capped by C-terminal helices contributed by UNC79 (FIG. 2A-C).

The crossover interface at the midpoint of the complex illustrates why both UNC79 and UNC80 are essential for NALCN channelosome assembly and function (FIGS. 2B and 2D). This conserved interface reveals a high degree of surface and electrostatic complementarity (FIG. 2F-H), burying an impressive ˜3000 A2 of solvent accessible surface area. In fact, an intricate mixture of polar and hydrophobic features are conserved along all of the UNC79-UNC80 interfaces (FIGS. 2G and 2H), dictated by the unusual size and geometry of the assembly. Thus, UNC79 cannot recapitulate the UNC80 interactions, or vice versa, so the channelosome will not form in the absence of either subunit. It is unknown if UNC79 or UNC80 may exist in isolation within a cell, but exposure of such large hydrophobic surfaces might be expected to impact protein expression, folding, or stability of the individual subunits. In support of this notion, all cellular UNC79 and UNC80 appears to be bound to NALCN¹², and protein expression levels and localization are also correlated^{3,17,30}.

### Example 7. The UNC79-UNC80 Subcomplex Impacts NALCN Activity

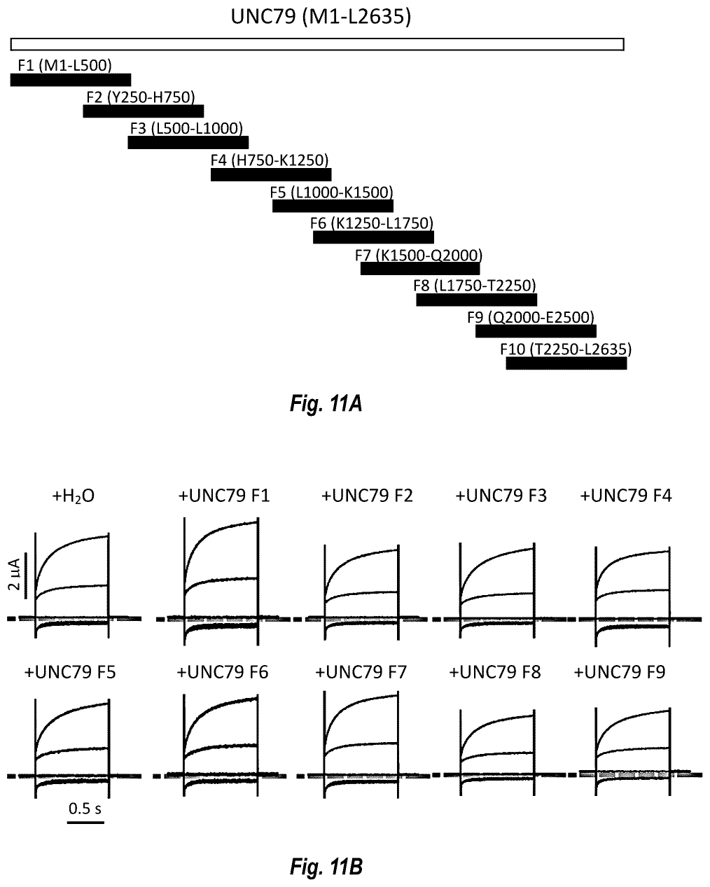

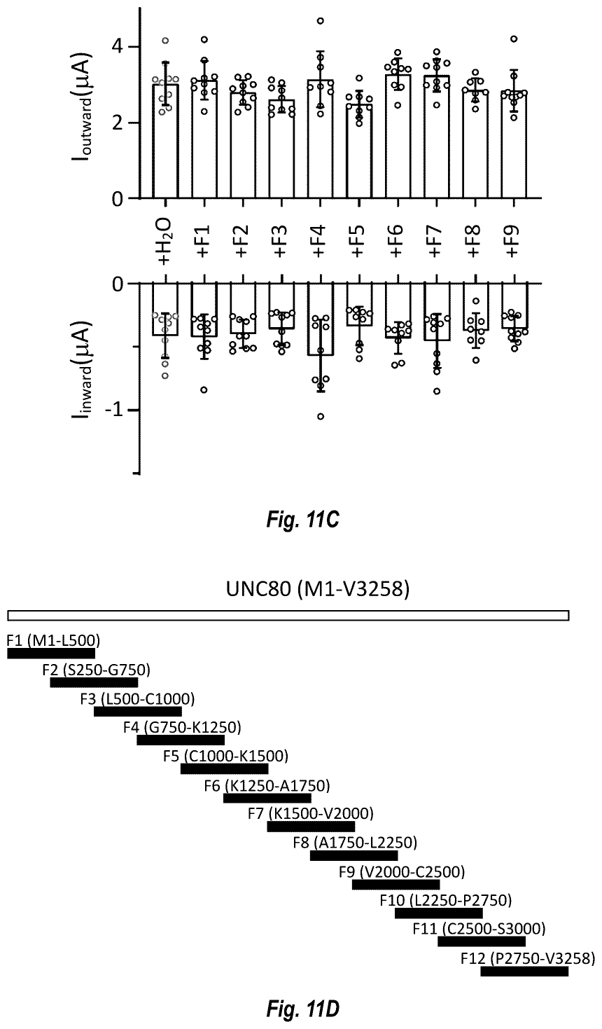

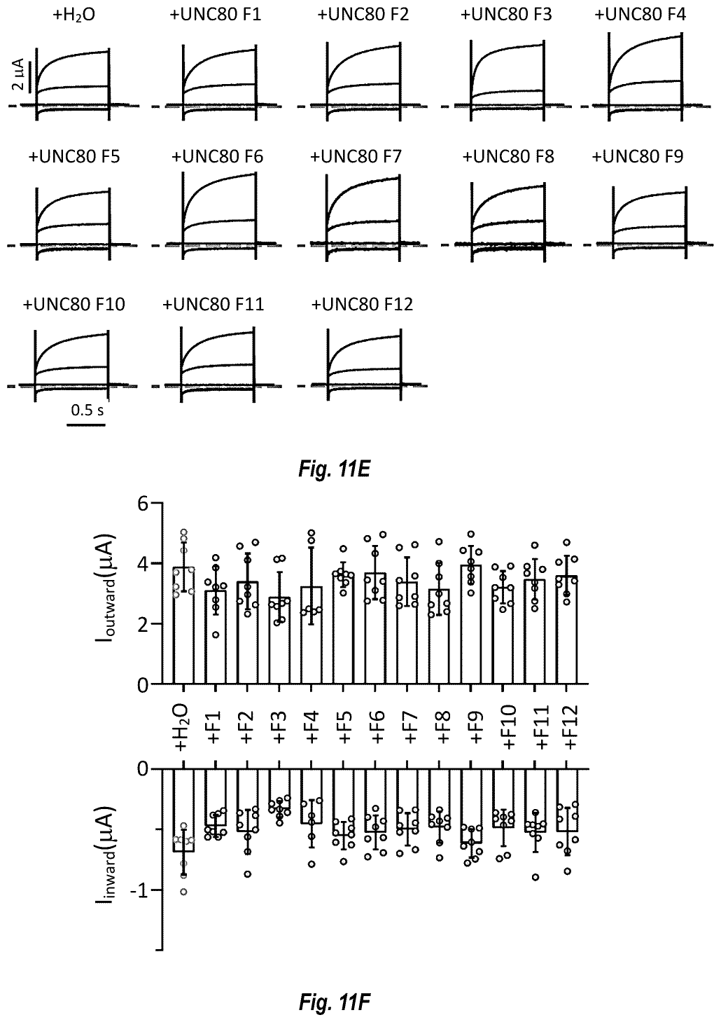

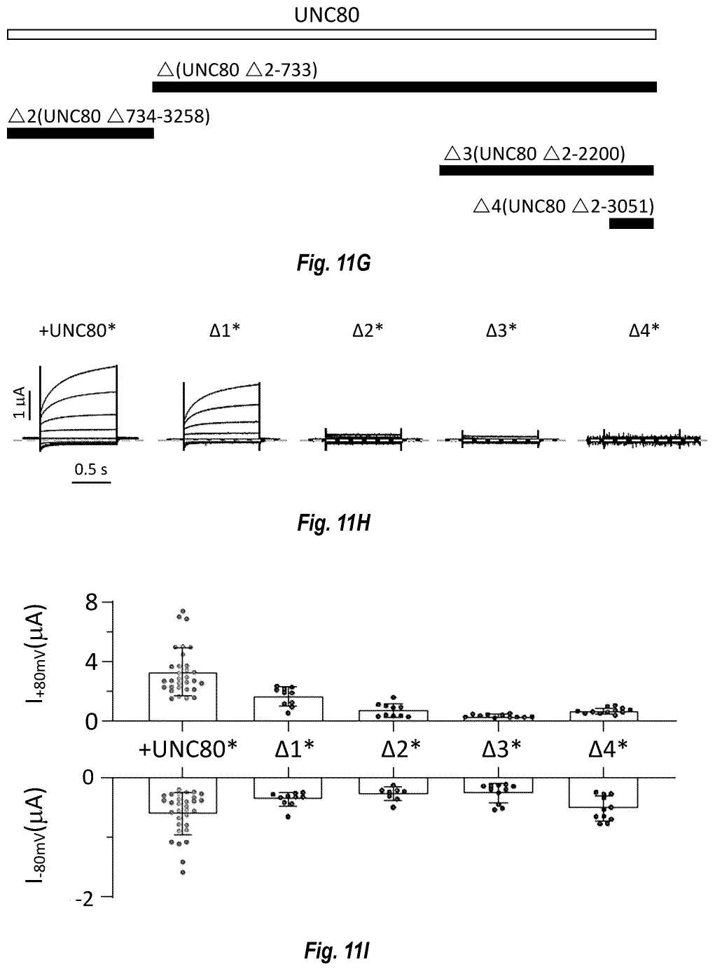

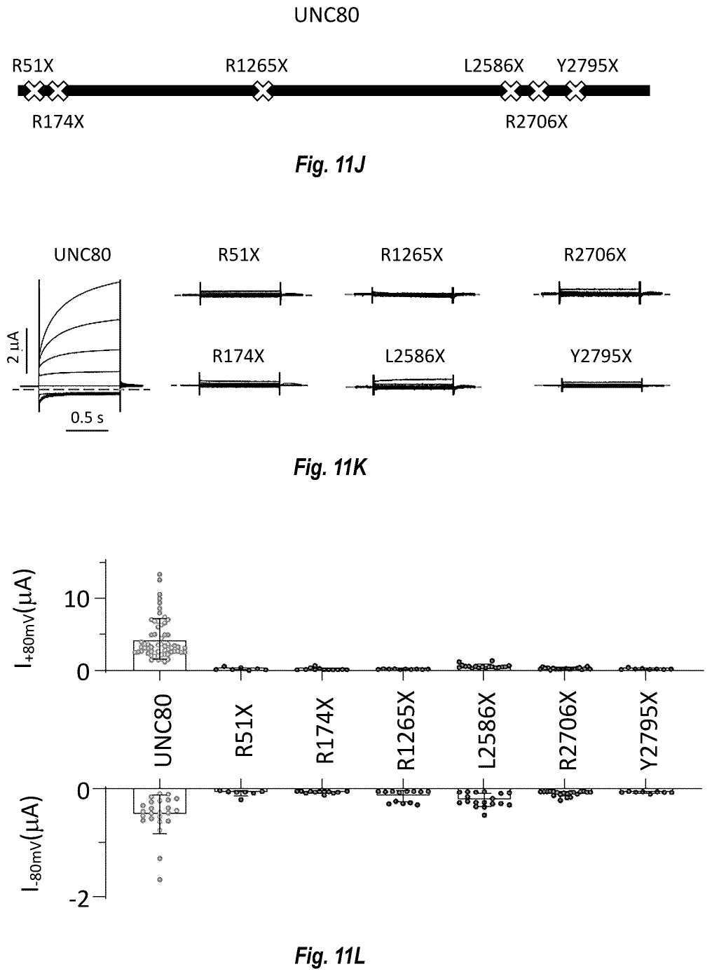

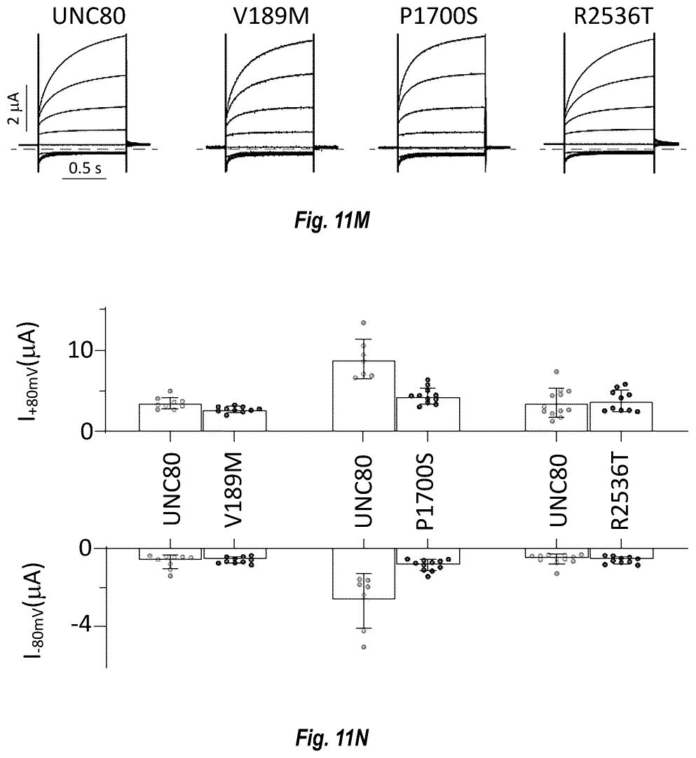

To interrogate the UNC79-UNC80 subcomplex, we first systematically co-expressed overlapping ˜500-residue fragments from each subunit in the context of the NALCN channelosome in Xenopus laevis oocytes (FIG. 11A-F). No fragment produced a dominant gating phenotype, expected as a change in inward or outward current, highlighting the fidelity with which full-length UNC79 and UNC80 subunits co-assemble (FIG. 11A-F). We next evaluated constructs with truncations or internal deletions and discovered that NALCN channelosome function tolerates deletion of either the C-terminal region of UNC79 (Δ2401-2635) or the N-terminal region of UNC80 (Δ1-733), respectively (FIGS. 2E, 2Q-T, and 11G-I)¹⁰, indicating that portions of the C—N interface appear dispensable (FIG. 2A). By contrast, NALCN channelosome function is sensitive to deletions at the N—C interface (FIGS. 2A, 2E, 2Q-T, and 11G-I), consistent with prior suggestions that UNC79 forms an essential interaction with UNC80¹². We subsequently split UNC79 at three positions and co-expressed complementary fragments along with intact NALCN, FAM155A and UNC80. NALCN channelosome function was permissive to fragmentation of UNC79 at Leu467 or Cys800, but severing UNC79 at Lys1400 resulted in a loss of function (FIG. 2E, 2Q-T), indicating that disruption of the crossover interface is not tolerated (FIG. 2A).

In humans, biallelic mutations in UNC80 cause an autosomal recessive disease where pathogenic mutations can occur as nonsense mutations^{12,28,29}. Three nonsense UNC80 mutations (Arg51X, Arg174X, and Arg2706X) failed to evoke currents when co-expressed with NALCN, FAM155A and UNC79 in oocytes (FIGS. 2E and 11J-L). Structurally, these mutations are all predicted to be detrimental to UNC80 folding or assembly of the NALCN channelosome (FIGS. 2A and 2E), as are previously evaluated (INC80 nonsense mutations^{12,28,29}. Two biallelic UNC80 missense mutations (Val189Met and Pro1700Ser) were evaluated and showed a small reduction in current amplitudes, indicating a partial loss of NALCN channelosome function (FIG. 11M-N). The Val189Met mutation maps within the hydrophobic core of UNC80 and may be destabilizing, whereas the Pro1700Ser mutation is near an UNC79-NALCN interface that appears important for channelosome function (FIG. 11E), as described below. Additionally, the biallelic UNC80 variant Q341H-P342S recently shown to disrupt NALCN channelosome function¹² is expected to insult and destabilize the C—N interface within the UNC79-UNC80 subcomplex (FIG. 11E). Overall, we conclude that UNC79 and UNC80 form an essential and interdependent subcomplex where disruptive mutations can negatively impact NALCN channelosome assembly, trafficking or function.

### Example 8. NALCN Linker Contacts to the UNC79-UNC80 Subcomplex are Essential

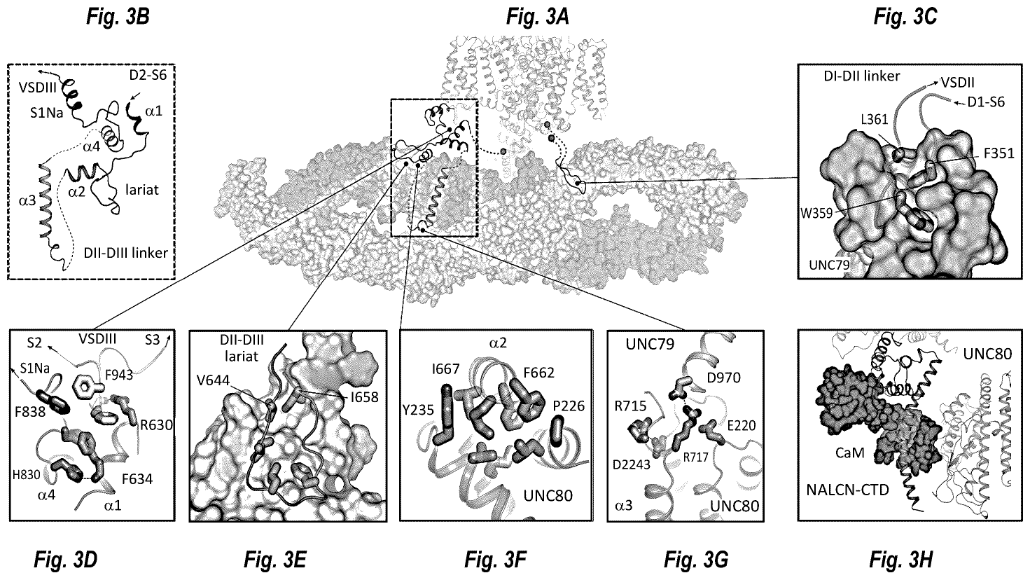

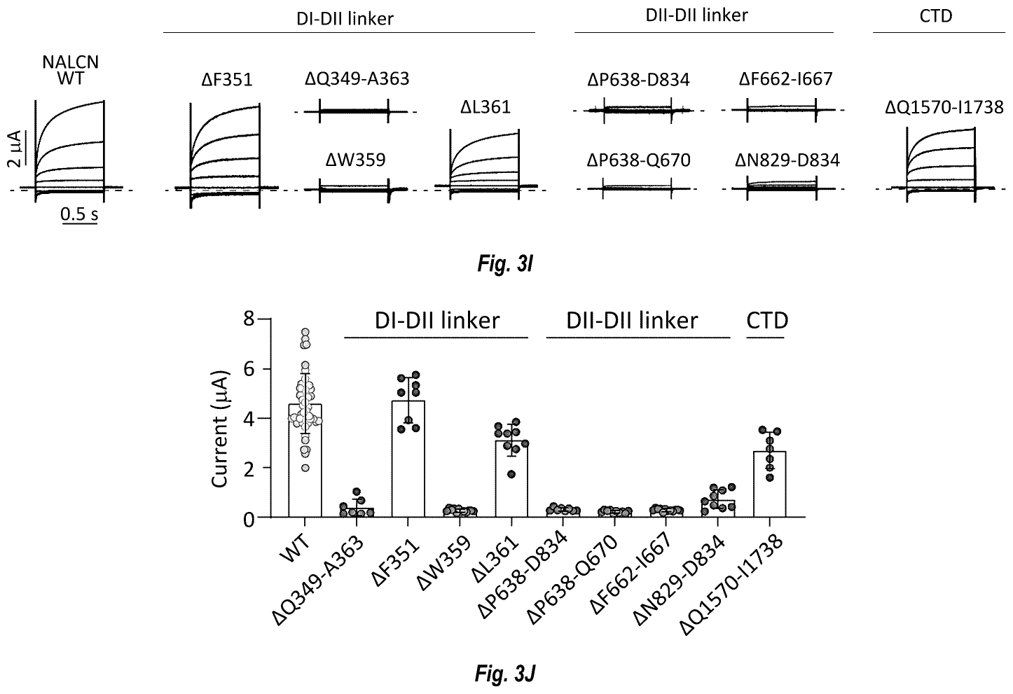

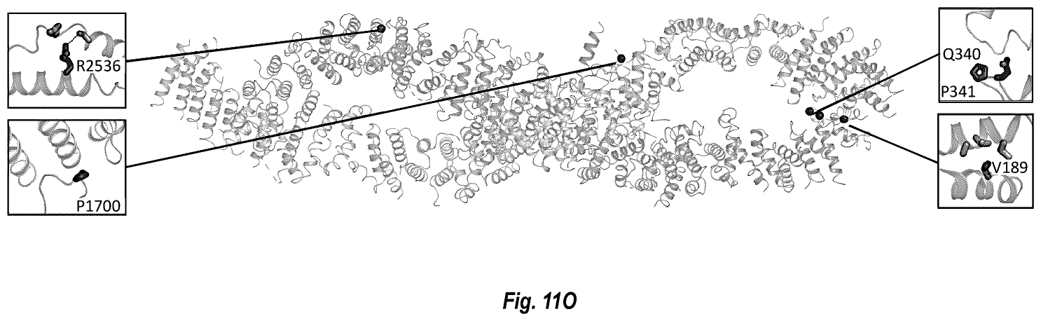

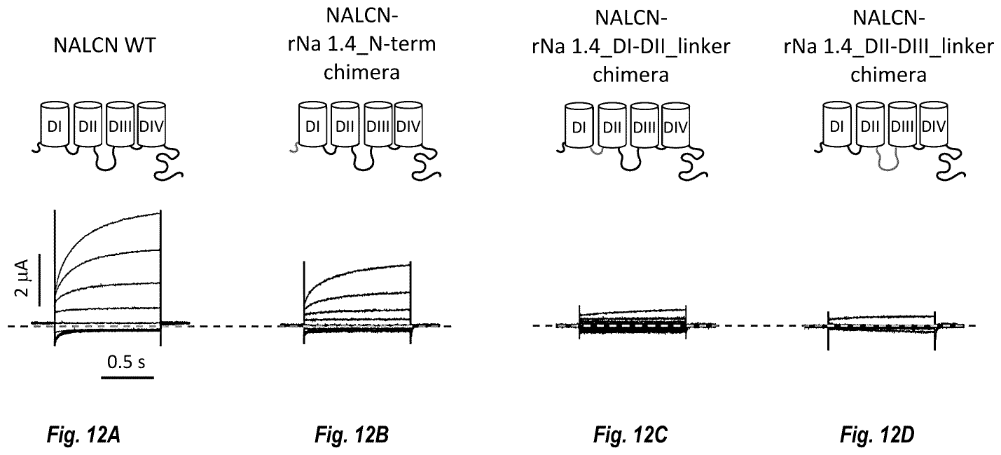

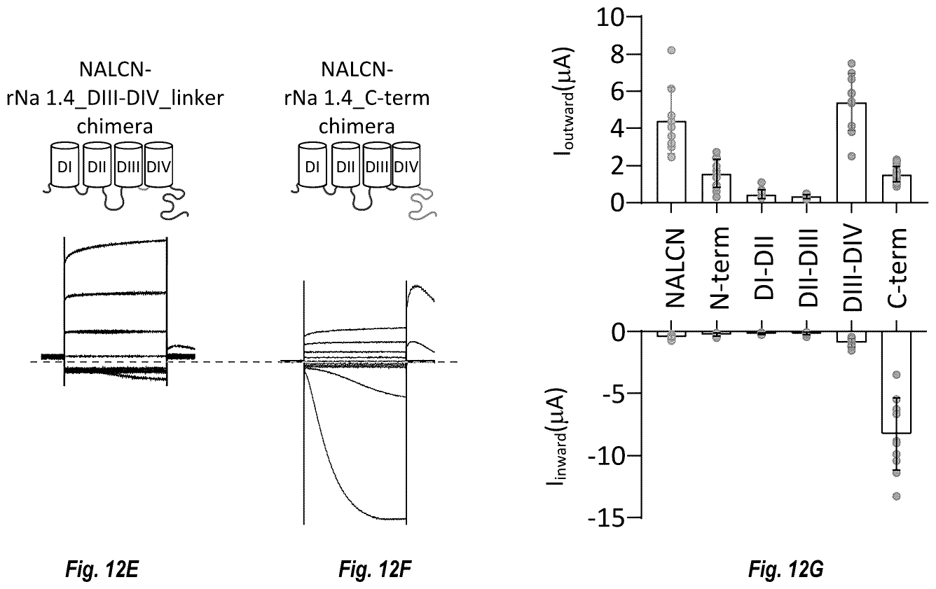

The NALCN subunit employs its intracellular DI-DII and DII-DIII linkers to interact on opposite sides of the UNC79-UNC80 crossover interface, architecturally reminiscent of strings on a marionet puppet (FIG. 3A-C). A small portion of the DI-DII linker engages a membrane proximal surface of UNC79, while two discrete regions from the DII-DIII linker form an elaborated clamp-like interaction on UNC80 (FIG. 3A-G). These securing interactions are unlike those previously seen in any other ion channel-auxiliary subunit complex, and collectively bury >2000 A² of surface area. We systematically substituted the DI-DII, DII-DIII, and DIII-DIV linkers as well as the N- or C-termini of Nav1.4 into NALCN and found an absolute requirement for the DI-DII and DII-DIII linkers to support NALCN channelosome activity (FIG. 12A-G).

The NALCN DI-DII linker is ˜40 residues (Met335-Ala375) with only a resolved hairpin loop that inserts Phe351, Trp359 and Leu361 into a conserved hydrophobic cleft presented in-between HEAT-repeat (HR)-64 and HR-65 of UNC79 (FIGS. 3A, 3C, and 6A). Deletion of this hairpin loop (ΔGln349-Ala363) ablated NALCN channelosome function, whereas single residue deletions (ΔPhe351, ΔTrp359 and ΔLeu361) established a critical role for Trp395 to anchor this key NALCN-linker-UNC79 interface (FIGS. 3I-J, and 13A-B).

The NALCN DII-DIII linker is ˜230 residues (Ser617-Arg844) with resolved portions folded into a complex topology (FIGS. 3A-G and 6B). A hinge module is formed by proximal (α1, Pro628-Arg640) and distal (a4, Met817-Lys841) α-helical containing linker elements that are nucleated around the intracellular S2-S3 loop of VSD3 (FIGS. 3A, 3B, and 3D). A lasso-like loop (Pro638-Arg659) emerges from the hinge module to bury multiple hydrophobic side-chains across HR-24 and HR-25 on UNC80, followed by a short amphipathic α-helix (a2, Glu660-Arg669) that docks within a conserved surface grove on UNC80 parallel to HR-22 (FIGS. 3D and 3F). After a disordered stretch, the DII-DIII linker emerges at Val711 in a crevasse between UNC79 and UNC80, where Arg717 forms the only salt-bridge interaction identified to cross all three subunits (FIG. 3G). Another amphipathic α-helix (α3, Asn718-Ser740) from the DII-DIII linker packs near the inserted Ub-like domain of UNC80, and then following unresolved structure, the linker connects back to the hinge module at the base of VSD3 (FIGS. 3A and 3B). Although some linker manipulations did not completely abolish NALCN channelosome function, targeted disruption of the hinge module (ΔAsn829-Asp834) or deletion of the membrane proximal amphipathic «2-helix ablated activity (FIGS. 3I, 3J, 12C, and 12D), identifying essential structural components of this key NALCN-UNC80 interface.

### Example 9. Calmodulin Binds to the Carboxy-Terminal Domain of NALCN

Our studies unexpectedly identified the C-terminal lobe of CaM bound to a degenerate IQ-like-motif (Ile1572-Arg1573) located on the last resolved α-helix of NALCN (Leu1550-Arg1598) just below the EF-hand-like domain (Ser1463-Gln1549) within the CTD (FIG. 3H). Poorly defined in cryo-EM maps relative to neighboring regions (FIG. 6C-D), the C-terminal lobe of CaM is wedged against the Ub-like domain and HR-20 of UNC80, where CaM binding appears to restrict local contacts between NALCN and UNC80 (FIG. 3H). The N-terminal lobe of CaM and intra-lobe linker nestle directly alongside the EF-hand-like domain of the CTD (FIG. 3H). Targeted mutations or deletions within the CTD region were found to differentially modify NALCN channelosome currents measured in Xenopus oocytes, but none ablated activity (FIGS. 3I-J, 12A-G, and 14A-D). In contrast to the NALCN DI-DII and DII-DIII linkers, neither the CTD or CaM appear strictly essential for NALCN channelosome function, but our results indicate that these do contribute to the current phenotype.

### Example 10. NALCN Channelosome has an Intrinsically Low Open Probability

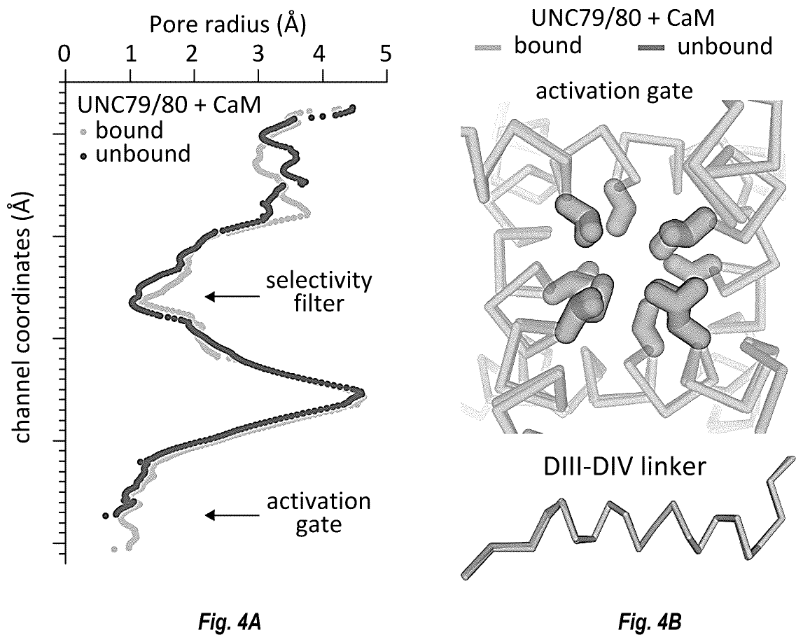

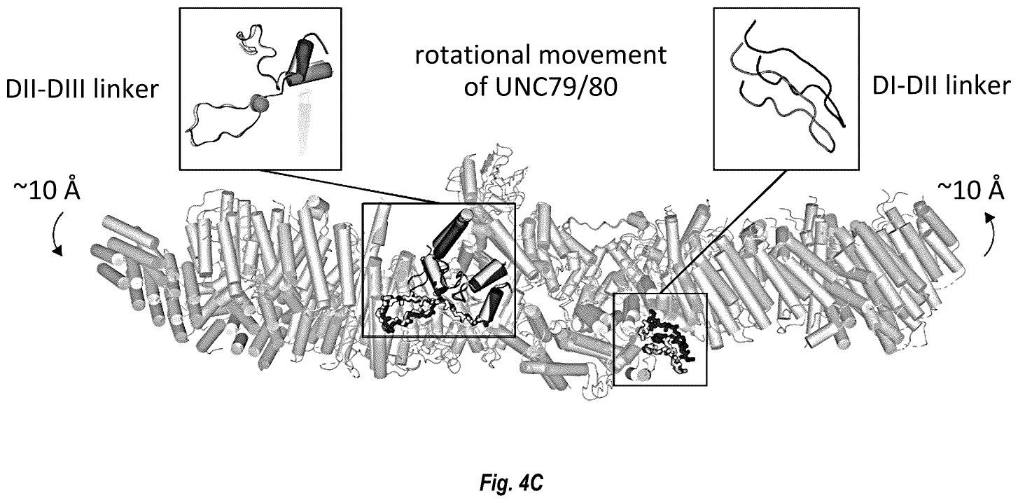

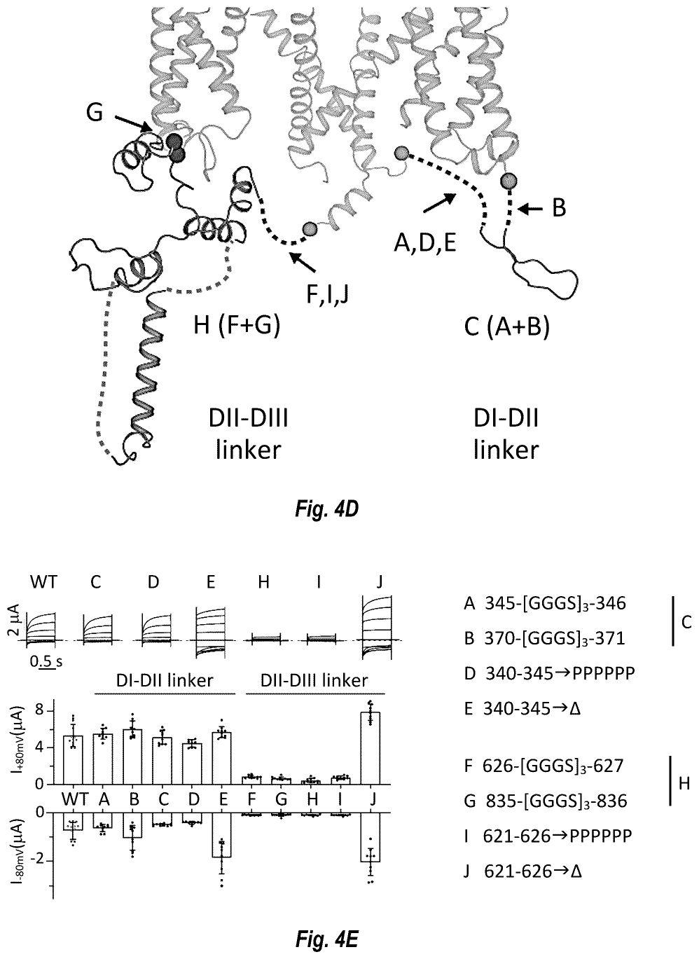

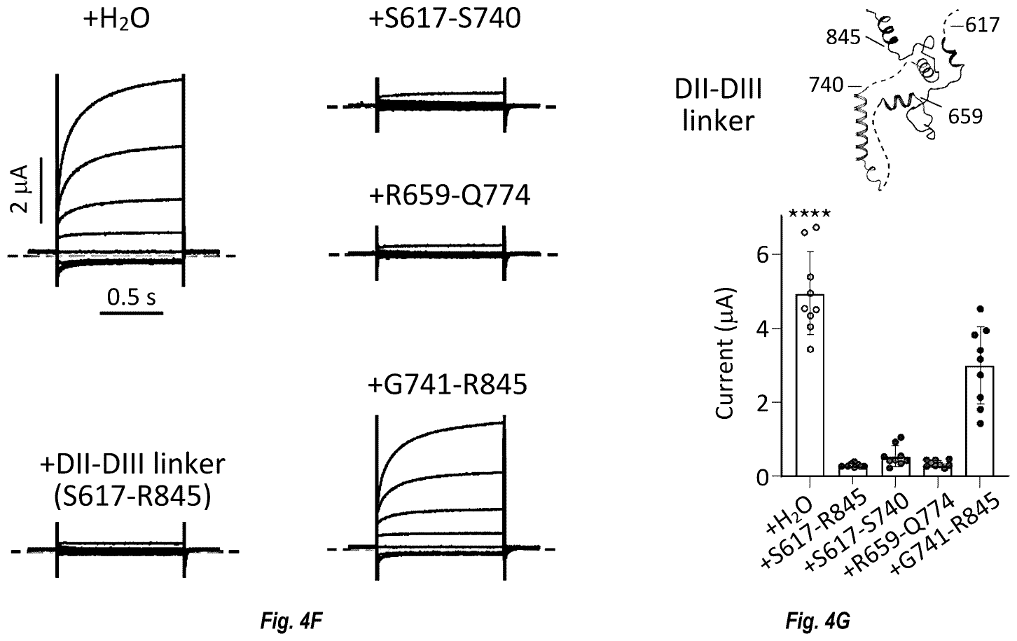

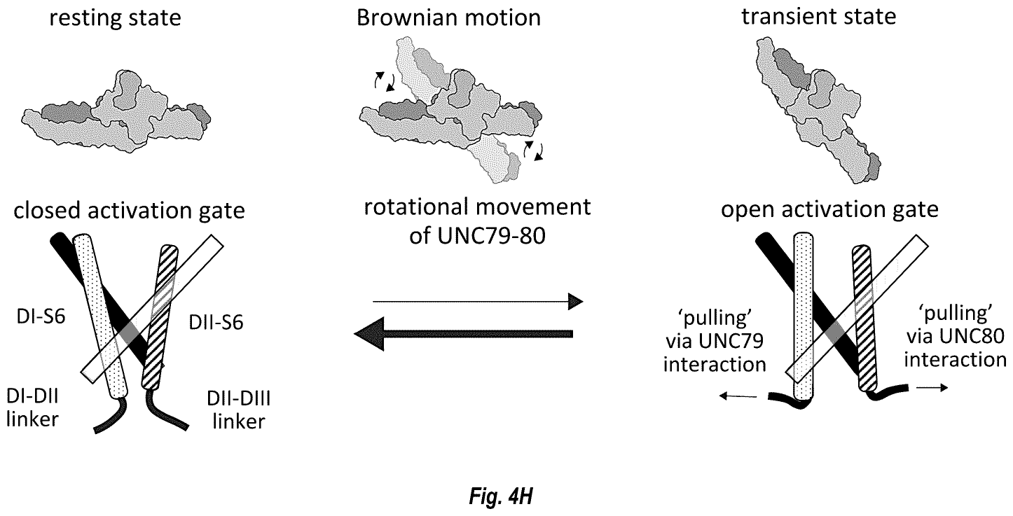

Despite the apparent constitutive activity of the NALCN channelosome (FIG. 1A)¹⁰, the pore structure is unequivocally in a non-conductive state because the S6-gate is narrow and hydrophobically sealed (FIG. 4A-B). In fact, the S6-helices superimpose with a root-mean-square deviation of <0.5 Å relative to the unliganded NALCN-FAM155A subcomplex, revealing nearly identical closed pore structures (FIG. 4B)²⁴⁻²⁶. Although intuitively unexpected, these structural observations may reflect the underlying physiology of the NALCN channelosome.

Cell-attached recordings of the NALCN channelosome transfected in HEK 293 cells using slow-voltage ramps revealed a single-channel conductance of 27.2+/−1.1 pS (mean+/−s.e.m.) with a reversal potential of 5.7+/−1.1 mV (mean+/−s.e.m.) (data not shown). Estimates of single-channel open probability (P_{O}) indicated a uniformly low P_{O} (0.04+/−0.01) across multiple voltages, in which dairy plot analysis revealed that the single-trial average P_{O} switched discretely between epochs of no activity to those with P_{O} of approximately 0.1 (data not shown). Thus, the likelihood of NALCN channelosome openings is low even during periods of high activity, consistent with the observed non-conductive pore structure (FIG. 4A).

We undertook single-channel channelosome recordings with the NALCN Y578S patient mutation, which revealed prolonged channel openings and an increase in P_{O}, with no change in the unitary conductance (data not shown). Diary plot analysis showed that the Y578S NALCN channelosome also switched between epochs of no activity and high activity, in which the P_{O} during the active sweeps was increased compared with wild-type NALCN (data not shown).

### Example 11. Linker Modifications Differentially Impact NALCN Channelosome Gating

Careful evaluation of 3D-classifications from our NALCN channelosome sample did not identify any reconstruction with an open S6 gate. Instead, two distinct classes emerged that revealed a slight lateral rigid-body rotation (˜0.5°) of the intracellular UNC79-UNC80 assembly relative to the NALCN pore-forming subunit, with a nearly stationary DII-DIII linker anchor point. This observation leads to speculation that motions of the UNC79-UNC80 subcomplex relative to NALCN-FAM155A may impact channel gating through physical linkages to the pore.

Having established that the resolved DI-DII linker hairpin loop structure (X349-Ala363) is required for NALCN channelosome activity, we sought to modify its connectivity relative to the DI-S6 or DII VSD2. Insertion of simple twelve-residue sequences preceding or following the hairpin structure (i.e. [Gly-Gly-Gly-Ser]3) (SEQ ID NO: 87) did not impact NALCN channelosome current phenotypes in Xenopus oocytes (FIGS. 4C and 5E-J-). Substitution of six residues in-between the DI-S6 and the hairpin with a polyproline sequence (P340-P345) had little impact, but the deletion of these same residues (ΔX340-X345) produced a marked increase in macroscopic currents (FIGS. 4D-E and 13A-B).

The DII-DIII linker is required for NALCN channelosome activity, so we also sought to modify its connectivity relative to the DII-S6 and DIII VSD3, respectively. All tested [Gly-Gly-Gly-Ser]3 sequence (SEQ ID NO: 87) insertions ablated NALCN channelosome activity (FIGS. 4D-E and 13C-D). Substitution of the six residues in-between the DII-S6 and the hinge module with polyproline sequence (P621-P626) also abolished currents, whereas deletion of these six residues (ΔX621-X626) markedly increased currents (FIGS. 4D-E and 13C-D). Collectively, our results indicate that the DII-DIII linker has a prominent role in relaying putative UNC79-UNC80 movements into NALCN S6-gating, so we expressed constructs of the isolated DII-DIII linker in trans (FIG. 4F-G). A dominant negative impact on channelosome activity was observed in these competition experiments, establishing the DII-DIII linker-UNC80 interface as a key nexus for NALCN channelosome assembly, gating and modulation (FIG. 4F-G).

### Discussion

The NALCN-FAM155A-UNC79-UNC80-CaM channelosome structure has revealed unexpected features with intriguing physiological implications. UNC79 and UNC80 have emerged as highly specialized auxiliary subunits where the unique geometry of the UNC79-UNC80 subcomplex likely underscores early recruitment into the NALCN complex during evolution, rationalizing prior failures to recognize their structural organization as HEAT-repeat proteins. The large interaction surfaces observed between UNC79 and UNC80 in the subcomplex also affirms prior associations and explains why destabilization or truncation of either subunit could result in NALCN channelosome mislocalization, malfunction, or disease (FIGS. 2A-H, 2Q-T, and 11A-O). These insights provide a structural basis for understanding why UNC80 disease mutations phenocopy NALCN-based channelopathies. However, it remains unclear if UNC79 and UNC80 arose by gene duplication, but the integration of a Ub-like fold into UNC80 would presumably suggest this to be an early event. Moreover, the unusual architecture of the UNC79-UNC80 subcomplex raises speculation that it provides a scaffolding function for other proteins reported to impact NALCN activity such as Src kinase or G-proteins, or that it may serve still undescribed cellular roles.

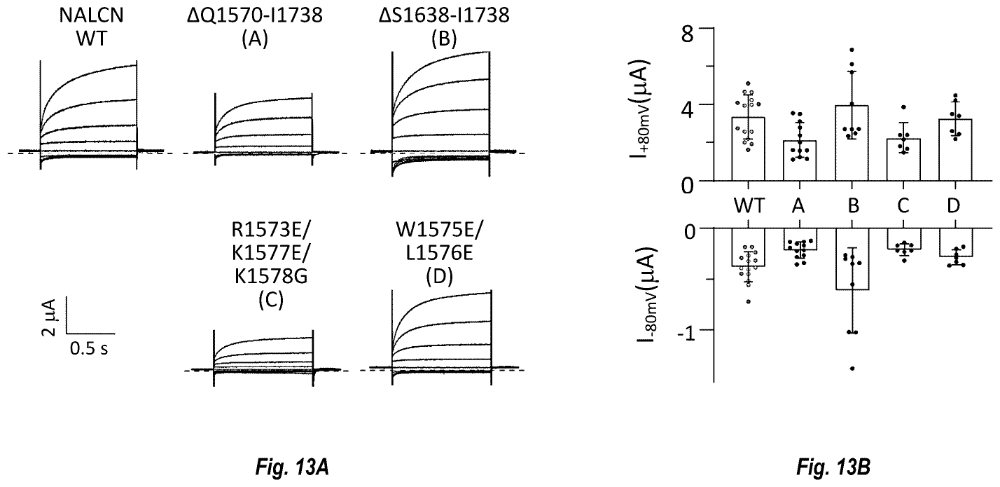

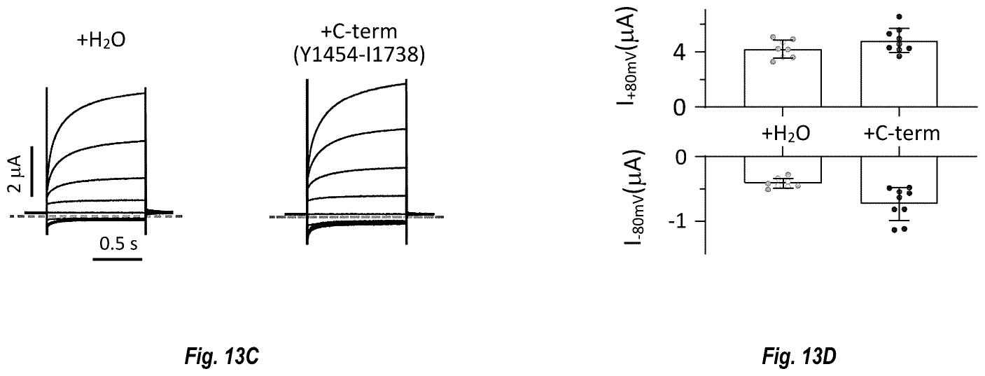

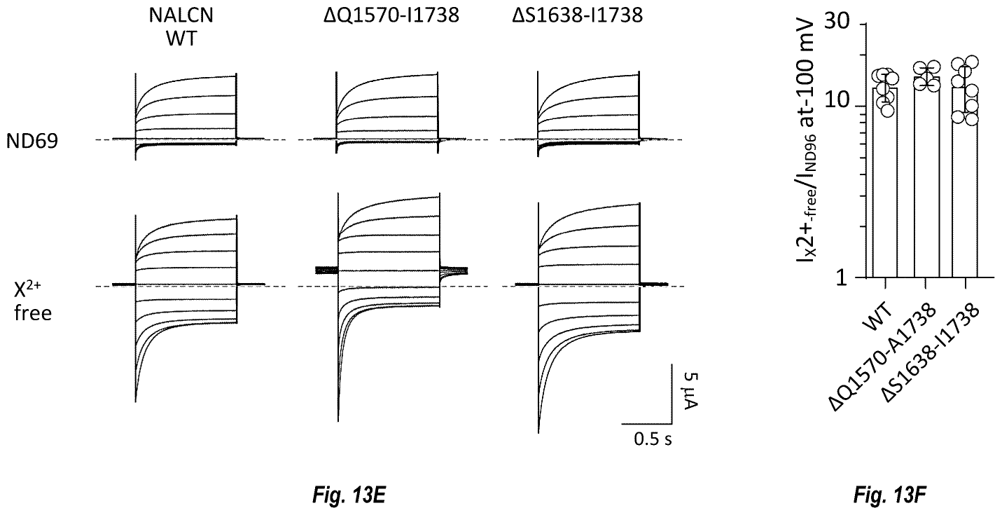

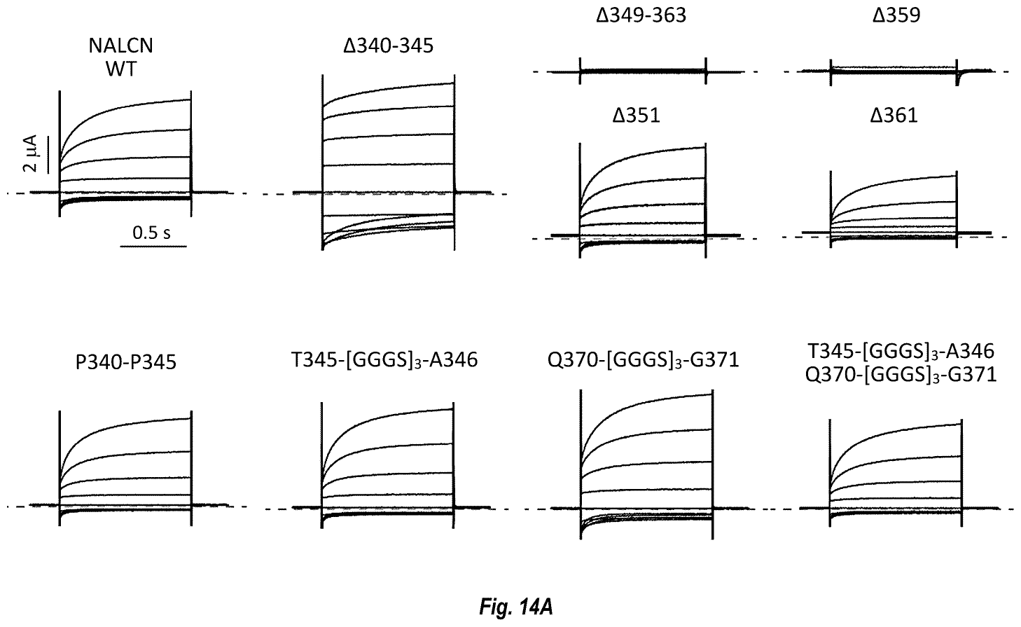

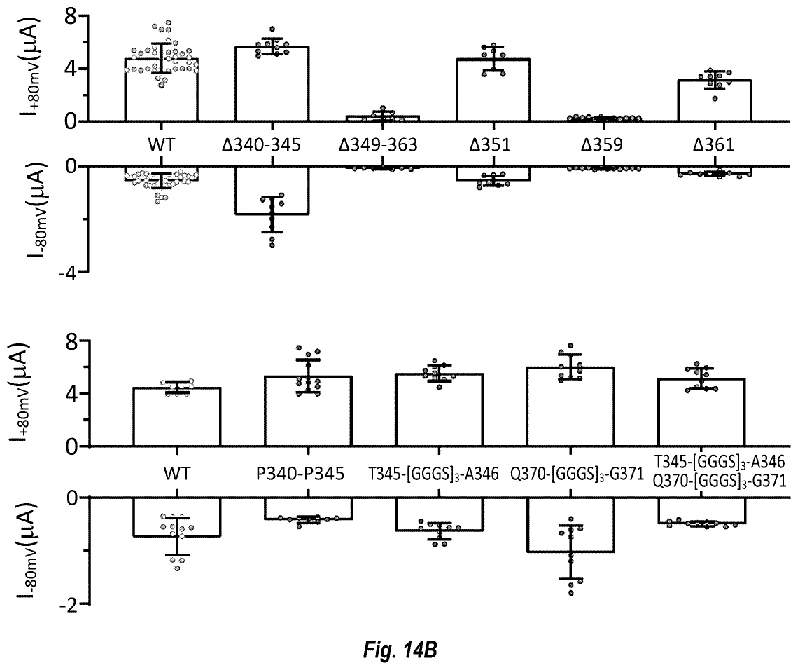

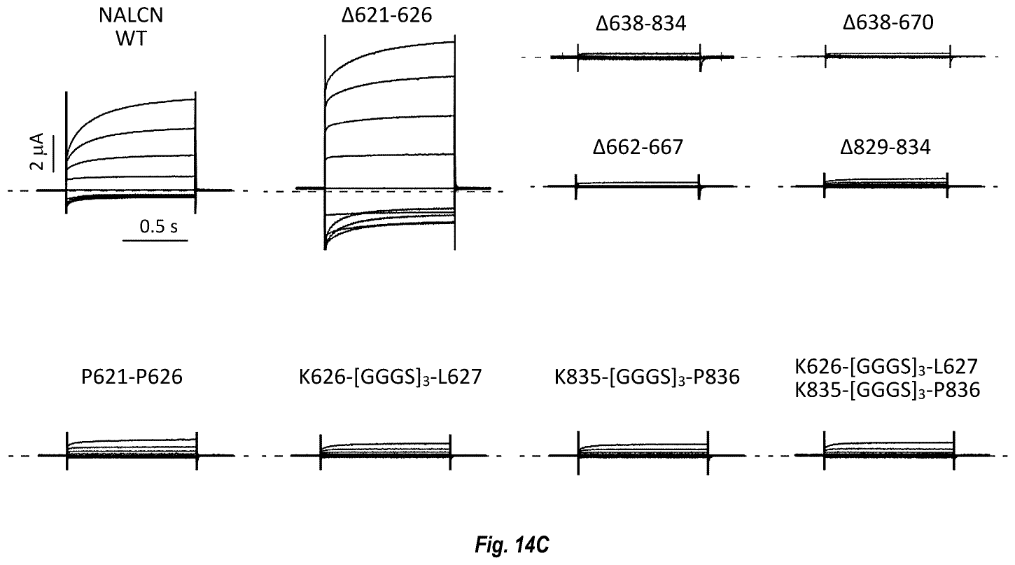

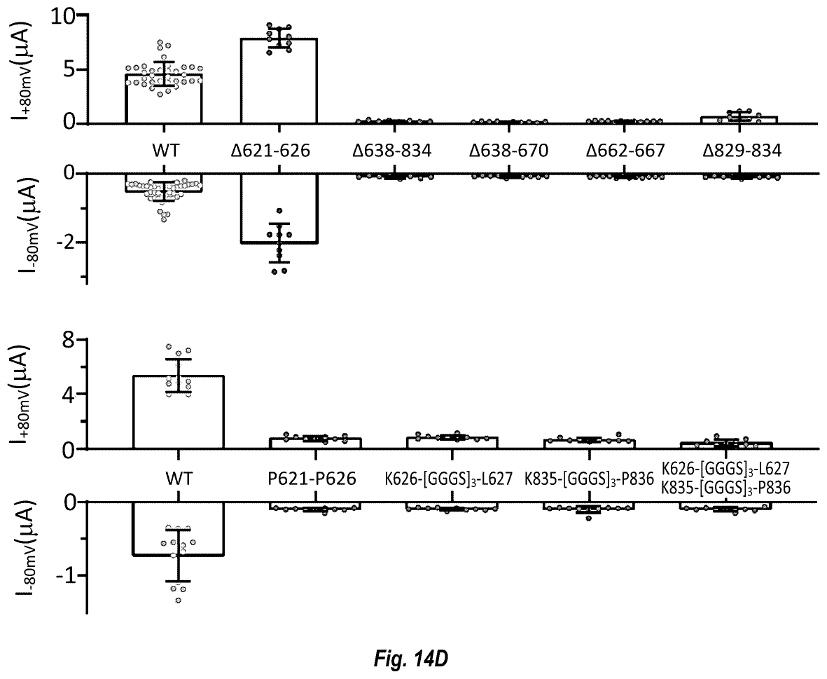

CaM has not been previously implicated in the assembly or function of the NALCN channelosome. In our cryo-EM sample, the C-terminal lobe of CaM binds to an IQ-like motif, suggesting a general mode of CaM association across the NALCN, Nav and Cav channel superfamily (FIG. 3H). CaM binds at a conserved interface between UNC80 and the NALCN pore-forming subunit, but deletion of the IQ-like motif or C-terminal region of NALCN still allows for robust channel activity (FIGS. 3I and 14A-B). In contrast to prior reports, C-terminal deletions of NALCN do not impact modulation by extracellular Ca²⁺ in our experiments (FIG. 13E-F). Our findings do not rule out an important role for the NALCN C-terminus or CaM to modulate gating, but instead point to the potential for cell type specific regulation of the NALCN channelosome.

The NALCN channelosome is constitutively active, but the non-conductive pore structure unequivocally establishes that a hydrophobic S6 region forms the channel gate, even in the presence of FAM155A, UNC79, UNC80 and CaM (FIG. 4A-B). Single channel analyses demonstrated a low P_{O} of approximately 0.4, in which the channelosome enters long sojourns with unmeasurable activity, perhaps indicating a putative long lived closed or inactivated state. These features are consistent with expectations for a major Na⁺ leak conductance, which should have a low basal activity, with the potential for tightly regulated modulation from external or cellular inputs. These findings rationalize the effect of disease-causing missense mutations that cluster around the NALCN pore module, where gain of function mutations such as Y578S may destabilize the closed S6 gate to increase P_{O} and cause detrimental Na influx.

Our studies provide insight into NALCN channelosome assembly and a provisional framework for a pivoting-beam gating model. FAM155A locks onto NALCN from the extracellular side, likely promoting stabilization of the pore-forming subunit^{4,25}. From the intracellular side, the UNC79-UNC80 subcomplex docks onto the essential DI-DII and DII-DIII linkers of NALCN (FIG. 3A-G). The DII-DIII linker forms a relatively rigid contact with UNC80, while the DI-DII linker-UNC79 contact permits a larger range of motion (FIG. 4C), and both contacts serve to orient the UNC79-UNC80 beam-like structure underneath NALCN (FIG. 3A-J). For gating, the DII-DIII linker hinge-module may serve as a pivot point for rigid-body-like motions of the UNC79-UNC80 assembly relative to the NALCN subunit (FIGS. 4C and H). In the non-conductive state, the S6-gate likely remains closed because sufficient tension to the DI-S6 and DII-S6 helices is lacking (FIG. 4H), as the S6-proximal linker regions are disordered in cryo-EM maps (FIG. 5F). A key role for these linker regions is illustrated by their targeted deletion which produces a gain of function (FIG. 4D-E), presumably by increasing strain on the connections between the S6-gate and the UNC79-UNC80 subcomplex. Thus, we propose a pivoting-beam model in which motion of the UNC79-UNC80 subcomplex can eventually produce sufficient tension along the intracellular linkers to unbuckle the DI-S6 and DII-S6 helices to allow the S6-gate to dilate for ion conduction (FIG. 4H). Hydrophobic pore collapse will then presumably promote reestablishment of a non-conductive state until motion of the UNC79-UNC80 assembly can again achieve sufficient tension on the S6-gate (FIG. 4H). Although speculative, this gating model may also begin to account for prior observations that VSD1 and VSD2 can modify NALCN activity^{10,25}, and suggests how the CTD or CaM binding might influence motions of the UNC79-UNC80 beam-like structure relative to the NALCN-FAM155A pore-forming subcomplex to modulate activity.

In summary, the unprecedented architecture of the NALCN channelosome highlights its distinction as an orphan channel in humans and early evolutionary history within the four-domain ion channel superfamily. Our investigation points to a unique gating mechanism, rationalizes the impact of UNC80 and NALCN disease-causing mutations, and identifies sites ripe for selective inhibitor discovery to potentially treat NALCN channelopathies and other electrical disorders.

### REFERENCES

1 Lear, B. C. et al. The ion channel narrow abdomen is critical for neural output of the Drosophila circadian pacemaker. Neuron 48, 965-976, doi: 10.1016/j.neuron.2005.10.030 (2005).
2 Lu, B. et al. The neuronal channel NALCN contributes resting sodium permeability and is required for normal respiratory rhythm. Cell 129, 371-383, doi: 10.1016/j.cell.2007.02.041 (2007).
3 Jospin, M. et al. UNC-80 and the NCA ion channels contribute to endocytosis defects in synaptojanin mutants. Curr Biol 17, 1595-1600, doi: 10.1016/j.cub.2007.08.036 (2007).
4 Xie, L. et al. NLF-1 delivers a sodium leak channel to regulate neuronal excitability and modulate rhythmic locomotion. Neuron 77, 1069-1082, doi: 10.1016/j.neuron.2013.01.018 (2013).
5 Flourakis, M. et al. A Conserved Bicycle Model for Circadian Clock Control of Membrane Excitability. Cell 162, 836-848, doi: 10.1016/j.cell.2015.07.036 (2015).
6 Lutas, A., Lahmann, C., Soumillon, M. & Yellen, G. The leak channel NALCN controls tonic firing and glycolytic sensitivity of substantia nigra pars reticulata neurons. Elife 5, doi: 10.7554/eLife.15271 (2016).
7 Shi, Y. et al. Nalon Is a “Leak” Sodium Channel That Regulates Excitability of Brainstem Chemosensory Neurons and Breathing. J Neurosci 36, 8174-8187, doi: 10.1523/JNEUROSCI.1096-16.2016 (2016).
8 Philippart, F. & Khaliq, Z. M. Gi/o protein-coupled receptors in dopamine neurons inhibit the sodium leak channel NALCN. Elife 7, doi: 10.7554/eLife.40984 (2018).
9 Lu, B. et al. Extracellular calcium controls background current and neuronal excitability via an UNC79-UNC80-NALCN cation channel complex. Neuron 68, 488-499, doi:10.1016/j.neuron.2010.09.014 (2010).
10 Chua, C. H., Wulf, M., Weidling, C., Rasmussen, L. P. & Pless, S. A. The NALCN channel complex is voltage sensitive and directly modulated by extracellular calcium. Sci Adv 6, eaaz3154 (2020).
11 Bouasse, M., Impheng, H., Servant, Z., Lory, P. & Monteil, A. Functional expression of CLIFAHDD and IHPRF pathogenic variants of the NALCN channel in neuronal cells reveals both gain- and loss-of-function properties. Sci Rep 9, 11791, doi: 10.1038/s41598-019-48071-x (2019).
12 Wie, J. et al. Intellectual disability-associated UNC80 mutations reveal inter-subunit interaction and dendritic function of the NALCN channel complex. Nat Commun 11, 3351, doi: 10.1038/s41467-020-17105-8 (2020).
13 Nakayama, M., Iida, M., Koseki, H. & Ohara, O. A gene-targeting approach for functional characterization of KIAA genes encoding extremely large proteins. FASEB J 20, 1718-1720, doi: 10.1096/fj.06-5952fje (2006).
14 Cochet-Bissuel, M., Lory, P. & Monteil, A. The sodium leak channel, NALCN, in health and disease. Front Cell Neurosci 8, 132, doi: 10.3389/fncel.2014.00132 (2014).
15 Bend, E. G. et al. NALCN channelopathies: Distinguishing gain-of-function and loss-of-function mutations. Neurology 87, 1131-1139, doi: 10.1212/WNL.0000000000003095 (2016).
16 Bramswig, N. C. et al. Genetic variants in components of the NALCN-UNC80-UNC79 ion channel complex cause a broad clinical phenotype (NALCN channelopathies). Hum Genet 137, 753-768, doi: 10.1007/s00439-018-1929-5 (2018).
17 Humphrey, J. A. et al. A putative cation channel and its novel regulator: cross-species conservation of effects general on anesthesia. Curr Biol 17, 624-629, doi: 10.1016/j.cub.2007.02.037 (2007).
18 Funato, H. et al. Forward-genetics analysis of sleep in randomly mutagenized mice. Nature 539, 378-383, doi: 10.1038/nature20142 (2016).
19 Eigenbrod, O. et al. Rapid molecular evolution of pain insensitivity in multiple African rodents. Science 364, 852-859, doi: 10.1126/science.aau0236 (2019).
20 Saro, G. et al. Specific Ion Channels Control Sensory Gain, Sensitivity, and Kinetics in a Tonic Thermonociceptor. Cell Rep 30, 397-408 e394, doi: 10.1016/j.celrep.2019.12.029 (2020).
21 Zhang, D. et al. Sodium leak channel contributes to neuronal sensitization in neuropathic pain. Prog Neurobiol 202, 102041, doi:10.1016/j.pneurobio.2021.102041 (2021).
22 Lee, J. H., Cribbs, L. L. & Perez-Reyes, E. Cloning of a novel four repeat protein related to voltage-gated sodium and calcium channels. FEBS Lett 445, 231-236, doi: 10.1016/s0014-5793(99)00082-4 (1999).
23 Stephens, R. F., Guan, W., Zhorov, B. S. & Spafford, J. D. Selectivity filters and cysteine-rich extracellular loops in voltage-gated sodium, calcium, and NALCN channels. Front Physiol 6, 153, doi: 10.3389/fphys.2015.00153 (2015).
24 Kang, Y., Wu, J. X. & Chen, L. Structure of voltage-modulated sodium-selective NALCN-FAM155A channel complex. Nat Commun 11, 6199, doi: 10.1038/s41467-020-20002-9 (2020).
25 Kschonsak, M. et al. Structure of the human sodium leak channel NALCN. Nature 587, 313-318, doi:10.1038/s41586-020-2570-8 (2020).
26 Xie, J. et al. Structure of the human sodium leak channel NALCN in complex with FAM155A. Nat Commun 11, 5831, doi: 10.1038/s41467-020-19667-z (2020).
27 Speca, D. J. et al. Conserved role of unc-79 in ethanol responses in lightweight mutant mice. PLOS Genet 6, doi: 10.1371/journal.pgen. 1001057 (2010).
28 Stray-Pedersen, A. et al. Biallelic Mutations in UNC80 Cause Persistent Hypotonia, Encephalopathy, Growth Retardation, and Severe Intellectual Disability. Am J Hum Genet 98, 202-209, doi:10.1016/j.ajhg.2015.11.004 (2016).
29 Perez, Y. et al. UNC80 mutation causes a syndrome of hypotonia, severe intellectual disability, dyskinesia and dysmorphism, similar to that caused by mutations in its interacting cation channel NALCN. J Med Genet 53, 397-402, doi: 10.1136/jmedgenet-2015-103352 (2016).
30 Yeh, E. et al. A putative cation channel, NCA-1, and a novel protein, UNC-80, transmit neuronal activity in C. elegans. PLOS Biol 6, e55, doi: 10.1371/journal.pbio.0060055 (2008).
31 Lu, B. et al. Peptide neurotransmitters activate a cation channel complex of NALCN and UNC-80. Nature 457, 741-744, doi: 10.1038/nature07579 (2009).
32 Holm, L. Benchmarking fold detection by DaliLite v.5. Bioinformatics 35, 5326-5327, doi:10.1093/bioinformatics/btz536 (2019).
33 Mastronarde, D. N. Automated electron microscope tomography using robust prediction of specimen movements. J Struct Biol 152, 36-51, doi: 10.1016/j.jsb.2005.07.007 (2005).
34 Scheres, S. H. RELION: implementation of a Bayesian approach to cryo-EM structure determination. J Struct Biol 180, 519-530, doi: 10.1016/j.jsb.2012.09.006 (2012).
35 Grant, T., Rohou, A. & Grigorieff, N. cisTEM, user-friendly software for single-particle image processing. Elife 7, doi: 10.7554/eLife.35383 (2018).
36 Zheng, S. Q. et al. MotionCor2: anisotropic correction of beam-induced motion for improved cryo-electron microscopy. Nat Methods 14, 331-332, doi: 10.1038/nmeth.4193 (2017).
37 Rohou, A. & Grigorieff, N. CTFFIND4: Fast and accurate defocus estimation from electron micrographs. J Struct Biol 192, 216-221, doi: 10.1016/j.jsb.2015.08.008 (2015).
38 Tegunov, D. & Cramer, P. Real-time cryo-electron microscopy data preprocessing with Warp. Nat Methods 16, 1146-1152, doi: 10.1038/s41592-019-0580-y (2019).
39 Rosenthal, P. B. & Henderson, R. Optimal determination of particle orientation, absolute hand, and contrast loss in single-particle electron cryomicroscopy. J Mol Biol 333, 721-745, doi:10.1016/j.jmb.2003.07.013 (2003).
40 Cardone, G., Heymann, J. B. & Steven, A. C. One number does not fit all: mapping local variations in resolution in cryo-EM reconstructions. J Struct Biol 184, 226-236, doi:10.1016/j.jsb.2013.08.002 (2013).
41 Afonine, P. V. et al. Real-space refinement in PHENIX for cryo-EM and crystallography. Acta Crystallogr D Struct Biol 74, 531-544, doi: 10.1107/S2059798318006551 (2018).
42 Emsley, P., Lohkamp, B., Scott, W. G. & Cowtan, K. Features and development of Coot. Acta Crystallogr D Biol Crystallogr 66, 486-501, doi: 10.1107/S0907444910007493 (2010).
43 Goddard, T. D. et al. UCSF ChimeraX: Meeting modern challenges in visualization and analysis. Protein Sci 27, 14-25, doi: 10.1002/pro.3235 (2018).
44 Croll, T. I. ISOLDE: a physically realistic environment for model building into low-resolution electron-density maps. Acta Crystallogr D Struct Biol 74, 519-530, doi: 10.1107/S2059798318002425 (2018).
45 Afonine, P. V. et al. New tools for the analysis and validation of cryo-EM maps and atomic models. Acta Crystallogr D Struct Biol 74, 814-840, doi: 10.1107/S2059798318009324 (2018).
46 Williams, C. J. et al. MolProbity: More and better reference data for improved all-atom structure validation. Protein Sci 27, 293-315, doi: 10.1002/pro.3330 (2018).
47 Pettersen, E. F. et al. UCSF Chimera—a visualization system for exploratory research and analysis. J Comput Chem 25, 1605-1612, doi: 10.1002/jcc.20084 (2004).
48 Sievers, F. et al. Fast, scalable generation of high-quality protein multiple sequence alignments using Clustal Omega. Mol Syst Biol 7, 539, doi: 10.1038/msb.2011.75 (2011).
49 Waterhouse, A. M., Procter, J. B., Martin, D. M., Clamp, M. & Barton, G. J. Jalview Version 2—a multiple sequence alignment editor and analysis workbench. Bioinformatics 25, 1189-1191, doi:10.1093/bioinformatics/btp033 (2009).
50 Robert, X. & Gouet, P. Deciphering key features in protein structures with the new ENDscript server. Nucleic Acids Res 42, W320-324, doi: 10.1093/nar/gku316 (2014).

Table 4 below provides a listing of certain sequences referenced herein. Note that, in the case of certain peptide segments, such as NALCN DI-DII and NALCN DII-DIII linker segments (e.g., SEQ ID Nos: 18-30), in constructing an open reading frame for expression in vivo in a host cell, one may add an N-terminal methionine to the sequence of the table below. In other cases, where a peptide can be synthesized in vitro, an N-terminal methionine may not be necessary. For this reason, it is not included in the NALCN linker peptide sequences of the table below. Certain protein fragment sequences listed in the table below are listed including an N-terminal methionine prior to the native portion of the sequence (e.g., SEQ ID Nos: 56 and 58). If the methionine is not needed to make the fragment, it can be excluded.

TABLE 4
Description of certain sequences.

| Seq Id | | |
|---|---|---|
| No | Description | Sequence |
| 1 | Human NALCN | MLKRKQSSREAQPVTDFGPDESLSDNADILWINKPWVHS |
| | UniProt Q8IZF0 | LLRICAIISVISVCMNTPMTFEHYPPLQYVTFTLDTLLMFLY |
| | | TAEMIAKMHIRGIVKGDSSYVKDRWCVFDGFMVFCLWVS |
| | | LVLQVFEIADIVDQMSPWGMLRIPRPLIMIRAFRIYFRFELP |
| | | RTRITNILKRSGEQIWSVSIFLLFFLLLYGILGVQMFGTFTY |
| | | HCVVNDTKPGNVTWNSLAIPDTHCSPELEEGYQCPPGFKC |
| | | MDLEDLGLSRQELGYSGFNEIGTSIFTVYEAASQEGWVFL |
| | | MYRAIDSFPRWRSYFYFITLIFFLAWLVKNVFIAVIIETFAEI |
| | | RVQFQQMWGSRSSTTSTATTQMFHEDAAGGWQLVAVDV |
| | | NKPQGRAPACLQKMMRSSVFHMFILSMVTVDVIVAASNY |
| | | YKGENFRRQYDEFYLAEVAFTVLFDLEALLKIWCLGFTGY |
| | | ISSSLHKFELLLVIGTTLHVYPDLYHSQFTYFQVLRVVRLIK |
| | | ISPALEDFVYKIFGPGKKLGSLVVFTASLLIVMSAISLQMFC |
| | | FVEELDRFTTFPRAFMSMFQILTQEGWVDVMDQTLNAVG |
| | | HMWAPVVAIYFILYHLFATLILLSLFVAVILDNLELDEDLK |
| | | KLKQLKQSEANADTKEKLPLRLRIFEKFPNRPQMVKISKLP |
| | | SDFTVPKIRESFMKQFIDRQQQDTCCLLRSLPTTSSSSCDHS |
| | | KRSAIEDNKYIDQKLRKSVFSIRARNLLEKETAVTKILRAC |
| | | TRQRMLSGSFEGQPAKERSILSVQHHIRQERRSLRHGSNSQ |
| | | RISRGKSLETLTQDHSNTVRYRNAQREDSEIKMIQEKKEQA |
| | | EMKRKVQEEELRENHPYFDKPLFIVGREHRFRNFCRVVVR |
| | | ARFNASKTDPVTGAVKNTKYHQLYDLLGLVTYLDWVMII |
| | | VTICSCISMMFESPFRRVMHAPTLQIAEYVFVIFMSIELNLKI |
| | | MADGLFFTPTAVIRDFGGVMDIFIYLVSLIFLCWMPQNVPA |
| | | ESGAQLLMVLRCLRPLRIFKLVPQMRKVVRELFSGFKEIFL |
| | | VSILLLTLMLVFASFGVQLFAGKLAKCNDPNIIRREDCNGIF |
| | | RINVSVSKNLNLKLRPGEKKPGFWVPRVWANPRNFNFDN |
| | | VGNAMLALFEVLSLKGWVEVRDVIIHRVGPIHGIYIHVFVF |
| | | LGCMIGLTLFVGVVIANFNENKGTALLTVDQRRWEDLKSR |
| | | LKIAQPLHLPPRPDNDGFRAKMYDITQHPFFKRTIALLVLA |
| | | QSVLLSVKWDVEDPVTVPLATMSVVFTFIFVLEVTMKIIA |
| | | MSPAGFWQSRRNRYDLLVTSLGVVWVVLHFALLNAYTY |
| | | MMGACVIVFRFFSICGKHVTLKMLLLTVVVSMYKSFFIIVG |
| | | MFLLLLCYAFAGVVLFGTVKYGENINRHANFSSAGKAITV |
| | | LFRIVTGEDWNKIMHDCMVQPPFCTPDEFTYWATDCGNY |
| | | AGALMYFCSFYVIIAYIMLNLLVAIIVENFSLFYSTEEDQLL |
| | | SYNDLRHFQIIWNMVDDKREGVIPTFRVKFLLRLLRGRLE |
| | | VDLDKDKLLFKHMCYEMERLHNGGDVTFHDVLSMLSYR |
| | | SVDIRKSLQLEELLAREQLEYTIEEEVAKQTIRMWLKKCLK |
| | | RIRAKQQQSCSIIHSLRESQQQELSRFLNPPSIETTQPSEDTN |
| | | ANSQDNSMQPETSSQQQLLSPTLSDRGGSRQDAADAGKPQ |
| | | RKFGQWRLPSAPKPISHSVSSVNLRFGGRTTMKSVVCKMN |
| | | PMTDAASCGSEVKKWWTRQLTVESDESGDDLLDI |
| 2 | Human FAM155 | MTRGAWMCRQYDDGLKIWLAAPRENEKPFIDSERAQKW |
| | UniProt B1AL88 | RLSLASLLFFTVLLSDHLWFCAEAKLTRARDKEHQQQQRQ |
| | | QQQQQQQQRQRQQQQQQRRQQEPSWPALLASMGESSPA |
| | | AQAHRLLSASSSPTLPPSPGDGGGGGGKGNRGKDDRGKA |
| | | LFLGNSAKPVWRLETCYPQGASSGQCFTVENADAVCARN |
| | | WSRGAAGGDGQEVRSKHPTPLWNLSDFYLSFCNSYTLWE |
| | | LFSGLSSPNTLNCSLDVVLKEGGEMTTCRQCVEAYQDYDH |
| | | HAQEKYEEFESVLHKYLQSEEYSVKSCPEDCKIVYKAWLC |
| | | SQYFEVTQFNCRKTIPCKQYCLEVQTRCPFILPDNDEVIYG |
| | | GLSSFICTGLYETFLTNDEPECCDVRREEKSNNPSKGTVEK |
| | | SGSCHRTSLTVSSATRLCNSRLKLCVLVLILLHTVLTASAA |
| | | QNTAGLSFGGINTLEENSTNEE |
| 3 | Human UNC79 | MSTKAEQFASKIRYLQEYHNRVLHNIYPVPSGTDIANTLKY |
| | UniProt Q9P2D8 | FSQTLLSILSRTGKKENQDASNLTVPMTMCLFPVPFPLTPSL |
| | | RPQVSSINPTVTRSLLYSVLRDAPSERGPQSRDAQLSDYPSL |
| | | DYQGLYVTLVTLLDLVPLLQHGQHDLGQSIFYTTTCLLPFL |
| | | NDDILSTLPYTMISTLATFPPFLHKDIIEYLSTSFLPMAILGSS |
| | | RREGVPAHVNLSASSMLMIAMQYTSNPVYHCQLLECLMK |
| | | YKQEVWKDLLYVIAYGPSQVKPPAVQMLFHYWPNLKPPG |
| | | AISEYRGLQYTAWNPIHCQHIECHNAINKPAVKMCIDPSLS |
| | | VALGDKPPPLYLCEECSERIAGDHSEWLIDVLLPQAEISAIC |
| | | QKKNCSSHVRRAVVTCFSAGCCGRHGNRPVRYCKRCHSN |
| | | HHSNEVGAAAETHLYQTSPPPINTRECGAEELVCAVEAVIS |
| | | LLKEAEFHAEQREHELNRRRQLGLSSSHHSLDNADFDNKD |
| | | DDKHDQRLLSQFGIWFLVSLCTPSENTPTESLARLVAMVF |
| | | QWFHSTAYMMDDEVGSLVEKLKPQFVTKWLKTVCDVRF |
| | | DVMVMCLLPKPMEFARVGGYWDKSCSTVTQLKEGLNRIL |
| | | CLIPYNVINQSVWECIMPEWLEAIRTEVPDNQLKEFREVLS |
| | | KMFDIELCPLPFSMEEMFGFISCRFTGYPSSVQEQALLWLH |
| | | VLSELDIMVPLQLLISMFSDGVNSVKELANQRKSRVSELAG |
| | | NLASRRVSVASDPGRRVQHNMLSPFHSPFQSPFRSPLRSPF |
| | | RSPFKNFGHPGGRTIDFDCEDDEMNLNCFILMFDLLLKQM |
| | | ELQDDGITMGLEHSLSKDIISIINNVFQAPWGGSHTCQKDE |
| | | KAIECNLCQSSILCYQLACELLERLAPKEESRLVEPTDSLED |
| | | SLLSSRPEFIIGPEGEEEENPASKHGENPGNCTEPVEHAAVK |
| | | NDTERKFCYQQLPVTLRLIYTIFQEMAKFEEPDILENMLNC |
| | | LKILCLHGECLYIARKDHPQFLAYIQDHMLIASLWRVVKSE |
| | | FSQLSSLAVPLLLHALSLPHGADIFWTIINGNFNSKDWKMR |
| | | FEAVEKVAVICRFLDIHSVTKNHLLKYSLAHAFCCFLTAVE |
| | | DVNPAVATRAGLLLDTIKRPALQGLCLCLDFQFDTVVKDR |
| | | PTILSKLLLLHFLKQDIPALSWEFFVNRFETLSLEAQLHLDC |
| | | NKEFPFPTTITAVRTNVANLSDAALWKIKRARFARNRQKS |
| | | VRSLRDSVKGPVESKRALSLPETLTSKIRQQSPENDNTIKDL |
| | | LPEDAGIDHQTVHQLITVLMKFMAKDESSAESDISSAKAFN |
| | | TVKRHLYVLLGYDQQEGCFMIAPQKMRLSTCFNAFIAGIA |
| | | QVMDYNINLGKHLLPLVVQVLKYCSCPQLRHYFQQPPRCS |
| | | LWSLKPHIRQMWLKALLVILYKYPYRDCDISKILLHLIHIT |
| | | VNTLNAQYHSCKPHATAGPLYSDNSNISRYSEKEKGEIELA |
| | | EYRETGALQDSLLHCVREESIPKKKLRSFKQKSLDIGNADS |
| | | LLFTLDEHRRKSCIDRCDIEKPPTQAAYIAQRPNDPGRSRQ |
| | | NSATRPDNSEIPENPAMEGFPDARRPVIPEVRLNCMETFEV |
| | | KVDSPVKPAPKEDLDLIDLSSDSTSGPEKHSILSTSDSDSLV |
| | | FEPLPPLRIVESDEEEETMNQGDDGPSGKNAASSPSVPSHPS |
| | | VLSLSTAPLVQVSVEDCSKDFSSKDSGNNQSAGNTDSALIT |
| | | LEDPMDAEGSSKPEELPEFSCGSPLTLKQKRDLLQKSFALP |
| | | EMSLDDHPDPGTEGEKPGELMPSSGAKTVLLKVPEDAENP |
| | | TESEKPDTSAESDTEQNPERKVEEDGAEESEFKIQIVPRQRK |
| | | QRKIAVSAIQREYLDISFNILDKLGEQKDPDPSTKGLSTLEM |
| | | PRESSSAPTLDAGVPETSSHSSISTQYRQMKRGSLGVLTMS |
| | | QLMKRQLEHQSSAPHNISNWDTEQIQPGKRQCNVPTCLNP |
| | | DLEGQPLRMRGATKSSLLSAPSIVSMFVPAPEEFTDEQPTV |
| | | MTDKCHDCGAILEEYDEETLGLAIVVLSTFIHLSPDLAAPL |
| | | LLDIMQSVGRLASSTTFSNQAESMMVPGNAAGVAKQFLR |
| | | CIFHQLAPNGIFPQLFQSTIKDGTFLRTLASSLMDFNELSSIA |
| | | ALSQLLEGLNNKKNLPAGGAMIRCLENIATFMEALPMDSP |
| | | SSLWTTISNQFQTFFAKLPCVLPLKCSLDSSLRIMICLLKIPS |
| | | TNATRSLLEPFSKLLSFVIQNAVFTLAYLVELCGLCYRAFT |
| | | KERDKFYLSRSVVLELLQALKLKSPLPDTNLLLLVQFICAD |
| | | AGTKLAESTILSKQMIASVPGCGTAAMECVRQYINEVLDF |
| | | MADMHTLTKLKSHMKTCSQPLHEDTFGGHLKVGLAQIAA |
| | | MDISRGNHRDNKAVIRYLPWLYHPPSAMQQGPKEFIECVS |
| | | HIRLLSWLLLGSLTHNAVCPNASSPCLPIPLDAGSHVADHLI |
| | | VILIGFPEQSKTSVLHMCSLFHAFIFAQLWTVYCEQSAVAT |
| | | NLQNQNEFSFTAILTALEFWSRVTPSILQLMAHNKVMVEM |
| | | VCLHVISLMEALQECNSTIFVKLIPMWLPMIQSNIKHLSAG |
| | | LQLRLQAIQNHVNHHSLRTLPGSGQSSAGLAALRKWLQCT |
| | | QFKMAQVEIQSSEAASQFYPL |
| 4 | Human UNC80 | MVKRKSSEGQEQDGGRGIPLPIQTFLWRQTSAFLRPKLGK |
| | Uniprot Q8N2C7 | QYEASCVSFERVLVENKLHGLSPALSEAIQSISRWELVQAA |
| | | LPHVLHCTATLLSNRNKLGHQDKLGVAETKLLHTLHWML |
| | | LEAPQDCNNERFGGTDRGSSWGGSSSAFIHQVENQGSPGQ |
| | | PCQSSSNDEEENNRRKIFQNSMATVELFVFLFAPLVHRIKE |
| | | SDLTFRLASGLVIWQPMWEHRQPGVSGFTALVKPIRNIITA |
| | | KRSSPINSQSRTCESPNQDARHLEGLQVVCETFQSDSISPKA |
| | | TISGCHRGNSFDGSLSSQTSQERGPSHSRASLVIPPCQRSRY |
| | | ATYFDVAVLRCLLQPHWSEEGTQWSLMYYLQRLRHMLEE |
| | | KPEKPPEPDIPLLPRPRSSSMVAAAPSLVNTHKTQDLTMKC |
| | | NEEEKSLSSEAFSKVSLTNLRRSAVPDLSSDLGMNIFKKFK |
| | | SRKEDRERKGSIPFHHTGKRRPRRMGVPFLLHEDHLDVSPT |
| | | RSTFSFGSFSGLGEDRRGIEKGGWQTTILGKLTRRGSSDAA |
| | | TEMESLSARHSHSHHTLVSDLPDPSNSHGENTVKEVRSQIS |
| | | TITVATFNTTLASFNVGYADFFNEHMRKLCNQVPIPEMPHE |
| | | PLACANLPRSLTDSCINYSYLEDTEHIDGTNNFVHKNGML |
| | | DLSVVLKAVYLVLNHDISSRICDVALNIVECLLQLGVVPCV |
| | | EKNRKKSENKENETLEKRPSEGAFQFKGVSGSSTCGFGGP |
| | | AVSGAGDGGGEEGGGGDGGGGGGDGGGGGGGGGGPYE |
| | | KNDKNQEKDESTPVSNHRLALTMLIKIVKSLGCAYGCGEG |
| | | HRGLSGDRLRHQVFRENAQNCLTKLYKLDKMQFRQTMR |
| | | DYVNKDSLNNVVDFLHALLGFCMEPVTDNKAGFGNNFTT |
| | | VDNKSTAQNVEGIIVSAMFKSLITRCASTTHELHSPENLGL |
| | | YCDIRQLVQFIKEAHGNVFRRVALSALLDSAEKLAPGKKV |
| | | EENEQESKPAGSKRSEAGSIVDKGQVSSAPEECRSFMSGRP |
| | | SQTPEHDEQMQGANLGRKDFWRKMFKSQSAASDTSSQSE |
| | | QDTSECTTAHSGTTSDRRARSRSRRISLRKKLKLPIGKRNW |
| | | LKRSSLSGLADGVEDLLDISSVDRLSFIRQSSKVKFTSAVKL |
| | | SEGGPGSGMENGRDEEENFFKRLGCHSFDDHLSPNQDGGK |
| | | SKNVVNLGAIRQGMKRFQFLLNCCEPGTIPDASILAAALDL |
| | | EAPVVARAALFLECARFVHRCNRGNWPEWMKGHHVNIT |
| | | KKGLSRGRSPIVGNKRNQKLQWNAAKLFYQWGDAIGVRL |
| | | NELCHGESESPANLLGLIYDEETKRRLRKEDEEEDFLDDST |
| | | VNPSKCGCPFALKMAACQLLLEITTFLRETFSCLPRPRTEPL |
| | | VDLESCRLRLDPELDRHRYERKISFAGVLDENEDSKDSLHS |
| | | SSHTLKSDAGVEEKKEGSPWSASEPSIEPEGMSNAGAEEN |
| | | YHRNMSWLHVMILLCNQQSFICTHVDYCHPHCYLHHSRS |
| | | CARLVRAIKLLYGDSVDSLRESSNISSVALRGKKQKECSDK |
| | | SCLRTPSLKKRVSDANLEGKKDSGMLKYIRLQVMSLSPAP |
| | | LSLLIKAAPILTEEMYGDIQPAAWELLLSMDEHMAGAAAA |
| | | MFLLCAVKVPEAVSDMLMSEFHHPETVQRLNAVLKFHTL |
| | | WRFRYQVWPRMEEGAQQIFKIPPPSINFTLPSPVLGMPSVP |
| | | MFDPPWVPQCSGSVQDPINEDQSKSFSARAVSRSHQRAEHI |
| | | LKNLQQEEEKKRLGREASLITAIPITQEACYEPTCTPNSEPE |
| | | EEVEEVTNLASRRLSVSPSCTSSTSHRNYSFRRGSVWSVRS |
| | | AVSAEDEEHTTEHTPNHHVPQPPQAVFPACICAAVLPIVHL |
| | | MEDGEVREDGVAVSAVAQQVLWNCLIEDPSTVLRHFLEK |
| | | LTISNRQDELMYMLRKLLLNIGDFPAQTSHILFNYLVGLIM |
| | | YFVRTPCEWGMDAISATLTFLWEVVGYVEGLFFKDLKQT |
| | | MKKEQCEVKLLVTASMPGTKTLVVHGQNECDIPTQLPVH |
| | | EDTQFEALLKECLEFFNIPESQSTHYFLMDKRWNLIHYNKT |
| | | YVRDIYPFRRSVSPQLNLVHMHPEKGQELIQKQVFTRKLEE |
| | | VGRVLFLISLTQKIPTAHKQSHVSMLQEDLLRLPSFPRSAID |
| | | AEFSLFSDPQAGKELFGLDTLQKSLWIQLLEEMFLGMPSEF |
| | | PWGDEIMLFLNVFNGALILHPEDSALLRQYAATVINTAVH |
| | | FNHLFSLSGYQWILPTMLQVYSDYESNPQLRQAIEFACHQF |
| | | YILHRKPFVLQLFASVAPLLEFPDAANNGPSKGVSAQCLFD |
| | | LLQSLEGETTDILDILELVKAEKPLKSLDFCYGNEDLTFSIS |
| | | EAIKLCVTVVAYAPESFRSLQMLMVLEALVPCYLQKLKRQ |
| | | TSQVETVPAAREEIAATAALATSLQALLYSVEVLTRPMTAP |
| | | QMSRCDQGHKGTTTANHTMSSGVNTRYQEQGAKLHFIRE |
| | | NLHLLEEGQGIPREELDERIAREEFRRPRESLLNICTEFYKH |
| | | CGPRLKILQNLAGEPRVIALELLDVKSHMRLAEIAHSLLKL |
| | | APYDTQTMESRGLRRYIMEMLPITDWTAEAVRPALILILKR |
| | | LDRMFNKIHKMPTLRRQVEWEPASNLIEGVCLTLQRQPIIS |
| | | FLPHLRSLINVCVNLVMGVVGPSSVADGLPLLHLSPYLSPP |
| | | LPFSTAVVRLVALQIQALKEDFPLSHVISPFTNQERREGML |
| | | LNLLIPFVLTVGSGSKDSPWLEQPEVQLLLQTVINVLLPPRII |
| | | STSRSKNFMLESSPAHCSTPGDAGKDLRREGLAESTSQAA |
| | | YLALKVILVCFERQLGSQWYWLSLQVKEMALRKVGGLAL |
| | | WDFLDFIVRTRIPIFVLLRPFIQCKLLAQPAENHEELSARQHI |
| | | ADQLERRFIPRPLCKSSLIAEFNSELKILKEAVHSGSAYQGK |
| | | TSISTVGTSTSAYRLSLATMSRSNTGTGTVWEQDSEPSQQA |
| | | SQDTLSRTDEEDEENDSISMPSVVSEQEAYLLSAIGRRRFSS |
| | | HVSSMSVPQAEVGMLPSQSEPNVLDDSQGLAAEGSLSRVA |
| | | SIQSEPGQQNLLVQQPLGRKRGLRQLRRPLLSRQKTQTEPR |
| | | NRQGARLSTTRRSIQPKTKPSADQKRSVTFIEAQPEPAAAP |
| | | TDALPATGQLQGCSPAPSRKPEAMDEPVLTSSPAIVVADLH |
| | | SVSPKQSENFPTEEGEKEEDTEAQGATAHSPLSAQLSDPDD |
| | | FTGLETSSLLQHGDTVLHISEENGMENPLLSSQFTFTPTELG |
| | | KTDAVLDESHV |
| 5 | Human calmodulin | MADQLTEEQIAEFKEAFSLFDKDGDGTITTKELGTVMRSL |
| | UniProt P0DP23 | GQNPTEAELQDMINEVDADGNGTIDFPEFLTMMARKMKD |
| | | TDSEEEIREAFRVFDKDGNGYISAAELRHVMTNLGEKLTD |
| | | EEVDEMIREADIDGDGQVNYEEFVQMMTAK |
| 6 | hNALCN_Δ349- | MLKRKQSSRVEAQPVTDFGPDESLSDNADILWINKPWVHS |
| | 363 | LLRICAIISVISVCMNTPMTFEHYPPLQYVTFTLDTLLMFLY |
| | | TAEMIAKMHIRGIVKGDSSYVKDRWCVFDGFMVFCLWVS |
| | | LVLQVFEIADIVDQMSPWGMLRIPRPLIMIRAFRIYFRFELP |
| | | RTRITNILKRSGEQIWSVSIFLLFFLLLYGILGVQMFGTFTY |
| | | HCVVNDTKPGNVTWNSLAIPDTHCSPELEEGYQCPPGFKC |
| | | MDLEDLGLSRQELGYSGFNEIGTSIFTVYEAASQEGWVFL |
| | | MYRAIDSFPRWRSYFYFITLIFFLAWLVKNVFIAVIIETFAEI |
| | | RVQFQQMWGSRSSTTSTATTVDVNKPQGRAPACLQKMM |
| | | RSSVFHMFILSMVTVDVIVAASNYYKGENFRRQYDEFYLA |
| | | EVAFTVLFDLEALLKIWCLGFTGYISSSLHKFELLLVIGTTL |
| | | HVYPDLYHSQFTYFQVLRVVRLIKISPALEDFVYKIFGPGK |
| | | KLGSLVVFTASLLIVMSAISLQMFCFVEELDRFTTFPRAFM |
| | | SMFQILTQEGWVDVMDQTLNAVGHMWAPVVAIYFILYHL |
| | | FATLILLSLFVAVILDNLELDEDLKKLKQLKQSEANADTKE |
| | | KLPLRLRIFEKFPNRPQMVKISKLPSDFTVPKIRESFMKQFI |
| | | DRQQQDTCCLLRSLPTTSSSSCDHSKRSAIEDNKYIDQKLR |
| | | KSVFSIRARNLLEKETAVTKILRACTRQRMLSGSFEGQPAK |
| | | ERSILSVQHHIRQERRSLRHGSNSQRISRGKSLETLTQDHSN |
| | | TVRYRNAQREDSEIKMIQEKKEQAEMKRKVQEEELRENHP |
| | | YFDKPLFIVGREHRFRNFCRVVVRARFNASKTDPVTGAVK |
| | | NTKYHQLYDLLGLVTYLDWVMIIVTICSCISMMFESPFRRV |
| | | MHAPTLQIAEYVFVIFMSIELNLKIMADGLFFTPTAVIRDFG |
| | | GVMDIFIYLVSLIFLCWMPQNVPAESGAQLLMVLRCLRPL |
| | | RIFKLVPQMRKVVRELFSGFKEIFLVSILLLTLMLVFASFGV |
| | | QLFAGKLAKCNDPNIIRREDCNGIFRINVSVSKNLNLKLRP |
| | | GEKKPGFWVPRVWANPRNFNFDNVGNAMLALFEVLSLK |
| | | GWVEVRDVIIHRVGPIHGIYIHVFVFLGCMIGLTLFVGVVIA |
| | | NFNENKGTALLTVDQRRWEDLKSRLKIAQPLHLPPRPDND |
| | | GFRAKMYDITQHPFFKRTIALLVLAQSVLLSVKWDVEDPV |
| | | TVPLATMSVVFTFIFVLEVTMKIIAMSPAGFWQSRRNRYDL |
| | | LVTSLGVVWVVLHFALLNAYTYMMGACVIVFRFFSICGK |
| | | HVTLKMLLLTVVVSMYKSFFIIVGMFLLLLCYAFAGVVLF |
| | | GTVKYGENINRHANFSSAGKAITVLFRIVTGEDWNKIMHD |
| | | CMVQPPFCTPDEFTYWATDCGNYAGALMYFCSFYVIIAYI |
| | | MLNLLVAIIVENFSLFYSTEEDQLLSYNDLRHFQIIWNMVD |
| | | DKREGVIPTFRVKFLLRLLRGRLEVDLDKDKLLFKHMCYE |
| | | MERLHNGGDVTFHDVLSMLSYRSVDIRKSLQLEELLAREQ |
| | | LEYTIEEEVAKQTIRMWLKKCLKRIRAKQQQSCSIIHSLRES |
| | | QQQELSRFLNPPSIETTQPSEDTNANSQDNSMQPETSSQQQ |
| | | LLSPTLSDRGGSRQDAADAGKPQRKFGQWRLPSAPKPISH |
| | | SVSSVNLRFGGRTTMKSVVCKMNPMTDAASCGSEVKKW |
| | | WTRQLTVESDESGDDLLDI |
| 7 | hNALCN-hNav1.4 | MLKRKQSSRVEAQPVTDFGPDESLSDNADILWINKPWVHS |
| | DI-DII linker | LLRICAIISVISVCMNTPMTFEHYPPLQYVTFTLDTLLMFLY |
| | chimera | TAEMIAKMHIRGIVKGDSSYVKDRWCVFDGFMVFCLWVS |
| | | LVLQVFEIADIVDQMSPWGMLRIPRPLIMIRAFRIYFRFELP |
| | | RTRITNILKRSGEQIWSVSIFLLFFLLLYGILGVQMFGTFTY |
| | | HCVVNDTKPGNVTWNSLAIPDTHCSPELEEGYQCPPGFKC |
| | | MDLEDLGLSRQELGYSGFNEIGTSIFTVYEAASQEGWVFL |
| | | MYRAIDSFPRWRSYFYFITLIFFLAWLVKNVFIAVIIETFAEI |
| | | RVQFQQMDKEKEEEFQQMLEKFKKHQEELEKAKAAQALE |
| | | GGEADGDPAHGKDCNGSLDTSQGEKGAPRQSSSGDSGISD |
| | | AMEELEEAHQKCPPWWYKCAHKVLIWNCCAPWLKFKAC |
| | | LQKMMRSSVFHMFILSMVTVDVIVAASNYYKGENFRRQY |
| | | DEFYLAEVAFTVLFDLEALLKIWCLGFTGYISSSLHKFELLL |
| | | VIGTTLHVYPDLYHSQFTYFQVLRVVRLIKISPALEDFVYKI |
| | | FGPGKKLGSLVVFTASLLIVMSAISLQMFCFVEELDRFTTFP |
| | | RAFMSMFQILTQEGWVDVMDQTLNAVGHMWAPVVAIYF |
| | | ILYHLFATLILLSLFVAVILDNLELDEDLKKLKQLKQSEAN |
| | | ADTKEKLPLRLRIFEKFPNRPQMVKISKLPSDFTVPKIRESF |
| | | MKQFIDRQQQDTCCLLRSLPTTSSSSCDHSKRSAIEDNKYI |
| | | DQKLRKSVFSIRARNLLEKETAVTKILRACTRQRMLSGSFE |
| | | GQPAKERSILSVQHHIRQERRSLRHGSNSQRISRGKSLETLT |
| | | QDHSNTVRYRNAQREDSEIKMIQEKKEQAEMKRKVQEEE |
| | | LRENHPYFDKPLFIVGREHRFRNFCRVVVRARFNASKTDP |
| | | VTGAVKNTKYHQLYDLLGLVTYLDWVMIIVTICSCISMMF |
| | | ESPFRRVMHAPTLQIAEYVFVIFMSIELNLKIMADGLFFTPT |
| | | AVIRDFGGVMDIFIYLVSLIFLCWMPQNVPAESGAQLLMV |
| | | LRCLRPLRIFKLVPQMRKVVRELFSGFKEIFLVSILLLTLML |
| | | VFASFGVQLFAGKLAKCNDPNIIRREDCNGIFRINVSVSKN |
| | | LNLKLRPGEKKPGFWVPRVWANPRNFNFDNVGNAMLALF |
| | | EVLSLKGWVEVRDVIIHRVGPIHGIYIHVFVFLGCMIGLTLF |
| | | VGVVIANFNENKGTALLTVDQRRWEDLKSRLKIAQPLHLP |
| | | PRPDNDGFRAKMYDITQHPFFKRTIALLVLAQSVLLSVKW |
| | | DVEDPVTVPLATMSVVFTFIFVLEVTMKIIAMSPAGFWQSR |
| | | RNRYDLLVTSLGVVWVVLHFALLNAYTYMMGACVIVFRF |
| | | FSICGKHVTLKMLLLTVVVSMYKSFFIIVGMFLLLLCYAFA |
| | | GVVLFGTVKYGENINRHANFSSAGKAITVLFRIVTGEDWN |
| | | KIMHDCMVQPPFCTPDEFTYWATDCGNYAGALMYFCSFY |
| | | VIIAYIMLNLLVAIIVENFSLFYSTEEDQLLSYNDLRHFQIIW |
| | | NMVDDKREGVIPTFRVKFLLRLLRGRLEVDLDKDKLLFKH |
| | | MCYEMERLHNGGDVTFHDVLSMLSYRSVDIRKSLQLEELL |
| | | AREQLEYTIEEEVAKQTIRMWLKKCLKRIRAKQQQSCSIIH |
| | | SLRESQQQELSRFLNPPSIETTQPSEDTNANSQDNSMQPETS |
| | | SQQQLLSPTLSDRGGSRQDAADAGKPQRKFGQWRLPSAP |
| | | KPISHSVSSVNLRFGGRTTMKSVVCKMNPMTDAASCGSEV |
| | | KKWWTRQLTVESDESGDDLLDI |
| 8 | hNALCN-GGGS | MLKRKQSSRVEAQPVTDFGPDESLSDNADILWINKPWVHS |
| | DI-DII linker | LLRICAIISVISVCMNTPMTFEHYPPLQYVTFTLDTLLMFLY |
| | chimera | TAEMIAKMHIRGIVKGDSSYVKDRWCVFDGFMVFCLWVS |
| | | LVLQVFEIADIVDQMSPWGMLRIPRPLIMIRAFRIYFRFELP |
| | | RTRITNILKRSGEQIWSVSIFLLFFLLLYGILGVQMFGTFTY |
| | | HCVVNDTKPGNVTWNSLAIPDTHCSPELEEGYQCPPGFKC |
| | | MDLEDLGLSRQELGYSGFNEIGTSIFTVYEAASQEGWVFL |
| | | MYRAIDSFPRWRSYFYFITLIFFLAWLVKNVFIAVIIETFAEI |
| | | RVQFQQMGGGSGGGSGGGSGGGSGGGSGGGSGGGSGGG |
| | | SGGGSGGGACLQKMMRSSVFHMFILSMVTVDVIVAASNY |
| | | YKGENFRRQYDEFYLAEVAFTVLFDLEALLKIWCLGFTGY |
| | | ISSSLHKFELLLVIGTTLHVYPDLYHSQFTYFQVLRVVRLIK |
| | | ISPALEDFVYKIFGPGKKLGSLVVFTASLLIVMSAISLQMFC |
| | | FVEELDRFTTFPRAFMSMFQILTQEGWVDVMDQTLNAVG |
| | | HMWAPVVAIYFILYHLFATLILLSLFVAVILDNLELDEDLK |
| | | KLKQLKQSEANADTKEKLPLRLRIFEKFPNRPQMVKISKLP |
| | | SDFTVPKIRESFMKQFIDRQQQDTCCLLRSLPTTSSSSCDHS |
| | | KRSAIEDNKYIDQKLRKSVFSIRARNLLEKETAVTKILRAC |
| | | TRQRMLSGSFEGQPAKERSILSVQHHIRQERRSLRHGSNSQ |
| | | RISRGKSLETLTQDHSNTVRYRNAQREDSEIKMIQEKKEQA |
| | | EMKRKVQEEELRENHPYFDKPLFIVGREHRFRNFCRVVVR |
| | | ARFNASKTDPVTGAVKNTKYHQLYDLLGLVTYLDWVMII |
| | | VTICSCISMMFESPFRRVMHAPTLQIAEYVFVIFMSIELNLKI |
| | | MADGLFFTPTAVIRDFGGVMDIFIYLVSLIFLCWMPQNVPA |
| | | ESGAQLLMVLRCLRPLRIFKLVPQMRKVVRELFSGFKEIFL |
| | | VSILLLTLMLVFASFGVQLFAGKLAKCNDPNIIRREDCNGIF |
| | | RINVSVSKNLNLKLRPGEKKPGFWVPRVWANPRNFNFDN |
| | | VGNAMLALFEVLSLKGWVEVRDVIIHRVGPIHGIYIHVFVF |
| | | LGCMIGLTLFVGVVIANFNENKGTALLTVDQRRWEDLKSR |
| | | LKIAQPLHLPPRPDNDGFRAKMYDITQHPFFKRTIALLVLA |
| | | QSVLLSVKWDVEDPVTVPLATMSVVFTFIFVLEVTMKIIA |
| | | MSPAGFWQSRRNRYDLLVTSLGVVWVVLHFALLNAYTY |
| | | MMGACVIVFRFFSICGKHVTLKMLLLTVVVSMYKSFFIIVG |
| | | MFLLLLCYAFAGVVLFGTVKYGENINRHANFSSAGKAITV |
| | | LFRIVTGEDWNKIMHDCMVQPPFCTPDEFTYWATDCGNY |
| | | AGALMYFCSFYVIIAYIMLNLLVAIIVENFSLFYSTEEDQLL |
| | | SYNDLRHFQIIWNMVDDKREGVIPTFRVKFLLRLLRGRLE |
| | | VDLDKDKLLFKHMCYEMERLHNGGDVTFHDVLSMLSYR |
| | | SVDIRKSLQLEELLAREQLEYTIEEEVAKQTIRMWLKKCLK |
| | | RIRAKQQQSCSIIHSLRESQQQELSRFLNPPSIETTQPSEDTN |
| | | ANSQDNSMQPETSSQQQLLSPTLSDRGGSRQDAADAGKPQ |
| | | RKFGQWRLPSAPKPISHSVSSVNLRFGGRTTMKSVVCKMN |
| | | PMTDAASCGSEVKKWWTRQLTVESDESGDDLLDI |
| 9 | hNALCN_Δ638- | MLKRKQSSRVEAQPVTDFGPDESLSDNADILWINKPWVHS |
| | 670 | LLRICAIISVISVCMNTPMTFEHYPPLQYVTFTLDTLLMFLY |
| | | TAEMIAKMHIRGIVKGDSSYVKDRWCVFDGFMVFCLWVS |
| | | LVLQVFEIADIVDQMSPWGMLRIPRPLIMIRAFRIYFRFELP |
| | | RTRITNILKRSGEQIWSVSIFLLFFLLLYGILGVQMFGTFTY |
| | | HCVVNDTKPGNVTWNSLAIPDTHCSPELEEGYQCPPGFKC |
| | | MDLEDLGLSRQELGYSGFNEIGTSIFTVYEAASQEGWVFL |
| | | MYRAIDSFPRWRSYFYFITLIFFLAWLVKNVFIAVIIETFAEI |
| | | RVQFQQMWGSRSSTTSTATTQMFHEDAAGGWQLVAVDV |
| | | NKPQGRAPACLQKMMRSSVFHMFILSMVTVDVIVAASNY |
| | | YKGENFRRQYDEFYLAEVAFTVLFDLEALLKIWCLGFTGY |
| | | ISSSLHKFELLLVIGTTLHVYPDLYHSQFTYFQVLRVVRLIK |
| | | ISPALEDFVYKIFGPGKKLGSLVVFTASLLIVMSAISLQMFC |
| | | FVEELDRFTTFPRAFMSMFQILTQEGWVDVMDQTLNAVG |
| | | HMWAPVVAIYFILYHLFATLILLSLFVAVILDNLELDEDLK |
| | | KLKQLKQSEANADTKEKLPLRLRIFEKFQQDTCCLLRSLPT |
| | | TSSSSCDHSKRSAIEDNKYIDQKLRKSVFSIRARNLLEKETA |
| | | VTKILRACTRQRMLSGSFEGQPAKERSILSVQHHIRQERRS |
| | | LRHGSNSQRISRGKSLETLTQDHSNTVRYRNAQREDSEIK |
| | | MIQEKKEQAEMKRKVQEEELRENHPYFDKPLFIVGREHRF |
| | | RNFCRVVVRARFNASKTDPVTGAVKNTKYHQLYDLLGLV |
| | | TYLDWVMIIVTICSCISMMFESPFRRVMHAPTLQIAEYVFVI |
| | | FMSIELNLKIMADGLFFTPTAVIRDFGGVMDIFIYLVSLIFLC |
| | | WMPQNVPAESGAQLLMVLRCLRPLRIFKLVPQMRKVVRE |
| | | LFSGFKEIFLVSILLLTLMLVFASFGVQLFAGKLAKCNDPNI |
| | | IRREDCNGIFRINVSVSKNLNLKLRPGEKKPGFWVPRVWA |
| | | NPRNFNFDNVGNAMLALFEVLSLKGWVEVRDVIIHRVGPI |
| | | HGIYIHVFVFLGCMIGLTLFVGVVIANFNENKGTALLTVDQ |
| | | RRWEDLKSRLKIAQPLHLPPRPDNDGFRAKMYDITQHPFF |
| | | KRTIALLVLAQSVLLSVKWDVEDPVTVPLATMSVVFTFIFV |
| | | LEVTMKIIAMSPAGFWQSRRNRYDLLVTSLGVVWVVLHF |
| | | ALLNAYTYMMGACVIVFRFFSICGKHVTLKMLLLTVVVS |
| | | MYKSFFIIVGMFLLLLCYAFAGVVLFGTVKYGENINRHAN |
| | | FSSAGKAITVLFRIVTGEDWNKIMHDCMVQPPFCTPDEFTY |
| | | WATDCGNYAGALMYFCSFYVIIAYIMLNLLVAIIVENFSLF |
| | | YSTEEDQLLSYNDLRHFQIIWNMVDDKREGVIPTFRVKFLL |
| | | RLLRGRLEVDLDKDKLLFKHMCYEMERLHNGGDVTFHD |
| | | VLSMLSYRSVDIRKSLQLEELLAREQLEYTIEEEVAKQTIR |
| | | MWLKKCLKRIRAKQQQSCSIIHSLRESQQQELSRFLNPPSIE |
| | | TTQPSEDTNANSQDNSMQPETSSQQQLLSPTLSDRGGSRQ |
| | | DAADAGKPQRKFGQWRLPSAPKPISHSVSSVNLRFGGRTT |
| | | MKSVVCKMNPMTDAASCGSEVKKWWTRQLTVESDESGD |
| | | DLLDI |
| 10 | hNALCN_Δ794- | MLKRKQSSRVEAQPVTDFGPDESLSDNADILWINKPWVHS |
| | 828 | LLRICAIISVISVCMNTPMTFEHYPPLQYVTFTLDTLLMFLY |
| | | TAEMIAKMHIRGIVKGDSSYVKDRWCVFDGFMVFCLWVS |
| | | LVLQVFEIADIVDQMSPWGMLRIPRPLIMIRAFRIYFRFELP |
| | | RTRITNILKRSGEQIWSVSIFLLFFLLLYGILGVQMFGTFTY |
| | | HCVVNDTKPGNVTWNSLAIPDTHCSPELEEGYQCPPGFKC |
| | | MDLEDLGLSRQELGYSGFNEIGTSIFTVYEAASQEGWVFL |
| | | MYRAIDSFPRWRSYFYFITLIFFLAWLVKNVFIAVIIETFAEI |
| | | RVQFQQMWGSRSSTTSTATTQMFHEDAAGGWQLVAVDV |
| | | NKPQGRAPACLQKMMRSSVFHMFILSMVTVDVIVAASNY |
| | | YKGENFRRQYDEFYLAEVAFTVLFDLEALLKIWCLGFTGY |
| | | ISSSLHKFELLLVIGTTLHVYPDLYHSQFTYFQVLRVVRLIK |
| | | ISPALEDFVYKIFGPGKKLGSLVVFTASLLIVMSAISLQMFC |
| | | FVEELDRFTTFPRAFMSMFQILTQEGWVDVMDQTLNAVG |
| | | HMWAPVVAIYFILYHLFATLILLSLFVAVILDNLELDEDLK |
| | | KLKQLKQSEANADTKEKLPLRLRIFEKFPNRPQMVKISKLP |
| | | SDFTVPKIRESFMKQFIDRQQQDTCCLLRSLPTTSSSSCDHS |
| | | KRSAIEDNKYIDQKLRKSVFSIRARNLLEKETAVTKILRAC |
| | | TRQRMLSGSFEGQPAKERSILSVQHHIRQERRSLRHGSNSQ |
| | | RISRGKSLETLTQDHSNTVNHPYFDKPLFIVGREHRFRNFC |
| | | RVVVRARFNASKTDPVTGAVKNTKYHQLYDLLGLVTYLD |
| | | WVMIIVTICSCISMMFESPFRRVMHAPTLQIAEYVFVIFMSI |
| | | ELNLKIMADGLFFTPTAVIRDFGGVMDIFIYLVSLIFLCWM |
| | | PQNVPAESGAQLLMVLRCLRPLRIFKLVPQMRKVVRELFS |
| | | GFKEIFLVSILLLTLMLVFASFGVQLFAGKLAKCNDPNIIRR |
| | | EDCNGIFRINVSVSKNLNLKLRPGEKKPGFWVPRVWANPR |
| | | NFNFDNVGNAMLALFEVLSLKGWVEVRDVIIHRVGPIHGI |
| | | YIHVFVFLGCMIGLTLFVGVVIANFNENKGTALLTVDQRR |
| | | WEDLKSRLKIAQPLHLPPRPDNDGFRAKMYDITQHPFFKR |
| | | TIALLVLAQSVLLSVKWDVEDPVTVPLATMSVVFTFIFVLE |
| | | VTMKIIAMSPAGFWQSRRNRYDLLVTSLGVVWVVLHFAL |
| | | LNAYTYMMGACVIVFRFFSICGKHVTLKMLLLTVVVSMY |
| | | KSFFIIVGMFLLLLCYAFAGVVLFGTVKYGENINRHANFSS |
| | | AGKAITVLFRIVTGEDWNKIMHDCMVQPPFCTPDEFTYWA |
| | | TDCGNYAGALMYFCSFYVIIAYIMLNLLVAIIVENFSLFYST |
| | | EEDQLLSYNDLRHFQIIWNMVDDKREGVIPTFRVKFLLRLL |
| | | RGRLEVDLDKDKLLFKHMCYEMERLHNGGDVTFHDVLS |
| | | MLSYRSVDIRKSLQLEELLAREQLEYTIEEEVAKQTIRMWL |
| | | KKCLKRIRAKQQQSCSIIHSLRESQQQELSRFLNPPSIETTQP |
| | | SEDTNANSQDNSMQPETSSQQQLLSPTLSDRGGSRQDAAD |
| | | AGKPQRKFGQWRLPSAPKPISHSVSSVNLRFGGRTTMKSV |
| | | VCKMNPMTDAASCGSEVKKWWTRQLTVESDESGDDLLDI |
| 11 | hNALCN_Δ829- | MLKRKQSSRVEAQPVTDFGPDESLSDNADILWINKPWVHS |
| | 834 | LLRICAIISVISVCMNTPMTFEHYPPLQYVTFTLDTLLMFLY |
| | | TAEMIAKMHIRGIVKGDSSYVKDRWCVFDGFMVFCLWVS |
| | | LVLQVFEIADIVDQMSPWGMLRIPRPLIMIRAFRIYFRFELP |
| | | RTRITNILKRSGEQIWSVSIFLLFFLLLYGILGVQMFGTFTY |
| | | HCVVNDTKPGNVTWNSLAIPDTHCSPELEEGYQCPPGFKC |
| | | MDLEDLGLSRQELGYSGFNEIGTSIFTVYEAASQEGWVFL |
| | | MYRAIDSFPRWRSYFYFITLIFFLAWLVKNVFIAVIIETFAEI |
| | | RVQFQQMWGSRSSTTSTATTQMFHEDAAGGWQLVAVDV |
| | | NKPQGRAPACLQKMMRSSVFHMFILSMVTVDVIVAASNY |
| | | YKGENFRRQYDEFYLAEVAFTVLFDLEALLKIWCLGFTGY |
| | | ISSSLHKFELLLVIGTTLHVYPDLYHSQFTYFQVLRVVRLIK |
| | | ISPALEDFVYKIFGPGKKLGSLVVFTASLLIVMSAISLQMFC |
| | | FVEELDRFTTFPRAFMSMFQILTQEGWVDVMDQTLNAVG |
| | | HMWAPVVAIYFILYHLFATLILLSLFVAVILDNLELDEDLK |
| | | KLKQLKQSEANADTKEKLPLRLRIFEKFPNRPQMVKISKLP |
| | | SDFTVPKIRESFMKQFIDRQQQDTCCLLRSLPTTSSSSCDHS |
| | | KRSAIEDNKYIDQKLRKSVFSIRARNLLEKETAVTKILRAC |
| | | TRQRMLSGSFEGQPAKERSILSVQHHIRQERRSLRHGSNSQ |
| | | RISRGKSLETLTQDHSNTVRYRNAQREDSEIKMIQEKKEQA |
| | | EMKRKVQEEELREKPLFIVGREHRFRNFCRVVVRARFNAS |
| | | KTDPVTGAVKNTKYHQLYDLLGLVTYLDWVMIIVTICSCI |
| | | SMMFESPFRRVMHAPTLQIAEYVFVIFMSIELNLKIMADGL |
| | | FFTPTAVIRDFGGVMDIFIYLVSLIFLCWMPQNVPAESGAQ |
| | | LLMVLRCLRPLRIFKLVPQMRKVVRELFSGFKEIFLVSILLL |
| | | TLMLVFASFGVQLFAGKLAKCNDPNIIRREDCNGIFRINVS |
| | | VSKNLNLKLRPGEKKPGFWVPRVWANPRNFNFDNVGNA |
| | | MLALFEVLSLKGWVEVRDVIIHRVGPIHGIYIHVFVFLGCM |
| | | IGLTLFVGVVIANFNENKGTALLTVDQRRWEDLKSRLKIA |
| | | QPLHLPPRPDNDGFRAKMYDITQHPFFKRTIALLVLAQSVL |
| | | LSVKWDVEDPVTVPLATMSVVFTFIFVLEVTMKIIAMSPA |
| | | GFWQSRRNRYDLLVTSLGVVWVVLHFALLNAYTYMMGA |
| | | CVIVFRFFSICGKHVTLKMLLLTVVVSMYKSFFIIVGMFLLL |
| | | LCYAFAGVVLFGTVKYGENINRHANFSSAGKAITVLFRIVT |
| | | GEDWNKIMHDCMVQPPFCTPDEFTYWATDCGNYAGALM |
| | | YFCSFYVIIAYIMLNLLVAIIVENFSLFYSTEEDQLLSYNDLR |
| | | HFQIIWNMVDDKREGVIPTFRVKFLLRLLRGRLEVDLDKD |
| | | KLLFKHMCYEMERLHNGGDVTFHDVLSMLSYRSVDIRKS |
| | | LQLEELLAREQLEYTIEEEVAKQTIRMWLKKCLKRIRAKQ |
| | | QQSCSIIHSLRESQQQELSRFLNPPSIETTQPSEDTNANSQDN |
| | | SMQPETSSQQQLLSPTLSDRGGSRQDAADAGKPQRKFGQ |
| | | WRLPSAPKPISHSVSSVNLRFGGRTTMKSVVCKMNPMTDA |
| | | ASCGSEVKKWWTRQLTVESDESGDDLLDI |
| 12 | hNALCN-hNav1.4 | MLKRKQSSRVEAQPVTDFGPDESLSDNADILWINKPWVHS |
| | DII-DIII linker | LLRICAIISVISVCMNTPMTFEHYPPLQYVTFTLDTLLMFLY |
| | chimera1 | TAEMIAKMHIRGIVKGDSSYVKDRWCVFDGFMVFCLWVS |
| | | LVLQVFEIADIVDQMSPWGMLRIPRPLIMIRAFRIYFRFELP |
| | | RTRITNILKRSGEQIWSVSIFLLFFLLLYGILGVQMFGTFTY |
| | | HCVVNDTKPGNVTWNSLAIPDTHCSPELEEGYQCPPGFKC |
| | | MDLEDLGLSRQELGYSGFNEIGTSIFTVYEAASQEGWVFL |
| | | MYRAIDSFPRWRSYFYFITLIFFLAWLVKNVFIAVIIETFAEI |
| | | RVQFQQMWGSRSSTTSTATTQMFHEDAAGGWQLVAVDV |
| | | NKPQGRAPACLQKMMRSSVFHMFILSMVTVDVIVAASNY |
| | | YKGENFRRQYDEFYLAEVAFTVLFDLEALLKIWCLGFTGY |
| | | ISSSLHKFELLLVIGTTLHVYPDLYHSQFTYFQVLRVVRLIK |
| | | ISPALEDFVYKIFGPGKKLGSLVVFTASLLIVMSAISLQMFC |
| | | FVEELDRFTTFPRAFMSMFQILTQEGWVDVMDQTLNAVG |
| | | HMWAPVVAIYFILYHLFATLILLSLFVAVILDNLELDEDLK |
| | | KLKQLKQSNLQIAIGRIKLGIGFAKAFLLGLLHGKILSPKDI |
| | | MLSLGEADGAGEAGEAGETAPEDEKKEPPEEDLKKDNHIL |
| | | NHMGLADGPPSSLELDHLNFINNPYLTIQVPIASEESDLEMP |
| | | TEEETDTFSEPEDSKKPPQPLYDGNSSVCSTADYKPPEEDP |
| | | EEQAEENPEGEQPEECFTEACVQRWPCLYVDISQGRGKKW |
| | | WRFRNFCRVVVRARFNASKTDPVTGAVKNTKYHQLYDLL |
| | | GLVTYLDWVMIIVTICSCISMMFESPFRRVMHAPTLQIAEY |
| | | VFVIFMSIELNLKIMADGLFFTPTAVIRDFGGVMDIFIYLVS |
| | | LIFLCWMPQNVPAESGAQLLMVLRCLRPLRIFKLVPQMRK |
| | | VVRELFSGFKEIFLVSILLLTLMLVFASFGVQLFAGKLAKC |
| | | NDPNIIRREDCNGIFRINVSVSKNLNLKLRPGEKKPGFWVP |
| | | RVWANPRNFNFDNVGNAMLALFEVLSLKGWVEVRDVIIH |
| | | RVGPIHGIYIHVFVFLGCMIGLTLFVGVVIANFNENKGTAL |
| | | LTVDQRRWEDLKSRLKIAQPLHLPPRPDNDGFRAKMYDIT |
| | | QHPFFKRTIALLVLAQSVLLSVKWDVEDPVTVPLATMSVV |
| | | FTFIFVLEVTMKIIAMSPAGFWQSRRNRYDLLVTSLGVVW |
| | | VVLHFALLNAYTYMMGACVIVFRFFSICGKHVTLKMLLLT |
| | | VVVSMYKSFFIIVGMFLLLLCYAFAGVVLFGTVKYGENIN |
| | | RHANFSSAGKAITVLFRIVTGEDWNKIMHDCMVQPPFCTP |
| | | DEFTYWATDCGNYAGALMYFCSFYVIIAYIMLNLLVAIIVE |
| | | NFSLFYSTEEDQLLSYNDLRHFQIIWNMVDDKREGVIPTFR |
| | | VKFLLRLLRGRLEVDLDKDKLLFKHMCYEMERLHNGGDV |
| | | TFHDVLSMLSYRSVDIRKSLQLEELLAREQLEYTIEEEVAK |
| | | QTIRMWLKKCLKRIRAKQQQSCSIIHSLRESQQQELSRFLN |
| | | PPSIETTQPSEDTNANSQDNSMQPETSSQQQLLSPTLSDRG |
| | | GSRQDAADAGKPQRKFGQWRLPSAPKPISHSVSSVNLRFG |
| | | GRTTMKSVVCKMNPMTDAASCGSEVKKWWTRQLTVESD |
| | | ESGDDLLDI |
| 13 | hNALCN-hNav1.4 | MLKRKQSSRVEAQPVTDFGPDESLSDNADILWINKPWVHS |
| | DII-DIII linker | LLRICAIISVISVCMNTPMTFEHYPPLQYVTFTLDTLLMFLY |
| | chimera2 | TAEMIAKMHIRGIVKGDSSYVKDRWCVFDGFMVFCLWVS |
| | | LVLQVFEIADIVDQMSPWGMLRIPRPLIMIRAFRIYFRFELP |
| | | RTRITNILKRSGEQIWSVSIFLLFFLLLYGILGVQMFGTFTY |
| | | HCVVNDTKPGNVTWNSLAIPDTHCSPELEEGYQCPPGFKC |
| | | MDLEDLGLSRQELGYSGFNEIGTSIFTVYEAASQEGWVFL |
| | | MYRAIDSFPRWRSYFYFITLIFFLAWLVKNVFIAVIIETFAEI |
| | | RVQFQQMWGSRSSTTSTATTQMFHEDAAGGWQLVAVDV |
| | | NKPQGRAPACLQKMMRSSVFHMFILSMVTVDVIVAASNY |
| | | YKGENFRRQYDEFYLAEVAFTVLFDLEALLKIWCLGFTGY |
| | | ISSSLHKFELLLVIGTTLHVYPDLYHSQFTYFQVLRVVRLIK |
| | | ISPALEDFVYKIFGPGKKLGSLVVFTASLLIVMSAISLQMFC |
| | | FVEELDRFTTFPRAFMSMFQILTQEGWVDVMDQTLNAVG |
| | | HMWAPVVAIYFILYHLFATLILLSLFVAVILDNLELDEDLK |
| | | KLKQLKQSEANADTKEKLPLRLRIFEKFPNRPQMVKISKLP |
| | | SDFTVPKIRESFMKQFIDRQQQDDEKKEPPEEDLKKDNHIL |
| | | NHMGLADGPPSSLELDHLNFINNPYLTIQVPIASEESDLEMP |
| | | TEEETDTFSEPEDSKKPPQPLYDGNSSVCSTADYKPPEEDP |
| | | EEQAEENPEGEQPEECFTEACVQRWPCLYVDISQGRGKKW |
| | | WRFRNFCRVVVRARFNASKTDPVTGAVKNTKYHQLYDLL |
| | | GLVTYLDWVMIIVTICSCISMMFESPFRRVMHAPTLQIAEY |
| | | VFVIFMSIELNLKIMADGLFFTPTAVIRDFGGVMDIFIYLVS |
| | | LIFLCWMPQNVPAESGAQLLMVLRCLRPLRIFKLVPQMRK |
| | | VVRELFSGFKEIFLVSILLLTLMLVFASFGVQLFAGKLAKC |
| | | NDPNIIRREDCNGIFRINVSVSKNLNLKLRPGEKKPGFWVP |
| | | RVWANPRNFNFDNVGNAMLALFEVLSLKGWVEVRDVIIH |
| | | RVGPIHGIYIHVFVFLGCMIGLTLFVGVVIANFNENKGTAL |
| | | LTVDQRRWEDLKSRLKIAQPLHLPPRPDNDGFRAKMYDIT |
| | | QHPFFKRTIALLVLAQSVLLSVKWDVEDPVTVPLATMSVV |
| | | FTFIFVLEVTMKIIAMSPAGFWQSRRNRYDLLVTSLGVVW |
| | | VVLHFALLNAYTYMMGACVIVFRFFSICGKHVTLKMLLLT |
| | | VVVSMYKSFFIIVGMFLLLLCYAFAGVVLFGTVKYGENIN |
| | | RHANFSSAGKAITVLFRIVTGEDWNKIMHDCMVQPPFCTP |
| | | DEFTYWATDCGNYAGALMYFCSFYVIIAYIMLNLLVAIIVE |
| | | NFSLFYSTEEDQLLSYNDLRHFQIIWNMVDDKREGVIPTFR |
| | | VKFLLRLLRGRLEVDLDKDKLLFKHMCYEMERLHNGGDV |
| | | TFHDVLSMLSYRSVDIRKSLQLEELLAREQLEYTIEEEVAK |
| | | QTIRMWLKKCLKRIRAKQQQSCSIIHSLRESQQQELSRFLN |
| | | PPSIETTQPSEDTNANSQDNSMQPETSSQQQLLSPTLSDRG |
| | | GSRQDAADAGKPQRKFGQWRLPSAPKPISHSVSSVNLRFG |
| | | GRTTMKSVVCKMNPMTDAASCGSEVKKWWTRQLTVESD |
| | | ESGDDLLDI |
| 14 | hNALCN-hNav1.4 | MLKRKQSSRVEAQPVTDFGPDESLSDNADILWINKPWVHS |
| | DII-DIII linker | LLRICAIISVISVCMNTPMTFEHYPPLQYVTFTLDTLLMFLY |
| | chimera3 | TAEMIAKMHIRGIVKGDSSYVKDRWCVFDGFMVFCLWVS |
| | | LVLQVFEIADIVDQMSPWGMLRIPRPLIMIRAFRIYFRFELP |
| | | RTRITNILKRSGEQIWSVSIFLLFFLLLYGILGVQMFGTFTY |
| | | HCVVNDTKPGNVTWNSLAIPDTHCSPELEEGYQCPPGFKC |
| | | MDLEDLGLSRQELGYSGFNEIGTSIFTVYEAASQEGWVFL |
| | | MYRAIDSFPRWRSYFYFITLIFFLAWLVKNVFIAVIIETFAEI |
| | | RVQFQQMWGSRSSTTSTATTQMFHEDAAGGWQLVAVDV |
| | | NKPQGRAPACLQKMMRSSVFHMFILSMVTVDVIVAASNY |
| | | YKGENFRRQYDEFYLAEVAFTVLFDLEALLKIWCLGFTGY |
| | | ISSSLHKFELLLVIGTTLHVYPDLYHSQFTYFQVLRVVRLIK |
| | | ISPALEDFVYKIFGPGKKLGSLVVFTASLLIVMSAISLQMFC |
| | | FVEELDRFTTFPRAFMSMFQILTQEGWVDVMDQTLNAVG |
| | | HMWAPVVAIYFILYHLFATLILLSLFVAVILDNLELDEDLK |
| | | KLKQLKQSEANADTKEKLPLRLRIFEKFPNRPQMVKISKLP |
| | | SDFTVPKIRESFMKQFIDRQQQDDEKKEPPEEDLKKDNHIL |
| | | NHMGLADGPPSSLELDHLNFINNPYLTIQVPIASEESDLEMP |
| | | TEEETDTFSEPEDSKKPPQPLYDGNSSVCSTADYKAQREDS |
| | | EIKMIQEKKEQAEMKRKVQEEELRENHPYFDKPLFIVGRE |
| | | HRFRNFCRVVVRARFNASKTDPVTGAVKNTKYHQLYDLL |
| | | GLVTYLDWVMIIVTICSCISMMFESPFRRVMHAPTLQIAEY |
| | | VFVIFMSIELNLKIMADGLFFTPTAVIRDFGGVMDIFIYLVS |
| | | LIFLCWMPQNVPAESGAQLLMVLRCLRPLRIFKLVPQMRK |
| | | VVRELFSGFKEIFLVSILLLTLMLVFASFGVQLFAGKLAKC |
| | | NDPNIIRREDCNGIFRINVSVSKNLNLKLRPGEKKPGFWVP |
| | | RVWANPRNFNFDNVGNAMLALFEVLSLKGWVEVRDVIIH |
| | | RVGPIHGIYIHVFVFLGCMIGLTLFVGVVIANFNENKGTAL |
| | | LTVDQRRWEDLKSRLKIAQPLHLPPRPDNDGFRAKMYDIT |
| | | QHPFFKRTIALLVLAQSVLLSVKWDVEDPVTVPLATMSVV |
| | | FTFIFVLEVTMKIIAMSPAGFWQSRRNRYDLLVTSLGVVW |
| | | VVLHFALLNAYTYMMGACVIVFRFFSICGKHVTLKMLLLT |
| | | VVVSMYKSFFIIVGMFLLLLCYAFAGVVLFGTVKYGENIN |
| | | RHANFSSAGKAITVLFRIVTGEDWNKIMHDCMVQPPFCTP |
| | | DEFTYWATDCGNYAGALMYFCSFYVIIAYIMLNLLVAIIVE |
| | | NFSLFYSTEEDQLLSYNDLRHFQIIWNMVDDKREGVIPTFR |
| | | VKFLLRLLRGRLEVDLDKDKLLFKHMCYEMERLHNGGDV |
| | | TFHDVLSMLSYRSVDIRKSLQLEELLAREQLEYTIEEEVAK |
| | | QTIRMWLKKCLKRIRAKQQQSCSIIHSLRESQQQELSRFLN |
| | | PPSIETTQPSEDTNANSQDNSMQPETSSQQQLLSPTLSDRG |
| | | GSRQDAADAGKPQRKFGQWRLPSAPKPISHSVSSVNLRFG |
| | | GRTTMKSVVCKMNPMTDAASCGSEVKKWWTRQLTVESD |
| | | ESGDDLLDI |
| 15 | hNALCN-hNav1.4 | MLKRKQSSRVEAQPVTDFGPDESLSDNADILWINKPWVHS |
| | DII-DIII linker | LLRICAIISVISVCMNTPMTFEHYPPLQYVTFTLDTLLMFLY |
| | chimera4 | TAEMIAKMHIRGIVKGDSSYVKDRWCVFDGFMVFCLWVS |
| | | LVLQVFEIADIVDQMSPWGMLRIPRPLIMIRAFRIYFRFELP |
| | | RTRITNILKRSGEQIWSVSIFLLFFLLLYGILGVQMFGTFTY |
| | | HCVVNDTKPGNVTWNSLAIPDTHCSPELEEGYQCPPGFKC |
| | | MDLEDLGLSRQELGYSGFNEIGTSIFTVYEAASQEGWVFL |
| | | MYRAIDSFPRWRSYFYFITLIFFLAWLVKNVFIAVIIETFAEI |
| | | RVQFQQMWGSRSSTTSTATTQMFHEDAAGGWQLVAVDV |
| | | NKPQGRAPACLQKMMRSSVFHMFILSMVTVDVIVAASNY |
| | | YKGENFRRQYDEFYLAEVAFTVLFDLEALLKIWCLGFTGY |
| | | ISSSLHKFELLLVIGTTLHVYPDLYHSQFTYFQVLRVVRLIK |
| | | ISPALEDFVYKIFGPGKKLGSLVVFTASLLIVMSAISLQMFC |
| | | FVEELDRFTTFPRAFMSMFQILTQEGWVDVMDQTLNAVG |
| | | HMWAPVVAIYFILYHLFATLILLSLFVAVILDNLELDEDLK |
| | | KLKQLKQSEANADTKEKLPLRLRIFEKFPNRPQMVKISKLP |
| | | SDFTVPKIRESFMKQFIDRQQQDTCCLLRSLPTTSSSSCDHS |
| | | KRSAIEDNKYIDQKLRKSVFSIRARNLLEKETAVTKILRAC |
| | | TRQRMLSGSFEGQPAKERSILSVQHHIRQERRSLRHGSNSQ |
| | | RISRGKSLETLTQDHSNTVRYRNPPEEDPEEQAEENPEGEQ |
| | | PEECFTEACVQRWPCLYVDISQGRGKKWWRFRNFCRVVV |
| | | RARFNASKTDPVTGAVKNTKYHQLYDLLGLVTYLDWVMI |
| | | IVTICSCISMMFESPFRRVMHAPTLQIAEYVFVIFMSIELNLK |
| | | IMADGLFFTPTAVIRDFGGVMDIFIYLVSLIFLCWMPQNVP |
| | | AESGAQLLMVLRCLRPLRIFKLVPQMRKVVRELFSGFKEIF |
| | | LVSILLLTLMLVFASFGVQLFAGKLAKCNDPNIIRREDCNGI |
| | | FRINVSVSKNLNLKLRPGEKKPGFWVPRVWANPRNFNFDN |
| | | VGNAMLALFEVLSLKGWVEVRDVIIHRVGPIHGIYIHVFVF |
| | | LGCMIGLTLFVGVVIANFNENKGTALLTVDQRRWEDLKSR |
| | | LKIAQPLHLPPRPDNDGFRAKMYDITQHPFFKRTIALLVLA |
| | | QSVLLSVKWDVEDPVTVPLATMSVVFTFIFVLEVTMKIIA |
| | | MSPAGFWQSRRNRYDLLVTSLGVVWVVLHFALLNAYTY |
| | | MMGACVIVFRFFSICGKHVTLKMLLLTVVVSMYKSFFIIVG |
| | | MFLLLLCYAFAGVVLFGTVKYGENINRHANFSSAGKAITV |
| | | LFRIVTGEDWNKIMHDCMVQPPFCTPDEFTYWATDCGNY |
| | | AGALMYFCSFYVIIAYIMLNLLVAIIVENFSLFYSTEEDQLL |
| | | SYNDLRHFQIIWNMVDDKREGVIPTFRVKFLLRLLRGRLE |
| | | VDLDKDKLLFKHMCYEMERLHNGGDVTFHDVLSMLSYR |
| | | SVDIRKSLQLEELLAREQLEYTIEEEVAKQTIRMWLKKCLK |
| | | RIRAKQQQSCSIIHSLRESQQQELSRFLNPPSIETTQPSEDTN |
| | | ANSQDNSMQPETSSQQQLLSPTLSDRGGSRQDAADAGKPQ |
| | | RKFGQWRLPSAPKPISHSVSSVNLRFGGRTTMKSVVCKMN |
| | | PMTDAASCGSEVKKWWTRQLTVESDESGDDLLDI |
| 16 | MBP-modified | MKIEEGKLVIWINGDKGYNGLAEVGKKFEKDTGIKVTVEH |
| | hNALCN DII-DIII | PDKLEEKFPQVAATGDGPDIIFWAHDRFGGYAQSGLLAEIT |
| | linker (MBP portion | PDKAFQDKLYPFTWDAVRYNGKLIAYPIAVEALSLIYNKD |
| | underlined) | LLPNPPKTWEEIPALDKELKAKGKSALMENLQEPYFTWPLI |
| | | AADGGYAFKYENGKYDIKDVGVDNAGAKAGLTFLVDLIK |
| | | NKHMNADTDYSIAEAAFNKGETAMTINGPWAWSNIDTSK |
| | | VNYGVTVLPTFKGQPSKPFVGVLSAGINAASPNKELAKEF |
| | | LENYLLTDEGLEAVNKDKPLGAVALKSYEEELAKDPRIAA |
| | | TMENAQKGEIMPNIPQMSAFWYAVRTAVINAASGRQTVD |
| | | EALKDAQTNSSSNNNNNNNNNNGENLYFQGSSEANADTK |
| | | EKLPLRLRIFEKFPNRPQMVKISKLPSDFTVPKIRESFMKQFI |
| | | DRQQQDTCCLLRSLPTTSSSSCDHSKRSAIEDNKYIDQKLR |
| | | KSVFSIRARNLLEKETAVTKILRACTRQRMLSGSFEGQPAK |
| | | ERSILSVQHHIRQERRSLRHGSNSQRISRGKSLETLTQDHSN |
| | | TVRYRNAQREDSEIKMIQEKKEQAEMKRKVQEEELRENHP |
| | | YFDKPLFIVGREHR |
| 17 | Palmitoyl peptide- | MMLCCMRRTKQGTGGSGGTGGSGGSEANADTKEKLPLRL |
| | modified hNALCN | RIFEKFPNRPQMVKISKLPSDFTVPKIRESFMKQFIDRQQQD |
| | DII-DIII linker | TCCLLRSLPTTSSSSCDHSKRSAIEDNKYIDQKLRKSVFSIR |
| | (modification | ARNLLEKETAVTKILRACTRQRMLSGSFEGQPAKERSILSV |
| | underlined) | QHHIRQERRSLRHGSNSQRISRGKSLETLTQDHSNTVRYRN |
| | | AQREDSEIKMIQEKKEQAEMKRKVQEEELRENHPYFDKPL |
| | | FIVGREHR |
| 18 | Unmodified | SEANADTKEKLPLRLRIFEKFPNRPQMVKISKLPSDFTVPKI |
| | hNALCN DII-DIII | RESFMKQFIDRQQQDTCCLLRSLPTTSSSSCDHSKRSAIEDN |
| | linker (residues | KYIDQKLRKSVFSIRARNLLEKETAVTKILRACTRQRMLSG |
| | S617-R845) | SFEGQPAKERSILSVQHHIRQERRSLRHGSNSQRISRGKSLE |
| | | TLTQDHSNTVRYRNAQREDSEIKMIQEKKEQAEMKRKVQ |
| | | EEELRENHPYFDKPLFIVGREHR |
| 19 | hNALCN DI-DII | QMFHEDAAGGWQLVA |
| | linker peptide | |
| | (residues Q349- | |
| | A363 | |
| 20 | hNALCN DII-DIII | PNRPQMVKISKLPSDFTVPKIRESFMKQFIDRQ |
| | linker peptide | |
| | (residues P638- | |
| | Q670) | |
| 21 | hNALCN DII-DIII | RYRNAQREDSEIKMIQEKKEQAEMKRKVQEEELRE |
| | linker peptide | |
| | (residues R794- | |
| | E828) | |
| 22 | hNALCN DII-DIII | NHPYFDK |
| | linker peptide | |
| | (residues N829- | |
| | K835) | |
| 23 | hNALCN DII-DIII | SEANADTKEKLPLRLRIFEKFPNRPQMVKISKLPSDFTVPKI |
| | linker peptide | RESFMKQFIDRQQQDTCCLLRSLPTTSSSSCDHSKRSAIEDN |
| | (residues S617- | KYIDQKLRKSVFSIRARNLLEKETAVTKILRACTRQRMLS |
| | S740) | |
| 24 | hNALCN DII-DIII | RESFMKQFIDRQQQDTCCLLRSLPTTSSSSCDHSKRSAIEDN |
| | linker peptide | KYIDQKLRKSVFSIRARNLLEKETAVTKILRACTRQRMLSG |
| | (residues R659- | SFEGQPAKERSILSVQHHIRQERRSLRHGSNSQ |
| | Q774) | |
| 25 | hNALCN DII-DIII | GSFEGQPAKERSILSVQHHIRQERRSLRHGSNSQRISRGKSL |
| | linker peptide | ETLTQDHSNTVRYRNAQREDSEIKMIQEKKEQAEMKRKV |
| | (residues G741- | QEEELRENHPYFDKPLFIVGREHR |
| | R845) | |
| 26 | hNALCN DI-DII | MWGSRSSTTSTATTQMFHEDAAGGWQLVAVDVNKPQGR |
| | linker peptide | APA |
| | (residues M335- | |
| | A375) | |
| 27 | hNALCN DII-DIII | SEANADTKEKLPLRLRIFEKFPNRPQMVKISKLPSDFTVPKI |
| | linker peptide | RESFMKQFIDRQQQDTCC |
| | (residues S617- | |
| | C676) | |
| 28 | hNALCN DII-DIII | LLRSLPTTSSSSCDHSKRSAIEDNKYIDQKLRKSVFSIRARN |
| | linker peptide | LLEKETAVTKILRACTRQRMLS |
| | (residues L677- | |
| | S740) | |
| 29 | hNALCN DII-DIII | GSFEGQPAKERSILSVQHHIRQERRSLRHGSNSQRISRGKSL |
| | linker peptide | ETLTQDH |
| | (residues G741- | |
| | H789) | |
| 30 | hNALCN DII-DIII | SNTVRYRNAQREDSEIKMIQEKKEQAEMKRKVQEEELREN |
| | linker peptide | HPYFDKPLFIVGREHR |
| | (residues S790- | |
| | R845) | |
| 31 | hNALCN residues | YSTEEDQLLSYNDLRHFQIIWNMVDDKREGVIPTFRVKFLL |
| | Y1454-I1738 | RLLRGRLEVDLDKDKLLFKHMCYEMERLHNGGDVTFHD |
| | | VLSMLSYRSVDIRKSLQLEELLAREQLEYTIEEEVAKQTIR |
| | | MWLKKCLKRIRAKQQQSCSIIHSLRESQQQELSRFLNPPSIE |
| | | TTQPSEDTNANSQDNSMQPETSSQQQLLSPTLSDRGGSRQ |
| | | DAADAGKPQRKFGQWRLPSAPKPISHSVSSVNLRFGGRTT |
| | | MKSVVCKMNPMTDAASCGSEVKKWWTRQLTVESDESGD |
| | | DLLDI |
| 32 | hNALCN eGFP-2X | MLKRKQSSRVEAQPVTDFGPDESLSDNADILWINKPWVHS |
| | Flag | LLRICAIISVISVCMNTPMTFEHYPPLQYVTFTLDTLLMFLY |
| | | TAEMIAKMHIRGIVKGDSSYVKDRWCVFDGFMVFCLWVS |
| | | LVLQVFEIADIVDQMSPWGMLRIPRPLIMIRAFRIYFRFELP |
| | | RTRITNILKRSGEQIWSVSIFLLFFLLLYGILGVQMFGTFTY |
| | | HCVVNDTKPGNVTWNSLAIPDTHCSPELEEGYQCPPGFKC |
| | | MDLEDLGLSRQELGYSGFNEIGTSIFTVYEAASQEGWVFL |
| | | MYRAIDSFPRWRSYFYFITLIFFLAWLVKNVFIAVIIETFAEI |
| | | RVQFQQMWGSRSSTTSTATTQMFHEDAAGGWQLVAVDV |
| | | NKPQGRAPACLQKMMRSSVFHMFILSMVTVDVIVAASNY |
| | | YKGENFRRQYDEFYLAEVAFTVLFDLEALLKIWCLGFTGY |
| | | ISSSLHKFELLLVIGTTLHVYPDLYHSQFTYFQVLRVVRLIK |
| | | ISPALEDFVYKIFGPGKKLGSLVVFTASLLIVMSAISLQMFC |
| | | FVEELDRFTTFPRAFMSMFQILTQEGWVDVMDQTLNAVG |
| | | HMWAPVVAIYFILYHLFATLILLSLFVAVILDNLELDEDLK |
| | | KLKQLKQSEANADTKEKLPLRLRIFEKFPNRPQMVKISKLP |
| | | SDFTVPKIRESFMKQFIDRQQQDTCCLLRSLPTTSSSSCDHS |
| | | KRSAIEDNKYIDQKLRKSVFSIRARNLLEKETAVTKILRAC |
| | | TRQRMLSGSFEGQPAKERSILSVQHHIRQERRSLRHGSNSQ |
| | | RISRGKSLETLTQDHSNTVRYRNAQREDSEIKMIQEKKEQA |
| | | EMKRKVQEEELRENHPYFDKPLFIVGREHRFRNFCRVVVR |
| | | ARFNASKTDPVTGAVKNTKYHQLYDLLGLVTYLDWVMII |
| | | VTICSCISMMFESPFRRVMHAPTLQIAEYVFVIFMSIELNLKI |
| | | MADGLFFTPTAVIRDFGGVMDIFIYLVSLIFLCWMPQNVPA |
| | | ESGAQLLMVLRCLRPLRIFKLVPQMRKVVRELFSGFKEIFL |
| | | VSILLLTLMLVFASFGVQLFAGKLAKCNDPNIIRREDCNGIF |
| | | RINVSVSKNLNLKLRPGEKKPGFWVPRVWANPRNFNFDN |
| | | VGNAMLALFEVLSLKGWVEVRDVIIHRVGPIHGIYIHVFVF |
| | | LGCMIGLTLFVGVVIANFNENKGTALLTVDQRRWEDLKSR |
| | | LKIAQPLHLPPRPDNDGFRAKMYDITQHPFFKRTIALLVLA |
| | | QSVLLSVKWDVEDPVTVPLATMSVVFTFIFVLEVTMKIIA |
| | | MSPAGFWQSRRNRYDLLVTSLGVVWVVLHFALLNAYTY |
| | | MMGACVIVFRFFSICGKHVTLKMLLLTVVVSMYKSFFIIVG |
| | | MFLLLLCYAFAGVVLFGTVKYGENINRHANFSSAGKAITV |
| | | LFRIVTGEDWNKIMHDCMVQPPFCTPDEFTYWATDCGNY |
| | | AGALMYFCSFYVIIAYIMLNLLVAIIVENFSLFYSTEEDQLL |
| | | SYNDLRHFQIIWNMVDDKREGVIPTFRVKFLLRLLRGRLE |
| | | VDLDKDKLLFKHMCYEMERLHNGGDVTFHDVLSMLSYR |
| | | SVDIRKSLQLEELLAREQLEYTIEEEVAKQTIRMWLKKCLK |
| | | RIRAKQQQSCSIIHSLRESQQQELSRFLNPPSIETTQPSEDTN |
| | | ANSQDNSMQPETSSQQQLLSPTLSDRGGSRQDAADAGKPQ |
| | | RKFGQWRLPSAPKPISHSVSSVNLRFGGRTTMKSVVCKMN |
| | | PMTDAASCGSEVKKWWTRQLTVESDESGDDLLDIGGSGG |
| | | SVSKGEELFTGVVPILVELDGDVNGHKFSVSGEGEGDATY |
| | | GKLTLKFICTTGKLPVPWPTLVTTLTYGVQCFSRYPDHMK |
| | | QHDFFKSAMPEGYVQERTIFFKDDGNYKTRAEVKFEGDTL |
| | | VNRIELKGIDFKEDGNILGHKLEYNYNSHNVYIMADKQKN |
| | | GIKVNFKIRHNIEDGSVQLADHYQQNTPIGDGPVLLPDNHY |
| | | LSTQSALSKDPNEKRDHMVLLEFVTAAGITLGMDELYKGG |
| | | SGGSDYKDDDDKGSGDYKDDDDK |
| 33 | hNALCN Δ351 | MLKRKQSSRVEAQPVTDFGPDESLSDNADILWINKPWVHS |
| | | LLRICAIISVISVCMNTPMTFEHYPPLQYVTFTLDTLLMFLY |
| | | TAEMIAKMHIRGIVKGDSSYVKDRWCVFDGFMVFCLWVS |
| | | LVLQVFEIADIVDQMSPWGMLRIPRPLIMIRAFRIYFRFELP |
| | | RTRITNILKRSGEQIWSVSIFLLFFLLLYGILGVQMFGTFTY |
| | | HCVVNDTKPGNVTWNSLAIPDTHCSPELEEGYQCPPGFKC |
| | | MDLEDLGLSRQELGYSGFNEIGTSIFTVYEAASQEGWVFL |
| | | MYRAIDSFPRWRSYFYFITLIFFLAWLVKNVFIAVIIETFAEI |
| | | RVQFQQMWGSRSSTTSTATTQMHEDAAGGWQLVAVDVN |
| | | KPQGRAPACLQKMMRSSVFHMFILSMVTVDVIVAASNYY |
| | | KGENFRRQYDEFYLAEVAFTVLFDLEALLKIWCLGFTGYIS |
| | | SSLHKFELLLVIGTTLHVYPDLYHSQFTYFQVLRVVRLIKIS |
| | | PALEDFVYKIFGPGKKLGSLVVFTASLLIVMSAISLQMFCF |
| | | VEELDRFTTFPRAFMSMFQILTQEGWVDVMDQTLNAVGH |
| | | MWAPVVAIYFILYHLFATLILLSLFVAVILDNLELDEDLKK |
| | | LKQLKQSEANADTKEKLPLRLRIFEKFPNRPQMVKISKLPS |
| | | DFTVPKIRESFMKQFIDRQQQDTCCLLRSLPTTSSSSCDHSK |
| | | RSAIEDNKYIDQKLRKSVFSIRARNLLEKETAVTKILRACTR |
| | | QRMLSGSFEGQPAKERSILSVQHHIRQERRSLRHGSNSQRIS |
| | | RGKSLETLTQDHSNTVRYRNAQREDSEIKMIQEKKEQAEM |
| | | KRKVQEEELRENHPYFDKPLFIVGREHRFRNFCRVVVRAR |
| | | FNASKTDPVTGAVKNTKYHQLYDLLGLVTYLDWVMIIVTI |
| | | CSCISMMFESPFRRVMHAPTLQIAEYVFVIFMSIELNLKIMA |
| | | DGLFFTPTAVIRDFGGVMDIFIYLVSLIFLCWMPQNVPAES |
| | | GAQLLMVLRCLRPLRIFKLVPQMRKVVRELFSGFKEIFLVS |
| | | ILLLTLMLVFASFGVQLFAGKLAKCNDPNIIRREDCNGIFRI |
| | | NVSVSKNLNLKLRPGEKKPGFWVPRVWANPRNFNFDNVG |
| | | NAMLALFEVLSLKGWVEVRDVIIHRVGPIHGIYIHVFVFLG |
| | | CMIGLTLFVGVVIANFNENKGTALLTVDQRRWEDLKSRLK |
| | | IAQPLHLPPRPDNDGFRAKMYDITQHPFFKRTIALLVLAQS |
| | | VLLSVKWDVEDPVTVPLATMSVVFTFIFVLEVTMKIIAMSP |
| | | AGFWQSRRNRYDLLVTSLGVVWVVLHFALLNAYTYMMG |
| | | ACVIVFRFFSICGKHVTLKMLLLTVVVSMYKSFFIIVGMFL |
| | | LLLCYAFAGVVLFGTVKYGENINRHANFSSAGKAITVLFRI |
| | | VTGEDWNKIMHDCMVQPPFCTPDEFTYWATDCGNYAGA |
| | | LMYFCSFYVIIAYIMLNLLVAIIVENFSLFYSTEEDQLLSYN |
| | | DLRHFQIIWNMVDDKREGVIPTFRVKFLLRLLRGRLEVDL |
| | | DKDKLLFKHMCYEMERLHNGGDVTFHDVLSMLSYRSVDI |
| | | RKSLQLEELLAREQLEYTIEEEVAKQTIRMWLKKCLKRIRA |
| | | KQQQSCSIIHSLRESQQQELSRFLNPPSIETTQPSEDTNANSQ |
| | | DNSMQPETSSQQQLLSPTLSDRGGSRQDAADAGKPQRKFG |
| | | QWRLPSAPKPISHSVSSVNLRFGGRTTMKSVVCKMNPMTD |
| | | AASCGSEVKKWWTRQLTVESDESGDDLLDI |
| 34 | hNALCN_Δ359 | MLKRKQSSRVEAQPVTDFGPDESLSDNADILWINKPWVHS |
| | | LLRICAIISVISVCMNTPMTFEHYPPLQYVTFTLDTLLMFLY |
| | | TAEMIAKMHIRGIVKGDSSYVKDRWCVFDGFMVFCLWVS |
| | | LVLQVFEIADIVDQMSPWGMLRIPRPLIMIRAFRIYFRFELP |
| | | RTRITNILKRSGEQIWSVSIFLLFFLLLYGILGVQMFGTFTY |
| | | HCVVNDTKPGNVTWNSLAIPDTHCSPELEEGYQCPPGFKC |
| | | MDLEDLGLSRQELGYSGFNEIGTSIFTVYEAASQEGWVFL |
| | | MYRAIDSFPRWRSYFYFITLIFFLAWLVKNVFIAVIIETFAEI |
| | | RVQFQQMWGSRSSTTSTATTQMFHEDAAGGQLVAVDVN |
| | | KPQGRAPACLQKMMRSSVFHMFILSMVTVDVIVAASNYY |
| | | KGENFRRQYDEFYLAEVAFTVLFDLEALLKIWCLGFTGYIS |
| | | SSLHKFELLLVIGTTLHVYPDLYHSQFTYFQVLRVVRLIKIS |
| | | PALEDFVYKIFGPGKKLGSLVVFTASLLIVMSAISLQMFCF |
| | | VEELDRFTTFPRAFMSMFQILTQEGWVDVMDQTLNAVGH |
| | | MWAPVVAIYFILYHLFATLILLSLFVAVILDNLELDEDLKK |
| | | LKQLKQSEANADTKEKLPLRLRIFEKFPNRPQMVKISKLPS |
| | | DFTVPKIRESFMKQFIDRQQQDTCCLLRSLPTTSSSSCDHSK |
| | | RSAIEDNKYIDQKLRKSVFSIRARNLLEKETAVTKILRACTR |
| | | QRMLSGSFEGQPAKERSILSVQHHIRQERRSLRHGSNSQRIS |
| | | RGKSLETLTQDHSNTVRYRNAQREDSEIKMIQEKKEQAEM |
| | | KRKVQEEELRENHPYFDKPLFIVGREHRFRNFCRVVVRAR |
| | | FNASKTDPVTGAVKNTKYHQLYDLLGLVTYLDWVMIIVTI |
| | | CSCISMMFESPFRRVMHAPTLQIAEYVFVIFMSIELNLKIMA |
| | | DGLFFTPTAVIRDFGGVMDIFIYLVSLIFLCWMPQNVPAES |
| | | GAQLLMVLRCLRPLRIFKLVPQMRKVVRELFSGFKEIFLVS |
| | | ILLLTLMLVFASFGVQLFAGKLAKCNDPNIIRREDCNGIFRI |
| | | NVSVSKNLNLKLRPGEKKPGFWVPRVWANPRNFNFDNVG |
| | | NAMLALFEVLSLKGWVEVRDVIIHRVGPIHGIYIHVFVFLG |
| | | CMIGLTLFVGVVIANFNENKGTALLTVDQRRWEDLKSRLK |
| | | IAQPLHLPPRPDNDGFRAKMYDITQHPFFKRTIALLVLAQS |
| | | VLLSVKWDVEDPVTVPLATMSVVFTFIFVLEVTMKIIAMSP |
| | | AGFWQSRRNRYDLLVTSLGVVWVVLHFALLNAYTYMMG |
| | | ACVIVFRFFSICGKHVTLKMLLLTVVVSMYKSFFIIVGMFL |
| | | LLLCYAFAGVVLFGTVKYGENINRHANFSSAGKAITVLFRI |
| | | VTGEDWNKIMHDCMVQPPFCTPDEFTYWATDCGNYAGA |
| | | LMYFCSFYVIIAYIMLNLLVAIIVENFSLFYSTEEDQLLSYN |
| | | DLRHFQIIWNMVDDKREGVIPTFRVKFLLRLLRGRLEVDL |
| | | DKDKLLFKHMCYEMERLHNGGDVTFHDVLSMLSYRSVDI |
| | | RKSLQLEELLAREQLEYTIEEEVAKQTIRMWLKKCLKRIRA |
| | | KQQQSCSIIHSLRESQQQELSRFLNPPSIETTQPSEDTNANSQ |
| | | DNSMQPETSSQQQLLSPTLSDRGGSRQDAADAGKPQRKFG |
| | | QWRLPSAPKPISHSVSSVNLRFGGRTTMKSVVCKMNPMTD |
| | | AASCGSEVKKWWTRQLTVESDESGDDLLDI |
| 35 | hNALCN_Δ361 | MLKRKQSSRVEAQPVTDFGPDESLSDNADILWINKPWVHS |
| | | LLRICAIISVISVCMNTPMTFEHYPPLQYVTFTLDTLLMFLY |
| | | TAEMIAKMHIRGIVKGDSSYVKDRWCVFDGFMVFCLWVS |
| | | LVLQVFEIADIVDQMSPWGMLRIPRPLIMIRAFRIYFRFELP |
| | | RTRITNILKRSGEQIWSVSIFLLFFLLLYGILGVQMFGTFTY |
| | | HCVVNDTKPGNVTWNSLAIPDTHCSPELEEGYQCPPGFKC |
| | | MDLEDLGLSRQELGYSGFNEIGTSIFTVYEAASQEGWVFL |
| | | MYRAIDSFPRWRSYFYFITLIFFLAWLVKNVFIAVIIETFAEI |
| | | RVQFQQMWGSRSSTTSTATTQMFHEDAAGGWQVAVDVN |
| | | KPQGRAPACLQKMMRSSVFHMFILSMVTVDVIVAASNYY |
| | | KGENFRRQYDEFYLAEVAFTVLFDLEALLKIWCLGFTGYIS |
| | | SSLHKFELLLVIGTTLHVYPDLYHSQFTYFQVLRVVRLIKIS |
| | | PALEDFVYKIFGPGKKLGSLVVFTASLLIVMSAISLQMFCF |
| | | VEELDRFTTFPRAFMSMFQILTQEGWVDVMDQTLNAVGH |
| | | MWAPVVAIYFILYHLFATLILLSLFVAVILDNLELDEDLKK |
| | | LKQLKQSEANADTKEKLPLRLRIFEKFPNRPQMVKISKLPS |
| | | DFTVPKIRESFMKQFIDRQQQDTCCLLRSLPTTSSSSCDHSK |
| | | RSAIEDNKYIDQKLRKSVFSIRARNLLEKETAVTKILRACTR |
| | | QRMLSGSFEGQPAKERSILSVQHHIRQERRSLRHGSNSQRIS |
| | | RGKSLETLTQDHSNTVRYRNAQREDSEIKMIQEKKEQAEM |
| | | KRKVQEEELRENHPYFDKPLFIVGREHRFRNFCRVVVRAR |
| | | FNASKTDPVTGAVKNTKYHQLYDLLGLVTYLDWVMIIVTI |
| | | CSCISMMFESPFRRVMHAPTLQIAEYVFVIFMSIELNLKIMA |
| | | DGLFFTPTAVIRDFGGVMDIFIYLVSLIFLCWMPQNVPAES |
| | | GAQLLMVLRCLRPLRIFKLVPQMRKVVRELFSGFKEIFLVS |
| | | ILLLTLMLVFASFGVQLFAGKLAKCNDPNIIRREDCNGIFRI |
| | | NVSVSKNLNLKLRPGEKKPGFWVPRVWANPRNFNFDNVG |
| | | NAMLALFEVLSLKGWVEVRDVIIHRVGPIHGIYIHVFVFLG |
| | | CMIGLTLFVGVVIANFNENKGTALLTVDQRRWEDLKSRLK |
| | | IAQPLHLPPRPDNDGFRAKMYDITQHPFFKRTIALLVLAQS |
| | | VLLSVKWDVEDPVTVPLATMSVVFTFIFVLEVTMKIIAMSP |
| | | AGFWQSRRNRYDLLVTSLGVVWVVLHFALLNAYTYMMG |
| | | ACVIVFRFFSICGKHVTLKMLLLTVVVSMYKSFFIIVGMFL |
| | | LLLCYAFAGVVLFGTVKYGENINRHANFSSAGKAITVLFRI |
| | | VTGEDWNKIMHDCMVQPPFCTPDEFTYWATDCGNYAGA |
| | | LMYFCSFYVIIAYIMLNLLVAIIVENFSLFYSTEEDQLLSYN |
| | | DLRHFQIIWNMVDDKREGVIPTFRVKFLLRLLRGRLEVDL |
| | | DKDKLLFKHMCYEMERLHNGGDVTFHDVLSMLSYRSVDI |
| | | RKSLQLEELLAREQLEYTIEEEVAKQTIRMWLKKCLKRIRA |
| | | KQQQSCSIIHSLRESQQQELSRFLNPPSIETTQPSEDTNANSQ |
| | | DNSMQPETSSQQQLLSPTLSDRGGSRQDAADAGKPQRKFG |
| | | QWRLPSAPKPISHSVSSVNLRFGGRTTMKSVVCKMNPMTD |
| | | AASCGSEVKKWWTRQLTVESDESGDDLLDI |
| 36 | hNALCN_Δ364- | MLKRKQSSRVEAQPVTDFGPDESLSDNADILWINKPWVHS |
| | 374 | LLRICAIISVISVCMNTPMTFEHYPPLQYVTFTLDTLLMFLY |
| | | TAEMIAKMHIRGIVKGDSSYVKDRWCVFDGFMVFCLWVS |
| | | LVLQVFEIADIVDQMSPWGMLRIPRPLIMIRAFRIYFRFELP |
| | | RTRITNILKRSGEQIWSVSIFLLFFLLLYGILGVQMFGTFTY |
| | | HCVVNDTKPGNVTWNSLAIPDTHCSPELEEGYQCPPGFKC |
| | | MDLEDLGLSRQELGYSGFNEIGTSIFTVYEAASQEGWVFL |
| | | MYRAIDSFPRWRSYFYFITLIFFLAWLVKNVFIAVIIETFAEI |
| | | RVQFQQMWGSRSSTTSTATTQMFHEDAAGGWQLVAACL |
| | | QKMMRSSVFHMFILSMVTVDVIVAASNYYKGENFRRQYD |
| | | EFYLAEVAFTVLFDLEALLKIWCLGFTGYISSSLHKFELLLV |
| | | IGTTLHVYPDLYHSQFTYFQVLRVVRLIKISPALEDFVYKIF |
| | | GPGKKLGSLVVFTASLLIVMSAISLQMFCFVEELDRFTTFP |
| | | RAFMSMFQILTQEGWVDVMDQTLNAVGHMWAPVVAIYF |
| | | ILYHLFATLILLSLFVAVILDNLELDEDLKKLKQLKQSEAN |
| | | ADTKEKLPLRLRIFEKFPNRPQMVKISKLPSDFTVPKIRESF |
| | | MKQFIDRQQQDTCCLLRSLPTTSSSSCDHSKRSAIEDNKYI |
| | | DQKLRKSVFSIRARNLLEKETAVTKILRACTRQRMLSGSFE |
| | | GQPAKERSILSVQHHIRQERRSLRHGSNSQRISRGKSLETLT |
| | | QDHSNTVRYRNAQREDSEIKMIQEKKEQAEMKRKVQEEE |
| | | LRENHPYFDKPLFIVGREHRFRNFCRVVVRARFNASKTDP |
| | | VTGAVKNTKYHQLYDLLGLVTYLDWVMIIVTICSCISMMF |
| | | ESPFRRVMHAPTLQIAEYVFVIFMSIELNLKIMADGLFFTPT |
| | | AVIRDFGGVMDIFIYLVSLIFLCWMPQNVPAESGAQLLMV |
| | | LRCLRPLRIFKLVPQMRKVVRELFSGFKEIFLVSILLLTLML |
| | | VFASFGVQLFAGKLAKCNDPNIIRREDCNGIFRINVSVSKN |
| | | LNLKLRPGEKKPGFWVPRVWANPRNFNFDNVGNAMLALF |
| | | EVLSLKGWVEVRDVIIHRVGPIHGIYIHVFVFLGCMIGLTLF |
| | | VGVVIANFNENKGTALLTVDQRRWEDLKSRLKIAQPLHLP |
| | | PRPDNDGFRAKMYDITQHPFFKRTIALLVLAQSVLLSVKW |
| | | DVEDPVTVPLATMSVVFTFIFVLEVTMKIIAMSPAGFWQSR |
| | | RNRYDLLVTSLGVVWVVLHFALLNAYTYMMGACVIVFRF |
| | | FSICGKHVTLKMLLLTVVVSMYKSFFIIVGMFLLLLCYAFA |
| | | GVVLFGTVKYGENINRHANFSSAGKAITVLFRIVTGEDWN |
| | | KIMHDCMVQPPFCTPDEFTYWATDCGNYAGALMYFCSFY |
| | | VIIAYIMLNLLVAIIVENFSLFYSTEEDQLLSYNDLRHFQIIW |
| | | NMVDDKREGVIPTFRVKFLLRLLRGRLEVDLDKDKLLFKH |
| | | MCYEMERLHNGGDVTFHDVLSMLSYRSVDIRKSLQLEELL |
| | | AREQLEYTIEEEVAKQTIRMWLKKCLKRIRAKQQQSCSIIH |
| | | SLRESQQQELSRFLNPPSIETTQPSEDTNANSQDNSMQPETS |
| | | SQQQLLSPTLSDRGGSRQDAADAGKPQRKFGQWRLPSAP |
| | | KPISHSVSSVNLRFGGRTTMKSVVCKMNPMTDAASCGSEV |
| | | KKWWTRQLTVESDESGDDLLDI |
| 37 | hNALCN_Δ662- | MLKRKQSSRVEAQPVTDFGPDESLSDNADILWINKPWVHS |
| | 667 | LLRICAIISVISVCMNTPMTFEHYPPLQYVTFTLDTLLMFLY |
| | | TAEMIAKMHIRGIVKGDSSYVKDRWCVFDGFMVFCLWVS |
| | | LVLQVFEIADIVDQMSPWGMLRIPRPLIMIRAFRIYFRFELP |
| | | RTRITNILKRSGEQIWSVSIFLLFFLLLYGILGVQMFGTFTY |
| | | HCVVNDTKPGNVTWNSLAIPDTHCSPELEEGYQCPPGFKC |
| | | MDLEDLGLSRQELGYSGFNEIGTSIFTVYEAASQEGWVFL |
| | | MYRAIDSFPRWRSYFYFITLIFFLAWLVKNVFIAVIIETFAEI |
| | | RVQFQQMWGSRSSTTSTATTQMFHEDAAGGWQLVAVDV |
| | | NKPQGRAPACLQKMMRSSVFHMFILSMVTVDVIVAASNY |
| | | YKGENFRRQYDEFYLAEVAFTVLFDLEALLKIWCLGFTGY |
| | | ISSSLHKFELLLVIGTTLHVYPDLYHSQFTYFQVLRVVRLIK |
| | | ISPALEDFVYKIFGPGKKLGSLVVFTASLLIVMSAISLQMFC |
| | | FVEELDRFTTFPRAFMSMFQILTQEGWVDVMDQTLNAVG |
| | | HMWAPVVAIYFILYHLFATLILLSLFVAVILDNLELDEDLK |
| | | KLKQLKQSEANADTKEKLPLRLRIFEKFPNRPQMVKISKLP |
| | | SDFTVPKIRESDRQQQDTCCLLRSLPTTSSSSCDHSKRSAIE |
| | | DNKYIDQKLRKSVFSIRARNLLEKETAVTKILRACTRQRML |
| | | SGSFEGQPAKERSILSVQHHIRQERRSLRHGSNSQRISRGKS |
| | | LETLTQDHSNTVRYRNAQREDSEIKMIQEKKEQAEMKRK |
| | | VQEEELRENHPYFDKPLFIVGREHRFRNFCRVVVRARFNA |
| | | SKTDPVTGAVKNTKYHQLYDLLGLVTYLDWVMIIVTICSC |
| | | ISMMFESPFRRVMHAPTLQIAEYVFVIFMSIELNLKIMADG |
| | | LFFTPTAVIRDFGGVMDIFIYLVSLIFLCWMPQNVPAESGA |
| | | QLLMVLRCLRPLRIFKLVPQMRKVVRELFSGFKEIFLVSILL |
| | | LTLMLVFASFGVQLFAGKLAKCNDPNIIRREDCNGIFRINV |
| | | SVSKNLNLKLRPGEKKPGFWVPRVWANPRNFNFDNVGNA |
| | | MLALFEVLSLKGWVEVRDVIIHRVGPIHGIYIHVFVFLGCM |
| | | IGLTLFVGVVIANFNENKGTALLTVDQRRWEDLKSRLKIA |
| | | QPLHLPPRPDNDGFRAKMYDITQHPFFKRTIALLVLAQSVL |
| | | LSVKWDVEDPVTVPLATMSVVFTFIFVLEVTMKIIAMSPA |
| | | GFWQSRRNRYDLLVTSLGVVWVVLHFALLNAYTYMMGA |
| | | CVIVFRFFSICGKHVTLKMLLLTVVVSMYKSFFIIVGMFLLL |
| | | LCYAFAGVVLFGTVKYGENINRHANFSSAGKAITVLFRIVT |
| | | GEDWNKIMHDCMVQPPFCTPDEFTYWATDCGNYAGALM |
| | | YFCSFYVIIAYIMLNLLVAIIVENFSLFYSTEEDQLLSYNDLR |
| | | HFQIIWNMVDDKREGVIPTFRVKFLLRLLRGRLEVDLDKD |
| | | KLLFKHMCYEMERLHNGGDVTFHDVLSMLSYRSVDIRKS |
| | | LQLEELLAREQLEYTIEEEVAKQTIRMWLKKCLKRIRAKQ |
| | | QQSCSIIHSLRESQQQELSRFLNPPSIETTQPSEDTNANSQDN |
| | | SMQPETSSQQQLLSPTLSDRGGSRQDAADAGKPQRKFGQ |
| | | WRLPSAPKPISHSVSSVNLRFGGRTTMKSVVCKMNPMTDA |
| | | ASCGSEVKKWWTRQLTVESDESGDDLLDI |
| 38 | hNALCN_Δ1570- | MLKRKQSSRVEAQPVTDFGPDESLSDNADILWINKPWVHS |
| | 1738 | LLRICAIISVISVCMNTPMTFEHYPPLQYVTFTLDTLLMFLY |
| | | TAEMIAKMHIRGIVKGDSSYVKDRWCVFDGFMVFCLWVS |
| | | LVLQVFEIADIVDQMSPWGMLRIPRPLIMIRAFRIYFRFELP |
| | | RTRITNILKRSGEQIWSVSIFLLFFLLLYGILGVQMFGTFTY |
| | | HCVVNDTKPGNVTWNSLAIPDTHCSPELEEGYQCPPGFKC |
| | | MDLEDLGLSRQELGYSGFNEIGTSIFTVYEAASQEGWVFL |
| | | MYRAIDSFPRWRSYFYFITLIFFLAWLVKNVFIAVIIETFAEI |
| | | RVQFQQMWGSRSSTTSTATTQMFHEDAAGGWQLVAVDV |
| | | NKPQGRAPACLQKMMRSSVFHMFILSMVTVDVIVAASNY |
| | | YKGENFRRQYDEFYLAEVAFTVLFDLEALLKIWCLGFTGY |
| | | ISSSLHKFELLLVIGTTLHVYPDLYHSQFTYFQVLRVVRLIK |
| | | ISPALEDFVYKIFGPGKKLGSLVVFTASLLIVMSAISLQMFC |
| | | FVEELDRFTTFPRAFMSMFQILTQEGWVDVMDQTLNAVG |
| | | HMWAPVVAIYFILYHLFATLILLSLFVAVILDNLELDEDLK |
| | | KLKQLKQSEANADTKEKLPLRLRIFEKFPNRPQMVKISKLP |
| | | SDFTVPKIRESFMKQFIDRQQQDTCCLLRSLPTTSSSSCDHS |
| | | KRSAIEDNKYIDQKLRKSVFSIRARNLLEKETAVTKILRAC |
| | | TRQRMLSGSFEGQPAKERSILSVQHHIRQERRSLRHGSNSQ |
| | | RISRGKSLETLTQDHSNTVRYRNAQREDSEIKMIQEKKEQA |
| | | EMKRKVQEEELRENHPYFDKPLFIVGREHRFRNFCRVVVR |
| | | ARFNASKTDPVTGAVKNTKYHQLYDLLGLVTYLDWVMII |
| | | VTICSCISMMFESPFRRVMHAPTLQIAEYVFVIFMSIELNLKI |
| | | MADGLFFTPTAVIRDFGGVMDIFIYLVSLIFLCWMPQNVPA |
| | | ESGAQLLMVLRCLRPLRIFKLVPQMRKVVRELFSGFKEIFL |
| | | VSILLLTLMLVFASFGVQLFAGKLAKCNDPNIIRREDCNGIF |
| | | RINVSVSKNLNLKLRPGEKKPGFWVPRVWANPRNFNFDN |
| | | VGNAMLALFEVLSLKGWVEVRDVIIHRVGPIHGIYIHVFVF |
| | | LGCMIGLTLFVGVVIANFNENKGTALLTVDQRRWEDLKSR |
| | | LKIAQPLHLPPRPDNDGFRAKMYDITQHPFFKRTIALLVLA |
| | | QSVLLSVKWDVEDPVTVPLATMSVVFTFIFVLEVTMKIIA |
| | | MSPAGFWQSRRNRYDLLVTSLGVVWVVLHFALLNAYTY |
| | | MMGACVIVFRFFSICGKHVTLKMLLLTVVVSMYKSFFIIVG |
| | | MFLLLLCYAFAGVVLFGTVKYGENINRHANFSSAGKAITV |
| | | LFRIVTGEDWNKIMHDCMVQPPFCTPDEFTYWATDCGNY |
| | | AGALMYFCSFYVIIAYIMLNLLVAIIVENFSLFYSTEEDQLL |
| | | SYNDLRHFQIIWNMVDDKREGVIPTFRVKFLLRLLRGRLE |
| | | VDLDKDKLLFKHMCYEMERLHNGGDVTFHDVLSMLSYR |
| | | SVDIRKSLQLEELLAREQLEYTIEEEVAK |
| 39 | hNALCN_Δ1638- | MLKRKQSSRVEAQPVTDFGPDESLSDNADILWINKPWVHS |
| | 1738 | LLRICAIISVISVCMNTPMTFEHYPPLQYVTFTLDTLLMFLY |
| | | TAEMIAKMHIRGIVKGDSSYVKDRWCVFDGFMVFCLWVS |
| | | LVLQVFEIADIVDQMSPWGMLRIPRPLIMIRAFRIYFRFELP |
| | | RTRITNILKRSGEQIWSVSIFLLFFLLLYGILGVQMFGTFTY |
| | | HCVVNDTKPGNVTWNSLAIPDTHCSPELEEGYQCPPGFKC |
| | | MDLEDLGLSRQELGYSGFNEIGTSIFTVYEAASQEGWVFL |
| | | MYRAIDSFPRWRSYFYFITLIFFLAWLVKNVFIAVIIETFAEI |
| | | RVQFQQMWGSRSSTTSTATTQMFHEDAAGGWQLVAVDV |
| | | NKPQGRAPACLQKMMRSSVFHMFILSMVTVDVIVAASNY |
| | | YKGENFRRQYDEFYLAEVAFTVLFDLEALLKIWCLGFTGY |
| | | ISSSLHKFELLLVIGTTLHVYPDLYHSQFTYFQVLRVVRLIK |
| | | ISPALEDFVYKIFGPGKKLGSLVVFTASLLIVMSAISLQMFC |
| | | FVEELDRFTTFPRAFMSMFQILTQEGWVDVMDQTLNAVG |
| | | HMWAPVVAIYFILYHLFATLILLSLFVAVILDNLELDEDLK |
| | | KLKQLKQSEANADTKEKLPLRLRIFEKFPNRPQMVKISKLP |
| | | SDFTVPKIRESFMKQFIDRQQQDTCCLLRSLPTTSSSSCDHS |
| | | KRSAIEDNKYIDQKLRKSVFSIRARNLLEKETAVTKILRAC |
| | | TRQRMLSGSFEGQPAKERSILSVQHHIRQERRSLRHGSNSQ |
| | | RISRGKSLETLTQDHSNTVRYRNAQREDSEIKMIQEKKEQA |
| | | EMKRKVQEEELRENHPYFDKPLFIVGREHRFRNFCRVVVR |
| | | ARFNASKTDPVTGAVKNTKYHQLYDLLGLVTYLDWVMII |
| | | VTICSCISMMFESPFRRVMHAPTLQIAEYVFVIFMSIELNLKI |
| | | MADGLFFTPTAVIRDFGGVMDIFIYLVSLIFLCWMPQNVPA |
| | | ESGAQLLMVLRCLRPLRIFKLVPQMRKVVRELFSGFKEIFL |
| | | VSILLLTLMLVFASFGVQLFAGKLAKCNDPNIIRREDCNGIF |
| | | RINVSVSKNLNLKLRPGEKKPGFWVPRVWANPRNFNFDN |
| | | VGNAMLALFEVLSLKGWVEVRDVIIHRVGPIHGIYIHVFVF |
| | | LGCMIGLTLFVGVVIANFNENKGTALLTVDQRRWEDLKSR |
| | | LKIAQPLHLPPRPDNDGFRAKMYDITQHPFFKRTIALLVLA |
| | | QSVLLSVKWDVEDPVTVPLATMSVVFTFIFVLEVTMKIIA |
| | | MSPAGFWQSRRNRYDLLVTSLGVVWVVLHFALLNAYTY |
| | | MMGACVIVFRFFSICGKHVTLKMLLLTVVVSMYKSFFIIVG |
| | | MFLLLLCYAFAGVVLFGTVKYGENINRHANFSSAGKAITV |
| | | LFRIVTGEDWNKIMHDCMVQPPFCTPDEFTYWATDCGNY |
| | | AGALMYFCSFYVIIAYIMLNLLVAIIVENFSLFYSTEEDQLL |
| | | SYNDLRHFQIIWNMVDDKREGVIPTFRVKFLLRLLRGRLE |
| | | VDLDKDKLLFKHMCYEMERLHNGGDVTFHDVLSMLSYR |
| | | SVDIRKSLQLEELLAREQLEYTIEEEVAKQTIRMWLKKCLK |
| | | RIRAKQQQSCSIIHSLRESQQQELSRFLNPPSIETTQPSEDTN |
| | | ANSQDNSMQPETS |
| 40 | hNALCN_W359A | MLKRKQSSRVEAQPVTDFGPDESLSDNADILWINKPWVHS |
| | | LLRICAIISVISVCMNTPMTFEHYPPLQYVTFTLDTLLMFLY |
| | | TAEMIAKMHIRGIVKGDSSYVKDRWCVFDGFMVFCLWVS |
| | | LVLQVFEIADIVDQMSPWGMLRIPRPLIMIRAFRIYFRFELP |
| | | RTRITNILKRSGEQIWSVSIFLLFFLLLYGILGVQMFGTFTY |
| | | HCVVNDTKPGNVTWNSLAIPDTHCSPELEEGYQCPPGFKC |
| | | MDLEDLGLSRQELGYSGFNEIGTSIFTVYEAASQEGWVFL |
| | | MYRAIDSFPRWRSYFYFITLIFFLAWLVKNVFIAVIIETFAEI |
| | | RVQFQQMWGSRSSTTSTATTQMFHEDAAGGAQLVAVDV |
| | | NKPQGRAPACLQKMMRSSVFHMFILSMVTVDVIVAASNY |
| | | YKGENFRRQYDEFYLAEVAFTVLFDLEALLKIWCLGFTGY |
| | | ISSSLHKFELLLVIGTTLHVYPDLYHSQFTYFQVLRVVRLIK |
| | | ISPALEDFVYKIFGPGKKLGSLVVFTASLLIVMSAISLQMFC |
| | | FVEELDRFTTFPRAFMSMFQILTQEGWVDVMDQTLNAVG |
| | | HMWAPVVAIYFILYHLFATLILLSLFVAVILDNLELDEDLK |
| | | KLKQLKQSEANADTKEKLPLRLRIFEKFPNRPQMVKISKLP |
| | | SDFTVPKIRESFMKQFIDRQQQDTCCLLRSLPTTSSSSCDHS |
| | | KRSAIEDNKYIDQKLRKSVFSIRARNLLEKETAVTKILRAC |
| | | TRQRMLSGSFEGQPAKERSILSVQHHIRQERRSLRHGSNSQ |
| | | RISRGKSLETLTQDHSNTVRYRNAQREDSEIKMIQEKKEQA |
| | | EMKRKVQEEELRENHPYFDKPLFIVGREHRFRNFCRVVVR |
| | | ARFNASKTDPVTGAVKNTKYHQLYDLLGLVTYLDWVMII |
| | | VTICSCISMMFESPFRRVMHAPTLQIAEYVFVIFMSIELNLKI |
| | | MADGLFFTPTAVIRDFGGVMDIFIYLVSLIFLCWMPQNVPA |
| | | ESGAQLLMVLRCLRPLRIFKLVPQMRKVVRELFSGFKEIFL |
| | | VSILLLTLMLVFASFGVQLFAGKLAKCNDPNIIRREDCNGIF |
| | | RINVSVSKNLNLKLRPGEKKPGFWVPRVWANPRNFNFDN |
| | | VGNAMLALFEVLSLKGWVEVRDVIIHRVGPIHGIYIHVFVF |
| | | LGCMIGLTLFVGVVIANFNENKGTALLTVDQRRWEDLKSR |
| | | LKIAQPLHLPPRPDNDGFRAKMYDITQHPFFKRTIALLVLA |
| | | QSVLLSVKWDVEDPVTVPLATMSVVFTFIFVLEVTMKIIA |
| | | MSPAGFWQSRRNRYDLLVTSLGVVWVVLHFALLNAYTY |
| | | MMGACVIVFRFFSICGKHVTLKMLLLTVVVSMYKSFFIIVG |
| | | MFLLLLCYAFAGVVLFGTVKYGENINRHANFSSAGKAITV |
| | | LFRIVTGEDWNKIMHDCMVQPPFCTPDEFTYWATDCGNY |
| | | AGALMYFCSFYVIIAYIMLNLLVAIIVENFSLFYSTEEDQLL |
| | | SYNDLRHFQIIWNMVDDKREGVIPTFRVKFLLRLLRGRLE |
| | | VDLDKDKLLFKHMCYEMERLHNGGDVTFHDVLSMLSYR |
| | | SVDIRKSLQLEELLAREQLEYTIEEEVAKQTIRMWLKKCLK |
| | | RIRAKQQQSCSIIHSLRESQQQELSRFLNPPSIETTQPSEDTN |
| | | ANSQDNSMQPETSSQQQLLSPTLSDRGGSRQDAADAGKPQ |
| | | RKFGQWRLPSAPKPISHSVSSVNLRFGGRTTMKSVVCKMN |
| | | PMTDAASCGSEVKKWWTRQLTVESDESGDDLLDI |
| 41 | hNALCN_W359E | MLKRKQSSRVEAQPVTDFGPDESLSDNADILWINKPWVHS |
| | | LLRICAIISVISVCMNTPMTFEHYPPLQYVTFTLDTLLMFLY |
| | | TAEMIAKMHIRGIVKGDSSYVKDRWCVFDGFMVFCLWVS |
| | | LVLQVFEIADIVDQMSPWGMLRIPRPLIMIRAFRIYFRFELP |
| | | RTRITNILKRSGEQIWSVSIFLLFFLLLYGILGVQMFGTFTY |
| | | HCVVNDTKPGNVTWNSLAIPDTHCSPELEEGYQCPPGFKC |
| | | MDLEDLGLSRQELGYSGFNEIGTSIFTVYEAASQEGWVFL |
| | | MYRAIDSFPRWRSYFYFITLIFFLAWLVKNVFIAVIIETFAEI |
| | | RVQFQQMWGSRSSTTSTATTQMFHEDAAGGEQLVAVDV |
| | | NKPQGRAPACLQKMMRSSVFHMFILSMVTVDVIVAASNY |
| | | YKGENFRRQYDEFYLAEVAFTVLFDLEALLKIWCLGFTGY |
| | | ISSSLHKFELLLVIGTTLHVYPDLYHSQFTYFQVLRVVRLIK |
| | | ISPALEDFVYKIFGPGKKLGSLVVFTASLLIVMSAISLQMFC |
| | | FVEELDRFTTFPRAFMSMFQILTQEGWVDVMDQTLNAVG |
| | | HMWAPVVAIYFILYHLFATLILLSLFVAVILDNLELDEDLK |
| | | KLKQLKQSEANADTKEKLPLRLRIFEKFPNRPQMVKISKLP |
| | | SDFTVPKIRESFMKQFIDRQQQDTCCLLRSLPTTSSSSCDHS |
| | | KRSAIEDNKYIDQKLRKSVFSIRARNLLEKETAVTKILRAC |
| | | TRQRMLSGSFEGQPAKERSILSVQHHIRQERRSLRHGSNSQ |
| | | RISRGKSLETLTQDHSNTVRYRNAQREDSEIKMIQEKKEQA |
| | | EMKRKVQEEELRENHPYFDKPLFIVGREHRFRNFCRVVVR |
| | | ARFNASKTDPVTGAVKNTKYHQLYDLLGLVTYLDWVMII |
| | | VTICSCISMMFESPFRRVMHAPTLQIAEYVFVIFMSIELNLKI |
| | | MADGLFFTPTAVIRDFGGVMDIFIYLVSLIFLCWMPQNVPA |
| | | ESGAQLLMVLRCLRPLRIFKLVPQMRKVVRELFSGFKEIFL |
| | | VSILLLTLMLVFASFGVQLFAGKLAKCNDPNIIRREDCNGIF |
| | | RINVSVSKNLNLKLRPGEKKPGFWVPRVWANPRNFNFDN |
| | | VGNAMLALFEVLSLKGWVEVRDVIIHRVGPIHGIYIHVFVF |
| | | LGCMIGLTLFVGVVIANFNENKGTALLTVDQRRWEDLKSR |
| | | LKIAQPLHLPPRPDNDGFRAKMYDITQHPFFKRTIALLVLA |
| | | QSVLLSVKWDVEDPVTVPLATMSVVFTFIFVLEVTMKIIA |
| | | MSPAGFWQSRRNRYDLLVTSLGVVWVVLHFALLNAYTY |
| | | MMGACVIVFRFFSICGKHVTLKMLLLTVVVSMYKSFFIIVG |
| | | MFLLLLCYAFAGVVLFGTVKYGENINRHANFSSAGKAITV |
| | | LFRIVTGEDWNKIMHDCMVQPPFCTPDEFTYWATDCGNY |
| | | AGALMYFCSFYVIIAYIMLNLLVAIIVENFSLFYSTEEDQLL |
| | | SYNDLRHFQIIWNMVDDKREGVIPTFRVKFLLRLLRGRLE |
| | | VDLDKDKLLFKHMCYEMERLHNGGDVTFHDVLSMLSYR |
| | | SVDIRKSLQLEELLAREQLEYTIEEEVAKQTIRMWLKKCLK |
| | | RIRAKQQQSCSIIHSLRESQQQELSRFLNPPSIETTQPSEDTN |
| | | ANSQDNSMQPETSSQQQLLSPTLSDRGGSRQDAADAGKPQ |
| | | RKFGQWRLPSAPKPISHSVSSVNLRFGGRTTMKSVVCKMN |
| | | PMTDAASCGSEVKKWWTRQLTVESDESGDDLLDI |
| 42 | hNALCN-P340- | MLKRKQSSRVEAQPVTDFGPDESLSDNADILWINKPWVHS |
| | P345 | LLRICAIISVISVCMNTPMTFEHYPPLQYVTFTLDTLLMFLY |
| | (S340P/S341P/T342P/ | TAEMIAKMHIRGIVKGDSSYVKDRWCVFDGFMVFCLWVS |
| | T343P/S344P/T345P) | LVLQVFEIADIVDQMSPWGMLRIPRPLIMIRAFRIYFRFELP |
| | | RTRITNILKRSGEQIWSVSIFLLFFLLLYGILGVQMFGTFTY |
| | | HCVVNDTKPGNVTWNSLAIPDTHCSPELEEGYQCPPGFKC |
| | | MDLEDLGLSRQELGYSGFNEIGTSIFTVYEAASQEGWVFL |
| | | MYRAIDSFPRWRSYFYFITLIFFLAWLVKNVFIAVIIETFAEI |
| | | RVQFQQMWGSRPPPPPPATTQMFHEDAAGGWQLVAVDV |
| | | NKPQGRAPACLQKMMRSSVFHMFILSMVTVDVIVAASNY |
| | | YKGENFRRQYDEFYLAEVAFTVLFDLEALLKIWCLGFTGY |
| | | ISSSLHKFELLLVIGTTLHVYPDLYHSQFTYFQVLRVVRLIK |
| | | ISPALEDFVYKIFGPGKKLGSLVVFTASLLIVMSAISLQMFC |
| | | FVEELDRFTTFPRAFMSMFQILTQEGWVDVMDQTLNAVG |
| | | HMWAPVVAIYFILYHLFATLILLSLFVAVILDNLELDEDLK |
| | | KLKQLKQSEANADTKEKLPLRLRIFEKFPNRPQMVKISKLP |
| | | SDFTVPKIRESFMKQFIDRQQQDTCCLLRSLPTTSSSSCDHS |
| | | KRSAIEDNKYIDQKLRKSVFSIRARNLLEKETAVTKILRAC |
| | | TRQRMLSGSFEGQPAKERSILSVQHHIRQERRSLRHGSNSQ |
| | | RISRGKSLETLTQDHSNTVRYRNAQREDSEIKMIQEKKEQA |
| | | EMKRKVQEEELRENHPYFDKPLFIVGREHRFRNFCRVVVR |
| | | ARFNASKTDPVTGAVKNTKYHQLYDLLGLVTYLDWVMII |
| | | VTICSCISMMFESPFRRVMHAPTLQIAEYVFVIFMSIELNLKI |
| | | MADGLFFTPTAVIRDFGGVMDIFIYLVSLIFLCWMPQNVPA |
| | | ESGAQLLMVLRCLRPLRIFKLVPQMRKVVRELFSGFKEIFL |
| | | VSILLLTLMLVFASFGVQLFAGKLAKCNDPNIIRREDCNGIF |
| | | RINVSVSKNLNLKLRPGEKKPGFWVPRVWANPRNFNFDN |
| | | VGNAMLALFEVLSLKGWVEVRDVIIHRVGPIHGIYIHVFVF |
| | | LGCMIGLTLFVGVVIANFNENKGTALLTVDQRRWEDLKSR |
| | | LKIAQPLHLPPRPDNDGFRAKMYDITQHPFFKRTIALLVLA |
| | | QSVLLSVKWDVEDPVTVPLATMSVVFTFIFVLEVTMKIIA |
| | | MSPAGFWQSRRNRYDLLVTSLGVVWVVLHFALLNAYTY |
| | | MMGACVIVFRFFSICGKHVTLKMLLLTVVVSMYKSFFIIVG |
| | | MFLLLLCYAFAGVVLFGTVKYGENINRHANFSSAGKAITV |
| | | LFRIVTGEDWNKIMHDCMVQPPFCTPDEFTYWATDCGNY |
| | | AGALMYFCSFYVIIAYIMLNLLVAIIVENFSLFYSTEEDQLL |
| | | SYNDLRHFQIIWNMVDDKREGVIPTFRVKFLLRLLRGRLE |
| | | VDLDKDKLLFKHMCYEMERLHNGGDVTFHDVLSMLSYR |
| | | SVDIRKSLQLEELLAREQLEYTIEEEVAKQTIRMWLKKCLK |
| | | RIRAKQQQSCSIIHSLRESQQQELSRFLNPPSIETTQPSEDTN |
| | | ANSQDNSMQPETSSQQQLLSPTLSDRGGSRQDAADAGKPQ |
| | | RKFGQWRLPSAPKPISHSVSSVNLRFGGRTTMKSVVCKMN |
| | | PMTDAASCGSEVKKWWTRQLTVESDESGDDLLDI |
| 43 | hNALCN_ΔS340- | MLKRKQSSRVEAQPVTDFGPDESLSDNADILWINKPWVHS |
| | T345 | LLRICAIISVISVCMNTPMTFEHYPPLQYVTFTLDTLLMFLY |
| | | TAEMIAKMHIRGIVKGDSSYVKDRWCVFDGFMVFCLWVS |
| | | LVLQVFEIADIVDQMSPWGMLRIPRPLIMIRAFRIYFRFELP |
| | | RTRITNILKRSGEQIWSVSIFLLFFLLLYGILGVQMFGTFTY |
| | | HCVVNDTKPGNVTWNSLAIPDTHCSPELEEGYQCPPGFKC |
| | | MDLEDLGLSRQELGYSGFNEIGTSIFTVYEAASQEGWVFL |
| | | MYRAIDSFPRWRSYFYFITLIFFLAWLVKNVFIAVIIETFAEI |
| | | RVQFQQMWGSRATTQMFHEDAAGGWQLVAVDVNKPQG |
| | | RAPACLQKMMRSSVFHMFILSMVTVDVIVAASNYYKGEN |
| | | FRRQYDEFYLAEVAFTVLFDLEALLKIWCLGFTGYISSSLH |
| | | KFELLLVIGTTLHVYPDLYHSQFTYFQVLRVVRLIKISPALE |
| | | DFVYKIFGPGKKLGSLVVFTASLLIVMSAISLQMFCFVEEL |
| | | DRFTTFPRAFMSMFQILTQEGWVDVMDQTLNAVGHMWA |
| | | PVVAIYFILYHLFATLILLSLFVAVILDNLELDEDLKKLKQL |
| | | KQSEANADTKEKLPLRLRIFEKFPNRPQMVKISKLPSDFTV |
| | | PKIRESFMKQFIDRQQQDTCCLLRSLPTTSSSSCDHSKRSAI |
| | | EDNKYIDQKLRKSVFSIRARNLLEKETAVTKILRACTRQRM |
| | | LSGSFEGQPAKERSILSVQHHIRQERRSLRHGSNSQRISRGK |
| | | SLETLTQDHSNTVRYRNAQREDSEIKMIQEKKEQAEMKRK |
| | | VQEEELRENHPYFDKPLFIVGREHRFRNFCRVVVRARFNA |
| | | SKTDPVTGAVKNTKYHQLYDLLGLVTYLDWVMIIVTICSC |
| | | ISMMFESPFRRVMHAPTLQIAEYVFVIFMSIELNLKIMADG |
| | | LFFTPTAVIRDFGGVMDIFIYLVSLIFLCWMPQNVPAESGA |
| | | QLLMVLRCLRPLRIFKLVPQMRKVVRELFSGFKEIFLVSILL |
| | | LTLMLVFASFGVQLFAGKLAKCNDPNIIRREDCNGIFRINV |
| | | SVSKNLNLKLRPGEKKPGFWVPRVWANPRNFNFDNVGNA |
| | | MLALFEVLSLKGWVEVRDVIIHRVGPIHGIYIHVFVFLGCM |
| | | IGLTLFVGVVIANFNENKGTALLTVDQRRWEDLKSRLKIA |
| | | QPLHLPPRPDNDGFRAKMYDITQHPFFKRTIALLVLAQSVL |
| | | LSVKWDVEDPVTVPLATMSVVFTFIFVLEVTMKIIAMSPA |
| | | GFWQSRRNRYDLLVTSLGVVWVVLHFALLNAYTYMMGA |
| | | CVIVFRFFSICGKHVTLKMLLLTVVVSMYKSFFIIVGMFLLL |
| | | LCYAFAGVVLFGTVKYGENINRHANFSSAGKAITVLFRIVT |
| | | GEDWNKIMHDCMVQPPFCTPDEFTYWATDCGNYAGALM |
| | | YFCSFYVIIAYIMLNLLVAIIVENFSLFYSTEEDQLLSYNDLR |
| | | HFQIIWNMVDDKREGVIPTFRVKFLLRLLRGRLEVDLDKD |
| | | KLLFKHMCYEMERLHNGGDVTFHDVLSMLSYRSVDIRKS |
| | | LQLEELLAREQLEYTIEEEVAKQTIRMWLKKCLKRIRAKQ |
| | | QQSCSIIHSLRESQQQELSRFLNPPSIETTQPSEDTNANSQDN |
| | | SMQPETSSQQQLLSPTLSDRGGSRQDAADAGKPQRKFGQ |
| | | WRLPSAPKPISHSVSSVNLRFGGRTTMKSVVCKMNPMTDA |
| | | ASCGSEVKKWWTRQLTVESDESGDDLLDI |
| 44 | hNALCN_T345- | MLKRKQSSRVEAQPVTDFGPDESLSDNADILWINKPWVHS |
| | [GGGS]₃-A346 | LLRICAIISVISVCMNTPMTFEHYPPLQYVTFTLDTLLMFLY |
| | | TAEMIAKMHIRGIVKGDSSYVKDRWCVFDGFMVFCLWVS |
| | | LVLQVFEIADIVDQMSPWGMLRIPRPLIMIRAFRIYFRFELP |
| | | RTRITNILKRSGEQIWSVSIFLLFFLLLYGILGVQMFGTFTY |
| | | HCVVNDTKPGNVTWNSLAIPDTHCSPELEEGYQCPPGFKC |
| | | MDLEDLGLSRQELGYSGFNEIGTSIFTVYEAASQEGWVFL |
| | | MYRAIDSFPRWRSYFYFITLIFFLAWLVKNVFIAVIIETFAEI |
| | | RVQFQQMWGSRSSTTSTGGGSGGGSGGGSATTQMFHEDA |
| | | AGGWQLVAVDVNKPQGRAPACLQKMMRSSVFHMFILSM |
| | | VTVDVIVAASNYYKGENFRRQYDEFYLAEVAFTVLFDLEA |
| | | LLKIWCLGFTGYISSSLHKFELLLVIGTTLHVYPDLYHSQFT |
| | | YFQVLRVVRLIKISPALEDFVYKIFGPGKKLGSLVVFTASLL |
| | | IVMSAISLQMFCFVEELDRFTTFPRAFMSMFQILTQEGWVD |
| | | VMDQTLNAVGHMWAPVVAIYFILYHLFATLILLSLEVAVI |
| | | LDNLELDEDLKKLKQLKQSEANADTKEKLPLRLRIFEKFPN |
| | | RPQMVKISKLPSDFTVPKIRESFMKQFIDRQQQDTCCLLRS |
| | | LPTTSSSSCDHSKRSAIEDNKYIDQKLRKSVFSIRARNLLEK |
| | | ETAVTKILRACTRQRMLSGSFEGQPAKERSILSVQHHIRQE |
| | | RRSLRHGSNSQRISRGKSLETLTQDHSNTVRYRNAQREDSE |
| | | IKMIQEKKEQAEMKRKVQEEELRENHPYFDKPLFIVGREH |
| | | RFRNFCRVVVRARFNASKTDPVTGAVKNTKYHQLYDLLG |
| | | LVTYLDWVMIIVTICSCISMMFESPFRRVMHAPTLQIAEYV |
| | | FVIFMSIELNLKIMADGLFFTPTAVIRDFGGVMDIFIYLVSLI |
| | | FLCWMPQNVPAESGAQLLMVLRCLRPLRIFKLVPQMRKV |
| | | VRELFSGFKEIFLVSILLLTLMLVFASFGVQLFAGKLAKCN |
| | | DPNIIRREDCNGIFRINVSVSKNLNLKLRPGEKKPGFWVPR |
| | | VWANPRNFNFDNVGNAMLALFEVLSLKGWVEVRDVIIHR |
| | | VGPIHGIYIHVFVFLGCMIGLTLFVGVVIANFNENKGTALL |
| | | TVDQRRWEDLKSRLKIAQPLHLPPRPDNDGFRAKMYDITQ |
| | | HPFFKRTIALLVLAQSVLLSVKWDVEDPVTVPLATMSVVF |
| | | TFIFVLEVTMKIIAMSPAGFWQSRRNRYDLLVTSLGVVWV |
| | | VLHFALLNAYTYMMGACVIVFRFFSICGKHVTLKMLLLTV |
| | | VVSMYKSFFIIVGMFLLLLCYAFAGVVLFGTVKYGENINR |
| | | HANFSSAGKAITVLFRIVTGEDWNKIMHDCMVQPPFCTPD |
| | | EFTYWATDCGNYAGALMYFCSFYVIIAYIMLNLLVAIIVEN |
| | | FSLFYSTEEDQLLSYNDLRHFQIIWNMVDDKREGVIPTFRV |
| | | KFLLRLLRGRLEVDLDKDKLLFKHMCYEMERLHNGGDVT |
| | | FHDVLSMLSYRSVDIRKSLQLEELLAREQLEYTIEEEVAKQ |
| | | TIRMWLKKCLKRIRAKQQQSCSIIHSLRESQQQELSRFLNPP |
| | | SIETTQPSEDTNANSQDNSMQPETSSQQQLLSPTLSDRGGS |
| | | RQDAADAGKPQRKFGQWRLPSAPKPISHSVSSVNLRFGGR |
| | | TTMKSVVCKMNPMTDAASCGSEVKKWWTRQLTVESDES |
| | | GDDLLDI |
| 45 | hNALCN_Q370- | MLKRKQSSRVEAQPVTDFGPDESLSDNADILWINKPWVHS |
| | [GGGS]₃-G371 | LLRICAIISVISVCMNTPMTFEHYPPLQYVTFTLDTLLMFLY |
| | | TAEMIAKMHIRGIVKGDSSYVKDRWCVFDGFMVFCLWVS |
| | | LVLQVFEIADIVDQMSPWGMLRIPRPLIMIRAFRIYFRFELP |
| | | RTRITNILKRSGEQIWSVSIFLLFFLLLYGILGVQMFGTFTY |
| | | HCVVNDTKPGNVTWNSLAIPDTHCSPELEEGYQCPPGFKC |
| | | MDLEDLGLSRQELGYSGFNEIGTSIFTVYEAASQEGWVFL |
| | | MYRAIDSFPRWRSYFYFITLIFFLAWLVKNVFIAVIIETFAEI |
| | | RVQFQQMWGSRSSTTSTATTQMFHEDAAGGWQLVAVDV |
| | | NKPQGGGSGGGSGGGSGRAPACLQKMMRSSVFHMFILSM |
| | | VTVDVIVAASNYYKGENFRRQYDEFYLAEVAFTVLFDLEA |
| | | LLKIWCLGFTGYISSSLHKFELLLVIGTTLHVYPDLYHSQFT |
| | | YFQVLRVVRLIKISPALEDFVYKIFGPGKKLGSLVVFTASLL |
| | | IVMSAISLQMFCFVEELDRFTTFPRAFMSMFQILTQEGWVD |
| | | VMDQTLNAVGHMWAPVVAIYFILYHLFATLILLSLFVAVI |
| | | LDNLELDEDLKKLKQLKQSEANADTKEKLPLRLRIFEKFPN |
| | | RPQMVKISKLPSDFTVPKIRESFMKQFIDRQQQDTCCLLRS |
| | | LPTTSSSSCDHSKRSAIEDNKYIDQKLRKSVFSIRARNLLEK |
| | | ETAVTKILRACTRQRMLSGSFEGQPAKERSILSVQHHIRQE |
| | | RRSLRHGSNSQRISRGKSLETLTQDHSNTVRYRNAQREDSE |
| | | IKMIQEKKEQAEMKRKVQEEELRENHPYFDKPLFIVGREH |
| | | RFRNFCRVVVRARFNASKTDPVTGAVKNTKYHQLYDLLG |
| | | LVTYLDWVMIIVTICSCISMMFESPFRRVMHAPTLQIAEYV |
| | | FVIFMSIELNLKIMADGLFFTPTAVIRDFGGVMDIFIYLVSLI |
| | | FLCWMPQNVPAESGAQLLMVLRCLRPLRIFKLVPQMRKV |
| | | VRELFSGFKEIFLVSILLLTLMLVFASFGVQLFAGKLAKCN |
| | | DPNIIRREDCNGIFRINVSVSKNLNLKLRPGEKKPGFWVPR |
| | | VWANPRNFNFDNVGNAMLALFEVLSLKGWVEVRDVIIHR |
| | | VGPIHGIYIHVFVFLGCMIGLTLFVGVVIANFNENKGTALL |
| | | TVDQRRWEDLKSRLKIAQPLHLPPRPDNDGFRAKMYDITQ |
| | | HPFFKRTIALLVLAQSVLLSVKWDVEDPVTVPLATMSVVF |
| | | TFIFVLEVTMKIIAMSPAGFWQSRRNRYDLLVTSLGVVWV |
| | | VLHFALLNAYTYMMGACVIVFRFFSICGKHVTLKMLLLTV |
| | | VVSMYKSFFIIVGMFLLLLCYAFAGVVLFGTVKYGENINR |
| | | HANFSSAGKAITVLFRIVTGEDWNKIMHDCMVQPPFCTPD |
| | | EFTYWATDCGNYAGALMYFCSFYVIIAYIMLNLLVAIIVEN |
| | | FSLFYSTEEDQLLSYNDLRHFQIIWNMVDDKREGVIPTFRV |
| | | KFLLRLLRGRLEVDLDKDKLLFKHMCYEMERLHNGGDVT |
| | | FHDVLSMLSYRSVDIRKSLQLEELLAREQLEYTIEEEVAKQ |
| | | TIRMWLKKCLKRIRAKQQQSCSIIHSLRESQQQELSRFLNPP |
| | | SIETTQPSEDTNANSQDNSMQPETSSQQQLLSPTLSDRGGS |
| | | RQDAADAGKPQRKFGQWRLPSAPKPISHSVSSVNLRFGGR |
| | | TTMKSVVCKMNPMTDAASCGSEVKKWWTRQLTVESDES |
| | | GDDLLDI |
| 46 | hNALCN_T345- | MLKRKQSSRVEAQPVTDFGPDESLSDNADILWINKPWVHS |
| | [GGGS]₃- | LLRICAIISVISVCMNTPMTFEHYPPLQYVTFTLDTLLMFLY |
| | A346 + Q370- | TAEMIAKMHIRGIVKGDSSYVKDRWCVFDGFMVFCLWVS |
| | [GGGS]₃-G371 | LVLQVFEIADIVDQMSPWGMLRIPRPLIMIRAFRIYFRFELP |
| | | RTRITNILKRSGEQIWSVSIFLLFFLLLYGILGVQMFGTFTY |
| | | HCVVNDTKPGNVTWNSLAIPDTHCSPELEEGYQCPPGFKC |
| | | MDLEDLGLSRQELGYSGFNEIGTSIFTVYEAASQEGWVFL |
| | | MYRAIDSFPRWRSYFYFITLIFFLAWLVKNVFIAVIIETFAEI |
| | | RVQFQQMWGSRSSTTSTGGGSGGGSGGGSATTQMFHEDA |
| | | AGGWQLVAVDVNKPQGGGSGGGSGGGSGRAPACLQKM |
| | | MRSSVFHMFILSMVTVDVIVAASNYYKGENFRRQYDEFYL |
| | | AEVAFTVLFDLEALLKIWCLGFTGYISSSLHKFELLLVIGTT |
| | | LHVYPDLYHSQFTYFQVLRVVRLIKISPALEDFVYKIFGPG |
| | | KKLGSLVVFTASLLIVMSAISLQMFCFVEELDRFTTFPRAF |
| | | MSMFQILTQEGWVDVMDQTLNAVGHMWAPVVAIYFILY |
| | | HLFATLILLSLFVAVILDNLELDEDLKKLKQLKQSEANADT |
| | | KEKLPLRLRIFEKFPNRPQMVKISKLPSDFTVPKIRESFMKQ |
| | | FIDRQQQDTCCLLRSLPTTSSSSCDHSKRSAIEDNKYIDQKL |
| | | RKSVFSIRARNLLEKETAVTKILRACTRQRMLSGSFEGQPA |
| | | KERSILSVQHHIRQERRSLRHGSNSQRISRGKSLETLTQDHS |
| | | NTVRYRNAQREDSEIKMIQEKKEQAEMKRKVQEEELREN |
| | | HPYFDKPLFIVGREHRFRNFCRVVVRARFNASKTDPVTGA |
| | | VKNTKYHQLYDLLGLVTYLDWVMIIVTICSCISMMFESPFR |
| | | RVMHAPTLQIAEYVFVIFMSIELNLKIMADGLFFTPTAVIRD |
| | | FGGVMDIFIYLVSLIFLCWMPQNVPAESGAQLLMVLRCLR |
| | | PLRIFKLVPQMRKVVRELFSGFKEIFLVSILLLTLMLVFASF |
| | | GVQLFAGKLAKCNDPNIIRREDCNGIFRINVSVSKNLNLKL |
| | | RPGEKKPGFWVPRVWANPRNFNFDNVGNAMLALFEVLSL |
| | | KGWVEVRDVIIHRVGPIHGIYIHVFVFLGCMIGLTLFVGVVI |
| | | ANFNENKGTALLTVDQRRWEDLKSRLKIAQPLHLPPRPDN |
| | | DGFRAKMYDITQHPFFKRTIALLVLAQSVLLSVKWDVEDP |
| | | VTVPLATMSVVFTFIFVLEVTMKIIAMSPAGFWQSRRNRY |
| | | DLLVTSLGVVWVVLHFALLNAYTYMMGACVIVFRFFSICG |
| | | KHVTLKMLLLTVVVSMYKSFFIIVGMFLLLLCYAFAGVVL |
| | | FGTVKYGENINRHANFSSAGKAITVLFRIVTGEDWNKIMH |
| | | DCMVQPPFCTPDEFTYWATDCGNYAGALMYFCSFYVIIAY |
| | | IMLNLLVAIIVENFSLFYSTEEDQLLSYNDLRHFQIIWNMV |
| | | DDKREGVIPTFRVKFLLRLLRGRLEVDLDKDKLLFKHMCY |
| | | EMERLHNGGDVTFHDVLSMLSYRSVDIRKSLQLEELLARE |
| | | QLEYTIEEEVAKQTIRMWLKKCLKRIRAKQQQSCSIIHSLR |
| | | ESQQQELSRFLNPPSIETTQPSEDTNANSQDNSMQPETSSQ |
| | | QQLLSPTLSDRGGSRQDAADAGKPQRKFGQWRLPSAPKPI |
| | | SHSVSSVNLRFGGRTTMKSVVCKMNPMTDAASCGSEVKK |
| | | WWTRQLTVESDESGDDLLDI |
| 47 | hNALCN_P621-626 | MLKRKQSSRVEAQPVTDFGPDESLSDNADILWINKPWVHS |
| | (A621P/D622P/T623P/ | LLRICAIISVISVCMNTPMTFEHYPPLQYVTFTLDTLLMFLY |
| | K624P/E625P/K626P) | TAEMIAKMHIRGIVKGDSSYVKDRWCVFDGFMVFCLWVS |
| | | LVLQVFEIADIVDQMSPWGMLRIPRPLIMIRAFRIYFRFELP |
| | | RTRITNILKRSGEQIWSVSIFLLFFLLLYGILGVQMFGTFTY |
| | | HCVVNDTKPGNVTWNSLAIPDTHCSPELEEGYQCPPGFKC |
| | | MDLEDLGLSRQELGYSGFNEIGTSIFTVYEAASQEGWVFL |
| | | MYRAIDSFPRWRSYFYFITLIFFLAWLVKNVFIAVIIETFAEI |
| | | RVQFQQMWGSRSSTTSTATTQMFHEDAAGGWQLVAVDV |
| | | NKPQGRAPACLQKMMRSSVFHMFILSMVTVDVIVAASNY |
| | | YKGENFRRQYDEFYLAEVAFTVLFDLEALLKIWCLGFTGY |
| | | ISSSLHKFELLLVIGTTLHVYPDLYHSQFTYFQVLRVVRLIK |
| | | ISPALEDFVYKIFGPGKKLGSLVVFTASLLIVMSAISLQMFC |
| | | FVEELDRFTTFPRAFMSMFQILTQEGWVDVMDQTLNAVG |
| | | HMWAPVVAIYFILYHLFATLILLSLFVAVILDNLELDEDLK |
| | | KLKQLKQSEANPPPPPPLPLRLRIFEKFPNRPQMVKISKLPS |
| | | DFTVPKIRESFMKQFIDRQQQDTCCLLRSLPTTSSSSCDHSK |
| | | RSAIEDNKYIDQKLRKSVFSIRARNLLEKETAVTKILRACTR |
| | | QRMLSGSFEGQPAKERSILSVQHHIRQERRSLRHGSNSQRIS |
| | | RGKSLETLTQDHSNTVRYRNAQREDSEIKMIQEKKEQAEM |
| | | KRKVQEEELRENHPYFDKPLFIVGREHRFRNFCRVVVRAR |
| | | FNASKTDPVTGAVKNTKYHQLYDLLGLVTYLDWVMIIVTI |
| | | CSCISMMFESPFRRVMHAPTLQIAEYVFVIFMSIELNLKIMA |
| | | DGLFFTPTAVIRDFGGVMDIFIYLVSLIFLCWMPQNVPAES |
| | | GAQLLMVLRCLRPLRIFKLVPQMRKVVRELFSGFKEIFLVS |
| | | ILLLTLMLVFASFGVQLFAGKLAKCNDPNIIRREDCNGIFRI |
| | | NVSVSKNLNLKLRPGEKKPGFWVPRVWANPRNFNFDNVG |
| | | NAMLALFEVLSLKGWVEVRDVIIHRVGPIHGIYIHVFVFLG |
| | | CMIGLTLFVGVVIANFNENKGTALLTVDQRRWEDLKSRLK |
| | | IAQPLHLPPRPDNDGFRAKMYDITQHPFFKRTIALLVLAQS |
| | | VLLSVKWDVEDPVTVPLATMSVVFTFIFVLEVTMKIIAMSP |
| | | AGFWQSRRNRYDLLVTSLGVVWVVLHFALLNAYTYMMG |
| | | ACVIVFRFFSICGKHVTLKMLLLTVVVSMYKSFFIIVGMFL |
| | | LLLCYAFAGVVLFGTVKYGENINRHANFSSAGKAITVLFRI |
| | | VTGEDWNKIMHDCMVQPPFCTPDEFTYWATDCGNYAGA |
| | | LMYFCSFYVIIAYIMLNLLVAIIVENFSLFYSTEEDQLLSYN |
| | | DLRHFQIIWNMVDDKREGVIPTFRVKFLLRLLRGRLEVDL |
| | | DKDKLLFKHMCYEMERLHNGGDVTFHDVLSMLSYRSVDI |
| | | RKSLQLEELLAREQLEYTIEEEVAKQTIRMWLKKCLKRIRA |
| | | KQQQSCSIIHSLRESQQQELSRFLNPPSIETTQPSEDTNANSQ |
| | | DNSMQPETSSQQQLLSPTLSDRGGSRQDAADAGKPQRKFG |
| | | QWRLPSAPKPISHSVSSVNLRFGGRTTMKSVVCKMNPMTD |
| | | AASCGSEVKKWWTRQLTVESDESGDDLLDI |
| 48 | hNALCN_ΔA621- | MLKRKQSSRVEAQPVTDFGPDESLSDNADILWINKPWVHS |
| | K626 | LLRICAIISVISVCMNTPMTFEHYPPLQYVTFTLDTLLMFLY |
| | | TAEMIAKMHIRGIVKGDSSYVKDRWCVFDGFMVFCLWVS |
| | | LVLQVFEIADIVDQMSPWGMLRIPRPLIMIRAFRIYFRFELP |
| | | RTRITNILKRSGEQIWSVSIFLLFFLLLYGILGVQMFGTFTY |
| | | HCVVNDTKPGNVTWNSLAIPDTHCSPELEEGYQCPPGFKC |
| | | MDLEDLGLSRQELGYSGFNEIGTSIFTVYEAASQEGWVFL |
| | | MYRAIDSFPRWRSYFYFITLIFFLAWLVKNVFIAVIIETFAEI |
| | | RVQFQQMWGSRSSTTSTATTQMFHEDAAGGWQLVAVDV |
| | | NKPQGRAPACLQKMMRSSVFHMFILSMVTVDVIVAASNY |
| | | YKGENFRRQYDEFYLAEVAFTVLFDLEALLKIWCLGFTGY |
| | | ISSSLHKFELLLVIGTTLHVYPDLYHSQFTYFQVLRVVRLIK |
| | | ISPALEDFVYKIFGPGKKLGSLVVFTASLLIVMSAISLQMFC |
| | | FVEELDRFTTFPRAFMSMFQILTQEGWVDVMDQTLNAVG |
| | | HMWAPVVAIYFILYHLFATLILLSLFVAVILDNLELDEDLK |
| | | KLKQLKQSEANLPLRLRIFEKFPNRPQMVKISKLPSDFTVP |
| | | KIRESFMKQFIDRQQQDTCCLLRSLPTTSSSSCDHSKRSAIE |
| | | DNKYIDQKLRKSVFSIRARNLLEKETAVTKILRACTRQRML |
| | | SGSFEGQPAKERSILSVQHHIRQERRSLRHGSNSQRISRGKS |
| | | LETLTQDHSNTVRYRNAQREDSEIKMIQEKKEQAEMKRK |
| | | VQEEELRENHPYFDKPLFIVGREHRFRNFCRVVVRARFNA |
| | | SKTDPVTGAVKNTKYHQLYDLLGLVTYLDWVMIIVTICSC |
| | | ISMMFESPFRRVMHAPTLQIAEYVFVIFMSIELNLKIMADG |
| | | LFFTPTAVIRDFGGVMDIFIYLVSLIFLCWMPQNVPAESGA |
| | | QLLMVLRCLRPLRIFKLVPQMRKVVRELFSGFKEIFLVSILL |
| | | LTLMLVFASFGVQLFAGKLAKCNDPNIIRREDCNGIFRINV |
| | | SVSKNLNLKLRPGEKKPGFWVPRVWANPRNFNFDNVGNA |
| | | MLALFEVLSLKGWVEVRDVIIHRVGPIHGIYIHVFVFLGCM |
| | | IGLTLFVGVVIANFNENKGTALLTVDQRRWEDLKSRLKIA |
| | | QPLHLPPRPDNDGFRAKMYDITQHPFFKRTIALLVLAQSVL |
| | | LSVKWDVEDPVTVPLATMSVVFTFIFVLEVTMKIIAMSPA |
| | | GFWQSRRNRYDLLVTSLGVVWVVLHFALLNAYTYMMGA |
| | | CVIVFRFFSICGKHVTLKMLLLTVVVSMYKSFFIIVGMFLLL |
| | | LCYAFAGVVLFGTVKYGENINRHANFSSAGKAITVLFRIVT |
| | | GEDWNKIMHDCMVQPPFCTPDEFTYWATDCGNYAGALM |
| | | YFCSFYVIIAYIMLNLLVAIIVENFSLFYSTEEDQLLSYNDLR |
| | | HFQIIWNMVDDKREGVIPTFRVKFLLRLLRGRLEVDLDKD |
| | | KLLFKHMCYEMERLHNGGDVTFHDVLSMLSYRSVDIRKS |
| | | LQLEELLAREQLEYTIEEEVAKQTIRMWLKKCLKRIRAKQ |
| | | QQSCSIIHSLRESQQQELSRFLNPPSIETTQPSEDTNANSQDN |
| | | SMQPETSSQQQLLSPTLSDRGGSRQDAADAGKPQRKFGQ |
| | | WRLPSAPKPISHSVSSVNLRFGGRTTMKSVVCKMNPMTDA |
| | | ASCGSEVKKWWTRQLTVESDESGDDLLDI |
| 49 | hNALCN_K626- | MLKRKQSSRVEAQPVTDFGPDESLSDNADILWINKPWVHS |
| | [GGGS]₃-L627 | LLRICAIISVISVCMNTPMTFEHYPPLQYVTFTLDTLLMFLY |
| | | TAEMIAKMHIRGIVKGDSSYVKDRWCVFDGFMVFCLWVS |
| | | LVLQVFEIADIVDQMSPWGMLRIPRPLIMIRAFRIYFRFELP |
| | | RTRITNILKRSGEQIWSVSIFLLFFLLLYGILGVQMFGTFTY |
| | | HCVVNDTKPGNVTWNSLAIPDTHCSPELEEGYQCPPGFKC |
| | | MDLEDLGLSRQELGYSGFNEIGTSIFTVYEAASQEGWVFL |
| | | MYRAIDSFPRWRSYFYFITLIFFLAWLVKNVFIAVIIETFAEI |
| | | RVQFQQMWGSRSSTTSTATTQMFHEDAAGGWQLVAVDV |
| | | NKPQGRAPACLQKMMRSSVFHMFILSMVTVDVIVAASNY |
| | | YKGENFRRQYDEFYLAEVAFTVLFDLEALLKIWCLGFTGY |
| | | ISSSLHKFELLLVIGTTLHVYPDLYHSQFTYFQVLRVVRLIK |
| | | ISPALEDFVYKIFGPGKKLGSLVVFTASLLIVMSAISLQMFC |
| | | FVEELDRFTTFPRAFMSMFQILTQEGWVDVMDQTLNAVG |
| | | HMWAPVVAIYFILYHLFATLILLSLFVAVILDNLELDEDLK |
| | | KLKQLKQSEANADTKEKGGGSGGGSGGGSLPLRLRIFEKF |
| | | PNRPQMVKISKLPSDFTVPKIRESFMKQFIDRQQQDTCCLL |
| | | RSLPTTSSSSCDHSKRSAIEDNKYIDQKLRKSVFSIRARNLL |
| | | EKETAVTKILRACTRQRMLSGSFEGQPAKERSILSVQHHIR |
| | | QERRSLRHGSNSQRISRGKSLETLTQDHSNTVRYRNAQRE |
| | | DSEIKMIQEKKEQAEMKRKVQEEELRENHPYFDKPLFIVG |
| | | REHRFRNFCRVVVRARFNASKTDPVTGAVKNTKYHQLYD |
| | | LLGLVTYLDWVMIIVTICSCISMMFESPFRRVMHAPTLQIA |
| | | EYVFVIFMSIELNLKIMADGLFFTPTAVIRDFGGVMDIFIYL |
| | | VSLIFLCWMPQNVPAESGAQLLMVLRCLRPLRIFKLVPQM |
| | | RKVVRELFSGFKEIFLVSILLLTLMLVFASFGVQLFAGKLA |
| | | KCNDPNIIRREDCNGIFRINVSVSKNLNLKLRPGEKKPGFW |
| | | VPRVWANPRNFNFDNVGNAMLALFEVLSLKGWVEVRDVI |
| | | IHRVGPIHGIYIHVFVFLGCMIGLTLFVGVVIANFNENKGTA |
| | | LLTVDQRRWEDLKSRLKIAQPLHLPPRPDNDGFRAKMYDI |
| | | TQHPFFKRTIALLVLAQSVLLSVKWDVEDPVTVPLATMSV |
| | | VFTFIFVLEVTMKIIAMSPAGFWQSRRNRYDLLVTSLGVV |
| | | WVVLHFALLNAYTYMMGACVIVFRFFSICGKHVTLKMLL |
| | | LTVVVSMYKSFFIIVGMFLLLLCYAFAGVVLFGTVKYGENI |
| | | NRHANFSSAGKAITVLFRIVTGEDWNKIMHDCMVQPPFCT |
| | | PDEFTYWATDCGNYAGALMYFCSFYVIIAYIMLNLLVAIIV |
| | | ENFSLFYSTEEDQLLSYNDLRHFQIIWNMVDDKREGVIPTF |
| | | RVKFLLRLLRGRLEVDLDKDKLLFKHMCYEMERLHNGGD |
| | | VTFHDVLSMLSYRSVDIRKSLQLEELLAREQLEYTIEEEVA |
| | | KQTIRMWLKKCLKRIRAKQQQSCSIIHSLRESQQQELSRFL |
| | | NPPSIETTQPSEDTNANSQDNSMQPETSSQQQLLSPTLSDR |
| | | GGSRQDAADAGKPQRKFGQWRLPSAPKPISHSVSSVNLRF |
| | | GGRTTMKSVVCKMNPMTDAASCGSEVKKWWTRQLTVES |
| | | DESGDDLLDI |
| 50 | hNALCN_K835- | MLKRKQSSRVEAQPVTDFGPDESLSDNADILWINKPWVHS |
| | [GGGS]₃-P836 | LLRICAIISVISVCMNTPMTFEHYPPLQYVTFTLDTLLMFLY |
| | | TAEMIAKMHIRGIVKGDSSYVKDRWCVFDGFMVFCLWVS |
| | | LVLQVFEIADIVDQMSPWGMLRIPRPLIMIRAFRIYFRFELP |
| | | RTRITNILKRSGEQIWSVSIFLLFFLLLYGILGVQMFGTFTY |
| | | HCVVNDTKPGNVTWNSLAIPDTHCSPELEEGYQCPPGFKC |
| | | MDLEDLGLSRQELGYSGFNEIGTSIFTVYEAASQEGWVFL |
| | | MYRAIDSFPRWRSYFYFITLIFFLAWLVKNVFIAVIIETFAEI |
| | | RVQFQQMWGSRSSTTSTATTQMFHEDAAGGWQLVAVDV |
| | | NKPQGRAPACLQKMMRSSVFHMFILSMVTVDVIVAASNY |
| | | YKGENFRRQYDEFYLAEVAFTVLFDLEALLKIWCLGFTGY |
| | | ISSSLHKFELLLVIGTTLHVYPDLYHSQFTYFQVLRVVRLIK |
| | | ISPALEDFVYKIFGPGKKLGSLVVFTASLLIVMSAISLQMFC |
| | | FVEELDRFTTFPRAFMSMFQILTQEGWVDVMDQTLNAVG |
| | | HMWAPVVAIYFILYHLFATLILLSLFVAVILDNLELDEDLK |
| | | KLKQLKQSEANADTKEKLPLRLRIFEKFPNRPQMVKISKLP |
| | | SDFTVPKIRESFMKQFIDRQQQDTCCLLRSLPTTSSSSCDHS |
| | | KRSAIEDNKYIDQKLRKSVFSIRARNLLEKETAVTKILRAC |
| | | TRQRMLSGSFEGQPAKERSILSVQHHIRQERRSLRHGSNSQ |
| | | RISRGKSLETLTQDHSNTVRYRNAQREDSEIKMIQEKKEQA |
| | | EMKRKVQEEELRENHPYFDKGGGSGGGSGGGSPLFIVGRE |
| | | HRFRNFCRVVVRARFNASKTDPVTGAVKNTKYHQLYDLL |
| | | GLVTYLDWVMIIVTICSCISMMFESPFRRVMHAPTLQIAEY |
| | | VFVIFMSIELNLKIMADGLFFTPTAVIRDFGGVMDIFIYLVS |
| | | LIFLCWMPQNVPAESGAQLLMVLRCLRPLRIFKLVPQMRK |
| | | VVRELFSGFKEIFLVSILLLTLMLVFASFGVQLFAGKLAKC |
| | | NDPNIIRREDCNGIFRINVSVSKNLNLKLRPGEKKPGFWVP |
| | | RVWANPRNFNFDNVGNAMLALFEVLSLKGWVEVRDVIIH |
| | | RVGPIHGIYIHVFVFLGCMIGLTLFVGVVIANFNENKGTAL |
| | | LTVDQRRWEDLKSRLKIAQPLHLPPRPDNDGFRAKMYDIT |
| | | QHPFFKRTIALLVLAQSVLLSVKWDVEDPVTVPLATMSVV |
| | | FTFIFVLEVTMKIIAMSPAGFWQSRRNRYDLLVTSLGVVW |
| | | VVLHFALLNAYTYMMGACVIVFRFFSICGKHVTLKMLLLT |
| | | VVVSMYKSFFIIVGMFLLLLCYAFAGVVLFGTVKYGENIN |
| | | RHANFSSAGKAITVLFRIVTGEDWNKIMHDCMVQPPFCTP |
| | | DEFTYWATDCGNYAGALMYFCSFYVIIAYIMLNLLVAIIVE |
| | | NFSLFYSTEEDQLLSYNDLRHFQIIWNMVDDKREGVIPTFR |
| | | VKFLLRLLRGRLEVDLDKDKLLFKHMCYEMERLHNGGDV |
| | | TFHDVLSMLSYRSVDIRKSLQLEELLAREQLEYTIEEEVAK |
| | | QTIRMWLKKCLKRIRAKQQQSCSIIHSLRESQQQELSRFLN |
| | | PPSIETTQPSEDTNANSQDNSMQPETSSQQQLLSPTLSDRG |
| | | GSRQDAADAGKPQRKFGQWRLPSAPKPISHSVSSVNLRFG |
| | | GRTTMKSVVCKMNPMTDAASCGSEVKKWWTRQLTVESD |
| | | ESGDDLLDI |
| 51 | hNALCN_K626- | MLKRKQSSRVEAQPVTDFGPDESLSDNADILWINKPWVHS |
| | [GGGS]₃- | LLRICAIISVISVCMNTPMTFEHYPPLQYVTFTLDTLLMFLY |
| | L627 + K835- | TAEMIAKMHIRGIVKGDSSYVKDRWCVFDGFMVFCLWVS |
| | [GGGS]₃-P836 | LVLQVFEIADIVDQMSPWGMLRIPRPLIMIRAFRIYFRFELP |
| | | RTRITNILKRSGEQIWSVSIFLLFFLLLYGILGVQMFGTFTY |
| | | HCVVNDTKPGNVTWNSLAIPDTHCSPELEEGYQCPPGFKC |
| | | MDLEDLGLSRQELGYSGFNEIGTSIFTVYEAASQEGWVFL |
| | | MYRAIDSFPRWRSYFYFITLIFFLAWLVKNVFIAVIIETFAEI |
| | | RVQFQQMWGSRSSTTSTATTQMFHEDAAGGWQLVAVDV |
| | | NKPQGRAPACLQKMMRSSVFHMFILSMVTVDVIVAASNY |
| | | YKGENFRRQYDEFYLAEVAFTVLFDLEALLKIWCLGFTGY |
| | | ISSSLHKFELLLVIGTTLHVYPDLYHSQFTYFQVLRVVRLIK |
| | | ISPALEDFVYKIFGPGKKLGSLVVFTASLLIVMSAISLQMFC |
| | | FVEELDRFTTFPRAFMSMFQILTQEGWVDVMDQTLNAVG |
| | | HMWAPVVAIYFILYHLFATLILLSLFVAVILDNLELDEDLK |
| | | KLKQLKQSEANADTKEKGGGSGGGSGGGSLPLRLRIFEKF |
| | | PNRPQMVKISKLPSDFTVPKIRESFMKQFIDRQQQDTCCLL |
| | | RSLPTTSSSSCDHSKRSAIEDNKYIDQKLRKSVFSIRARNLL |
| | | EKETAVTKILRACTRQRMLSGSFEGQPAKERSILSVQHHIR |
| | | QERRSLRHGSNSQRISRGKSLETLTQDHSNTVRYRNAQRE |
| | | DSEIKMIQEKKEQAEMKRKVQEEELRENHPYFDKGGGSG |
| | | GGSGGGSPLFIVGREHRFRNFCRVVVRARFNASKTDPVTG |
| | | AVKNTKYHQLYDLLGLVTYLDWVMIIVTICSCISMMFESP |
| | | FRRVMHAPTLQIAEYVFVIFMSIELNLKIMADGLFFTPTAVI |
| | | RDFGGVMDIFIYLVSLIFLCWMPQNVPAESGAQLLMVLRC |
| | | LRPLRIFKLVPQMRKVVRELFSGFKEIFLVSILLLTLMLVFA |
| | | SFGVQLFAGKLAKCNDPNIIRREDCNGIFRINVSVSKNLNL |
| | | KLRPGEKKPGFWVPRVWANPRNFNFDNVGNAMLALFEVL |
| | | SLKGWVEVRDVIIHRVGPIHGIYIHVFVFLGCMIGLTLFVG |
| | | VVIANFNENKGTALLTVDQRRWEDLKSRLKIAQPLHLPPR |
| | | PDNDGFRAKMYDITQHPFFKRTIALLVLAQSVLLSVKWDV |
| | | EDPVTVPLATMSVVFTFIFVLEVTMKIIAMSPAGFWQSRRN |
| | | RYDLLVTSLGVVWVVLHFALLNAYTYMMGACVIVFRFFSI |
| | | CGKHVTLKMLLLTVVVSMYKSFFIIVGMFLLLLCYAFAGV |
| | | VLFGTVKYGENINRHANFSSAGKAITVLFRIVTGEDWNKI |
| | | MHDCMVQPPFCTPDEFTYWATDCGNYAGALMYFCSFYVI |
| | | IAYIMLNLLVAIIVENFSLFYSTEEDQLLSYNDLRHFQIIWN |
| | | MVDDKREGVIPTFRVKFLLRLLRGRLEVDLDKDKLLFKH |
| | | MCYEMERLHNGGDVTFHDVLSMLSYRSVDIRKSLQLEELL |
| | | AREQLEYTIEEEVAKQTIRMWLKKCLKRIRAKQQQSCSIIH |
| | | SLRESQQQELSRFLNPPSIETTQPSEDTNANSQDNSMQPETS |
| | | SQQQLLSPTLSDRGGSRQDAADAGKPQRKFGQWRLPSAP |
| | | KPISHSVSSVNLRFGGRTTMKSVVCKMNPMTDAASCGSEV |
| | | KKWWTRQLTVESDESGDDLLDI |
| 52 | hUNC79_1-2400* | MSTKAEQFASKIRYLQEYHNRVLHNIYPVPSGTDIANTLKY |
| | | FSQTLLSILSRTGKKENQDASNLTVPMTMCLFPVPFPLTPSL |
| | | RPQVSSINPTVTRSLLYSVLRDAPSERGPQSRDAQLSDYPSL |
| | | DYQGLYVTLVTLLDLVPLLQHGQHDLGQSIFYTTTCLLPFL |
| | | NDDILSTLPYTMISTLATFPPFLHKDIIEYLSTSFLPMAILGSS |
| | | RREGVPAHVNLSASSMLMIAMQYTSNPVYHCQLLECLMK |
| | | YKQEVWKDLLYVIAYGPSQVKPPAVQMLFHYWPNLKPPG |
| | | AISEYRGLQYTAWNPIHCQHIECHNAINKPAVKMCIDPSLS |
| | | VALGDKPPPLYLCEECSERIAGDHSEWLIDVLLPQAEISAIC |
| | | QKKNCSSHVRRAVVTCFSAGCCGRHGNRPVRYCKRCHSN |
| | | HHSNEVGAAAETHLYQTSPPPINTRECGAEELVCAVEAVIS |
| | | LLKEAEFHAEQREHELNRRRQLGLSSSHHSLDNADFDNKD |
| | | DDKHDQRLLSQFGIWFLVSLCTPSENTPTESLARLVAMVF |
| | | QWFHSTAYMMDDEVGSLVEKLKPQFVTKWLKTVCDVRF |
| | | DVMVMCLLPKPMEFARVGGYWDKSCSTVTQLKEGLNRIL |
| | | CLIPYNVINQSVWECIMPEWLEAIRTEVPDNQLKEFREVLS |
| | | KMFDIELCPLPFSMEEMFGFISCRFTGYPSSVQEQALLWLH |
| | | VLSELDIMVPLQLLISMFSDGVNSVKELANQRKSRVSELAG |
| | | NLASRRVSVASDPGRRVQHNMLSPFHSPFQSPFRSPLRSPF |
| | | RSPFKNFGHPGGRTIDFDCEDDEMNLNCFILMFDLLLKQM |
| | | ELQDDGITMGLEHSLSKDIISIINNVFQAPWGGSHTCQKDE |
| | | KAIECNLCQSSILCYQLACELLERLAPKEESRLVEPTDSLED |
| | | SLLSSRPEFIIGPEGEEEENPASKHGENPGNCTEPVEHAAVK |
| | | NDTERKFCYQQLPVTLRLIYTIFQEMAKFEEPDILFNMLNC |
| | | LKILCLHGECLYIARKDHPQFLAYIQDHMLIASLWRVVKSE |
| | | FSQLSSLAVPLLLHALSLPHGADIFWTIINGNFNSKDWKMR |
| | | FEAVEKVAVICRFLDIHSVTKNHLLKYSLAHAFCCFLTAVE |
| | | DVNPAVATRAGLLLDTIKRPALQGLCLCLDFQFDTVVKDR |
| | | PTILSKLLLLHFLKQDIPALSWEFFVNRFETLSLEAQLHLDC |
| | | NKEFPFPTTITAVRTNVANLSDAALWKIKRARFARNRQKS |
| | | VRSLRDSVKGPVESKRALSLPETLTSKIRQQSPENDNTIKDL |
| | | LPEDAGIDHQTVHQLITVLMKFMAKDESSAESDISSAKAFN |
| | | TVKRHLYVLLGYDQQEGCFMIAPQKMRLSTCFNAFIAGIA |
| | | QVMDYNINLGKHLLPLVVQVLKYCSCPQLRHYFQQPPRCS |
| | | LWSLKPHIRQMWLKALLVILYKYPYRDCDISKILLHLIHIT |
| | | VNTLNAQYHSCKPHATAGPLYSDNSNISRYSEKEKGEIELA |
| | | EYRETGALQDSLLHCVREESIPKKKLRSFKQKSLDIGNADS |
| | | LLFTLDEHRRKSCIDRCDIEKPPTQAAYIAQRPNDPGRSRQ |
| | | NSATRPDNSEIPENPAMEGFPDARRPVIPEVRLNCMETFEV |
| | | KVDSPVKPAPKEDLDLIDLSSDSTSGPEKHSILSTSDSDSLV |
| | | FEPLPPLRIVESDEEEETMNQGDDGPSGKNAASSPSVPSHPS |
| | | VLSLSTAPLVQVSVEDCSKDFSSKDSGNNQSAGNTDSALIT |
| | | LEDPMDAEGSSKPEELPEFSCGSPLTLKQKRDLLQKSFALP |
| | | EMSLDDHPDPGTEGEKPGELMPSSGAKTVLLKVPEDAENP |
| | | TESEKPDTSAESDTEQNPERKVEEDGAEESEFKIQIVPRQRK |
| | | QRKIAVSAIQREYLDISFNILDKLGEQKDPDPSTKGLSTLEM |
| | | PRESSSAPTLDAGVPETSSHSSISTQYRQMKRGSLGVLTMS |
| | | QLMKRQLEHQSSAPHNISNWDTEQIQPGKRQCNVPTCLNP |
| | | DLEGQPLRMRGATKSSLLSAPSIVSMFVPAPEEFTDEQPTV |
| | | MTDKCHDCGAILEEYDEETLGLAIVVLSTFIHLSPDLAAPL |
| | | LLDIMQSVGRLASSTTFSNQAESMMVPGNAAGVAKQFLR |
| | | CIFHQLAPNGIFPQLFQSTIKDGTFLRTLASSLMDFNELSSIA |
| | | ALSQLLEGLNNKKNLPAGGAMIRCLENIATFMEALPMDSP |
| | | SSLWTTISNQFQTFFAKLPCVLPLKCSLDSSLRIMICLLKIPS |
| | | TNATRSLLEPFSKLLSFVIQNAVFTLAYLVELCGLCYRAFT |
| | | KERDKFYLSRSVVLELLQALKLKSPLPDTNLLLLVQFICAD |
| | | AGTKLAESTILSKQMIASVPGCGTAAMECVRQYINEVLDF |
| | | MADMHTLTKLKSHMKTCSQPLHEDTFGGHLKVGLAQIAA |
| | | MDISRGNHRDNKAVIRYLPWLYHPPSAMQQGGSGGSVSK |
| | | GEELFTGVVPILVELDGDVNGHKFSVSGEGEGDATYGKLT |
| | | LKFICTTGKLPVPWPTLVTTLTYGVQCFSRYPDHMKQHDF |
| | | FKSAMPEGYVQERTIFFKDDGNYKTRAEVKFEGDTLVNRI |
| | | ELKGIDFKEDGNILGHKLEYNYNSHNVYIMADKQKNGIKV |
| | | NFKIRHNIEDGSVQLADHYQQNTPIGDGPVLLPDNHYLSTQ |
| | | SALSKDPNEKRDHMVLLEFVTAAGITLGMDELYKGGSGG |
| | | SDYKDDDDKGSGDYKDDDDK |
| 53 | hUNC79_1-467* | MSTKAEQFASKIRYLQEYHNRVLHNIYPVPSGTDIANTLKY |
| | | FSQTLLSILSRTGKKENQDASNLTVPMTMCLFPVPFPLTPSL |
| | | RPQVSSINPTVTRSLLYSVLRDAPSERGPQSRDAQLSDYPSL |
| | | DYQGLYVTLVTLLDLVPLLQHGQHDLGQSIFYTTTCLLPFL |
| | | NDDILSTLPYTMISTLATFPPFLHKDIIEYLSTSFLPMAILGSS |
| | | RREGVPAHVNLSASSMLMIAMQYTSNPVYHCQLLECLMK |
| | | YKQEVWKDLLYVIAYGPSQVKPPAVQMLFHYWPNLKPPG |
| | | AISEYRGLQYTAWNPIHCQHIECHNAINKPAVKMCIDPSLS |
| | | VALGDKPPPLYLCEECSERIAGDHSEWLIDVLLPQAEISAIC |
| | | QKKNCSSHVRRAVVTCFSAGCCGRHGNRPVRYCKRCHSN |
| | | HHSNEVGAAAETHLYQTSPPPINTRECGAEELVCAVEAVIS |
| | | LLKEAEFHAEQREHELGGSGGSVSKGEELFTGVVPILVELD |
| | | GDVNGHKFSVSGEGEGDATYGKLTLKFICTTGKLPVPWPT |
| | | LVTTLTYGVQCFSRYPDHMKQHDFFKSAMPEGYVQERTIF |
| | | FKDDGNYKTRAEVKFEGDTLVNRIELKGIDFKEDGNILGH |
| | | KLEYNYNSHNVYIMADKQKNGIKVNFKIRHNIEDGSVQLA |
| | | DHYQQNTPIGDGPVLLPDNHYLSTQSALSKDPNEKRDHM |
| | | VLLEFVTAAGITLGMDELYKGGSGGSDYKDDDDKGSGDY |
| | | KDDDDK |
| 54 | hUNC79_468- | MNRRRQLGLSSSHHSLDNADFDNKDDDKHDQRLLSQFGI |
| | 2635* | WFLVSLCTPSENTPTESLARLVAMVFQWFHSTAYMMDDE |
| | | VGSLVEKLKPQFVTKWLKTVCDVRFDVMVMCLLPKPMEF |
| | | ARVGGYWDKSCSTVTQLKEGLNRILCLIPYNVINQSVWEC |
| | | IMPEWLEAIRTEVPDNQLKEFREVLSKMFDIELCPLPFSME |
| | | EMFGFISCRFTGYPSSVQEQALLWLHVLSELDIMVPLQLLIS |
| | | MFSDGVNSVKELANQRKSRVSELAGNLASRRVSVASDPG |
| | | RRVQHNMLSPFHSPFQSPFRSPLRSPFRSPFKNFGHPGGRTI |
| | | DFDCEDDEMNLNCFILMFDLLLKQMELQDDGITMGLEHSL |
| | | SKDIISIINNVFQAPWGGSHTCQKDEKAIECNLCQSSILCYQ |
| | | LACELLERLAPKEESRLVEPTDSLEDSLLSSRPEFIIGPEGEE |
| | | EENPASKHGENPGNCTEPVEHAAVKNDTERKFCYQQLPVT |
| | | LRLIYTIFQEMAKFEEPDILFNMLNCLKILCLHGECLYIARK |
| | | DHPQFLAYIQDHMLIASLWRVVKSEFSQLSSLAVPLLLHAL |
| | | SLPHGADIFWTIINGNFNSKDWKMRFEAVEKVAVICRFLDI |
| | | HSVTKNHLLKYSLAHAFCCFLTAVEDVNPAVATRAGLLL |
| | | DTIKRPALQGLCLCLDFQFDTVVKDRPTILSKLLLLHFLKQ |
| | | DIPALSWEFFVNRFETLSLEAQLHLDCNKEFPFPTTITAVRT |
| | | NVANLSDAALWKIKRARFARNRQKSVRSLRDSVKGPVES |
| | | KRALSLPETLTSKIRQQSPENDNTIKDLLPEDAGIDHQTVH |
| | | QLITVLMKFMAKDESSAESDISSAKAFNTVKRHLYVLLGY |
| | | DQQEGCFMIAPQKMRLSTCFNAFIAGIAQVMDYNINLGKH |
| | | LLPLVVQVLKYCSCPQLRHYFQQPPRCSLWSLKPHIRQMW |
| | | LKALLVILYKYPYRDCDISKILLHLIHITVNTLNAQYHSCKP |
| | | HATAGPLYSDNSNISRYSEKEKGEIELAEYRETGALQDSLL |
| | | HCVREESIPKKKLRSFKQKSLDIGNADSLLFTLDEHRRKSCI |
| | | DRCDIEKPPTQAAYIAQRPNDPGRSRQNSATRPDNSEIPENP |
| | | AMEGFPDARRPVIPEVRLNCMETFEVKVDSPVKPAPKEDL |
| | | DLIDLSSDSTSGPEKHSILSTSDSDSLVFEPLPPLRIVESDEEE |
| | | ETMNQGDDGPSGKNAASSPSVPSHPSVLSLSTAPLVQVSV |
| | | EDCSKDFSSKDSGNNQSAGNTDSALITLEDPMDAEGSSKPE |
| | | ELPEFSCGSPLTLKQKRDLLQKSFALPEMSLDDHPDPGTEG |
| | | EKPGELMPSSGAKTVLLKVPEDAENPTESEKPDTSAESDTE |
| | | QNPERKVEEDGAEESEFKIQIVPRQRKQRKIAVSAIQREYL |
| | | DISFNILDKLGEQKDPDPSTKGLSTLEMPRESSSAPTLDAGV |
| | | PETSSHSSISTQYRQMKRGSLGVLTMSQLMKRQLEHQSSA |
| | | PHNISNWDTEQIQPGKRQCNVPTCLNPDLEGQPLRMRGAT |
| | | KSSLLSAPSIVSMFVPAPEEFTDEQPTVMTDKCHDCGAILE |
| | | EYDEETLGLAIVVLSTFIHLSPDLAAPLLLDIMQSVGRLASS |
| | | TTFSNQAESMMVPGNAAGVAKQFLRCIFHQLAPNGIFPQL |
| | | FQSTIKDGTFLRTLASSLMDFNELSSIAALSQLLEGLNNKK |
| | | NLPAGGAMIRCLENIATFMEALPMDSPSSLWTTISNQFQTF |
| | | FAKLPCVLPLKCSLDSSLRIMICLLKIPSTNATRSLLEPFSKL |
| | | LSFVIQNAVFTLAYLVELCGLCYRAFTKERDKFYLSRSVVL |
| | | ELLQALKLKSPLPDTNLLLLVQFICADAGTKLAESTILSKQ |
| | | MIASVPGCGTAAMECVRQYINEVLDFMADMHTLTKLKSH |
| | | MKTCSQPLHEDTFGGHLKVGLAQIAAMDISRGNHRDNKA |
| | | VIRYLPWLYHPPSAMQQGPKEFIECVSHIRLLSWLLLGSLT |
| | | HNAVCPNASSPCLPIPLDAGSHVADHLIVILIGFPEQSKTSV |
| | | LHMCSLFHAFIFAQLWTVYCEQSAVATNLQNQNEFSFTAI |
| | | LTALEFWSRVTPSILQLMAHNKVMVEMVCLHVISLMEAL |
| | | QECNSTIFVKLIPMWLPMIQSNIKHLSAGLQLRLQAIQNHV |
| | | NHHSLRTLPGSGQSSAGLAALRKWLQCTQFKMAQVEIQSS |
| | | EAASQFYPLGGSGGSVSKGEELFTGVVPILVELDGDVNGH |
| | | KFSVSGEGEGDATYGKLTLKFICTTGKLPVPWPTLVTTLTY |
| | | GVQCFSRYPDHMKQHDFFKSAMPEGYVQERTIFFKDDGN |
| | | YKTRAEVKFEGDTLVNRIELKGIDFKEDGNILGHKLEYNY |
| | | NSHNVYIMADKQKNGIKVNFKIRHNIEDGSVQLADHYQQ |
| | | NTPIGDGPVLLPDNHYLSTQSALSKDPNEKRDHMVLLEFV |
| | | TAAGITLGMDELYKGGSGGSDYKDDDDKGSGDYKDDDD |
| | | K |
| 55 | hUNC79_1-800* | MSTKAEQFASKIRYLQEYHNRVLHNIYPVPSGTDIANTLKY |
| | | FSQTLLSILSRTGKKENQDASNLTVPMTMCLFPVPFPLTPSL |
| | | RPQVSSINPTVTRSLLYSVLRDAPSERGPQSRDAQLSDYPSL |
| | | DYQGLYVTLVTLLDLVPLLQHGQHDLGQSIFYTTTCLLPFL |
| | | NDDILSTLPYTMISTLATFPPFLHKDIIEYLSTSFLPMAILGSS |
| | | RREGVPAHVNLSASSMLMIAMQYTSNPVYHCQLLECLMK |
| | | YKQEVWKDLLYVIAYGPSQVKPPAVQMLFHYWPNLKPPG |
| | | AISEYRGLQYTAWNPIHCQHIECHNAINKPAVKMCIDPSLS |
| | | VALGDKPPPLYLCEECSERIAGDHSEWLIDVLLPQAEISAIC |
| | | QKKNCSSHVRRAVVTCFSAGCCGRHGNRPVRYCKRCHSN |
| | | HHSNEVGAAAETHLYQTSPPPINTRECGAEELVCAVEAVIS |
| | | LLKEAEFHAEQREHELNRRRQLGLSSSHHSLDNADFDNKD |
| | | DDKHDQRLLSQFGIWFLVSLCTPSENTPTESLARLVAMVF |
| | | QWFHSTAYMMDDEVGSLVEKLKPQFVTKWLKTVCDVRF |
| | | DVMVMCLLPKPMEFARVGGYWDKSCSTVTQLKEGLNRIL |
| | | CLIPYNVINQSVWECIMPEWLEAIRTEVPDNQLKEFREVLS |
| | | KMFDIELCPLPFSMEEMFGFISCRFTGYPSSVQEQALLWLH |
| | | VLSELDIMVPLQLLISMFSDGVNSVKELANQRKSRVSELAG |
| | | NLASRRVSVASDPGRRVQHNMLSPFHSPFQSPFRSPLRSPF |
| | | RSPFKNFGHPGGRTIDFDCEDDEMNLNCGGSGGSVSKGEE |
| | | LFTGVVPILVELDGDVNGHKFSVSGEGEGDATYGKLTLKFI |
| | | CTTGKLPVPWPTLVTTLTYGVQCFSRYPDHMKQHDFFKSA |
| | | MPEGYVQERTIFFKDDGNYKTRAEVKFEGDTLVNRIELKG |
| | | IDFKEDGNILGHKLEYNYNSHNVYIMADKQKNGIKVNFKI |
| | | RHNIEDGSVQLADHYQQNTPIGDGPVLLPDNHYLSTQSAL |
| | | SKDPNEKRDHMVLLEFVTAAGITLGMDELYKGGSGGSDY |
| | | KDDDDKGSGDYKDDDDK |
| 56 | hUNC79_801- | MFILMFDLLLKQMELQDDGITMGLEHSLSKDIISIINNVFQA |
| | 2635* | PWGGSHTCQKDEKAIECNLCQSSILCYQLACELLERLAPKE |
| | | ESRLVEPTDSLEDSLLSSRPEFIIGPEGEEEENPASKHGENPG |
| | | NCTEPVEHAAVKNDTERKFCYQQLPVTLRLIYTIFQEMAK |
| | | FEEPDILFNMLNCLKILCLHGECLYIARKDHPQFLAYIQDH |
| | | MLIASLWRVVKSEFSQLSSLAVPLLLHALSLPHGADIFWTII |
| | | NGNFNSKDWKMRFEAVEKVAVICRFLDIHSVTKNHLLKY |
| | | SLAHAFCCFLTAVEDVNPAVATRAGLLLDTIKRPALQGLC |
| | | LCLDFQFDTVVKDRPTILSKLLLLHFLKQDIPALSWEFFVN |
| | | RFETLSLEAQLHLDCNKEFPFPTTITAVRTNVANLSDAALW |
| | | KIKRARFARNRQKSVRSLRDSVKGPVESKRALSLPETLTSK |
| | | IRQQSPENDNTIKDLLPEDAGIDHQTVHQLITVLMKFMAK |
| | | DESSAESDISSAKAFNTVKRHLYVLLGYDQQEGCFMIAPQ |
| | | KMRLSTCFNAFIAGIAQVMDYNINLGKHLLPLVVQVLKYC |
| | | SCPQLRHYFQQPPRCSLWSLKPHIRQMWLKALLVILYKYP |
| | | YRDCDISKILLHLIHITVNTLNAQYHSCKPHATAGPLYSDN |
| | | SNISRYSEKEKGEIELAEYRETGALQDSLLHCVREESIPKKK |
| | | LRSFKQKSLDIGNADSLLFTLDEHRRKSCIDRCDIEKPPTQA |
| | | AYIAQRPNDPGRSRQNSATRPDNSEIPENPAMEGFPDARRP |
| | | VIPEVRLNCMETFEVKVDSPVKPAPKEDLDLIDLSSDSTSG |
| | | PEKHSILSTSDSDSLVFEPLPPLRIVESDEEEETMNQGDDGP |
| | | SGKNAASSPSVPSHPSVLSLSTAPLVQVSVEDCSKDFSSKD |
| | | SGNNQSAGNTDSALITLEDPMDAEGSSKPEELPEFSCGSPL |
| | | TLKQKRDLLQKSFALPEMSLDDHPDPGTEGEKPGELMPSS |
| | | GAKTVLLKVPEDAENPTESEKPDTSAESDTEQNPERKVEE |
| | | DGAEESEFKIQIVPRQRKQRKIAVSAIQREYLDISFNILDKL |
| | | GEQKDPDPSTKGLSTLEMPRESSSAPTLDAGVPETSSHSSIS |
| | | TQYRQMKRGSLGVLTMSQLMKRQLEHQSSAPHNISNWDT |
| | | EQIQPGKRQCNVPTCLNPDLEGQPLRMRGATKSSLLSAPSI |
| | | VSMFVPAPEEFTDEQPTVMTDKCHDCGAILEEYDEETLGL |
| | | AIVVLSTFIHLSPDLAAPLLLDIMQSVGRLASSTTFSNQAES |
| | | MMVPGNAAGVAKQFLRCIFHQLAPNGIFPQLFQSTIKDGTF |
| | | LRTLASSLMDFNELSSIAALSQLLEGLNNKKNLPAGGAMIR |
| | | CLENIATFMEALPMDSPSSLWTTISNQFQTFFAKLPCVLPL |
| | | KCSLDSSLRIMICLLKIPSTNATRSLLEPFSKLLSFVIQNAVF |
| | | TLAYLVELCGLCYRAFTKERDKFYLSRSVVLELLQALKLK |
| | | SPLPDTNLLLLVQFICADAGTKLAESTILSKQMIASVPGCGT |
| | | AAMECVRQYINEVLDFMADMHTLTKLKSHMKTCSQPLHE |
| | | DTFGGHLKVGLAQIAAMDISRGNHRDNKAVIRYLPWLYH |
| | | PPSAMQQGPKEFIECVSHIRLLSWLLLGSLTHNAVCPNASS |
| | | PCLPIPLDAGSHVADHLIVILIGFPEQSKTSVLHMCSLFHAFI |
| | | FAQLWTVYCEQSAVATNLQNQNEFSFTAILTALEFWSRVT |
| | | PSILQLMAHNKVMVEMVCLHVISLMEALQECNSTIFVKLIP |
| | | MWLPMIQSNIKHLSAGLQLRLQAIQNHVNHHSLRTLPGSG |
| | | QSSAGLAALRKWLQCTQFKMAQVEIQSSEAASQFYPLGGS |
| | | GGSVSKGEELFTGVVPILVELDGDVNGHKFSVSGEGEGDA |
| | | TYGKLTLKFICTTGKLPVPWPTLVTTLTYGVQCFSRYPDH |
| | | MKQHDFFKSAMPEGYVQERTIFFKDDGNYKTRAEVKFEG |
| | | DTLVNRIELKGIDFKEDGNILGHKLEYNYNSHNVYIMADK |
| | | QKNGIKVNFKIRHNIEDGSVQLADHYQQNTPIGDGPVLLPD |
| | | NHYLSTQSALSKDPNEKRDHMVLLEFVTAAGITLGMDELY |
| | | KGGSGGSDYKDDDDKGSGDYKDDDDK |
| 57 | hUNC79_1-1400* | MSTKAEQFASKIRYLQEYHNRVLHNIYPVPSGTDIANTLKY |
| | | FSQTLLSILSRTGKKENQDASNLTVPMTMCLFPVPFPLTPSL |
| | | RPQVSSINPTVTRSLLYSVLRDAPSERGPQSRDAQLSDYPSL |
| | | DYQGLYVTLVTLLDLVPLLQHGQHDLGQSIFYTTTCLLPFL |
| | | NDDILSTLPYTMISTLATFPPFLHKDIIEYLSTSFLPMAILGSS |
| | | RREGVPAHVNLSASSMLMIAMQYTSNPVYHCQLLECLMK |
| | | YKQEVWKDLLYVIAYGPSQVKPPAVQMLFHYWPNLKPPG |
| | | AISEYRGLQYTAWNPIHCQHIECHNAINKPAVKMCIDPSLS |
| | | VALGDKPPPLYLCEECSERIAGDHSEWLIDVLLPQAEISAIC |
| | | QKKNCSSHVRRAVVTCFSAGCCGRHGNRPVRYCKRCHSN |
| | | HHSNEVGAAAETHLYQTSPPPINTRECGAEELVCAVEAVIS |
| | | LLKEAEFHAEQREHELNRRRQLGLSSSHHSLDNADFDNKD |
| | | DDKHDQRLLSQFGIWFLVSLCTPSENTPTESLARLVAMVF |
| | | QWFHSTAYMMDDEVGSLVEKLKPQFVTKWLKTVCDVRF |
| | | DVMVMCLLPKPMEFARVGGYWDKSCSTVTQLKEGLNRIL |
| | | CLIPYNVINQSVWECIMPEWLEAIRTEVPDNQLKEFREVLS |
| | | KMFDIELCPLPFSMEEMFGFISCRFTGYPSSVQEQALLWLH |
| | | VLSELDIMVPLQLLISMFSDGVNSVKELANQRKSRVSELAG |
| | | NLASRRVSVASDPGRRVQHNMLSPFHSPFQSPFRSPLRSPF |
| | | RSPFKNFGHPGGRTIDFDCEDDEMNLNCFILMFDLLLKQM |
| | | ELQDDGITMGLEHSLSKDIISIINNVFQAPWGGSHTCQKDE |
| | | KAIECNLCQSSILCYQLACELLERLAPKEESRLVEPTDSLED |
| | | SLLSSRPEFIIGPEGEEEENPASKHGENPGNCTEPVEHAAVK |
| | | NDTERKFCYQQLPVTLRLIYTIFQEMAKFEEPDILFNMLNC |
| | | LKILCLHGECLYIARKDHPQFLAYIQDHMLIASLWRVVKSE |
| | | FSQLSSLAVPLLLHALSLPHGADIFWTIINGNFNSKDWKMR |
| | | FEAVEKVAVICRFLDIHSVTKNHLLKYSLAHAFCCFLTAVE |
| | | DVNPAVATRAGLLLDTIKRPALQGLCLCLDFQFDTVVKDR |
| | | PTILSKLLLLHFLKQDIPALSWEFFVNRFETLSLEAQLHLDC |
| | | NKEFPFPTTITAVRTNVANLSDAALWKIKRARFARNRQKS |
| | | VRSLRDSVKGPVESKRALSLPETLTSKIRQQSPENDNTIKDL |
| | | LPEDAGIDHQTVHQLITVLMKFMAKDESSAESDISSAKAFN |
| | | TVKRHLYVLLGYDQQEGCFMIAPQKMRLSTCFNAFIAGIA |
| | | QVMDYNINLGKHLLPLVVQVLKYCSCPQLRHYFQQPPRCS |
| | | LWSLKPHIRQMWLKGGSGGSVSKGEELFTGVVPILVELDG |
| | | DVNGHKFSVSGEGEGDATYGKLTLKFICTTGKLPVPWPTL |
| | | VTTLTYGVQCFSRYPDHMKQHDFFKSAMPEGYVQERTIFF |
| | | KDDGNYKTRAEVKFEGDTLVNRIELKGIDFKEDGNILGHK |
| | | LEYNYNSHNVYIMADKQKNGIKVNFKIRHNIEDGSVQLAD |
| | | HYQQNTPIGDGPVLLPDNHYLSTQSALSKDPNEKRDHMVL |
| | | LEFVTAAGITLGMDELYKGGSGGSDYKDDDDKGSGDYKD |
| | | DDDK |
| 58 | hUNC79_1401- | MALLVILYKYPYRDCDISKILLHLIHITVNTLNAQYHSCKP |
| | 2635 | HATAGPLYSDNSNISRYSEKEKGEIELAEYRETGALQDSLL |
| | | HCVREESIPKKKLRSFKQKSLDIGNADSLLFTLDEHRRKSCI |
| | | DRCDIEKPPTQAAYIAQRPNDPGRSRQNSATRPDNSEIPENP |
| | | AMEGFPDARRPVIPEVRLNCMETFEVKVDSPVKPAPKEDL |
| | | DLIDLSSDSTSGPEKHSILSTSDSDSLVFEPLPPLRIVESDEEE |
| | | ETMNQGDDGPSGKNAASSPSVPSHPSVLSLSTAPLVQVSV |
| | | EDCSKDFSSKDSGNNQSAGNTDSALITLEDPMDAEGSSKPE |
| | | ELPEFSCGSPLTLKQKRDLLQKSFALPEMSLDDHPDPGTEG |
| | | EKPGELMPSSGAKTVLLKVPEDAENPTESEKPDTSAESDTE |
| | | QNPERKVEEDGAEESEFKIQIVPRQRKQRKIAVSAIQREYL |
| | | DISFNILDKLGEQKDPDPSTKGLSTLEMPRESSSAPTLDAGV |
| | | PETSSHSSISTQYRQMKRGSLGVLTMSQLMKRQLEHQSSA |
| | | PHNISNWDTEQIQPGKRQCNVPTCLNPDLEGQPLRMRGAT |
| | | KSSLLSAPSIVSMFVPAPEEFTDEQPTVMTDKCHDCGAILE |
| | | EYDEETLGLAIVVLSTFIHLSPDLAAPLLLDIMQSVGRLASS |
| | | TTFSNQAESMMVPGNAAGVAKQFLRCIFHQLAPNGIFPQL |
| | | FQSTIKDGTFLRTLASSLMDFNELSSIAALSQLLEGLNNKK |
| | | NLPAGGAMIRCLENIATFMEALPMDSPSSLWTTISNQFQTF |
| | | FAKLPCVLPLKCSLDSSLRIMICLLKIPSTNATRSLLEPFSKL |
| | | LSFVIQNAVFTLAYLVELCGLCYRAFTKERDKFYLSRSVVL |
| | | ELLQALKLKSPLPDTNLLLLVQFICADAGTKLAESTILSKQ |
| | | MIASVPGCGTAAMECVRQYINEVLDFMADMHTLTKLKSH |
| | | MKTCSQPLHEDTFGGHLKVGLAQIAAMDISRGNHRDNKA |
| | | VIRYLPWLYHPPSAMQQGPKEFIECVSHIRLLSWLLLGSLT |
| | | HNAVCPNASSPCLPIPLDAGSHVADHLIVILIGFPEQSKTSV |
| | | LHMCSLFHAFIFAQLWTVYCEQSAVATNLQNQNEFSFTAI |
| | | LTALEFWSRVTPSILQLMAHNKVMVEMVCLHVISLMEAL |
| | | QECNSTIFVKLIPMWLPMIQSNIKHLSAGLQLRLQAIQNHV |
| | | NHHSLRTLPGSGQSSAGLAALRKWLQCTQFKMAQVEIQSS |
| | | EAASQFYPL |
| 59 | hNALCN- | MASSSLPNLVPPGPHCLRPFTPESLAAIEQRAVEEEARLQR |
| | rNav1.4_N- | NKQMEIEEPERKPRSDLEAGKNLPLIYGDPPPEVIGIPLEDL |
| | term_chimera | DPYYSDKKTFIVLNKGKAIFRFSATPALYLLSPFSILLRICAII |
| | | SVISVCMNTPMTFEHYPPLQYVTFTLDTLLMFLYTAEMIA |
| | | KMHIRGIVKGDSSYVKDRWCVFDGFMVFCLWVSLVLQVF |
| | | EIADIVDQMSPWGMLRIPRPLIMIRAFRIYFRFELPRTRITNI |
| | | LKRSGEQIWSVSIFLLFFLLLYGILGVQMFGTFTYHCVVND |
| | | TKPGNVTWNSLAIPDTHCSPELEEGYQCPPGFKCMDLEDL |
| | | GLSRQELGYSGFNEIGTSIFTVYEAASQEGWVFLMYRAIDS |
| | | FPRWRSYFYFITLIFFLAWLVKNVFIAVIIETFAEIRVQFQQ |
| | | MWGSRSSTTSTATTQMFHEDAAGGWQLVAVDVNKPQGR |
| | | APACLQKMMRSSVFHMFILSMVTVDVIVAASNYYKGENF |
| | | RRQYDEFYLAEVAFTVLFDLEALLKIWCLGFTGYISSSLHK |
| | | FELLLVIGTTLHVYPDLYHSQFTYFQVLRVVRLIKISPALED |
| | | FVYKIFGPGKKLGSLVVFTASLLIVMSAISLQMFCFVEELD |
| | | RFTTFPRAFMSMFQILTQEGWVDVMDQTLNAVGHMWAP |
| | | VVAIYFILYHLFATLILLSLFVAVILDNLELDEDLKKLKQLK |
| | | QSEANADTKEKLPLRLRIFEKFPNRPQMVKISKLPSDFTVP |
| | | KIRESFMKQFIDRQQQDTCCLLRSLPTTSSSSCDHSKRSAIE |
| | | DNKYIDQKLRKSVFSIRARNLLEKETAVTKILRACTRQRML |
| | | SGSFEGQPAKERSILSVQHHIRQERRSLRHGSNSQRISRGKS |
| | | LETLTQDHSNTVRYRNAQREDSEIKMIQEKKEQAEMKRK |
| | | VQEEELRENHPYFDKPLFIVGREHRFRNFCRVVVRARFNA |
| | | SKTDPVTGAVKNTKYHQLYDLLGLVTYLDWVMIIVTICSC |
| | | ISMMFESPFRRVMHAPTLQIAEYVFVIFMSIELNLKIMADG |
| | | LFFTPTAVIRDFGGVMDIFIYLVSLIFLCWMPQNVPAESGA |
| | | QLLMVLRCLRPLRIFKLVPQMRKVVRELFSGFKEIFLVSILL |
| | | LTLMLVFASFGVQLFAGKLAKCNDPNIIRREDCNGIFRINV |
| | | SVSKNLNLKLRPGEKKPGFWVPRVWANPRNFNFDNVGNA |
| | | MLALFEVLSLKGWVEVRDVIIHRVGPIHGIYIHVFVFLGCM |
| | | IGLTLFVGVVIANFNENKGTALLTVDQRRWEDLKSRLKIA |
| | | QPLHLPPRPDNDGFRAKMYDITQHPFFKRTIALLVLAQSVL |
| | | LSVKWDVEDPVTVPLATMSVVFTFIFVLEVTMKIIAMSPA |
| | | GFWQSRRNRYDLLVTSLGVVWVVLHFALLNAYTYMMGA |
| | | CVIVFRFFSICGKHVTLKMLLLTVVVSMYKSFFIIVGMFLLL |
| | | LCYAFAGVVLFGTVKYGENINRHANFSSAGKAITVLFRIVT |
| | | GEDWNKIMHDCMVQPPFCTPDEFTYWATDCGNYAGALM |
| | | YFCSFYVIIAYIMLNLLVAIIVENFSLFYSTEEDQLLSYNDLR |
| | | HFQIIWNMVDDKREGVIPTFRVKFLLRLLRGRLEVDLDKD |
| | | KLLFKHMCYEMERLHNGGDVTFHDVLSMLSYRSVDIRKS |
| | | LQLEELLAREQLEYTIEEEVAKQTIRMWLKKCLKRIRAKQ |
| | | QQSCSIIHSLRESQQQELSRFLNPPSIETTQPSEDTNANSQDN |
| | | SMQPETSSQQQLLSPTLSDRGGSRQDAADAGKPQRKFGQ |
| | | WRLPSAPKPISHSVSSVNLRFGGRTTMKSVVCKMNPMTDA |
| | | ASCGSEVKKWWTRQLTVESDESGDDLLDI |
| 60 | hNALCN- | MLKRKQSSRVEAQPVTDFGPDESLSDNADILWINKPWVHS |
| | rNav1.4_DIII- | LLRICAIISVISVCMNTPMTFEHYPPLQYVTFTLDTLLMFLY |
| | DIV_linker_chimera | TAEMIAKMHIRGIVKGDSSYVKDRWCVFDGFMVFCLWVS |
| | | LVLQVFEIADIVDQMSPWGMLRIPRPLIMIRAFRIYFRFELP |
| | | RTRITNILKRSGEQIWSVSIFLLFFLLLYGILGVQMFGTFTY |
| | | HCVVNDTKPGNVTWNSLAIPDTHCSPELEEGYQCPPGFKC |
| | | MDLEDLGLSRQELGYSGFNEIGTSIFTVYEAASQEGWVFL |
| | | MYRAIDSFPRWRSYFYFITLIFFLAWLVKNVFIAVIIETFAEI |
| | | RVQFQQMWGSRSSTTSTATTQMFHEDAAGGWQLVAVDV |
| | | NKPQGRAPACLQKMMRSSVFHMFILSMVTVDVIVAASNY |
| | | YKGENFRRQYDEFYLAEVAFTVLFDLEALLKIWCLGFTGY |
| | | ISSSLHKFELLLVIGTTLHVYPDLYHSQFTYFQVLRVVRLIK |
| | | ISPALEDFVYKIFGPGKKLGSLVVFTASLLIVMSAISLQMFC |
| | | FVEELDRFTTFPRAFMSMFQILTQEGWVDVMDQTLNAVG |
| | | HMWAPVVAIYFILYHLFATLILLSLFVAVILDNLELDEDLK |
| | | KLKQLKQSEANADTKEKLPLRLRIFEKFPNRPQMVKISKLP |
| | | SDFTVPKIRESFMKQFIDRQQQDTCCLLRSLPTTSSSSCDHS |
| | | KRSAIEDNKYIDQKLRKSVFSIRARNLLEKETAVTKILRAC |
| | | TRQRMLSGSFEGQPAKERSILSVQHHIRQERRSLRHGSNSQ |
| | | RISRGKSLETLTQDHSNTVRYRNAQREDSEIKMIQEKKEQA |
| | | EMKRKVQEEELRENHPYFDKPLFIVGREHRFRNFCRVVVR |
| | | ARFNASKTDPVTGAVKNTKYHQLYDLLGLVTYLDWVMII |
| | | VTICSCISMMFESPFRRVMHAPTLQIAEYVFVIFMSIELNLKI |
| | | MADGLFFTPTAVIRDFGGVMDIFIYLVSLIFLCWMPQNVPA |
| | | ESGAQLLMVLRCLRPLRIFKLVPQMRKVVRELFSGFKEIFL |
| | | VSILLLTLMLVFASFGVQLFAGKLAKCNDPNIIRREDCNGIF |
| | | RINVSVSKNLNLKLRPGEKKPGFWVPRVWANPRNFNFDN |
| | | VGNAMLALFEVLSLKGWVEVRDVIIHRVGPIHGIYIHVFVF |
| | | LGCMIGLTLFVGVVIANFNENKGTFGGKDIFMTEEQKKYY |
| | | NAMKKLGSKKPQKPIPRPQNGFRAKMYDITQHPFFKRTIA |
| | | LLVLAQSVLLSVKWDVEDPVTVPLATMSVVFTFIFVLEVT |
| | | MKIIAMSPAGFWQSRRNRYDLLVTSLGVVWVVLHFALLN |
| | | AYTYMMGACVIVFRFFSICGKHVTLKMLLLTVVVSMYKS |
| | | FFIIVGMFLLLLCYAFAGVVLFGTVKYGENINRHANFSSAG |
| | | KAITVLFRIVTGEDWNKIMHDCMVQPPFCTPDEFTYWATD |
| | | CGNYAGALMYFCSFYVIIAYIMLNLLVAIIVENFSLFYSTEE |
| | | DQLLSYNDLRHFQIIWNMVDDKREGVIPTFRVKFLLRLLR |
| | | GRLEVDLDKDKLLFKHMCYEMERLHNGGDVTFHDVLSM |
| | | LSYRSVDIRKSLQLEELLAREQLEYTIEEEVAKQTIRMWLK |
| | | KCLKRIRAKQQQSCSIIHSLRESQQQELSRFLNPPSIETTQPS |
| | | EDTNANSQDNSMQPETSSQQQLLSPTLSDRGGSRQDAADA |
| | | GKPQRKFGQWRLPSAPKPISHSVSSVNLRFGGRTTMKSVV |
| | | CKMNPMTDAASCGSEVKKWWTRQLTVESDESGDDLLDI |
| 61 | hNALCN- | MLKRKQSSRVEAQPVTDFGPDESLSDNADILWINKPWVHS |
| | rNav1.4_C- | LLRICAIISVISVCMNTPMTFEHYPPLQYVTFTLDTLLMFLY |
| | term_chimera | TAEMIAKMHIRGIVKGDSSYVKDRWCVFDGFMVFCLWVS |
| | | LVLQVFEIADIVDQMSPWGMLRIPRPLIMIRAFRIYFRFELP |
| | | RTRITNILKRSGEQIWSVSIFLLFFLLLYGILGVQMFGTFTY |
| | | HCVVNDTKPGNVTWNSLAIPDTHCSPELEEGYQCPPGFKC |
| | | MDLEDLGLSRQELGYSGFNEIGTSIFTVYEAASQEGWVFL |
| | | MYRAIDSFPRWRSYFYFITLIFFLAWLVKNVFIAVIIETFAEI |
| | | RVQFQQMWGSRSSTTSTATTQMFHEDAAGGWQLVAVDV |
| | | NKPQGRAPACLQKMMRSSVFHMFILSMVTVDVIVAASNY |
| | | YKGENFRRQYDEFYLAEVAFTVLFDLEALLKIWCLGFTGY |
| | | ISSSLHKFELLLVIGTTLHVYPDLYHSQFTYFQVLRVVRLIK |
| | | ISPALEDFVYKIFGPGKKLGSLVVFTASLLIVMSAISLQMFC |
| | | FVEELDRFTTFPRAFMSMFQILTQEGWVDVMDQTLNAVG |
| | | HMWAPVVAIYFILYHLFATLILLSLFVAVILDNLELDEDLK |
| | | KLKQLKQSEANADTKEKLPLRLRIFEKFPNRPQMVKISKLP |
| | | SDFTVPKIRESFMKQFIDRQQQDTCCLLRSLPTTSSSSCDHS |
| | | KRSAIEDNKYIDQKLRKSVFSIRARNLLEKETAVTKILRAC |
| | | TRQRMLSGSFEGQPAKERSILSVQHHIRQERRSLRHGSNSQ |
| | | RISRGKSLETLTQDHSNTVRYRNAQREDSEIKMIQEKKEQA |
| | | EMKRKVQEEELRENHPYFDKPLFIVGREHRFRNFCRVVVR |
| | | ARFNASKTDPVTGAVKNTKYHQLYDLLGLVTYLDWVMII |
| | | VTICSCISMMFESPFRRVMHAPTLQIAEYVFVIFMSIELNLKI |
| | | MADGLFFTPTAVIRDFGGVMDIFIYLVSLIFLCWMPQNVPA |
| | | ESGAQLLMVLRCLRPLRIFKLVPQMRKVVRELFSGFKEIFL |
| | | VSILLLTLMLVFASFGVQLFAGKLAKCNDPNIIRREDCNGIF |
| | | RINVSVSKNLNLKLRPGEKKPGFWVPRVWANPRNFNFDN |
| | | VGNAMLALFEVLSLKGWVEVRDVIIHRVGPIHGIYIHVFVF |
| | | LGCMIGLTLFVGVVIANFNENKGTALLTVDQRRWEDLKSR |
| | | LKIAQPLHLPPRPDNDGFRAKMYDITQHPFFKRTIALLVLA |
| | | QSVLLSVKWDVEDPVTVPLATMSVVFTFIFVLEVTMKIIA |
| | | MSPAGFWQSRRNRYDLLVTSLGVVWVVLHFALLNAYTY |
| | | MMGACVIVFRFFSICGKHVTLKMLLLTVVVSMYKSFFIIVG |
| | | MFLLLLCYAFAGVVLFGTVKYGENINRHANFSSAGKAITV |
| | | LFRIVTGEDWNKIMHDCMVQPPFCTPDEFTYWATDCGNY |
| | | AGALMYFCSFYVIIAYIMLNLLVAIIVLENENVATEESSEPL |
| | | SEDDFEMFYETWEKFDPDATQFIDYSRLSDFVDTLQEPLKI |
| | | AKPNKIKLITLDLPMVPGDKIHCLDILFALTKEVLGDSGEM |
| | | DALKQTMEEKFMAANPSKVSYEPITTTLKRKQEEVCAIKIQ |
| | | RAYRRHLLQRSVKQASYMYRHSQDGNGDGAPEKEGLLA |
| | | NTMNKMYGHEKEGDGVQSQGEEEKASTEDAGPTVEPKPT |
| | | SSSDTALTPSPPPLPPSSSPPQGQTVRPGVKESLV |
| 62 | hNALCN_R1573E/ | MLKRKQSSRVEAQPVTDFGPDESLSDNADILWINKPWVHS |
| | K1577E/K1578G | LLRICAIISVISVCMNTPMTFEHYPPLQYVTFTLDTLLMFLY |
| | | TAEMIAKMHIRGIVKGDSSYVKDRWCVFDGFMVFCLWVS |
| | | LVLQVFEIADIVDQMSPWGMLRIPRPLIMIRAFRIYFRFELP |
| | | RTRITNILKRSGEQIWSVSIFLLFFLLLYGILGVQMFGTFTY |
| | | HCVVNDTKPGNVTWNSLAIPDTHCSPELEEGYQCPPGFKC |
| | | MDLEDLGLSRQELGYSGFNEIGTSIFTVYEAASQEGWVFL |
| | | MYRAIDSFPRWRSYFYFITLIFFLAWLVKNVFIAVIIETFAEI |
| | | RVQFQQMWGSRSSTTSTATTQMFHEDAAGGWQLVAVDV |
| | | NKPQGRAPACLQKMMRSSVFHMFILSMVTVDVIVAASNY |
| | | YKGENFRRQYDEFYLAEVAFTVLFDLEALLKIWCLGFTGY |
| | | ISSSLHKFELLLVIGTTLHVYPDLYHSQFTYFQVLRVVRLIK |
| | | ISPALEDFVYKIFGPGKKLGSLVVFTASLLIVMSAISLQMFC |
| | | FVEELDRFTTFPRAFMSMFQILTQEGWVDVMDQTLNAVG |
| | | HMWAPVVAIYFILYHLFATLILLSLFVAVILDNLELDEDLK |
| | | KLKQLKQSEANADTKEKLPLRLRIFEKFPNRPQMVKISKLP |
| | | SDFTVPKIRESFMKQFIDRQQQDTCCLLRSLPTTSSSSCDHS |
| | | KRSAIEDNKYIDQKLRKSVFSIRARNLLEKETAVTKILRAC |
| | | TRQRMLSGSFEGQPAKERSILSVQHHIRQERRSLRHGSNSQ |
| | | RISRGKSLETLTQDHSNTVRYRNAQREDSEIKMIQEKKEQA |
| | | EMKRKVQEEELRENHPYFDKPLFIVGREHRFRNFCRVVVR |
| | | ARFNASKTDPVTGAVKNTKYHQLYDLLGLVTYLDWVMII |
| | | VTICSCISMMFESPFRRVMHAPTLQIAEYVFVIFMSIELNLKI |
| | | MADGLFFTPTAVIRDFGGVMDIFIYLVSLIFLCWMPQNVPA |
| | | ESGAQLLMVLRCLRPLRIFKLVPQMRKVVRELFSGFKEIFL |
| | | VSILLLTLMLVFASFGVQLFAGKLAKCNDPNIIRREDCNGIF |
| | | RINVSVSKNLNLKLRPGEKKPGFWVPRVWANPRNFNFDN |
| | | VGNAMLALFEVLSLKGWVEVRDVIIHRVGPIHGIYIHVFVF |
| | | LGCMIGLTLFVGVVIANFNENKGTALLTVDQRRWEDLKSR |
| | | LKIAQPLHLPPRPDNDGFRAKMYDITQHPFFKRTIALLVLA |
| | | QSVLLSVKWDVEDPVTVPLATMSVVFTFIFVLEVTMKIIA |
| | | MSPAGFWQSRRNRYDLLVTSLGVVWVVLHFALLNAYTY |
| | | MMGACVIVFRFFSICGKHVTLKMLLLTVVVSMYKSFFIIVG |
| | | MFLLLLCYAFAGVVLFGTVKYGENINRHANFSSAGKAITV |
| | | LFRIVTGEDWNKIMHDCMVQPPFCTPDEFTYWATDCGNY |
| | | AGALMYFCSFYVIIAYIMLNLLVAIIVENFSLFYSTEEDQLL |
| | | SYNDLRHFQIIWNMVDDKREGVIPTFRVKFLLRLLRGRLE |
| | | VDLDKDKLLFKHMCYEMERLHNGGDVTFHDVLSMLSYR |
| | | SVDIRKSLQLEELLAREQLEYTIEEEVAKQTIEMWLEGCLK |
| | | RIRAKQQQSCSIIHSLRESQQQELSRFLNPPSIETTQPSEDTN |
| | | ANSQDNSMQPETSSQQQLLSPTLSDRGGSRQDAADAGKPQ |
| | | RKFGQWRLPSAPKPISHSVSSVNLRFGGRTTMKSVVCKMN |
| | | PMTDAASCGSEVKKWWTRQLTVESDESGDDLLDI |
| 63 | hNALCN_W1575E/ | MLKRKQSSRVEAQPVTDFGPDESLSDNADILWINKPWVHS |
| | L1576E | LLRICAIISVISVCMNTPMTFEHYPPLQYVTFTLDTLLMFLY |
| | | TAEMIAKMHIRGIVKGDSSYVKDRWCVFDGFMVFCLWVS |
| | | LVLQVFEIADIVDQMSPWGMLRIPRPLIMIRAFRIYFRFELP |
| | | RTRITNILKRSGEQIWSVSIFLLFFLLLYGILGVQMFGTFTY |
| | | HCVVNDTKPGNVTWNSLAIPDTHCSPELEEGYQCPPGFKC |
| | | MDLEDLGLSRQELGYSGFNEIGTSIFTVYEAASQEGWVFL |
| | | MYRAIDSFPRWRSYFYFITLIFFLAWLVKNVFIAVIIETFAEI |
| | | RVQFQQMWGSRSSTTSTATTQMFHEDAAGGWQLVAVDV |
| | | NKPQGRAPACLQKMMRSSVFHMFILSMVTVDVIVAASNY |
| | | YKGENFRRQYDEFYLAEVAFTVLFDLEALLKIWCLGFTGY |
| | | ISSSLHKFELLLVIGTTLHVYPDLYHSQFTYFQVLRVVRLIK |
| | | ISPALEDFVYKIFGPGKKLGSLVVFTASLLIVMSAISLQMFC |
| | | FVEELDRFTTFPRAFMSMFQILTQEGWVDVMDQTLNAVG |
| | | HMWAPVVAIYFILYHLFATLILLSLFVAVILDNLELDEDLK |
| | | KLKQLKQSEANADTKEKLPLRLRIFEKFPNRPQMVKISKLP |
| | | SDFTVPKIRESFMKQFIDRQQQDTCCLLRSLPTTSSSSCDHS |
| | | KRSAIEDNKYIDQKLRKSVFSIRARNLLEKETAVTKILRAC |
| | | TRQRMLSGSFEGQPAKERSILSVQHHIRQERRSLRHGSNSQ |
| | | RISRGKSLETLTQDHSNTVRYRNAQREDSEIKMIQEKKEQA |
| | | EMKRKVQEEELRENHPYFDKPLFIVGREHRFRNFCRVVVR |
| | | ARFNASKTDPVTGAVKNTKYHQLYDLLGLVTYLDWVMII |
| | | VTICSCISMMFESPFRRVMHAPTLQIAEYVFVIFMSIELNLKI |
| | | MADGLFFTPTAVIRDFGGVMDIFIYLVSLIFLCWMPQNVPA |
| | | ESGAQLLMVLRCLRPLRIFKLVPQMRKVVRELFSGFKEIFL |
| | | VSILLLTLMLVFASFGVQLFAGKLAKCNDPNIIRREDCNGIF |
| | | RINVSVSKNLNLKLRPGEKKPGFWVPRVWANPRNFNFDN |
| | | VGNAMLALFEVLSLKGWVEVRDVIIHRVGPIHGIYIHVFVF |
| | | LGCMIGLTLFVGVVIANFNENKGTALLTVDQRRWEDLKSR |
| | | LKIAQPLHLPPRPDNDGFRAKMYDITQHPFFKRTIALLVLA |
| | | QSVLLSVKWDVEDPVTVPLATMSVVFTFIFVLEVTMKIIA |
| | | MSPAGFWQSRRNRYDLLVTSLGVVWVVLHFALLNAYTY |
| | | MMGACVIVFRFFSICGKHVTLKMLLLTVVVSMYKSFFIIVG |
| | | MFLLLLCYAFAGVVLFGTVKYGENINRHANFSSAGKAITV |
| | | LFRIVTGEDWNKIMHDCMVQPPFCTPDEFTYWATDCGNY |
| | | AGALMYFCSFYVIIAYIMLNLLVAIIVENFSLFYSTEEDQLL |
| | | SYNDLRHFQIIWNMVDDKREGVIPTFRVKFLLRLLRGRLE |
| | | VDLDKDKLLFKHMCYEMERLHNGGDVTFHDVLSMLSYR |
| | | SVDIRKSLQLEELLAREQLEYTIEEEVAKQTIRMEEKKCLK |
| | | RIRAKQQQSCSIIHSLRESQQQELSRFLNPPSIETTQPSEDTN |
| | | ANSQDNSMQPETSSQQQLLSPTLSDRGGSRQDAADAGKPQ |
| | | RKFGQWRLPSAPKPISHSVSSVNLRFGGRTTMKSVVCKMN |
| | | PMTDAASCGSEVKKWWTRQLTVESDESGDDLLDI |
| 64 | UNC79_frag1 | MSTKAEQFASKIRYLQEYHNRVLHNIYPVPSGTDIANTLKY |
| | | FSQTLLSILSRTGKKENQDASNLTVPMTMCLFPVPFPLTPSL |
| | | RPQVSSINPTVTRSLLYSVLRDAPSERGPQSRDAQLSDYPSL |
| | | DYQGLYVTLVTLLDLVPLLQHGQHDLGQSIFYTTTCLLPFL |
| | | NDDILSTLPYTMISTLATFPPFLHKDIIEYLSTSFLPMAILGSS |
| | | RREGVPAHVNLSASSMLMIAMQYTSNPVYHCQLLECLMK |
| | | YKQEVWKDLLYVIAYGPSQVKPPAVQMLFHYWPNLKPPG |
| | | AISEYRGLQYTAWNPIHCQHIECHNAINKPAVKMCIDPSLS |
| | | VALGDKPPPLYLCEECSERIAGDHSEWLIDVLLPQAEISAIC |
| | | QKKNCSSHVRRAVVTCFSAGCCGRHGNRPVRYCKRCHSN |
| | | HHSNEVGAAAETHLYQTSPPPINTRECGAEELVCAVEAVIS |
| | | LLKEAEFHAEQREHELNRRRQLGLSSSHHSLDNADFDNKD |
| | | DDKHDQRLL |
| 65 | UNC79_frag2 | MYKQEVWKDLLYVIAYGPSQVKPPAVQMLFHYWP |
| | | NLKPPGAISEYRGLQYTAWNPIHCQHIECHNAINKPAVKM |
| | | CIDPSLSVALGDKPPPLYLCEECSERIAGDHSEWLIDVLLPQ |
| | | AEISAICQKKNCSSHVRRAVVTCFSAGCCGRHGNRPVRYC |
| | | KRCHSNHHSNEVGAAAETHLYQTSPPPINTRECGAEELVC |
| | | AVEAVISLLKEAEFHAEQREHELNRRRQLGLSSSHHSLDN |
| | | ADFDNKDDDKHDQRLLSQFGIWFLVSLCTPSENTPTESLA |
| | | RLVAMVFQWFHSTAYMMDDEVGSLVEKLKPQFVTKWLK |
| | | TVCDVRFDVMVMCLLPKPMEFARVGGYWDKSCSTVTQL |
| | | KEGLNRILCLIPYNVINQSVWECIMPEWLEAIRTEVPDNQL |
| | | KEFREVLSKMFDIELCPLPFSMEEMFGFISCRFTGYPSSVQE |
| | | QALLWLHVLSELDIMVPLQLLISMFSDGVNSVKELANQRK |
| | | SRVSELAGNLASRRVSVASDPGRRVQH |
| 66 | UNC79_frag3 | MLSQFGIWFLVSLCTPSENTPTESLARLVAMVFQWFHSTA |
| | | YMMDDEVGSLVEKLKPQFVTKWLKTVCDVRFDVMVMCL |
| | | LPKPMEFARVGGYWDKSCSTVTQLKEGLNRILCLIPYNVIN |
| | | QSVWECIMPEWLEAIRTEVPDNQLKEFREVLSKMFDIELCP |
| | | LPFSMEEMFGFISCRFTGYPSSVQEQALLWLHVLSELDIMV |
| | | PLQLLISMFSDGVNSVKELANQRKSRVSELAGNLASRRVS |
| | | VASDPGRRVQHNMLSPFHSPFQSPFRSPLRSPFRSPFKNFG |
| | | HPGGRTIDFDCEDDEMNLNCFILMFDLLLKQMELQDDGIT |
| | | MGLEHSLSKDIISIINNVFQAPWGGSHTCQKDEKAIECNLC |
| | | QSSILCYQLACELLERLAPKEESRLVEPTDSLEDSLLSSRPE |
| | | FIIGPEGEEEENPASKHGENPGNCTEPVEHAAVKNDTERKF |
| | | CYQQLPVTLRLIYTIFQEMAKFEEPDILFNMLNCLKILCLHG |
| | | ECLYIARKDHPQFL |
| 67 | UNC79_frag4 | MHNMLSPFHSPFQSPFRSPLRSPFRSPFKNFGHPGGRTIDFD |
| | | CEDDEMNLNCFILMFDLLLKQMELQDDGITMGLEHSLSKD |
| | | IISIINNVFQAPWGGSHTCQKDEKAIECNLCQSSILCYQLAC |
| | | ELLERLAPKEESRLVEPTDSLEDSLLSSRPEFIIGPEGEEEEN |
| | | PASKHGENPGNCTEPVEHAAVKNDTERKFCYQQLPVTLRL |
| | | IYTIFQEMAKFEEPDILFNMLNCLKILCLHGECLYIARKDHP |
| | | QFLAYIQDHMLIASLWRVVKSEFSQLSSLAVPLLLHALSLP |
| | | HGADIFWTIINGNFNSKDWKMRFEAVEKVAVICRFLDIHS |
| | | VTKNHLLKYSLAHAFCCFLTAVEDVNPAVATRAGLLLDTI |
| | | KRPALQGLCLCLDFQFDTVVKDRPTILSKLLLLHFLKQDIP |
| | | ALSWEFFVNRFETLSLEAQLHLDCNKEFPFPTTITAVRTNV |
| | | ANLSDAALWKIKRARFARNRQKSVRSLRDSVKGPVESKR |
| | | ALSLPETLTSK |
| 68 | UNC79_frag5 | MLAYIQDHMLIASLWRVVKSEFSQLSSLAVPLLLHALSLPH |
| | | GADIFWTIINGNFNSKDWKMRFEAVEKVAVICRFLDIHSVT |
| | | KNHLLKYSLAHAFCCFLTAVEDVNPAVATRAGLLLDTIKR |
| | | PALQGLCLCLDFQFDTVVKDRPTILSKLLLLHFLKQDIPALS |
| | | WEFFVNRFETLSLEAQLHLDCNKEFPFPTTITAVRTNVANL |
| | | SDAALWKIKRARFARNRQKSVRSLRDSVKGPVESKRALSL |
| | | PETLTSKIRQQSPENDNTIKDLLPEDAGIDHQTVHQLITVLM |
| | | KFMAKDESSAESDISSAKAFNTVKRHLYVLLGYDQQEGCF |
| | | MIAPQKMRLSTCFNAFIAGIAQVMDYNINLGKHLLPLVVQ |
| | | VLKYCSCPQLRHYFQQPPRCSLWSLKPHIRQMWLKALLVI |
| | | LYKYPYRDCDISKILLHLIHITVNTLNAQYHSCKPHATAGP |
| | | LYSDNSNISRYSEKEKGEIELAEYRETGALQDSLLHCVREE |
| | | SIPKKKLRSFKQK |
| 69 | UNC79_frag6 | MKIRQQSPENDNTIKDLLPEDAGIDHQTVHQLITVLMKFM |
| | | AKDESSAESDISSAKAFNTVKRHLYVLLGYDQQEGCFMIA |
| | | PQKMRLSTCFNAFIAGIAQVMDYNINLGKHLLPLVVQVLK |
| | | YCSCPQLRHYFQQPPRCSLWSLKPHIRQMWLKALLVILYK |
| | | YPYRDCDISKILLHLIHITVNTLNAQYHSCKPHATAGPLYS |
| | | DNSNISRYSEKEKGEIELAEYRETGALQDSLLHCVREESIPK |
| | | KKLRSFKQKSLDIGNADSLLFTLDEHRRKSCIDRCDIEKPPT |
| | | QAAYIAQRPNDPGRSRQNSATRPDNSEIPENPAMEGFPDAR |
| | | RPVIPEVRLNCMETFEVKVDSPVKPAPKEDLDLIDLSSDSTS |
| | | GPEKHSILSTSDSDSLVFEPLPPLRIVESDEEEETMNQGDDG |
| | | PSGKNAASSPSVPSHPSVLSLSTAPLVQVSVEDCSKDFSSK |
| | | DSGNNQSAGNTDSALITLEDPMDAEGSSKPEELPEFSCGSP |
| | | LTLKQKRDLL |
| 70 | UNC79_frag7 | MKSLDIGNADSLLFTLDEHRRKSCIDRCDIEKPPTQAAYIA |
| | | QRPNDPGRSRQNSATRPDNSEIPENPAMEGFPDARRPVIPE |
| | | VRLNCMETFEVKVDSPVKPAPKEDLDLIDLSSDSTSGPEKH |
| | | SILSTSDSDSLVFEPLPPLRIVESDEEEETMNQGDDGPSGKN |
| | | AASSPSVPSHPSVLSLSTAPLVQVSVEDCSKDFSSKDSGNN |
| | | QSAGNTDSALITLEDPMDAEGSSKPEELPEFSCGSPLTLKQ |
| | | KRDLLQKSFALPEMSLDDHPDPGTEGEKPGELMPSSGAKT |
| | | VLLKVPEDAENPTESEKPDTSAESDTEQNPERKVEEDGAEE |
| | | SEFKIQIVPRQRKQRKIAVSAIQREYLDISFNILDKLGEQKD |
| | | PDPSTKGLSTLEMPRESSSAPTLDAGVPETSSHSSISTQYRQ |
| | | MKRGSLGVLTMSQLMKRQLEHQSSAPHNISNWDTEQIQP |
| | | GKRQCNVPTCLNPDLEGQPLRMRGATKSSLLSAPSIVSMF |
| | | VPAPEEFTDEQ |
| 71 | UNC79_frag8 | MLQKSFALPEMSLDDHPDPGTEGEKPGELMPSSGAKTVLL |
| | | KVPEDAENPTESEKPDTSAESDTEQNPERKVEEDGAEESEF |
| | | KIQIVPRQRKQRKIAVSAIQREYLDISFNILDKLGEQKDPDP |
| | | STKGLSTLEMPRESSSAPTLDAGVPETSSHSSISTQYRQMK |
| | | RGSLGVLTMSQLMKRQLEHQSSAPHNISNWDTEQIQPGKR |
| | | QCNVPTCLNPDLEGQPLRMRGATKSSLLSAPSIVSMFVPAP |
| | | EEFTDEQPTVMTDKCHDCGAILEEYDEETLGLAIVVLSTFI |
| | | HLSPDLAAPLLLDIMQSVGRLASSTTFSNQAESMMVPGNA |
| | | AGVAKQFLRCIFHQLAPNGIFPQLFQSTIKDGTFLRTLASSL |
| | | MDFNELSSIAALSQLLEGLNNKKNLPAGGAMIRCLENIATF |
| | | MEALPMDSPSSLWTTISNQFQTFFAKLPCVLPLKCSLDSSL |
| | | RIMICLLKIPSTNATRSLLEPFSKLLSFVIQNAVFTLAYLVEL |
| | | CGLCYRAFT |
| 72 | UNC79_frag9 | MQPTVMTDKCHDCGAILEEYDEETLGLAIVVLSTFIHLSPD |
| | | LAAPLLLDIMQSVGRLASSTTFSNQAESMMVPGNAAGVA |
| | | KQFLRCIFHQLAPNGIFPQLFQSTIKDGTFLRTLASSLMDFN |
| | | ELSSIAALSQLLEGLNNKKNLPAGGAMIRCLENIATFMEAL |
| | | PMDSPSSLWTTISNQFQTFFAKLPCVLPLKCSLDSSLRIMIC |
| | | LLKIPSTNATRSLLEPFSKLLSFVIQNAVFTLAYLVELCGLC |
| | | YRAFTKERDKFYLSRSVVLELLQALKLKSPLPDTNLLLLVQ |
| | | FICADAGTKLAESTILSKQMIASVPGCGTAAMECVRQYINE |
| | | VLDFMADMHTLTKLKSHMKTCSQPLHEDTFGGHLKVGLA |
| | | QIAAMDISRGNHRDNKAVIRYLPWLYHPPSAMQQGPKEFI |
| | | ECVSHIRLLSWLLLGSLTHNAVCPNASSPCLPIPLDAGSHV |
| | | ADHLIVILIGFPEQSKTSVLHMCSLFHAFIFAQLWTVYCEQS |
| | | AVATNLQNQNE |
| 73 | UNC79_frag10 | MTKERDKFYLSRSVVLELLQALKLKSPLPDTNLLLLVQFIC |
| | | ADAGTKLAESTILSKQMIASVPGCGTAAMECVRQYINEVL |
| | | DFMADMHTLTKLKSHMKTCSQPLHEDTFGGHLKVGLAQI |
| | | AAMDISRGNHRDNKAVIRYLPWLYHPPSAMQQGPKEFIEC |
| | | VSHIRLLSWLLLGSLTHNAVCPNASSPCLPIPLDAGSHVAD |
| | | HLIVILIGFPEQSKTSVLHMCSLFHAFIFAQLWTVYCEQSAV |
| | | ATNLQNQNEFSFTAILTALEFWSRVTPSILQLMAHNKVMV |
| | | EMVCLHVISLMEALQECNSTIFVKLIPMWLPMIQSNIKHLS |
| | | AGLQLRLQAIQNHVNHHSLRTLPGSGQSSAGLAALRKWL |
| | | QCTQFKMAQVEIQSSEAASQFYPL |
| 74 | UNC80_frag1 | MVKRKSSEGQEQDGGRGIPLPIQTFLWRQTSAFLRPKLGK |
| | | QYEASCVSFERVLVENKLHGLSPALSEAIQSISRWELVQAA |
| | | LPHVLHCTATLLSNRNKLGHQDKLGVAETKLLHTLHWML |
| | | LEAPQDCNNERFGGTDRGSSWGGSSSAFIHQVENQGSPGQ |
| | | PCQSSSNDEEENNRRKIFQNSMATVELFVFLFAPLVHRIKE |
| | | SDLTFRLASGLVIWQPMWEHRQPGVSGFTALVKPIRNIITA |
| | | KRSSPINSQSRTCESPNQDARHLEGLQVVCETFQSDSISPKA |
| | | TISGCHRGNSFDGSLSSQTSQERGPSHSRASLVIPPCQRSRY |
| | | ATYFDVAVLRCLLQPHWSEEGTQWSLMYYLQRLRHMLEE |
| | | KPEKPPEPDIPLLPRPRSSSMVAAAPSLVNTHKTQDLTMKC |
| | | NEEEKSLSSEAFSKVSLTNLRRSAVPDLSSDLGMNIFKKFK |
| | | SRKEDRERKGSIPFHHTGKRRPRRMGVPFLLHEDHLDVSPT |
| | | RSTFSFGSFSGL |
| 75 | UNC80_frag2 | MSQSRTCESPNQDARHLEGLQVVCETFQSDSISPKATISGC |
| | | HRGNSFDGSLSSQTSQERGPSHSRASLVIPPCQRSRYATYF |
| | | DVAVLRCLLQPHWSEEGTQWSLMYYLQRLRHMLEEKPEK |
| | | PPEPDIPLLPRPRSSSMVAAAPSLVNTHKTQDLTMKCNEEE |
| | | KSLSSEAFSKVSLTNLRRSAVPDLSSDLGMNIFKKFKSRKE |
| | | DRERKGSIPFHHTGKRRPRRMGVPFLLHEDHLDVSPTRSTF |
| | | SFGSFSGLGEDRRGIEKGGWQTTILGKLTRRGSSDAATEME |
| | | SLSARHSHSHHTLVSDLPDPSNSHGENTVKEVRSQISTITVA |
| | | TFNTTLASFNVGYADFFNEHMRKLCNQVPIPEMPHEPLAC |
| | | ANLPRSLTDSCINYSYLEDTEHIDGTNNFVHKNGMLDLSV |
| | | VLKAVYLVLNHDISSRICDVALNIVECLLQLGVVPCVEKN |
| | | RKKSENKENETLEKRPSEGAFQFKGVSGSSTCGFGGPAVS |
| | | GAGDGGGEEGGGGDG |
| 76 | UNC80_frag3 | MLGEDRRGIEKGGWQTTILGKLTRRGSSDAATEMESLSAR |
| | | HSHSHHTLVSDLPDPSNSHGENTVKEVRSQISTITVATFNTT |
| | | LASFNVGYADFFNEHMRKLCNQVPIPEMPHEPLACANLPR |
| | | SLTDSCINYSYLEDTEHIDGTNNFVHKNGMLDLSVVLKAV |
| | | YLVLNHDISSRICDVALNIVECLLQLGVVPCVEKNRKKSEN |
| | | KENETLEKRPSEGAFQFKGVSGSSTCGFGGPAVSGAGDGG |
| | | GEEGGGGDGGGGGGDGGGGGGGGGGPYEKNDKNQEKD |
| | | ESTPVSNHRLALTMLIKIVKSLGCAYGCGEGHRGLSGDRL |
| | | RHQVFRENAQNCLTKLYKLDKMQFRQTMRDYVNKDSLN |
| | | NVVDFLHALLGFCMEPVTDNKAGFGNNFTTVDNKSTAQN |
| | | VEGIIVSAMFKSLITRCASTTHELHSPENLGLYCDIRQLVQF |
| | | IKEAHGNVFRRVALSALLDSAEKLAPGKKVEENEQESKPA |
| | | GSKRSEAGSIVDKGQVSSAPEEC |
| 77 | UNC80_frag4 | MGGGGGGDGGGGGGGGGGPYEKNDKNQEKDESTPVSNH |
| | | RLALTMLIKIVKSLGCAYGCGEGHRGLSGDRLRHQVFREN |
| | | AQNCLTKLYKLDKMQFRQTMRDYVNKDSLNNVVDFLHA |
| | | LLGFCMEPVTDNKAGFGNNFTTVDNKSTAQNVEGIIVSAM |
| | | FKSLITRCASTTHELHSPENLGLYCDIRQLVQFIKEAHGNVF |
| | | RRVALSALLDSAEKLAPGKKVEENEQESKPAGSKRSEAGSI |
| | | VDKGQVSSAPEECRSFMSGRPSQTPEHDEQMQGANLGRK |
| | | DFWRKMFKSQSAASDTSSQSEQDTSECTTAHSGTTSDRRA |
| | | RSRSRRISLRKKLKLPIGKRNWLKRSSLSGLADGVEDLLDI |
| | | SSVDRLSFIRQSSKVKFTSAVKLSEGGPGSGMENGRDEEEN |
| | | FFKRLGCHSFDDHLSPNQDGGKSKNVVNLGAIRQGMKRF |
| | | QFLLNCCEPGTIPDASILAAALDLEAPVVARAALFLECARF |
| | | VHRCNRGNWPEWMKGHHVNITK |
| 78 | UNC80_frag5 | MCRSFMSGRPSQTPEHDEQMQGANLGRKDFWRKMFKSQ |
| | | SAASDTSSQSEQDTSECTTAHSGTTSDRRARSRSRRISLRK |
| | | KLKLPIGKRNWLKRSSLSGLADGVEDLLDISSVDRLSFIRQ |
| | | SSKVKFTSAVKLSEGGPGSGMENGRDEEENFFKRLGCHSF |
| | | DDHLSPNQDGGKSKNVVNLGAIRQGMKRFQFLLNCCEPG |
| | | TIPDASILAAALDLEAPVVARAALFLECARFVHRCNRGNW |
| | | PEWMKGHHVNITKKGLSRGRSPIVGNKRNQKLQWNAAKL |
| | | FYQWGDAIGVRLNELCHGESESPANLLGLIYDEETKRRLR |
| | | KEDEEEDFLDDSTVNPSKCGCPFALKMAACQLLLEITTFLR |
| | | ETFSCLPRPRTEPLVDLESCRLRLDPELDRHRYERKISFAGV |
| | | LDENEDSKDSLHSSSHTLKSDAGVEEKKEGSPWSASEPSIE |
| | | PEGMSNAGAEENYHRNMSWLHVMILLCNQQSFICTHVDY |
| | | CHPHCYLHHSRSCARLVRAIK |
| 79 | UNC80_frag6 | MKKGLSRGRSPIVGNKRNQKLQWNAAKLFYQWGDAIGV |
| | | RLNELCHGESESPANLLGLIYDEETKRRLRKEDEEEDFLDD |
| | | STVNPSKCGCPFALKMAACQLLLEITTFLRETFSCLPRPRTE |
| | | PLVDLESCRLRLDPELDRHRYERKISFAGVLDENEDSKDSL |
| | | HSSSHTLKSDAGVEEKKEGSPWSASEPSIEPEGMSNAGAEE |
| | | NYHRNMSWLHVMILLCNQQSFICTHVDYCHPHCYLHHSR |
| | | SCARLVRAIKLLYGDSVDSLRESSNISSVALRGKKQKECSD |
| | | KSCLRTPSLKKRVSDANLEGKKDSGMLKYIRLQVMSLSPA |
| | | PLSLLIKAAPILTEEMYGDIQPAAWELLLSMDEHMAGAAA |
| | | AMFLLCAVKVPEAVSDMLMSEFHHPETVQRLNAVLKFHT |
| | | LWRFRYQVWPRMEEGAQQIFKIPPPSINFTLPSPVLGMPSV |
| | | PMFDPPWVPQCSGSVQDPINEDQSKSFSARAVSRSHQRAE |
| | | HILKNLQQEEEKKRLGREA |
| 80 | UNC80_frag7 | MKLLYGDSVDSLRESSNISSVALRGKKQKECSDKSCLRTPS |
| | | LKKRVSDANLEGKKDSGMLKYIRLQVMSLSPAPLSLLIKA |
| | | APILTEEMYGDIQPAAWELLLSMDEHMAGAAAAMFLLCA |
| | | VKVPEAVSDMLMSEFHHPETVQRLNAVLKFHTLWRFRYQ |
| | | VWPRMEEGAQQIFKIPPPSINFTLPSPVLGMPSVPMFDPPW |
| | | VPQCSGSVQDPINEDQSKSFSARAVSRSHQRAEHILKNLQQ |
| | | EEEKKRLGREASLITAIPITQEACYEPTCTPNSEPEEEVEEVT |
| | | NLASRRLSVSPSCTSSTSHRNYSFRRGSVWSVRSAVSAEDE |
| | | EHTTEHTPNHHVPQPPQAVFPACICAAVLPIVHLMEDGEV |
| | | REDGVAVSAVAQQVLWNCLIEDPSTVLRHFLEKLTISNRQ |
| | | DELMYMLRKLLLNIGDFPAQTSHILFNYLVGLIMYFVRTPC |
| | | EWGMDAISATLTFLWEVVGYVEGLFFKDLKQTMKKEQCE |
| | | VKLLVTASMPGTKTLVV |
| 81 | UNC80_frag8 | MASLITAIPITQEACYEPTCTPNSEPEEEVEEVTNLASRRLS |
| | | VSPSCTSSTSHRNYSFRRGSVWSVRSAVSAEDEEHTTEHTP |
| | | NHHVPQPPQAVFPACICAAVLPIVHLMEDGEVREDGVAVS |
| | | AVAQQVLWNCLIEDPSTVLRHFLEKLTISNRQDELMYMLR |
| | | KLLLNIGDFPAQTSHILFNYLVGLIMYFVRTPCEWGMDAIS |
| | | ATLTFLWEVVGYVEGLFFKDLKQTMKKEQCEVKLLVTAS |
| | | MPGTKTLVVHGQNECDIPTQLPVHEDTQFEALLKECLEFF |
| | | NIPESQSTHYFLMDKRWNLIHYNKTYVRDIYPFRRSVSPQL |
| | | NLVHMHPEKGQELIQKQVFTRKLEEVGRVLFLISLTQKIPT |
| | | AHKQSHVSMLQEDLLRLPSFPRSAIDAEFSLFSDPQAGKEL |
| | | FGLDTLQKSLWIQLLEEMFLGMPSEFPWGDEIMLFLNVEN |
| | | GALILHPEDSALLRQYAATVINTAVHFNHLFSLSGYQWILP |
| | | TMLQVYSDYESNPQL |
| 82 | UNC80_frag9 | MVHGQNECDIPTQLPVHEDTQFEALLKECLEFFNIPESQST |
| | | HYFLMDKRWNLIHYNKTYVRDIYPFRRSVSPQLNLVHMH |
| | | PEKGQELIQKQVFTRKLEEVGRVLFLISLTQKIPTAHKQSH |
| | | VSMLQEDLLRLPSFPRSAIDAEFSLFSDPQAGKELFGLDTL |
| | | QKSLWIQLLEEMFLGMPSEFPWGDEIMLFLNVENGALILHP |
| | | EDSALLRQYAATVINTAVHFNHLFSLSGYQWILPTMLQVY |
| | | SDYESNPQLRQAIEFACHQFYILHRKPFVLQLFASVAPLLEF |
| | | PDAANNGPSKGVSAQCLFDLLQSLEGETTDILDILELVKAE |
| | | KPLKSLDFCYGNEDLTFSISEAIKLCVTVVAYAPESFRSLQ |
| | | MLMVLEALVPCYLQKLKRQTSQVETVPAAREEIAATAAL |
| | | ATSLQALLYSVEVLTRPMTAPQMSRCDQGHKGTTTANHT |
| | | MSSGVNTRYQEQGAKLHFIRENLHLLEEGQGIPREELDERI |
| | | AREEFRRPRESLLNIC |
| 83 | UNC80_frag10 | MLRQAIEFACHQFYILHRKPFVLQLFASVAPLLEFPDAANN |
| | | GPSKGVSAQCLFDLLQSLEGETTDILDILELVKAEKPLKSL |
| | | DFCYGNEDLTFSISEAIKLCVTVVAYAPESFRSLQMLMVLE |
| | | ALVPCYLQKLKRQTSQVETVPAAREEIAATAALATSLQAL |
| | | LYSVEVLTRPMTAPQMSRCDQGHKGTTTANHTMSSGVNT |
| | | RYQEQGAKLHFIRENLHLLEEGQGIPREELDERIAREEFRRP |
| | | RESLLNICTEFYKHCGPRLKILQNLAGEPRVIALELLDVKSH |
| | | MRLAEIAHSLLKLAPYDTQTMESRGLRRYIMEMLPITDWT |
| | | AEAVRPALILILKRLDRMFNKIHKMPTLRRQVEWEPASNLI |
| | | EGVCLTLQRQPIISFLPHLRSLINVCVNLVMGVVGPSSVAD |
| | | GLPLLHLSPYLSPPLPFSTAVVRLVALQIQALKEDFPLSHVI |
| | | SPFTNQERREGMLLNLLIPFVLTVGSGSKDSPWLEQPEVQL |
| | | LLQTVINVLLP |
| 84 | UNC80_frag11 | MCTEFYKHCGPRLKILQNLAGEPRVIALELLDVKSHMRLA |
| | | EIAHSLLKLAPYDTQTMESRGLRRYIMEMLPITDWTAEAV |
| | | RPALILILKRLDRMFNKIHKMPTLRRQVEWEPASNLIEGVC |
| | | LTLQRQPIISFLPHLRSLINVCVNLVMGVVGPSSVADGLPLL |
| | | HLSPYLSPPLPFSTAVVRLVALQIQALKEDFPLSHVISPFTN |
| | | QERREGMLLNLLIPFVLTVGSGSKDSPWLEQPEVQLLLQT |
| | | VINVLLPPRIISTSRSKNFMLESSPAHCSTPGDAGKDLRREG |
| | | LAESTSQAAYLALKVILVCFERQLGSQWYWLSLQVKEMA |
| | | LRKVGGLALWDFLDFIVRTRIPIFVLLRPFIQCKLLAQPAEN |
| | | HEELSARQHIADQLERRFIPRPLCKSSLIAEFNSELKILKEAV |
| | | HSGSAYQGKTSISTVGTSTSAYRLSLATMSRSNTGTGTVW |
| | | EQDSEPSQQASQDTLSRTDEEDEENDSISMPSVVSEQEAYL |
| | | LSAIGRRRFS |
| 85 | UNC80_frag12 | MPPRIISTSRSKNFMLESSPAHCSTPGDAGKDLRREGLAEST |
| | | SQAAYLALKVILVCFERQLGSQWYWLSLQVKEMALRKVG |
| | | GLALWDFLDFIVRTRIPIFVLLRPFIQCKLLAQPAENHEELS |
| | | ARQHIADQLERRFIPRPLCKSSLIAEFNSELKILKEAVHSGS |
| | | AYQGKTSISTVGTSTSAYRLSLATMSRSNTGTGTVWEQDS |
| | | EPSQQASQDTLSRTDEEDEENDSISMPSVVSEQEAYLLSAI |
| | | GRRRFSSHVSSMSVPQAEVGMLPSQSEPNVLDDSQGLAAE |
| | | GSLSRVASIQSEPGQQNLLVQQPLGRKRGLRQLRRPLLSRQ |
| | | KTQTEPRNRQGARLSTTRRSIQPKTKPSADQKRSVTFIEAQ |
| | | PEPAAAPTDALPATGQLQGCSPAPSRKPEAMDEPVLTSSPA |
| | | IVVADLHSVSPKQSENFPTEEGEKEEDTEAQGATAHSPLSA |
| | | QLSDPDDFTGLETSSLLQHGDTVLHISEENGMENPLLSSQF |
| | | TFTPTELGKTDAVLDESHV |
| 86 | GGGS peptide | GGGS |
| | element | |
| 87 | GGGS hairpin | GGGSGGGSGGGS |
| | element | |

Although the foregoing has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1. A method of identifying a modulator of a complex of human NALCN (Na+ leak channel non-selective), FAM155 (Family with sequence similarity 155 member A), UNC79 (uncoordinated 79), and UNC80 (uncoordinated 80) (a human NALCN-FAM155-UNC79-UNC80 complex), comprising:
a) providing a human NALCN-FAM155-UNC79-UNC80 complex in vitro;
b) contacting the complex with a potential modulator of the complex;
c) performing an ion channel assay of the complex in the presence of the potential modulator; and
d) identifying the potential modulator as a modulator of the complex if the activity of the complex in the assay in the presence of the potential modulator is higher or lower than the activity of the complex in the assay in the absence of the potential modulator.

2. (canceled)

3. (canceled)

4. (canceled)

5. (canceled)

6. The method of claim 1, further comprising determining the binding affinity of the potential modulator identified in part (d) to the human NALCN-FAM155-UNC79-UNC80 complex, a human NALCN-FAM155 complex, a UNC79-UNC80 complex, or to one or more of human NALCN, FAM155, UNC79, or UNC80.

7. (canceled)

8. The method of claim 1, wherein the method is performed in the presence of an NALCN-DII-DIII linker peptide or an NALCN-DI-DII linker peptide or an identified modulator of the human NALCN-FAM155-UNC79-UNC80 complex.

9. The method of claim 8, wherein the linker peptide comprises an amino acid sequence of any one of SEQ ID Nos: 16-24, or comprises an amino acid sequence of any one of SEQ ID Nos: 16-18 or 23-24.

10. The method of claim 1, wherein the method further comprises determining whether the potential modulator modulates activity of a human NALCN-FAM155-UNC79-UNC80 complex, wherein the complex comprises a mutant human NALCN or a mutant human UNC79 or a mutant human UNC80.

11. The method of claim 10, wherein the mutant human NALCN comprises a substitution, insertion, or deletion in one or both of the DI-DII linker or the DII-DIII linker.

12. The method of claim 11, wherein;
the DI-DII linker or the DII-DIII linker of the mutant human NALCN is replaced by a corresponding region from a human or mammalian Nav or Cav protein, such as from human Nav1.4; or
the DI-DII linker or the DII-DIII linker comprise a deletion; or
the DI-DII linker or the DII-DIII linker comprise an insertion of a GGGS element.

13. The method of claim 10, wherein:
the mutant human NALCN comprises an amino acid sequence of any one of SEQ ID Nos: 7, 8, 12-15, 32-52, or 59-63; or
the method further comprises determining whether the potential modulator modulates activity of a human NALCN-FAM155-UNC79-UNC80 complex, wherein the complex comprises a mutant human UNC79 comprising an amino acid sequence of any one of SEQ ID Nos: 53, 54, 55, 58, or 64-73; or
the method further comprises determining whether the potential modulator modulates activity of a human NALCN-FAM155-UNC79-UNC80 complex, wherein the complex comprises a mutant human UNC80 comprising an amino acid sequence of any one of SEQ ID Nos: 74-85.

14. (canceled)

15. (canceled)

16. A method of identifying a molecule that binds to a complex of human NALCN (Na+ leak channel non-selective), human FAM155 (Family with sequence similarity 155 member A), human UNC79 (uncoordinated 79), and human UNC80 (uncoordinated 80): (a human NALCN-FAM155-UNC79-UNC80 complex), the method comprising:
a) providing a human NALCN-FAM155-UNC79-UNC80 complex in vitro;
b) contacting the complex with one or more test molecules, and separating bound from unbound test molecules; and
c) identifying a test molecule as binding to the human NALCN-FAM155-UNC79-UNC80 complex if the test molecule remains bound to the complex after separating bound from unbound test molecules.

17. A method of identifying a molecule that binds to a complex of human NALCN (Na+ leak channel non-selective), human FAM155 (Family with sequence similarity 155 member A), human UNC79 (uncoordinated 79), and human UNC80 (uncoordinated 80): (a human NALCN-FAM155-UNC79-UNC80 complex), the method comprising:
a) providing a human NALCN-FAM155-UNC79-UNC80 complex in vitro;
b) contacting the complex with an NALCN-DII-DIII linker peptide or an NALCN-DI-DII linker peptide, and further with a test molecule; and
c) identifying the test molecule as binding to the human NALCN-FAM155-UNC79-UNC80 complex if the test molecule competes with the NALCN-DII-DIII linker peptide and/or the NALCN-DI-DII linker peptide for binding to the complex.

18. A method of identifying a molecule that modulates formation of or stability of a complex of human NALCN (Na+ leak channel non-selective), human FAM155 (Family with sequence similarity 155 member A), human UNC79 (uncoordinated 79), and human UNC80 (uncoordinated 80): (a human NALCN-FAM155-UNC79-UNC80 complex) in vitro, the method comprising:
a) providing human NALCN, human FAM155, human UNC79, and human UNC80 under conditions in which human NALCN-FAM155-UNC79-UNC80 complex formation is promoted in vitro;
b) contacting the complex with a test molecule; and
c) identifying the test molecule as modulating formation of or stability of the human NALCN-FAM155-UNC79-UNC80 complex if the degree of complex formation in the presence of the test molecule is increased or reduced compared to in the absence of the test molecule.

19. (canceled)

20. The method of claim 16, wherein the method further comprises determining binding affinity of the test molecule to the complex is-determined by an ELISA, AlphaLISA, or FRET assay.

21. (canceled)

22. The method of claim 1, wherein:
at least one of the human NALCN, human FAM155, human UNC79, or human UNC80 of the human NALCN-FAM155-UNC79-UNC80 complex is labeled and/or is attached to a matrix, such as a bead, chip, or plate; or
the human NALCN-FAM155-UNC79-UNC80 complex is solubilized in a lipid bilayer, detergent, or lipid nanodisc.

23. (canceled)

24. The method of claim 16, wherein the method further comprises determining whether the molecule binds to a human NALCN-FAM155 complex, a human UNC79-UNC80 complex, or to one or more of human NALCN, FAM155, UNC79, or UNC80.

25. The method of claim 16, wherein the method further comprises determining whether the test molecule binds to a human NALCN-FAM155-UNC79-UNC80 complex wherein the complex comprises a mutant human NALCN.

26. The method of claim 25, wherein;
the mutant human NALCN comprises a substitution, insertion, or deletion in one or both of the DI-DII linker or the DII-DIII linker; or
the DI-DII linker or the DII-DIII linker is replaced by a corresponding loop from a human or mammalian Nay or Cay protein; or
the DI-DII linker or the DII-DIII linker comprises a deletion; or
the DI-DII linker or the DII-DIII linker comprises an insertion of a GGGS element; or
the mutant human NALCN comprises an amino acid sequence of any one of SEQ ID Nos: 7, 8, 12-15, 32-52, or 59-63.

27. (canceled)

28. (canceled)

29. The method of claim 1, wherein the human NALCN-FAM155-UNC79-UNC80 complex further comprises human calmodulin.

30. (canceled)

31. (canceled)

32. (canceled)

33. (canceled)

34. (canceled)

35. (canceled)

36. (canceled)

37. (canceled)

38. A kit for performing the method of claim 1, comprising at least one of: an identified modulator of the human NALCN-FAM155-UNC79-UNC80 complex, one or more reagents for conducting an ion channel assay, labeled NALCN, UNC79, UNC80, or FAM155 and instructions for use.

39. An isolated complex of human NALCN, UNC79, UNC80, and FAM155.

40. (canceled)

41. The kit of claim 38, wherein the labeled NALCN, UNC79, UNC80, or FAM155 is attached to a matrix selected from a bead, a chip, and a plate.

42. The isolated complex of claim 39, wherein the complex is comprised within a lipid bilayer or lipid nanodisc.

43. The isolated complex of claim 39, wherein:
(i) at least one of the human NALCN, UNC79, UNC80, and FAM155 includes a label, or
(ii) at least one of the human NALCN, UNC79, UNC80, and FAM155 is attached to a matrix selected from a bead, a chip, and a plate, or
(iii) at least one of the human NALCN, UNC79, UNC80, and FAM155 includes a label and is attached to a matrix selected from a bead, a chip, and a plate.
